(19) United States
(12) Patent Application Publication
(10) Pub. No.: US 2010/0093738 A1
(43) Pub. Date: Apr. 15, 2010

(54) Fungicidal Compounds and Fungicidal Compositions

(75) Inventors: Bernd Müller, Frankenthal (DE); Jens Renner, Bad Durkheim (DE); Egon Haden, Kleinniedesheim (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/444,371

(22) PCT Filed: Oct. 8, 2006
(86) PCT No.: PCT/EP07/60649

§ 371 Date: Apr. 3, 2009

(30) Foreign Application Priority Data
Oct. 6, 2006 (EP) 06121914.3
Oct. 13, 2006 (EP) 06122244.4

(51) Int. Cl. A01N 43/90; C07D 471/04; A01P 3/00; A61K 31/53; A61K 31/4985; A61K 31/5025; A61P 35/00; A61K 31/519

(52) U.S. Cl. 514/243; 546/122; 544/236; 544/279; 544/184; 544/350; 514/249; 514/248; 514/264.1

(57) ABSTRACT

The present invention relates to compounds of the formula I

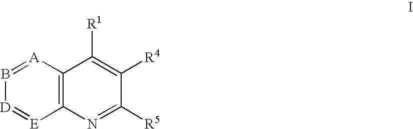

and their use for controlling phytopathogenic fungi, and also to fungicidal mixtures comprising a compound of the formula I, where the variables and substituents have the meanings defined in the claims and in the description.

The present invention relates to compounds of the formula I

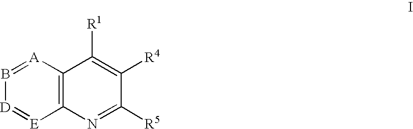

in which the substituents are as defined below:
R¹ is C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₂-cycloalkyl, C₃-C₁₂-halocycloalkyl, C₃-C₁₀-cycloalkenyl, C₃-C₁₀-halocycloalkenyl, phenyl, halophenyl, naphthyl, halonaphthyl or a five-, six-, seven-, eight-, nine- or ten-membered saturated, partially unsaturated or aromatic heterocycle which is attached via carbon and which contains one, two, three or four heteroatoms from the group consisting of oxygen, nitrogen and sulfur,
   where R¹ may carry one, two, three or four identical or different groups R^{a}, where R^{a} is as defined below:
   R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₂-C₆-alkenyloxy, C₃-C₆-alkynyloxy, C₁-C₈-alkoximino, C₂-C₁₀-alkenyloximino, C₂-C₁₀-alkynyloximino, C₆-C₁₄-aryl-C₁-C₈-alkyloximino, C₂-C₁₀-alkynyl, C₂-C₁₀-alkenyloxycarbonyl, C₂-C₁₀-alkynyloxycarbonyl, tri-C₁-C₆-alkylsilyl, phenyl, naphthyl, a five-, six-, seven-, eight-, nine- or ten-membered saturated, partially unsaturated or aromatic heterocycle which contains one, two, three or four heteroatoms from the group consisting of oxygen, nitrogen and sulfur;
   where the aliphatic, alicyclic and aromatic groups in R^{a} for their part may be partially or fully halogenated or may carry one, two or three groups R^{b}, where R^{b} is as defined below:
   R^{b} is halogen, cyano, nitro, hydroxyl, mercapto, amino, carboxyl, aminocarbonyl, aminothiocarbonyl, C₁-C₆-alkyl, C₁-C₆-haloalkyl, C₂-C₈-alkenyl, C₂-C₈-haloalkenyl, C₂-C₈-alkenyloxy, C₂-C₈-alkynyloxy, C₁-C₆-alkoxy, C₁-C₆-haloalkoxy, C₁-C₆-alkylthio, C₁-C₆-alkylamino, di-C₁-C₆-alkylamino, formyl, C₁-C₆-alkylcarbonyl, C₁-C₆-alkylsulfonyl, C₁-C₆-alkylsulfoxyl, C₁-C₆-alkoxycarbonyl, C₁-C₆-alkylcarbonyloxy, C₁-C₆-alkylaminocarbonyl, di-C₁-C₆-alkylaminocarbonyl, C₁-C₆-alkylaminothiocarbonyl, di-C₁-C₆-alkylaminocarbonyl, C₃-C₁₀-cyclo-alkyl, C₃-C₁₀-cycloalkoxy, saturated or partially unsaturated 5- or 6-membered heterocyclyl, saturated or unsaturated 5- or 6-membered heterocyclyloxy, 5- or 6-membered heteroaryl, 5- or 6-membered heteroaryloxy, 5- or 6-membered heteroarylthio, 5- or 6-membered heteroarylthio, phenyl, naphthyl, phenoxy, naphthoxy, phenylthio, naphthylthio, phenyl-C₁-C₆-alkoxy, phenyl-C₁-C₆-alkyl, where the cyclic systems may be partially or fully halogenated and/or substituted by alkyl and/or haloalkyl groups;
R⁴ is halogen, C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₁-C₈-alkoxy, C₁-C₈-haloalkoxy, C₃-C₈-cycloalkyl, C₃-C₈-halocycloalkyl, C₃-C₈-cycloalkyl-C₁-C₈-alkyl, C₁-C₈-alkylthio, C₆-C₁₄-aryl, C₆-C₁₄-aryloxy, C₆-C₁₄-arylthio, C₆-C₁₄-aryl-C₁-C₈-alkyl, C₆-C₁₄-aryl-C₁-C₈-alkoxy, C₆-C₁₄-aryl-C₁-C₈-alkylthio, 5- or 6-membered heteroaryl, heteroaryloxy, heteroarylthio, heteroaryl-C₁-C₈-alkyl, heteroaryl-C₁-C₈-alkoxy or heteroaryl-C₁-C₈-alkylthio, morpholino, piperidino or pyrrolidino;
   where R⁴ may carry one, two, three, four or five identical or different groups R^{c}, where R^{c} is as defined below:
   R^{c} is halogen, cyano, isocyano, hydroxyl, cyanato (OCN), thiocyanato, isothiocyanato, mercapto, C₁-C₈-alkyl, C₁-C₈-haloalkyl, C₁-C₈-hydroxyalkyl, C₁-C₈-alkoxy-C₁-C₈-alkyl, C₃-C₆-cycloalkyl-C₁-C₈-alkyl, C₂-C₁₀-alkenyl, C₂-C₁₀-haloalkenyl, C₂-C₁₀-alkynyl, C₂-C₁₀-haloalkynyl, C₁-C₆-alkoxy, C₁-C₆-haloalkoxy, C₂-C₁₀-alkenyloxy, C₂-C₁₀-haloalkenyloxy, C₂-C₁₀-alkynyloxy, C₂-C₁₀-haloalkynyloxy, C₃-C₆-cycloalkyl, C₃-C₆-halocycloalkyl, C₃-C₆-cyclo-alkenyl, C₃-C₆-halocycloalkenyl, C₃-C₆-cycloalkoxy, C₃-C₆-halocycloalkoxy, C₁-C₈-alkoxycarbonyl, C₁-C₈-haloalkoxycarbonyl, C₂-C₁₀-alkenyloxycarbonyl, C₂-C₁₀-haloalkenyloxycarbonyl, C₂-C₁₀-alkynyloxycarbonyl, C₂-C₁₀-haloalkynyloxycarbonyl, aminocarbonyl, C₁-C₈-alkylaminocarbonyl, di-C₁-C₈-alkylaminocarbonyl, hydroxyimino-C₁-C₈-alkyl, C₁-C₈-alkoximino-C₁-C₈-alkyl, C₂-C₁₀-alkenyloximinocarbonyl, C₂-C₁₀-alkenyloximo-C₁-C₈-alkyl, C₂-C₁₀-alkynyloximino-C₁-C₈-alkyl, C₂-C₁₀-alkenylcarbonyl, C₂-C₁₀-alkenylthiocarbonyl, C₂-C₁₀-alkynylcarbonyl, C₂-C₁₀-alkynylthiocarbonyl, tri-C₁-C₈-alkylsilyl, C₁-C₈-alkylthio, C₁-C₈-haloalkylthio, C₁-C₈-alkylsulfinyl, C₁-C₈-haloalkylsulfinyl, carboxyl-C₁-C₈-alkyl, phenyl, naphthyl, a five-, six-, seven-, eight-, nine- or ten-membered saturated, partially unsaturated or aromatic heterocycle which contains one, two, three or four heteroatoms from the group consisting of oxygen, nitrogen and sulfur, NR^{A}R^{B}, NHCOR^{A}, NHCONR^{A}R^{B}, CONR^{A}R^{B}, —OSO₂R^{A}, —COR^{A}, —CSR^{A}, —CR^{A}═NR^{B} or N═CR^{A}R^{B}, where R^{A} and R^{B} are as defined below:
      R^{A}, R^{B} independently of one another are hydrogen, C₁-C₈-alkyl, C₁-C₈-haloalkyl, C₁-C₈-alkoxy, C₁-C₈-haloalkoxy, C₁-C₈-alkylthio, C₃-C₈-cycloalkyl, C₃-C₈-halocycloalkyl, C₃-C₈-cycloalkyl-C₁-C₈-alkyl, phenyl or benzyl, where the phenyl and benzyl groups may be substituted by one, two, three, four or five groups independently selected from the group consisting of halogen, C₁-C₈-alkyl and C₁-C₈-alkoxy; phenoxy, benzyloxy, benzoyloxy, a five-, six-, seven-, eight-, nine- or ten-membered saturated, partially unsaturated or aromatic heterocycle which contains one, two, three or four heteroatoms from the group consisting of oxygen, nitrogen and sulfur;
   where R^{c} may carry one, two or three identical or different groups R^{cL}, where R^{cL} is as defined below:
   R^{cL} is halogen, cyano, nitro, hydroxyl, amino, carboxyl, C₁-C₆-alkyl, C₁-C₆-halo-alkyl, C₂-C₆-alkenyl, C₂-C₆-haloalkenyl, C₁-C₈-alkoxy, C₁-C₈-haloalkoxy, C₂-C₈-alkenyloxy, C₂-C₈-alkynyloxy, C₃-C₈-cycloalkyl, C₃-C₈-halocycloalkyl, C₃-C₈-cyclo-alkenyl, C₃-C₈-cycloalkyloxy, C₃-C₈-cycloalkenyloxy, C₁-C₈-alkoximino, C₂-C₈-alkenyloxyimino, C₂-C₈-alkynyloxyimino, C₁-C₈-alkoximino-C₁-C₈-alkyl, C₂-C₈-alkenyloximino-C₁-C₈-alkyl, C₂-C₈-alkynyloximino-C₁-C₈-alkyl, C₁-C₆-alkylcarbonyl, C₁-C₆-haloalkoxy, C₁-C₆-alkoxycarbonyl, C₁-C₆-alkylthio, C₁-C₆-alkylamino, di-C₁-C₆-alkylamino, C₆-C₁₄-aryl-C₁-C₈-alkyloximino, C₂-C₁₀-alkynyl, C₂-C₁₀-alkenyloxycarbonyl, C₂-C₁₀-alkynyloxycarbonyl, tri-C₁-C₆-alkylsilyl, phenyl, naphthyl, a five-, six-, seven-, eight-, nine- or ten-membered saturated, partially unsaturated or aromatic heterocycle which contains one, two, three or four heteroatoms from the group consisting of oxygen, nitrogen and sulfur;
R⁵ is halogen, cyano, C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₁-C₈-alkoxy, C₁-C₈-haloalkoxy;
   where R⁵ may carry one, two or three identical or different groups independently selected from the group consisting of cyano, nitro, C₁-C₂-alkoxy and C₁-C₄-alkoxycarbonyl;
A, B, D, E are each N or C—R⁶, where at least one and at most three variables are N and compounds in which simultaneously A, B and D are N and E is C—R⁶ and compounds in which simultaneously B, D and E are N and E is C—R⁶ are excluded; where R⁶ is as defined below:
   R⁶ is hydrogen, halogen, C₁-C₈-alkyl, C₁-C₈-alkoxy, C₁-C₈-haloalkyl, C₁-C₈-haloalkoxy;
      and to the agriculturally acceptable salts thereof. The invention furthermore relates to the use of these compounds as fungicides, the compositions comprising them, the methods for controlling harmful fungi using the compounds I, and also to processes and intermediates for their preparation.

The invention furthermore relates to fungicidal compositions comprising, as active components, fungicidal compositions for controlling phytopathogenic harmful fungi, which compositions comprise

1) at least one compound of the formula I

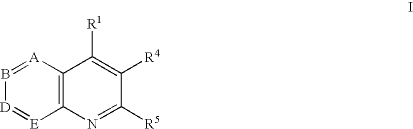

in which the substituents are as defined below:
R¹ is halogen, cyano, C₁-C₁₀-alkyl, C₁-C₁₀-haloalkyl, C₂-C₁₀-alkenyl, C₂-C₁₀-halo-alkenyl, C₂-C₁₀-alkynyl, C₃-C₁₀-haloalkynyl, C₃-C₁₂-cycloalkyl, C₃-C₁₂-halocycloalkyl, C₃-C₁₀-cycloalkenyl, C₃-C₁₀-halocycloalkenyl, phenyl, halophenyl, naphthyl, halonaphthyl, a five-, six-, seven-, eight-, nine- or ten-membered saturated, partially unsaturated or aromatic heterocycle which is attached via carbon and which contains one, two, three or four heteroatoms from the group consisting of oxygen, nitrogen and sulfur, C₁-C₈-alkoxy, C₁-C₈-haloalkoxy, C₁-C₈-alkylthio; where this R¹ may carry one, two, three or four identical or different groups R^{a}, where R^{a} is as defined in claim 1; or R¹ is NR²R³, where R² and R³ are as defined below:
   R² is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₈-halocycloalkoxy, C₅-C₁₀-bicycloalkyl, C₃-C₈-cycloalkyl-C₁-C₈-alkyl, C₆-C₁₄-aryl, C₆-C₁₄-aryl-C₁-C₈-alkyl, amino, C₁-C₈-alkylamino, di-C₁-C₈-alkylamino, five- or six-membered heteroaryl which contains one, two, three or four heteroatoms from the group consisting of O, N and S, five- or six-membered heteroaryl-C₁-C₈-alkyl, where the heteroaryl contains one, two, three or four heteroatoms from the group consisting of O, N and S;
   R³ is hydrogen, NR⁷R⁸ or one of the groups mentioned for R²; where R⁷ and R⁸ are as defined below:
      R⁷, R⁸ independently of one another are hydrogen, C₁-C₈-alkyl, C₁-C₈-haloalkyl, C₂-C₈-alkenyl, C₂-C₈-haloalkenyl, C₂-C₈-alkynyl, C₃-C₈-halo-alkynyl, C₃-C₈-cycloalkyl, C₃-C₈-halocycloalkyl, C₃-C₈-cycloalkyl-C₁-C₈-alkyl, C₆-C₁₄-aryl, C₆-C₁₄-aryl-C₁-C₈-alkyl, five- or six-membered heteroaryl or five- or six-membered heteroaryl-C₁-C₈-alkyl;
   where the alkyl, alkenyl, alkynyl and cycloalkyl groups in R² and/or R³ may carry one, two or three identical or different groups R^{d}, where R^{d} is as defined below:
   R^{d} is halogen, cyano, hydroxyl, C₁-C₈-alkyl, C₁-C₈-haloalkyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkoxy, C₃-C₈-halocycloalkoxy, C₃-C₈-cyclo-alkenyloxy, C₁-C₈-alkylcarbonyl, C₁-C₈-alkoxycarbonyl, C₁-C₈-alkylcarbonyloxy, C₁-C₈-alkoxycarbonyloxy, aminocarbonyloxy, C₁-C₈-alkylaminocarbonyloxy, di-C₁-C₈-alkylaminocarbonyloxy, C₁-C₈-alkylthiocarbonyloxy, C₁-C₈-alkoxy-thiocarbonyloxy, aminothiocarbonyloxy, C₁-C₈-alkylaminothiocarbonyloxy, di-C₁-C₈-alkylaminothiocarbonyloxy, C₁-C₈-alkylaminothiocarbonyl, di-C₁-C₈-alkylaminothiocarbonyl, C₁-C₈-alkylthio, tri-C₁-C₆-alkylsilyl, C₁-C₈-alkylamino, di-C₁-C₈-alkylamino;
   where the alkyl, alkenyl, alkynyl and cycloalkyl groups in R^{d} for their part may carry one, two or three identical or different groups R^{dL}, where R^{dL} has the meanings given for R^{cL};
   and where the aryl and heteroaryl groups in R² and/or R³ for their part may carry one, two or three identical or different groups R^{e}, where R^{e} is as defined below:
   R^{e} is halogen, hydroxyl, cyano, isocyano, thiocyanato, isothiocyanato, nitro, mercapto, C₁-C₈-alkyl, C₁-C₈-haloalkyl, C₁-C₈-hydroxyalkyl, C₁-C₈-alkoxy-C₁-C₈-alkyl, C₂-C₈-alkenyl, C₂-C₈-haloalkenyl, C₂-C₈-alkynyl, C₃-C₈-haloalkynyl, C₁-C₈-alkoxy, C₁-C₈-haloalkoxy, C₂-C₈-alkenyloxy, C₂-C₈-halo-alkenyloxy, C₂-C₈-alkynyloxy, C₃-C₈-haloalkynyloxy, C₃-C₈-cycloalkyl, C₃-C₈-halocycloalkyl, C₃-C₈-cycloalkyl-C₁-C₈-alkyl, C₁-C₈-alkylthio, C₁-C₈-haloalkylthio, phenoxy, benzyloxy, benzoyloxy, NR^{A}R^{B}, NHCOR^{A}, NHCONR^{A}R^{B}, CONR^{A}R^{B}, SO₂R^{A}, —OSO₂R^{A}, —COR^{A}, —CR^{A}═NR^{B} or N═CR^{A}R^{B}, where R^{A} and R^{B} are as defined in claim 1;
   R² and R³ together with the nitrogen atom to which they are attached may also form a five- or six-membered saturated, partially unsaturated or aromatic heterocycle which may contain one, two or three further heteroatoms from the group consisting of O, N and S as ring members, where the heterocycle may carry one, two or three identical or different groups R^{f}, where R^{f} has the meanings given for R^{d};
R⁴ is halogen, C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₁-C₈-alkoxy, C₁-C₈-haloalkoxy, C₃-C₈-cycloalkyl, C₃-C₈-halocycloalkyl, C₃-C₈-cycloalkyl-C₁-C₈-alkyl, C₁-C₈-alkylthio, C₆-C₁₄-aryl, C₆-C₁₄-aryloxy, C₆-C₁₄-arylthio, C₆-C₁₄-aryl-C₁-C₈-alkyl, C₆-C₁₄-aryl-C₁-C₈-alkoxy, C₆-C₁₄-aryl-C₁-C₈-alkylthio, 5- or 6-membered heteroaryl, heteroaryloxy, heteroarylthio, heteroaryl-C₁-C₈-alkyl, heteroaryl-C₁-C₈-alkoxy or heteroaryl-C₁-C₈-alkylthio, morpholino, piperidino, pyrrolidino;
   where R⁴ may carry one, two, three, four or five identical or different groups R^{c}, where R^{c} is as defined in claim 1;
R⁵ is halogen, cyano, C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₁-C₈-alkoxy, C₁-C₈-haloalkoxy or C₁-C₈-alkylthio;
   where R⁵ may carry one, two or three identical or different groups independently of one another selected from the group consisting of halogen, cyano, nitro, C₁-C₂-alkoxy and C₁-C₄-alkoxycarbonyl;
A, B, D, E are each N or C—R⁶, where at least one and at most three variables are N and compounds in which A, B and D are N and E is C—R⁶ and compounds in which B, D and E are N and E is C—R⁶ are excluded; where R⁶ is as defined below:
   R⁶ is hydrogen, halogen, C₁-C₈-alkyl, C₁-C₈-alkoxy, C₁-C₈-haloalkyl, C₁-C₈-haloalkoxy;
      or an agriculturally acceptable salt thereof; and
      2) at least one active compound II selected from the groups A) to F):
   A) azoles selected from the group consisting of bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thiabendazole, fuberidazole, ethaboxam, etridiazole and hymexazole;
   B) strobilurins selected from the group consisting of azoxystrobin, dimoxystrobin, enestroburin, fluoxastrobin, kresoxim-methyl, methominostrobin, orysastrobin, picoxystrobin, pyraclostrobin, trifloxystrobin, enestroburin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate and methyl 2-(ortho-(2,5-dimethylphenyloxymethylene)phenyl)-3-methoxyacrylate;
   C) carboxamides selected from the group consisting of carboxin, benalaxyl, benodanil, boscalid, fenfuram, fenhexamid, flutolanil, furametpyr, mepronil, metalaxyl, mefenoxam, ofurace, oxadixyl, oxycarboxin, penthiopyrad, thifluzamide, tiadinil, 3,4-dichloro-N-(2-cyanophenyl)isothiazole-5-carboxamide, dimethomorph, flumorph, flumetover, fluopicolide (picobenzamid), zoxamide, carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide, methyl 3-(4-chlorophenyl)-3-(2-isopropoxycarbonylamino-3-methylbutyrylamino)propionate, compounds of the formula III

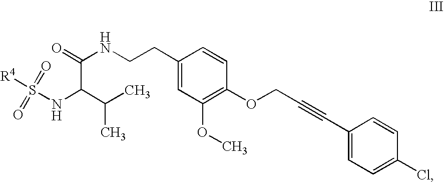

in which R⁴ is methyl or ethyl,
N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(3′,4′-dichloro-5-fluorobiphenyl-2-yl)-3-difluoromethyl-1-methylpyrazole-4-carboxamide, N-(2-cyanophenyl)-3,4-dichloroisothiazole-5-carboxamide and N-(2-bicyclopropyl-2-ylphenyl)-3-difluoromethyl-1-methyl-1H-pyrazole-4-carboxamide;
D) heterocyclic compounds selected from the group consisting of fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, probenazole, 5-chloro-7-(4-methylpiperidin-1-yl)-6-(2,4,6-trifluorophenyl)-[1,2,4]triazolo[1,5-a]pyrimidine, anilazine, diclomezine, pyroquilon, proquinazid, tricyclazole,
   the compound of the formula IV (2-butoxy-6-iodo-3-propylchromen-4-one)

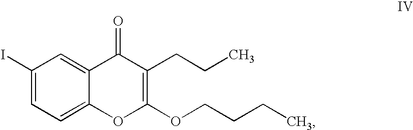

acibenzolar-S-methyl, captafol, captan, dazomet, folpet, fenoxanil, quinoxyfen and N,N-dimethyl-3-(3-bromo-6-fluoro-2-methylindole-1-sulfonyl)-[1,2,4]triazole-1-sulfonamide of the formula V

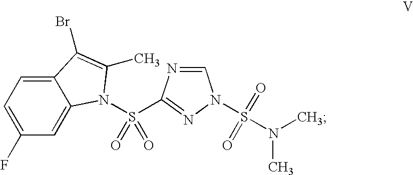

E) carbamates selected from the group consisting of mancozeb, maneb, metam, metiram, ferbam, propineb, thiram, zineb, ziram, diethofencarb, iprovalicarb, flubenthiavalicarb, propamocarb, 4-fluorophenyl N-(1-(1-(4-cyanophenyl)ethanesulfonyl)but-2-yl)carbamate, methyl 3-(4-chlorophenyl)-3-(2-isopropoxycarbonylamino-3-methylbutyrylamino)propanoate of the formula VI

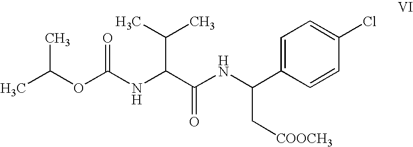

and carbamate oxime ethers of the formula VII

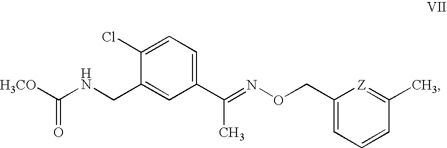

in which Z is N or CH;
F) other fungicides selected from the group consisting of guanidine, dodine, iminoctadine, guazatine, antibiotics: kasugamycin, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds: chlorothalonil, dichlofluanid, flusulfamide, hexachlorobenzene, phthalide, pencycuron, quintozene, thiophanate-methyl, tolyifluanid, inorganic active compounds: Bordeaux mixture, copper acetate, copper hydroxide, copper oxychloride, basic copper sulfate, sulfur, others: cyflufenamid, cymoxanil, dimethirimol, ethirimol, furalaxyl, metrafenone and spiroxamine;
   in a synergistically effective amount.

Moreover, the invention relates to a method for controlling harmful fungi using a composition of at least one compound I and at least one of the active compounds II, to the use of the compound(s) I with active compound(s) II for preparing such compositions, and also to compositions and seed comprising such compositions.

The prior art discloses numerous different classes of compounds which are effective in the control of phytopathogenic fungi. In many cases, the activity of the known compounds is unsatisfactory, and there is an ongoing search for novel active compounds which can be used as an alternative. Based on this it is the object of the present invention to provide compounds having at least comparable activity, preferably improved activity, and/or a broader activity spectrum.

Accordingly, we have found the compounds I according to the invention defined at the outset.

With a view to reducing the application rates and broadening the activity spectrum of the active compounds I and II, it was furthermore an object of the present invention to provide options where, at a reduced total amount of active compounds applied, it is possible to achieve an improved activity against harmful fungi, in particular for certain applications.

Accordingly we have found the compositions, defined at the outset, of the active compounds I and II. Moreover, we have found that simultaneous, that is joint or separate application of at least one compound I and of at least one active compound II, or successive application of the compound(s) I and at least one active compound II allows better control of harmful fungi than the individual compounds alone (synergistic compositions).

Some of the compounds of the formula I used as component 1), their preparation and their activity against harmful fungi are known from the literature (cf., for example, WO 2004/056824, WO2004/056825, WO 2004/056826, WO 2004/056829, WO 2005/123698, WO 2005/123733), or they can be prepared in the manner described therein. However, the activity of the compounds of the formula I is, in particular at low application rates, not always satisfactory.

The active compounds II mentioned above as component 2), their preparation and their action against harmful fungi are generally known (cf., for example, http://www.hclrss.demon.co.uk/index.html); most of them are commercially available.
Benalaxyl, methyl N-(phenylacetyl)-N-(2,6-xylyl)-DL-alaninate (DE 29 03 612);
metalaxyl, methyl N-(methoxyacetyl)-N-(2,6-xylyl)-DL-alaninate (GB 15 00 581);
ofurace, (RS)-α-(2-chloro-N-2,6-xylylacetamido)-γ-butyrolactone [CAS RN 58810-48-3];
oxadixyl, N-(2,6-dimethylphenyl)-2-methoxy-N-(2-oxo-3-oxazolidinyl)acetamide (GB 20 58 059);
aldimorph, “4-alkyl-2,5(or 2,6)-dimethylmorpholine”, comprising 65-75% of 2,6-dimethylmorpholine and 25-35% of 2,5-dimethylmorpholine, comprising more than 85% of 4-dodecyl-2,5(or 2,6)-dimethylmorpholine, where “alkyl” also includes octyl, decyl, tetradecyl and hexadecyl, with a cis/trans ratio of 1:1 [CAS RN 91315-15-0];
dodine, 1-dodecylguanidinium acetate (Plant Dis. Rep., Vol. 41, p. 1029 (1957));
dodemorph, 4-cyclododecyl-2,6-dimethylmorpholine (DE-A 1198125);
fenpropimorph, (RS)-cis-4-[3-(4-tert-butylphenyl)-2-methylpropyl]-2,6-dimethylmorpholine (DE-A 27 52 096);
fenpropidin, (RS)-1-[3-(4-tert-butylphenyl)-2-methylpropyl]piperidine (DE-A 27 52 096);
guazatine, mixture of the reaction products from the amidation of technical grade iminodi(octamethylene)diamine; comprising various guanidines and polyamines [CAS RN 108173-90-6];
iminoctadine, 1,1′-iminodi(octamethylene)diguanidine (Congr. Plant Pathol., 1., p. 27 (1968));
spiroxamine, (8-tert-butyl-1,4-dioxaspiro[4.5]dec-2-yl)diethylamine (EP-A 281 842);
tridemorph, 2,6-dimethyl-4-tridecylmorpholine (DE-A 11 64 152);
pyrimethanil, 4,6-dimethylpyrimidin-2-ylphenylamine (DD-A 151 404);
mepanipyrim, (4-methyl-6-prop-1-ynylpyrimidin-2-yl)phenylamine (EP-A 224 339);
cyprodinil, (4-cyclopropyl-6-methylpyrimidin-2-yl)phenylamine (EP-A 310 550);
cycloheximide, 4-{(2R)-2-[(1S,3S,5S)-3,5-dimethyl-2-oxocyclohexyl]-2-hydroxyethyl}piperidine-2,6-dione [CAS RN 66-81-9];
griseofulvin, 7-chloro-2′,4,6-trimethoxy-6′-methylspiro[benzofuran-2(3H), 1′-cyclohex-2′-ene]-3,4′-dione [CAS RN 126-07-8];
kasugamycin, 3-O-[2-amino-4-[(carboxylminomethyl)amino]-2,3,4,6-tetradeoxy-α-D-arabino-hexopyranosyl]-D-chiro-inositol [CAS RN 6980-18-3];
natamycin, (8E,14E,16E,18E,20E)-(1R,3S,5R,7R,12R,22R,24S,25R,26S)-22-(3-amino-3,6-dideoxy-β-D-mannopyranosyloxy)-1,3,26-trihydroxy-12-methyl-10-oxo-6,11,28-trioxatricyclo[22.3.1.0^{5,7}]octacosa-8,14,16,18,20-pentaene-25-carboxylic acid [CAS RN 7681-93-8];
polyoxin, 5-(2-amino-5-O-carbamoyl-2-deoxy-R^{c}-xylonamido)-1-(5-carboxy-1,2,3,4-tetrahydro-2,4-dioxopyrimidin-1-yl)-1,5-dideoxy-β-D-allofuranuronic acid [CAS RN 22976-86-9];
streptomycin, 1,1′-{1-R^{c}-(1,3,5/2,4,6)-4-[5-deoxy-2-O-(2-deoxy-2-methylamino-α-R^{c}-glucopyranosyl)-3-C-formyl-α-R^{c}-lyxofuranosyloxy]-2,5,6-trihydroxycyclohex-1,3-ylene}diguanidine (J. Am. Chem. Soc. Vol. 69, p. 1234 (1947));
bitertanol, β-([1,1′-biphenyl]-4-yloxy)-α-(1,1-dimethylethyl)-1H-1,2,4-triazole-1-ethanol (DE-A 23 24 020),
bromuconazole, 1-[[4-bromo-2-(2,4-dichlorophenyl)tetrahydro-2-furanyl]methyl]-1H-1,2,4-triazole (Proc. 1990 Br. Crop. Prot. Conf.-Pests Dis. Vol. 1, p. 459);
cyproconazole, 2-(4-chlorophenyl)-3-cyclopropyl-1-[1,2,4]triazol-1-ylbutan-2-ol (U.S. Pat. No. 4,664,696);
difenoconazole, 1-{2-[2-chloro-4-(4-chlorophenoxy)phenyl]-4-methyl-[1,3]dioxolan-2-ylmethyl}-1H-[1,2,4]triazole (GB-A 2 098 607);
diniconazole, (βE)-β-[(2,4-dichlorophenyl)methylene]-α-(1,1-dimethylethyl)-1H-1,2,4-triazole-1-ethanol (Noyaku Kagaku, 1983, Vol. 8, p. 575);
enilconazole (imazalil), 1-[2-(2,4-dichlorophenyl)-2-(2-propenyloxy)ethyl]-1H-imidazole (Fruits, 1973, Vol. 28, p. 545);
epoxiconazole, (2RS,3SR)-1-[3-(2-chlorophenyl)-2,3-epoxy-2-(4-fluorophenyl)propyl]-1H-1,2,4-triazole (EP-A 196 038);
fenbuconazole, α-[2-(4-chlorophenyl)ethyl]-α-phenyl-1H-1,2,4-triazole-1-propanenitrile (Proc. 1988 Br. Crop Prot. Conf.-Pests Dis. Vol. 1, p. 33);
fluquinconazole, 3-(2,4-dichlorophenyl)-6-fluoro-2-[1,2,4]-triazol-1-yl-3H-quinazolin-4-one (Proc. Br. Crop Prot. Conf.-Pests Dis., 5-3, 411 (1992));
flusilazole, 1-{[bis(4-fluorophenyl)methylsilanyl]methyl}-1H-[1,2,4]triazole (Proc. Br. Crop Prot. Conf.-Pests Dis., Vol. 1, 413 (1984));
flutriafol, α-(2-fluorophenyl)-α-(4-fluorophenyl)-1H-1,2,4-triazole-1-ethanol (EP-A 15 756);
hexaconazole, 2-(2,4-dichlorophenyl)-1-[1,2,4]triazol-1-ylhexan-2-ol (CAS RN 79983-71-4);
ipconazole, 2-[(4-chlorophenyl)methyl]-5-(1-methylethyl)-1-(1H-1,2,4-triazol-1-yl-methyl)cyclopentanol (EP-A 267 778),
metconazole, 5-(4-chlorobenzyl)-2,2-dimethyl-1-[1,2,4]triazol-1-ylmethylcyclopentanol (GB 857 383);
myclobutanil, 2-(4-chlorophenyl)-2-[1,2,4]triazol-1-ylmethylpentanenitrile (CAS RN 88671-89-0);
penconazole, 1-[2-(2,4-dichlorophenyl)pentyl]-1H-[1,2,4]triazole (Pesticide Manual, 12th Ed. 2000, p. 712);
propiconazole, 1-[[2-(2,4-dichlorophenyl)-4-propyl-1,3-dioxolan-2-yl]methyl]-1H-1,2,4-triazole (BE 835 579);
prochloraz, N-(propyl-[2-(2,4,6-trichlorophenoxy)ethyl])imidazole-1-carboxamide (U.S. Pat. No. 3,991,071);
prothioconazole, 2-[2-(1-chlorocyclopropyl)-3-(2-chlorophenyl)-2-hydroxypropyl]-2,4-dihydro[1,2,4]triazole-3-thione (WO 96/16048);
simeconazole, α-(4-fluorophenyl)-α-[(trimethylsilyl)methyl]-1H-1,2,4-triazole-1-ethanol [CAS RN 149508-90-7],
tebuconazole, 1-(4-chlorophenyl)-4,4-dimethyl-3-[1,2,4]triazol-1-ylmethylpentan-3-ol (EP-A 40 345);
tetraconazole, 1-[2-(2,4-dichlorophenyl)-3-(1,1,2,2-tetrafluoroethoxy)propyl]-1H-1,2,4-triazole (EP-A 234 242);
triadimefon, 1-(4-chlorophenoxy)-3,3-dimethyl-1-(1H-1,2,4-triazol-1-yl)-2-butanone (BE 793 867);
triadimenol, β-(4-chlorophenoxy)-α-(1,1-dimethylethyl)-1H-1,2,4-triazole-1-ethanol (DE-A 23 24 010);
triflumizole, (4-chloro-2-trifluoromethylphenyl)-(2-propoxy-1-[1,2,4]triazol-1-ylethylidene)-amine (JP-A 79/119 462);
triticonazole, (5E)-5-[(4-chlorophenyl)methylene]-2,2-dimethyl-1-(1H-1,2,4-triazol-1-ylmethyl)cyclopentanol (FR 26 41 277);
iprodione, N-isopropyl-3-(3,5-dichlorophenyl)-2,4-dioxoimidazolidine-1-carboxamide (GB 13 12 536);
myclozolin, (RS)-3-(3,5-dichlorophenyl)-5-methoxymethyl-5-methyl-1,3-oxazolidine-2,4-dione [CAS RN 54864-61-8];
procymidone, N-(3,5-dichlorophenyl)-1,2-dimethylcyclopropane-1,2-dicarboximide (U.S. Pat. No. 3,903,090);
vinclozolin, 3-(3,5-dichlorophenyl)-5-methyl-5-vinyloxazolidine-2,4-dione (DE-A 22 07 576);
ferbam, iron(3+) dimethyldithiocarbamate (U.S. Pat. No. 1,972,961);
nabam, disodium ethylenebis(dithiocarbamate) (U.S. Pat. No. 2,317,765);
maneb, manganese ethylenebis(dithiocarbamate) (U.S. Pat. No. 2,504,404);
mancozeb, manganese ethylenebis(dithiocarbamate) polymer complex zinc salt (GB 996 264);
metam, methyldithiocarbamic acid (U.S. Pat. No. 2,791,605);
metiram, zinc ammoniate ethylenebis(dithiocarbamate) (U.S. Pat. No. 3,248,400);
propineb, zinc propylenebis(dithiocarbamate) polymer (BE 611 960);
polycarbamate, bis(dimethylcarbamodithioato-κS,κS′)[μ-[[1,2-ethanediylbis[carbamodithioato-κS,κS′]](2-)]]di[zinc] [CAS RN 64440-88-6];
thiram, bis(dimethylthiocarbamoyl) disulfide (DE-A 642 532);
ziram, dimethyldithiocarbamate [CAS RN 137-30-4];
zineb, zinc ethylenebis(dithiocarbamate) (U.S. Pat. No. 2,457,674);
anilazine, 4,6-dichloro-N-(2-chlorophenyl)-1,3,5-triazine-2-amine (U.S. Pat. No. 2,720,480);
benomyl, N-butyl-2-acetylaminobenzoimidazole-1-carboxamide (U.S. Pat. No. 3,631,176);
boscalid, 2-chloro-N-(4′-chlorobiphenyl-2-yl)nicotinamide (EP-A 545 099);
carbendazim, methyl (1H-benzimidazol-2-yl)carbamate (U.S. Pat. No. 3,657,443);
carboxin, 5,6-dihydro-2-methyl-N-phenyl-1,4-oxathiin-3-carboxamide (U.S. Pat. No. 3,249,499);
oxycarboxin, 5,6-dihydro-2-methyl-1,4-oxathiin-3-carboxanilide 4,4-dioxide (U.S. Pat. No. 3,399,214);
cyazofamid, 4-chloro-2-cyano-N,N-dimethyl-5-(4-methylphenyl)-1H-imidazole-1-sulfonamide (CAS RN 120116-88-3];
dazomet, 3,5-dimethyl-1,3,5-thiadiazinane-2-thione (Bull. Soc. Chim. Fr. Vol. 15, p. 891 (1897));
diflufenzopyr, 2-{1-[4-(3,5-difluorophenyl)semicarbazono]ethyl}nicotinic acid [CAS RN 109293-97-2];
dithianon, 5,10-dioxo-5,10-dihydronaphtho[2,3-b][1,4]dithiin-2,3-dicarbonitrile (GB 857 383);
famoxadone, (RS)-3-anilino-5-methyl-5-(4-phenoxyphenyl)-1,3-oxazolidine-2,4-dione [CAS RN 131807-57-3];
fenamidone, (S)-1-anilino-4-methyl-2-methylthio-4-phenylimidazolin-5-one [CAS RN 161326-34-7];
fenarimol, α-(2-chlorophenyl)-α-(4-chlorophenyl)-5-pyrimidinemethanol (GB 12 18 623);
fuberidazole, 2-(2-furanyl)-1H-benzimidazole (DE-A 12 09 799);
flutolanil, α,α,α-trifluoro-3′-isopropoxy-o-toluanilide (JP 1104514);
furametpyr, 5-chloro-N-(1,3-dihydro-1,1,3-trimethyl-4-isobenzofuranyl)-1,3-dimethyl-1H-pyrazole-4-carboxamide [CAS RN 123572-88-3];
isoprothiolane, diisopropyl 1,3-dithiolan-2-ylidenemalonate (Proc. Insectic. Fungic. Conf. 8. Vol. 2, p. 715 (1975));
mepronil, 3′-isopropoxy-o-toluanilide (U.S. Pat. No. 3,937,840);
nuarimol, α-(2-chlorophenyl)-α-(4-fluorophenyl)-5-pyrimidinemethanol (GB 12 18 623);
fluopicolide (picobenzamid), 2,6-dichloro-N-(3-chloro-5-trifluoromethylpyridin-2-ylmethyl)benzamide (WO 99/42447);
probenazole, 3-allyloxy-1,2-benzothiazole 1,1-dioxide (Agric. Biol. Chem. Vol. 37, p. 737 (1973));
proquinazid, 6-iodo-2-propoxy-3-propylquinazoiin-4(3H)-one (WO 97/48684);
pyrifenox, 2′,4′-dichloro-2-(3-pyridyl)acetophenone (EZ)-O-methyloxime (EP 49 854);
pyroquilon, 1,2,5,6-tetrahydropyrrolo[3,2,1-ij]quinolin-4-one (GB 139 43 373);
quinoxyfen, 5,7-dichloro-4-(4-fluorophenoxy)quinoline (U.S. Pat. No. 5,240,940);
silthiofam, N-allyl-4,5-dimethyl-2-(trimethylsilyl)thiophene-3-carboxamide [CAS RN 175217-20-6];
thiabendazole, 2-(1,3-thiazol-4-yl)benzimidazole (U.S. Pat. No. 3,017,415);
thifluzamide, 2′,6′-dibromo-2-methyl-4′-trifluoromethoxy-4-trifluoromethyl-1,3-thiazole-5-carboxanifide [CAS RN 130000-40-7];
thiophanate-methyl, 1,2-phenylenebis(iminocarbonothioyl)bis(dimethylcarbamate) (DE-A 19 30 540);
tiadinil, 3′-chloro-4,4′-dimethyl-1,2,3-thiadiazole-5-carboxanilide [CAS RN 223580-51-6];
tricyclazole, 5-methyl-1,2,4-triazolo[3,4-b][1,3]benzothiazole [CAS RN 41814-78-2];
triforine, N,N′-{piperazine-1,4-diylbis[(trichloromethyl)methylene]}diformamide (DE-A 19 01 421);
5-chloro-7-(4-methylpiperidin-1-yl)-6-(2,4,6-trifluorophenyl)-[1,2,4]triazolo[1,5-a]pyrimidine (WO 98/46607);
Bordeaux mixture, mixture of CuSO₄×3Cu(OH)₂×3CaSO₄ [CAS RN 8011-63-0];
copper acetate, Cu(OCOCH₃)₂ [CAS RN 8011-63-0];
copper oxychloride, Cu₂Cl(OH)₃ [CAS RN 1332-40-7];
basic copper sulfate, CuSO₄ [CAS RN 1344-73-6];
binapacryl, (RS)-2-sec-butyl-4,6-dinitrophenyl 3-methylcrotonate [CAS RN 485-31-4];
dinocap, mixture of 2,6-dinitro-4-octylphenylcrotonate and 2,4-dinitro-6-octylphenylcrotonate, where “octyl” is a mixture of 1-methylheptyl, 1-ethylhexyl and 1-propylpentyl (U.S. Pat. No. 2,526,660);
dinobuton, (RS)-2-sec-butyl-4,6-dinitrophenyl isopropyl carbonate [CAS RN 973-21-7];
nitrothal-isopropyl, diisopropyl 5-nitroisophthalate (Proc. Br. Insectic. Fungic. Conf. 7., Vol. 2, p. 673 (1973));
fenpiclonil, 4-(2,3-dichlorophenyl)-1H-pyrrole-3-carbonitrile (Proc. 1988 Br. Crop Prot. Conf. -Pests Dis., Vol. 1, p. 65);
fludioxonil, 4-(2,2-difluorobenzo[1,3]dioxol-4-yl)-1H-pyrrole-3-carbonitrile (The Pesticide Manual, publ. The British Crop Protection Council, 10th ed. 1995, p. 482);
acibenzolar-S-methyl, methyl 1,2,3-benzothiadiazole-7-carbothioate [CAS RN 135158-54-2];
flubenthiavalicarb (benthiavalicarb), isopropyl {(S)-1-[(1R)-1-(6-fluorobenzothiazol-2-yl)-ethylcarbamoyl]-2-methylpropyl}carbamate (JP-A 09/323,984);
carpropamid, 2,2-dichloro-N-[1-(4-chlorophenyl)ethyl]-1-ethyl-3-methylcyclopropanecarboxamide [CAS RN 104030-54-8];
chlorothalonil, 2,4,5,6-tetrachloroisophthalonitrile (U.S. Pat. No. 3,290,353);
cyflufenamid, (Z)-N-[α-(cyclopropylmethoxyimino)-2,3-difluoro-6-(trifluoromethyl)benzyl]-2-phenylacetamide (WO 96/19442);
cymoxanil, 1-(2-cyano-2-methoxyiminoacetyl)-3-ethylurea (U.S. Pat. No. 3,957,847);
diclomezine, 6-(3,5-dichlorophenyl-p-tolyl)pyridazin-3(2H)-one (U.S. Pat. No. 4,052,395) diclocymet, (RS)-2-cyano-N-[(R)-1-(2,4-dichlorophenyl)ethyl]-3,3-dimethylbutyramide [CAS RN 139920-32-4];
diethofencarb, isopropyl 3,4-diethoxycarbanilate (EP-A 78 663);
edifenphos, O-ethyl S,S-diphenyl phosphorodithioate (DE-A 14 93 736)
ethaboxam, N-(cyano-2-thienylmethyl)-4-ethyl-2-(ethylamino)-5-thiazolecarboxamide (EP-A 639 574);
fenhexamid, N-(2,3-dichloro-4-hydroxyphenyl)-1-methylcyclohexanecarboxamide (Proc. Br. Crop Prot. Conf.-Pests Dis., 1998, Vol. 2, p. 327);
fentin acetate, triphenyltin (U.S. Pat. No. 3,499,086);
fenoxanil, N-(1-cyano-1,2-dimethylpropyl)-2-(2,4-dichlorophenoxy)propanamide (EP-A 262 393);
ferimzone, (Z)-2′-methylacetophenone-4,6-dimethylpyrimidin-2-ylhydrazone [CAS RN 89269-64-7];
fluazinam, 3-chloro-N-[3-chloro-2,6-dinitro-4-(trifluoromethyl)phenyl]-5-(trifluoromethyl)-2-pyridinamine (The Pesticide Manual, publ. The British Crop Protection Council, 10th ed. (1995), p. 474);
fosetyl, fosetyl-aluminum, ethylphosphonate (FR 22 54 276);
iprovalicarb, isopropyl [(1S)-2-methyl-1-(1-p-tolylethylcarbamoyl)propyl]carbamate (EP-A 472 996);
hexachlorobenzene (C. R. Seances Acad. Agric. Fr., Vol. 31, p. 24 (1945));
mandipropamid, (RS)-2-(4-chlorophenyl)-N-[3-methoxy-4-(prop-2-ynyloxy)phenethyl]-2-(prop-2-ynyloxy)acetamide (WO 03/042166);
metrafenone, 3′-bromo-2,3,4,6′-tetramethoxy-2′,6-dimethylbenzophenone (U.S. Pat. No. 5,945,567);
pencycuron, 1-(4-chlorobenzyl)-1-cyclopentyl-3-phenylurea (DE-A 27 32 257);
penthiopyrad, (RS)—N-[2-(1,3-dimethylbutyl)-3-thienyl]-1-methyl-3-(trifluoromethyl)-1H-pyrazole-4-carboxamide (JP 10/130,268);
propamocarb, isopropyl 3-(dimethylamino)propylcarbamate (DE-A 15 67 169);
phthalide (DE-A 16 43 347);
toloclofos-methyl, O-2,6-dichloro-p-tolyl-O,O-dimethyl phosphorothioate (GB 14 67 561);
quintozene, pentachloronitrobenzene (DE-A 682 048);
zoxamide, (RS)-3,5-dichloro-N-(3-chloro-1-ethyl-1-methyl-2-oxopropyl)-p-toluamide [CAS RN 156052-68-5];
captafol, N-(1,1,2,2-tetrachloroethylthio)cyclohex-4-ene-1,2-dicarboximide (Phytopathology, Vol. 52, p. 754 (1962));
captan, N-(trichloromethylthio)cyclohex-4-ene-1,2-dicarboximide (U.S. Pat. No. 2,553,770);
dichlofluanid, N-dichlorofluoromethylthio-N′,N′-dimethyl-N-phenylsulfamide (DE-A 11 93 498);
folpet, N-(trichloromethylthio)phthalimide (U.S. Pat. No. 2,553,770);
tolylfluanid, N-dichlorofluoromethylthio-N′,N′-dimethyl-N-p-tolylsulfamide (DE-A 11 93 498);
dimethomorph, 3-(4-chlorophenyl)-3-(3,4-dimethoxyphenyl)-1-morpholin-4-yl-propenone (EP-A 120 321);
flumetover, 2-(3,4-dimethoxyphenyl)-N-ethyl-α,α,α-trifluoro-N-methyl-p-toluamide [AGROW no. 243, 22 (1995)];
flumorph, 3-(4-fluorophenyl)-3-(3,4-dimethoxyphenyl)-1-morpholin-4-ylpropenone (EP-A 860 438);
N-(4′-bromobiphenyl-2-yl)-4-difluoromethyl-2-methylthiazole-5-carboxamide,
N-(4′-trifluoromethylbiphenyl-2-yl)-4-difluoromethyl-2-methylthiazole-5-carboxamide,
N-(4′-chloro-3′-fluorobiphenyl-2-yl)-4-difluoromethyl-2-methylthiazole-5-carboxamide (WO 03/66610);
N-(3′,4′-dichloro-4-fluorobiphenyl-2-yl)-3-difluoromethyl-1-methylpyrazole-4-carboxamide and N-(3′,4′-dichloro-5-fluorobiphenyl-2-yl)-3-difluoromethyl-1-methylpyrazole-4-carboxamide (WO 03/70705);
N-(2-cyanophenyl)-3,4-dichloroisothiazole-5-carboxamide (WO 99/24413);
N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide (WO 04/49804);
3-[5-(4-chlorophenyl)-2,3-dimethylisoxazolidin-3-yl]pyridine (EP-A 10 35 122);
2-butoxy-6-iodo-3-propylchromen-4-one (WO 03/14103);
N,N-dimethyl-3-(3-bromo-6-fluoro-2-methylindole-1-sulfonyl)-[1,2,4]triazole-1-sulfonamide (EP-A 10 31 571);
methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate (EP-A 12 01 648);
methyl 3-(4-chlorophenyl)-3-(2-isopropoxycarbonylamino-3-methylbutyrylamino)-propionate (EP-A 10 28 125);
azoxystrobin, methyl 2-{2-[6-(2-cyano-1-vinylpenta-1,3-dienyloxy)pyrimidin-4-yloxy]pheny}-3-methoxyacrylate (EP-A 382 375),
dimoxystrobin, (E)-2-(methoxyimino)-N-methyl-2-[α-(2,5-xylyloxy)-o-tolyl]acetamide (EP-A 477 631);
fluoxastrobin, (E)-{2-[6-(2-chlorophenoxy)-5-fluoropyrimidin-4-yloxy]phenyl}(5,6-dihydro-1,4,2-dioxazin-3-yl)methanone O-methyloxime (WO 97/27189);
kresoxim-methyl, methyl (E)-methoxyimino[α-(o-tolyloxy)-o-tolyl]acetate (EP-A 253 213);
metominostrobin, (E)-2-(methoxyimino)-N-methyl-2-(2-phenoxyphenyl)acetamide (EP-A 398 692);
orysastrobin, (2E)-2-(methoxyimino)-2-{2-[(3E,5E,6E)-5-(methoxyimino)-4,6-dimethyl-2,8-dioxa-3,7-diazanona-3,6-dien-1-yl]phenyl}-N-methylacetamide (WO 97/15552);
picoxystrobin, methyl 3-methoxy-2-[2-(6-trifluoromethylpyridin-2-yloxymethyl)phenyl]-acrylate (EP-A 278 595);
pyraclostrobin, methyl N-{2-[1-(4-chlorophenyl)-1H-pyrazol-3-yloxymethyl]phenyl}(N-methoxy)carbamate (WO 96/01256);
trifloxystrobin, methyl (E)-methoxyimino-{(E)-α-[1-(α,α,α-trifluoro-m-tolyl)ethylidene-aminooxy]-o-tolyl}acetate (EP-A 460 575);
methyl 2-[ortho-(2,5-dimethylphenyloxymethylene)phenyl]-3-methoxyacrylate (EP-A 226 917);
5-chloro-7-(4-methylpiperidin-1-yl)-6-(2,4,6-trifluorophenyl)-[1,2,4]triazolo[1,5-a]pyrimidine (WO 98/46608);
3,4-dichloro-N-(2-cyanophenyl)isothiazole-5-carboxamide (WO 99/24413); compounds of the formula III (WO 04/049804);
N-(2-(4-[3-(4-chlorophenyl)prop-2-ynyloxy]-3-methoxyphenyl)ethyl)-2-methanesulfonylamino-3-methylbutyramide and N-(2-(4-[3-(4-chlorophenyl)prop-2-ynyloxy]-3-methoxyphenyl)ethyl)-2-ethanesulfonylamino-3-methylbutyramide (WO 03/66609);
2-butoxy-6-iodo-3-propylchromen-4-one (WO 03/14103);
N,N-dimethyl-3-(3-bromo-6-fluoro-2-methylindole-1-sulfonyl)-[1,2,4]triazole-1-sulfonamide (WO 03/053145);
methyl 3-(4-chlorophenyl)-3-(2-isopropoxycarbonylamino-3-methylbutyrylamino)-propanoate (EP-A 1028125);
N-(2-bicyclopropyl-2-ylphenyl)-3-difluoromethyl-1-methyl-1H-pyrazole-4-carboxamide (WO 03/074491, WO 2006/01586).

Depending on the substitution pattern, the compounds of the formula I may have one or more centers of chirality, in which case they are present as enantiomer or diastereomer mixtures. The invention provides both the pure enantiomers or diastereomers or rotamers and mixtures thereof or fungicidal compositions comprising the same. The scope of the present invention includes in particular the (R) and (S) isomers and the racemates of compounds of the formula I having chiral centers. Suitable compounds of the formula I also include all possible stereoisomers (cis/trans isomers) and mixtures thereof. The compounds according to the invention or the compounds I used in the compositions according to the invention may be present in various crystal modifications, such compositions also being part of the subject of the present invention.

According to the present invention, agriculturally acceptable salts include in particular the salts of those cations or the acid addition salts of those acids whose cations and anions, respectively, have no adverse effect on the fungicidal action of the compounds according to the invention.

Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may carry from one to four (C₁-C₄)-alkyl substituents and/or one phenyl or benzyl substituent, preferably diisopropylammonium, tetramethylammonium, tetrabutylammonium, trimethylbenzylammonium, and also phosphonium ions, sulfonium ions, preferably tri(C₁-C₄)-alkylsulfonium, and sulfoxonium ions, preferably tri(C₁-C₄)-alkylsulfoxonium.

Anions of advantageously useful acid addition salts are, for example, chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and also the anions of (C₁-C₄)-alkanoic acids, preferably formate, acetate, propionate and butyrate. They can be formed by reacting the compounds of formula I according to the invention with an acid of the corresponding anion, preferably hydrochloric acid, hydrobromic acid, sulfuric acid, phosphoric acid or nitric acid.

In the definitions of the variables given in formula I, collective terms are used which are generally representative for the respective substituents or parts of substituents in composite groups. The term (Cₙ-Cₘ) denotes the possible number of carbon atoms in the respective substituent or part of a substituent:

halogen: fluorine, chlorine, bromine or iodine, preferably fluorine or chlorine;
alkyl: saturated, straight-chain or branched hydrocarbon radicals having preferably 1 to 4, 6, 8, 10 or 12 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: alkyl as mentioned above, where in these groups some or all of the hydrogen atoms may be replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl: unsaturated, straight-chain or branched hydrocarbon radicals having preferably 2 to 4, 2 to 6, 2 to 8 or 2 to 10 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: alkenyl as mentioned above, where in these groups some or all of the hydrogen atoms may be replaced by halogen atoms as mentioned above, in particular by fluorine, chlorine and bromine;
alkynyl: straight-chain or branched hydrocarbon groups having preferably 2 to 4, 2 to 6, 2 to 8 or 3 to 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
cycloalkyl: mono- or bicyclic saturated hydrocarbon groups having preferably 3 to 6, 3 to 8 or 3 to 10 carbon ring members, for example C₃-C₈-cycloalkyl, such as cyclopropyl, cyclobutyl, cyclopentyl, cyclohexyl, cycloheptyl and cyclooctyl;
a 5-, 6-, 7-, 8-, 9- or 10-membered saturated, partially unsaturated or aromatic heterocycle which contains 1, 2, 3 or 4 heteroatoms from the group consisting of oxygen; nitrogen and sulfur:
in particular:
a five- or six-membered saturated or partially unsaturated heterocycle (hereinbelow also referred to as heterocyclyl) which contains one, two, three or four heteroatoms from the group consisting of oxygen, nitrogen and sulfur as ring members: for example monocyclic saturated or partially unsaturated heterocycles which, in addition to carbon ring members, contain one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexa-hydropyridazinyl, 2-hexahydropyrimidinyl, 4-hexahydropyrimidinyl, 5-hexahydro-pyrimidinyl, 2-piperazinyl, 1,3,5-hexahydro-triazin-2-yl and 1,2,4-hexahydrotriazin-3-yl and the corresponding -ylidene radicals;
a seven-membered saturated or partially unsaturated heterocycle which contains one, two, three or four heteroatoms from the group consisting of oxygen, nitrogen and sulfur as ring members: for example mono- and bicyclic heterocycles having 7 ring members which, in addition to carbon ring members, contain one to three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding ylidene radicals;
a five- or six-membered aromatic heterocycle which contains one, two, three or four heteroatoms from the group consisting of oxygen, nitrogen or sulfur: mono- or bicyclic heteroaryl, for example 5-membered heteroaryl which is attached via carbon and which contains one to three nitrogen atoms or one or two nitrogen atoms and/or one sulfur or oxygen atom as ring members, such as 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 5-membered heteroaryl which is attached via nitrogen and contains one to three nitrogen atoms as ring members, such as pyrrol-1-yl, pyrazol-1-yl, imidazol-1-yl, 1,2,3-triazol-1-yl and 1,2,4-triazol-1-yl; 6-membered heteroaryl which contains one to three nitrogen atoms as ring members, such as pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;

Aryl: aromatic hydrocarbon cycles having in particular 6 to 14, preferably 6 to 10, carbon atoms. Preference according to the invention is generally given to phenyl (C₆H₅—) and naphthyl (C₁₀H₇—).

The compounds of the formula I according to the invention can be prepared by various routes analogously to processes of the prior art known per se, such as, for example, analogously to the syntheses described in WO 2004/056824, WO2004/056825, WO 2004/056826, WO 2004/056829, WO 2005/123698, WO 2005/123733. The compounds according to the invention can be prepared, for example, by the syntheses shown below:

They are obtained in an advantageous manner by coupling 5,7-dihalo derivatives of the formula II

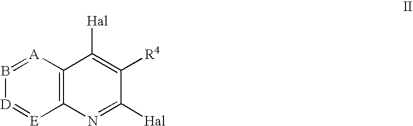

with organometallic reagents M^{w}(—R¹)_{w}, resulting in the introduction of the substituent R¹ which gives corresponding compounds of the formula I in which R⁵ is halogen:

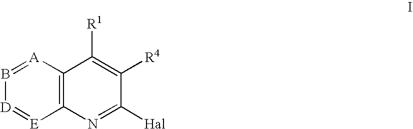

Here, the substituents and variables have the meanings or preferred meanings as given here for the compounds of the formula I, and Hal is halogen, preferably fluorine, chlorine or bromine, in particular chlorine. M is a metal ion of valency w, such as, for example, B, Zn, Mg or Sn. This reaction can be carried out, for example, analogously to the following processes: J. Chem. Soc. Perkin Trans. 1, 1187 (1994), ibid, 2345 (1996); WO-A 99/41255; Aust. J. Chem., Vol. 43, 733 (1990); J. Org. Chem., Vol. 43, 358 (1978); J. Chem. Soc. Chem. Commun. 866 (1979); Tetrahedron Lett., Vol. 34, 8267 (1993); ibid, Vol. 33, 413 (1992). According to one embodiment of this process, the reaction is carried out under transition metal catalysis, such as, for example Ni or Pd catalysis. Starting with compounds of the formula I in which R⁵ is halogen, it is possible to prepare corresponding compounds in which R⁵ is cyano, C₁-C₈-alkoxy or C₁-C₈-haloalkoxy. To this end, it is possible, for example, to react compounds of the formula I in which R⁵ is halogen, preferably chlorine, with compounds A^{a}B^{b}. A^{a} in the compounds A^{a}B^{b} is a cation, B^{b} is cyanide, a C₁-C₈-alkoxylate or a C₁-C₈-haloalkoxylate. Thus, depending on the group to be introduced, the compounds A^{a}B^{b} are inorganic cyanides (such as, for example, KCN, NH₄CN) or (halo)alkoxylates. The cation A^{a} is of little importance, and those of very different types may be suitable. For practical reasons, preference is usually given to ammonium, tetraalkylammonium salts, such as tetramethylammonium or tetraethylammonium salts, or alkali metal or alkaline earth metal salts. The reaction with A^{a}B^{b} is preferably carried out in an inert solvent. Suitable solvents include ethers, such as dioxane, diethyl ether, methyl tert-butyl ether and preferably tetrahydrofuran, halogenated hydrocarbons, such as dichloromethane or dichloroethane, aromatic hydrocarbons, such as toluene, and mixtures thereof. The reaction temperature is usually from 0 to 120° C., preferably from 10 to 40° C. [cf. J. Heterocycl. Chem., Vol. 12, pp. 861-863 (1975)]. The compounds of the formula I according to the invention in which R⁵ is C₁-C₈-alkyl, C₁-C₈-haloalkyl, C₂-C₈-alkenyl, C₂-C₈-haloalkenyl, C₂-C₈-alkynyl or C₃-C₈-haloalkynyl can, in an advantageous manner, also be prepared from compounds of the formula I in which R⁵ is halogen, in particular chlorine or bromine, by reacting these with an organometallic compound M-X^{a} in which M is lithium, magnesium or zinc and X^{a} is C₁-C₈-alkyl, C₁-C₈-haloalkyl, C₂-C₈-alkenyl, C₂-C₈-haloalkenyl, C₂-C₈-alkynyl or C₃-C₈-haloalkynyl. In this manner, using the corresponding compounds M-X^{a} is possible to prepare, in a particularly advantageous manner, compounds of the formula I in which R⁵ is C₁-C₈-alkyl, C₂-C₈-alkenyl or C₂-C₈-alkynyl. The reaction is preferably carried out in the presence of catalytic or in particular at, feast equimolar amounts of transition metal salts and/or transition metal compounds, in particular in the presence of Cu salts such as Cu(I) halides and especially Cu(I) iodide. The reaction is preferably carried out in an inert organic solvent, for example one of the ethers mentioned above, in particular tetrahydrofuran, an aliphatic or cycloaliphatic hydrocarbon such as hexane, cyclohexane and the like, an aromatic hydrocarbon, such as toluene, or in a mixture of these solvents. The temperatures preferred for the reaction are in the range of from −100 to +100° C., in particular in the range of from −80° C. to +40° C. Processes by which this can be achieved are known, for example from the prior art cited at the outset (see also, for example, WO 03/004465).

5,7-Dihalo derivatives of the formula II can be obtained, for example, by reacting the corresponding 5,7-dihydroxy derivative of the formula III

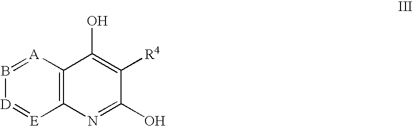

analogously to the methods described in WO-A 94/20501 with a halogenating agent. Here, the substituents and variables have the meanings or preferred meanings given herein for the compounds of the formula I. The halogenating agent used is preferably a phosphorus oxyhalide or a phosphorus(V) halide, such as, for example, phosphorus pentachloride, phosphorus oxybromide or phosphorus oxychloride or a mixture of phosphorus oxychloride and phosphorus pentachloride. It may be advantageous to add a hydrohalide of a tertiary amine, for example triethylamine hydrochloride, as co-catalyst. The halogenation reaction of compounds of the formula III to give compounds of the formula II is usually carried out at temperatures of from 0° C. to 150° C., preferably from 80° C. to 125° C. [see also EP-A 770 615]. The reaction can be carried out in the absence of a solvent or in an inert solvent, for example a halogenated hydrocarbon, such as dichloromethane, dichloroethane or an aromatic hydrocarbon, such as, for example, toluene or o-, m-, p-xylene or in a mixture of the solvents mentioned.

5,7-Dihydroxy derivatives of the formula III can be prepared analogously to routes familiar to the person skilled in the art.

Compounds of the formula I according to the invention in which R⁵ is C₁-C₈-alkyl, C₁-C₈-haloalkyl, C₂-C₈-alkenyl, C₂-C₈-haloalkenyl, C₂-C₈-alkynyl or C₃-C₈-haloalkynyl can also be prepared, for example, by coupling a halogen derivative of the formula IIa

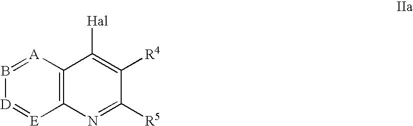

with an organometallic reagent M^{w}(—R¹)_{w}, thus introducing the substituent R¹ (see above), where Hal is halogen, R⁵ is C₁-C₈-alkyl, C₁-C₈-haloalkyl, C₂-C₈-alkenyl, C₂-C₈-haloalkenyl, C₂-C₈-alkynyl or C₃-C₈-haloalkynyl and the other substituents and variables have the meanings given for the compounds of the formula I.

Halogen derivatives of the formula IIa can be obtained by reacting the corresponding hydroxy derivative of the formula IIIa

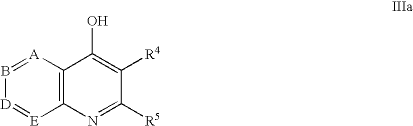

with a halogenating agent, where the substituents and variables have the meanings given for the compounds of the formula IIa.

The halogenation is carried out analogously to the procedure described above for the reaction of compounds of the formula III with a halogenating agent.

Hydroxyl derivatives of the formula IIIa can be prepared analogously to routes familiar to persons skilled in the art.

Compounds of the formula I in which R⁵ is C₁-C₈-alkyl can also be prepared by converting, in a first step, a compound of the formula I in which R⁵ is halogen, in particular chlorine or bromine, with a malonate of the formula IV

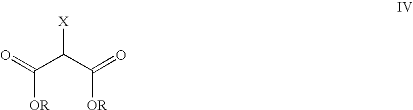

into a compound of the formula V

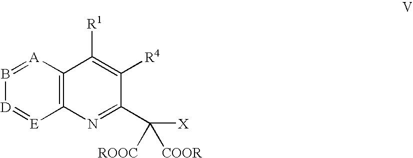

in which X is hydrogen or C₁-C₇-alkyl and R is C₁-C₄-alkyl and the substituents and variables are as defined for compounds of the formula I. The compound of the formula V is hydrolyzed and the hydrolysis product is decarboxylated [cf. U.S. Pat. No. 5,994,360]. The malonates IV are known form the literature, for example from J. Am. Chem. Soc., Vol. 64, 2714 (1942); J. Org. Chem., Vol. 39, 2172 (1974); Helv. Chim. Acta, Vol. 61, 1565 (1978)], or they can be prepared in accordance with the literature cited. The subsequent hydrolysis of the ester X is carried out under conditions known generally to the person skilled in the art. Depending on the various structural elements, alkaline or acidic hydrolysis of the compounds V may be advantageous. Under the conditions of ester hydrolysis, there may already be complete or partial decarboxylation to the compounds of the formula I. The decarboxylation is usually carried out at temperatures of from 20° C. to 180° C., preferably from 50° C. to 120° C., preferably in an inert solvent, if appropriate in the presence of an acid. Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane; anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; particularly preferably, the reaction is carried out in hydrochloric acid or acetic acid. It is also possible to use mixtures of the solvents mentioned.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which can be purified or freed from volatile components under reduced pressure and at moderately elevated temperature. If the intermediates and end products are obtained as solids, purification can also be carried out by recrystallization or digestion.

If individual compounds I according to the invention cannot be obtained by the routes described above, they can be prepared by derivatization of other compounds I or they can be obtained analogously to the compounds whose syntheses are described in the literature described at the outset.

If the synthesis yields mixtures of isomers, a separation is generally not necessarily required, however, since in some cases the individual isomers can be interconverted during work-up for use or during application (for example under the action of light, acids or bases). Such conversions may also take place after use, for example in the treatment of plants in the treated plants, or in the harmful fungi to be controlled.

As discussed above, the present invention provides compounds of the formula I as defined above. With a view to their intended use, preference is given to the following meanings of the substituents in formula I, in each case on their own or in combination:

According to one embodiment of the invention, R¹ is C₃-C₈-alkyl, C₃-C₈-haloalkyl, C₃-C₈-alkenyl, C₃-C₈-haloalkenyl, C₃-C₈-alkynyl, C₃-C₈-haloalkynyl, C₃-C₆-cycloalkyl (in particular C₅-C₆-cycloalkyl), C₅-C₆-halocycloalkyl, C₅-C₆-cycloalkenyl or C₅-C₆-halocycloalkenyl, where R¹ may carry one, two, three or four identical or different groups R^{a}. R¹ is in particular also C₁-C₈-alkyl or C₁-C₈-haloalkyl. Also preferably, R¹ is C₂-C₁₀-alkenyl or C₂-C₁₀-alkynyl. R¹ may in each case be substituted by R^{a}, as defined herein.

According to a further embodiment, R¹ is a five- or six-membered saturated heterocycle which is attached via carbon and which is unsubstituted or substituted by R^{a}, as defined herein, and which contains one, two, three or four heteroatoms from the group consisting of oxygen, nitrogen and sulfur.

According to yet another embodiment, R¹ is a five- or six-membered partially unsaturated heterocycle which is attached via carbon and which is unsubstituted or substituted by R^{a}, as defined herein, and which contains one, two, three or four heteroatoms from the group consisting of oxygen, nitrogen and sulfur.

According to yet another embodiment, R¹ is a five- or six-membered aromatic heterocycle which is attached via carbon and which is unsubstituted or substituted by R^{a}, as defined herein, and which contains one, two, three or four heteroatoms from the group consisting of oxygen, nitrogen and sulfur.

According to a further embodiment, R¹ is C₃-C₆-cycloalkyl which may be substituted by R^{a}, as defined herein, and in particular by C₁-C₄-alkyl.

According to one aspect, R^{a} is in each case independently halogen, cyano, C₁-C₆-alkyl, C₂-C₆-alkenyl, C₂-C₆-alkynyl, C₁-C₆-alkoxy, C₁-C₆-alkoxycarbonyl, C₁-C₆-alkoximino, C₂-C₆-alkenyloximino or C₂-C₆-alkynyloximino. It may be preferred for R^{a} to be independently chlorine, fluorine or bromine, cyano, C₁-C₄-alkyl, such as, in particular, methyl, ethyl, n-propyl and/or isopropyl, or C₁-C₆-alkoxy, such as, in particular, methoxy, ethoxy, n-propoxy and/or isopropoxy. As indicated above, R^{a} may be substituted by R^{b}.

According to a preferred aspect, R^{a} is in each case independently halogen, cyano, C₁-C₆-alkyl, C₂-C₆-alkenyl, C₂-C₆-alkynyl or C₁-C₆-alkoxy.

According to one embodiment, R⁴ is C₆-C₁₄-aryl, preferably phenyl or naphthyl, which is in each case unsubstituted or substituted by R^{c}, as defined herein.

According to one aspect, R⁴ is phenyl which has at least one substituent R^{c}. According to a further aspect, R⁴ is phenyl which has at least two substituents R^{c}. In special embodiments of the invention, in each case at least one substituent R^{c} is located in the ortho-position to the point of attachment of R⁴ to the skeleton of the formula I. It may be particularly preferred for the R^{c} in the ortho-position to be fluorine, chlorine, C₁-C₂-alkyl, such as methyl or ethyl, C₁-C₂-fluoroalkyl, such as trifluoroalkyl, or C₁-C₂-alkoxy, such as methoxy.

In a specific embodiment of the invention, R⁴ is a substituted phenyl group P,

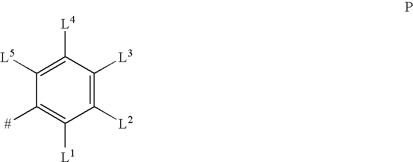

in which # is the point of attachment to the skeleton of the formula I and
L¹ is fluorine, chlorine, CH₃ or CF₃;
L², L⁴ independently of one another are hydrogen, fluorine or methoxy;
L³ is hydrogen, fluorine, chlorine, CN, CH₃, OCH₃, NH₂, NHCH₃, N(CH₃)₂, C(O)NH₂, COOCH₃ or C(O)CH₃; and
L⁵ is hydrogen, fluorine, chlorine or CH₃.

In a further embodiment of the invention, R⁴ is one of the following substituents: 2-fluoro-6-chlorophenyl, 2,6-difluorophenyl, 2,6-dichlorophenyl, 2-fluoro-6-methylphenyl, 2,4,6-trifluorophenyl, 2,6-difluoro-4-methoxyphenyl, 2-chloro-4-methoxyphenyl, pentafluorophenyl, 2-methyl-4-fluorophenyl, 2-trifluoromethylphenyl, 2-methoxy-6-fluorophenyl, 2-chlorophenyl, 2-fluorophenyl, 2,4-difluorophenyl, 2-fluoro-4-chlorophenyl, 2-chloro-4-fluorophenyl, 2-chloro-5-fluorophenyl, 2,3-difluorophenyl, 2,5-difluorophenyl, 2,3,4-trifluorophenyl, 2-methylphenyl, 2,4-dimethylphenyl, 2-methyl-4-chlorophenyl, 2-methyl-5-fluorophenyl, 2-fluoro-4-methylphenyl, 2,6-dimethylphenyl, 2,4,6-trimethylphenyl, 2,6-difluoro-4-methylphenyl, 2-trifluoromethyl-4-fluorophenyl, 2-trifluoromethyl-5-fluorophenyl, 2-trifluoromethyl-5-chlorophenyl or 2-fluoro-3-methoxyphenyl.

According to a further embodiment, R⁴ is phenyl which is unsubstituted or substituted by one, two or three substituents R^{c} independently of one another selected from the group consisting of fluorine, chlorine, bromine, cyano, nitro, formyl, methyl, ethyl, n-propyl, isopropyl, n-butyl, isobutyl, s-butyl, t-butyl, allyl, propargyl, methoxy, ethoxy, n-propoxy, isopropoxy, methylthio, ethylthio, n-propylthio, isopropylthio, methylsulfinyl, ethylsulfinyl, methylsulfonyl, ethylsulfonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulfinyl, trifluoromethylsulfonyl, trichloroethynyloxy, trifluoroethynyloxy, chloroallyloxy, iodopropargyloxy, methylamino, ethylamino, n-propylamino, isopropylamino, dimethylamino, diethylamino, acetyl, propionyl, acetyloxy, methoxycarbonyl, ethoxycarbonyl, hydroxyiminoethyl, ethoxyiminoethyl, cyclopropyl, cyclobutyl, cyclopentyl and cyclohexyl.

According to a further specific aspect of the invention, R⁴ is phenyl which contains one, two or three substituents in the 2-, 4- and/or 6-position independently of one another selected from the R^{c} mentioned here as being preferred, in particular independently of one another selected from the group consisting of fluorine and chlorine.

According to a further aspect of the invention, R⁴ is 2,4-, 2,5- or 2,6-disubstituted phenyl, 2-substituted phenyl or 2,4,6-trisubstituted phenyl, with substituents R^{c} independently of one another selected from the group consisting of fluorine, chlorine, bromine, cyano, nitro, OH, mercapto, methyl, ethyl, methoxy, methylthio, hydroxyiminomethyl, hydroximinoethyl, methoximinomethyl, methoximinoethyl, carboxyl and carboxymethyl.

According to a further aspect of the invention, R⁴ is 2-trifluoromethylphenyl, 2-chloro-5-nitrophenyl or 2-chloro-4-methoxyphenyl.

According to a further embodiment of the invention, R⁴ is a five- or six-membered aromatic heterocycle which contains one, two, three or four heteroatoms from the group consisting of O, N and S. Here, R⁴ is in particular an unsubstituted or substituted five- or six-membered aromatic heterocycle which contains one, two, three or four heteroatoms from the group consisting of O, N and S. The heteroaromatic group may in each case (also in the embodiments described below) be attached via a ring carbon atom or via a ring nitrogen atom to the skeleton of the formula I.

R^{c} is in each case independently selected from the meanings for R^{c} given above, unless indicated otherwise.

According to one aspect, R⁴ contains at least one ring nitrogen atom. Here, it may be preferred for the point of attachment of R⁴ to the skeleton of the formula I to be located in the ortho-position to the at least one nitrogen atom.

According to a further aspect of the invention, R⁴ is an unsubstituted or substituted 5-membered heteroaromatic radical which contains one, two, three or four heteroatoms from the group consisting of oxygen, nitrogen and sulfur, where R⁴ is unsubstituted or substituted by one, two, three or four identical or different substituents R^{c}. According to one aspect, the five-membered heteroaromatic radical is nitrogenous. The optionally substituted five-membered heteroaryl radical may in each case be attached via a ring carbon atom or via a ring nitrogen atom to the skeleton of the formula I.

According to a preferred embodiment of the present invention, the heteroaryl (R⁴) has at least one substituent which is located in the ortho-position to the point of attachment to the skeleton of the formula I to which R⁴ is attached. It may be particularly preferred for the R^{c} in the ortho-position to be fluorine, chlorine, (C₁-C₂)-alkyl, such as methyl or ethyl, C₁-C₂-fluoroalkyl, such as trifluoroalkyl, or C₁-C₂-alkoxy, such as methoxy.

In the compounds of the formula I or their precursors, if R^{c} is attached to a ring nitrogen of R⁴, it is particularly preferred for R^{c} to be in each case independently:

C₁-C₈-alkylcarbonyl, C₁-C₈-alkylthiocarbonyl; or
C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₃-C₈-cycloalkyl, C₃-C₈-halocycloalkyl, C₃-C₈-cycloalkenyl, C₃-C₈-halocycloalkenyl, C₁-C₈-alkoximinoalkyl, C₂-C₈-alkenyloximino-(C₁-C₈)-alkyl or C₂-C₈-alkynyloximino-C₁-C₈-alkyl. Particularly preferably, R^{c} is C₁-C₆-alkyl or C₁-C₆-haloalkyl, more preferably C₁-C₄-alkyl or C₁-C₄-haloalkyl, in particular methyl or ethyl, especially preferably methyl.

Here, R^{c}, if it is attached to a ring nitrogen of R⁴, is in each case particularly preferably C₁-C₄-alkyl, C₃-C₆-cycloalkyl, —COO(C₁-C₄), —CONH₂ or —CSNH₂, in particular methyl, ethyl, isopropyl, cyclopropyl or —COOCH₃.

If R^{c} is attached to a ring carbon of R⁴, R^{c} is preferably in each case independently:

C₁-C₈-alkylcarbonyl, C₁-C₈-alkylthiocarbonyl; or
C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl, C₃-C₈-halocycloalkenyl, C₁-C₈-alkoximinoalkyl, C₂-C₈-alkenyloximino-C₁-C₈-alkyl, C₂-C₈-alkynyloximino-C₁-C₈-alkyl; or
halogen, cyano, hydroxyl, nitro, amino, C₁-C₆-alkylamino, di-C₁-C₆-alkylamino, or
C₁-C₈-alkoxy, C₁-C₈-haloalkoxy, C₁-C₈-alkenyloxy, C₁-C₈-haloalkenyloxy, C₁-C₈-alkynyloxy, C₁-C₈-haloalkynyloxy, C₁-C₈-cycloalkoxy, C₁-C₈-halocycloalkoxy; or
fluorine, chlorine, methyl, methoxy.

In the compounds of the present invention, R⁴ (heteroaryl) may contain one to four or one to three or one or two identical or different substituents R^{c}. According to one embodiment, R⁴ contains identical substituents R^{c}. With particular preference, R⁴ contains one or two substituents R^{c}, particularly preferably one or two identical substituents R^{c}. More preferably, R⁴ has two identical substituents R^{c}.

According to one embodiment of the invention, R⁴ is thiazolyl, imidazolyl, pyrazolyl, 1,2,4-triazolyl or 1,2,3-triazolyl and especially pyrazol-1-yl, where the radicals mentioned above are unsubstituted or have 1, 2 or 3 substituents R^{c}.

In this embodiment, R⁴ is in particular one of the radicals Het-1 to Het-31 shown below:

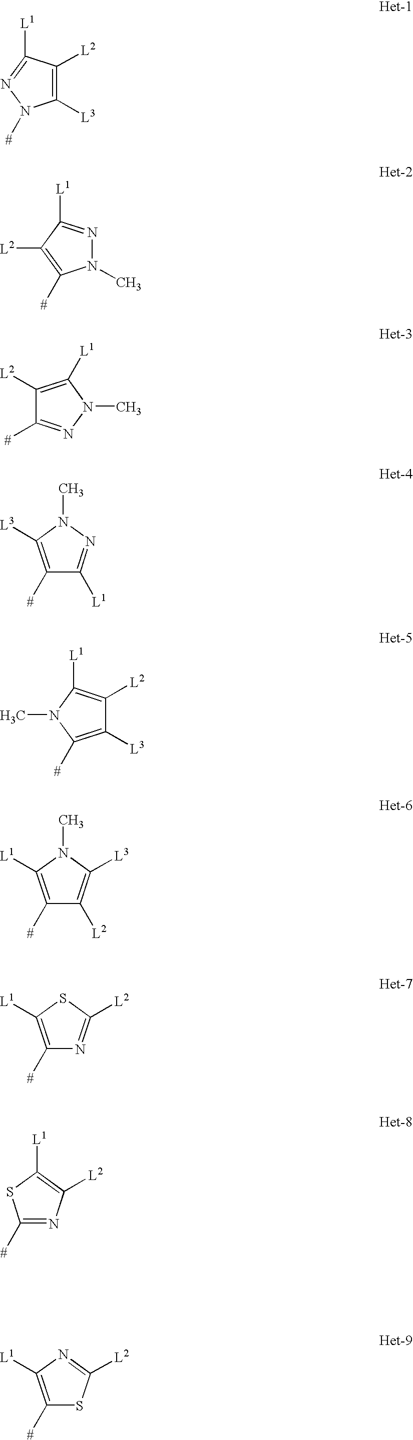

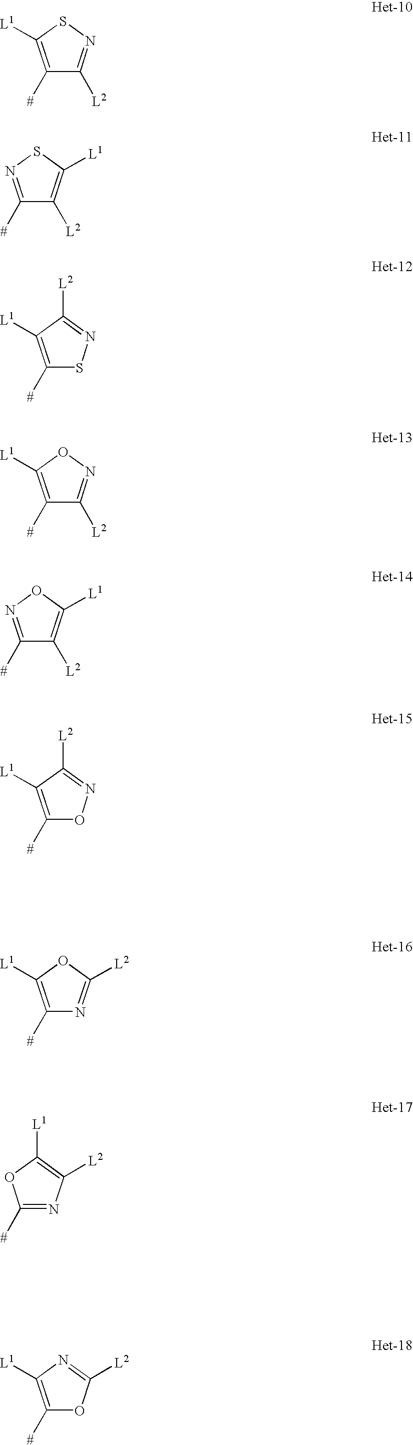

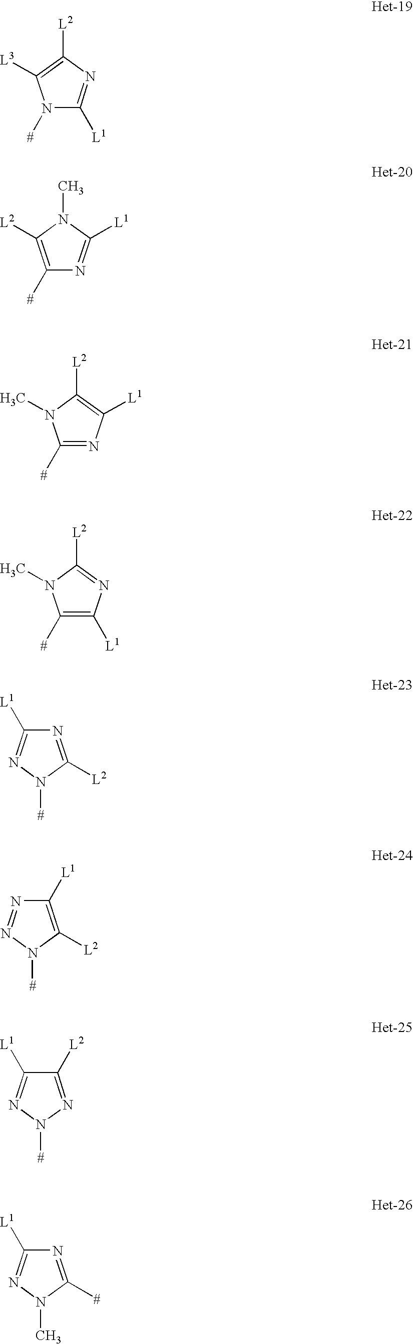

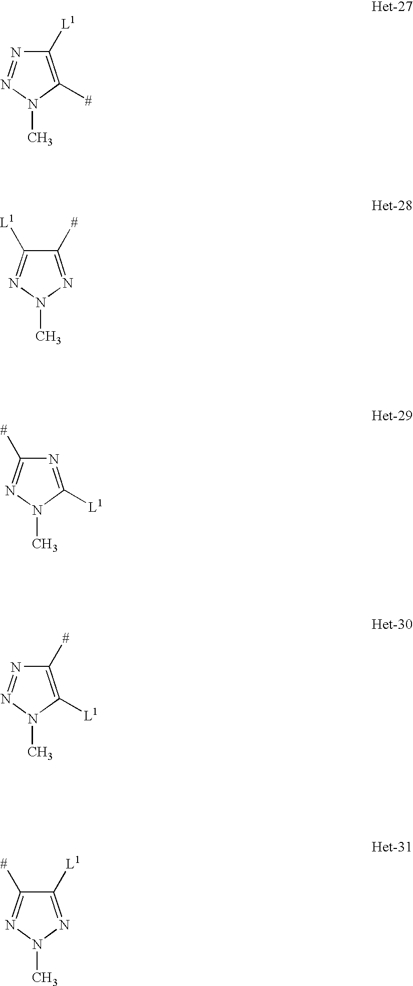

where
# denotes the point of attachment; and
L¹, L², and L³ independently of one another are hydrogen or have one of the meanings mentioned for R^{c}.

Preferably, the radicals L¹, L² and L³ independently of one another are selected from the group consisting of hydrogen, halogen, nitro, cyano, C₁-C₄-alkyl, C₁-C₄-haloalkyl, especially C₁-C₂-fluoroalkyl, C₁-C₄-alkoxy and C₁-C₄-alkoxycarbonyl. In particularly preferred embodiments, L¹, L² and L³ independently of one another are selected from the group consisting of hydrogen, nitro, cyano, fluorine, chlorine, bromine, methyl, ethyl, isopropyl, trifluoromethyl, fluoromethyl, methoxy and methoxycarbonyl.

Examples of Het-1 are 3,5-dimethylpyrazol-1-yl, 3,5-diisopropylpyrazol-1-yl, 3-methyl-5-isopropylpyrazol-1-yl, 3-isopropyl-5-methylpyrazol-1-yl, 3-ethyl-5-methylpyrazol-1-yl, 3,4,5-trimethylpyrazol-1-yl, 3-chloropyrazol-1-yl, 3-methylpyrazol-1-yl, 3-methyl-4-chloropyrazol-1-yl, 3-trifluoromethylpyrazol-1-yl, 3-trifluoromethyl-5-methoxypyrazol-1-yl, 3-trifluoromethyl-5-methylpyrazol-1-yl, 3-methyl-5-methoxypyrazol-1-yl, 3,5-dichloro-4-methylpyrazol-1-yl, 3,5-dimethyl-4-chloropyrazol-1-yl, 3,5-ditrifluoromethylpyrazol-1-yl and 3,4-dichloro-5-trichloromethylpyrazole.

Examples of Het-2 are 1,3-dimethylpyrazol-5-yl and 1-methyl-3-trifluoromethylpyrazol-5-yl.

Examples of Het-3 are 1,5-dimethylpyrazol-3-yl and 1-methyl-5-methoxypyrazol-3-yl.

Examples of Het-4 include, 1,3-dimethylpyrazol-4-yl, 1,5-dimethylpyrazol-4-yl, 1,3,5-trimethylpyrazol-4-yl, 1-methyl-3-trifluoromethylpyrazol-4-yl and 1-methyl-5-trifluoromethylpyrazol-4-yl.

Examples of Het-5 are 1-methylpyrrol-2-yl, 1,4-dimethylpyrrol-2-yl, 1-methyl-5-chloropyrrol-2-yl and 1-methyl-3,5-dichloropyrrol-2-yl.

Examples of Het-6 are 1,4-dimethylpyrazol-3-yl and 1-methylpyrazol-3-yl.

Examples of Het-7 include thiazol-4-yl, 2-methylthiazol-4-yl, 2-methyl-5-bromothiazol-4-yl, 2-methyl-5-chlorothiazol-4-yl and 2,5-dichlorothiazol-4-yl.

An example of Het-8 is thiazol-2-yl.

An example of Het-9 is thiazol-5-yl.

Examples of Het-10 include 3-methylisothiazol-4-yl and 3-methyl-5-chloroisothiazol-4-yl.

An example of Het-11 is isothiazol-3-yl.

An example of Het-12 is isothiazol-5-yl.

Examples of Het-13 include isoxazol-4-yl, 3,5-dimethylisoxazol-4-yl, 3-methylisoxazole and 3-chloroisoxazol-4-yl.

An example of Het-14 is isoxazol-3-yl.

An example of Het-15 is isoxazol-5-yl.

Examples of Het-16 include oxazol-4-yl, 2-methyloxazol-4-yl and 2,5-dimethyloxazol-4-yl.

An example of Het-17 is oxazol-2-yl.

An example of Het-18 is oxazol-5-yl.

Examples of Het-19 include 4,5-dichloroimidazol-1-yl and 4,5-dimethylimidazol-1-yl.

An example of Het-20 is 1-methylimidazol-4-yl.

An example of Het-21 is 1-methylimidazol-2-yl.

An example of Het-22 is 1-methylimidazol-5-yl.

Examples of Het-23 include 3-chloro-1,2,4-triazol-1-yl, 3-fluoro-1,2,4-triazol-1-yl, 3-bromo-1,2,4-triazol-1-yl, 3-trifluoromethyl-1,2,4-triazol-1-yl, 3,5-dimethyl-1,2,4-triazol-1-yl, 3,5-dichloro-1,2,4-triazol-1-yl, 3,5-dibromo-1,2,4-triazol-1-yl, 3,5-difluoro-1,2,4-triazol-1-yl and 3,5-ditrifluoromethyl-1,2,4-triazol-1-yl.

Examples of Het-24 include 4,5-dimethyl-1,2,3-triazol-1-yl, 4,5-dichloro-1,2,3-triazol-1-yl, 4,5-dibromo-1,2,3-triazol-1-yl, 4,5-difluoro-1,2,3-triazol-1-yl, 4,5-ditrifluoromethyl-1,2,3-triazol-1-yl, 5-methyl-1,2,3-triazol-1-yl, 5-chloro-1,2,3-triazol-1-yl, 5-fluoro-1,2,3-triazol-1-yl, 5-bromo-1,2,3-triazol-1-yl, 5-trifluoromethyl-1,2,3-triazol-1-yl.

An example of Het-25 is 1,2,3-triazol-2-yl.

An example of Het-26 is 1-methyl-1,2,4-triazol-5-yl.

An example of Het-27 is 1-methyl-1,2,3-triazol-5-yl.

An example of Het-28 is 2-methyl-1,2,3-triazol-4-yl.

An example of Het-29 is 1-methyl-1,2,4-triazol-3-yl.

An example of Het-30 is 1-methyl-1,2,3-triazol-4-yl.

An example of Het-31 is 2-methyl-1,2,3-triazol-5-yl.

According to a further preferred embodiment of the invention, R⁴ is thienyl which is unsubstituted or has 1, 2 or 3 substituents R^{c}. Accordingly, R⁴ is one of the radicals Het-32 or Het-33 below in which # denotes the point of attachment and L¹, L², and L³ independently of one another have the meanings given above for formulae Het-1 to Het-31.

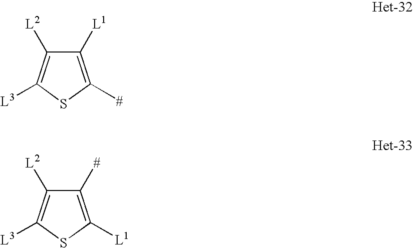

Examples of Het-32 are 2-thienyl, 5-methylthiophen-2-yl, 5-chlorothiophen-2-yl, 5-bromothiophen-2-yl, 3,5-dichlorothiophen-2-yl, 3,4,5-trichlorothiophen-2-yl and 5-bromothiophen-2-yl.

Examples of Het-33 are 3-thienyl, 2-methylthiophen-3-yl, 2,5-dichlorothiophen-3-yl, 2,4,5-trichlorothiophen-3-yl and 2,5-dibromothiophen-3-yl.

A further embodiment of the invention relates to compounds of the formula I in which R⁴ is 1,2,3-triazolyl or 1,2,4-triazolyl, where R⁴ may have two identical or different substituents R^{c}.

Preferred embodiments for these R⁴ are 3,5-dimethyl-1,2,4-triazol-1-yl, 3,5-dichloro-1,2,4-triazol-1-yl, 3,5-dibromo-1,2,4-triazol-1-yl, 3,5-difluoro-1,2,4-triazol-1-yl, 3,5-di-(trifluoromethyl)-1,2,4-triazol-1-yl, 3-methyl-1,2,4-triazol-1-yl, 3-chloro-1,2,4-triazol-1-yl, 3-fluoro-1,2,4-triazol-1-yl, 3-bromo-1,2,4-triazol-1-yl, 3-trifluoromethyl-1,2,4-triazol-1-yl, 4,5-dimethyl-1,2,3-triazol-1-yl, 4,5-dichloro-1,2,3-triazol-1-yl, 4,5-dibromo-1,2,3-triazol-1-yl, 4,5-difluoro-1,2,3-triazol-1-yl, 4,5-di(trifluoromethyl)-1,2,3-triazol-1-yl, 5-methyl-1,2,3-triazol-1-yl, 5-chloro-1,2,3-triazol-1-yl, 5-fluoro-1,2,3-triazol-1-yl, 5-bromo-1,2,3-triazol-1-yl, 5-trifluoromethyl-1,2,3-triazol-1-yl.

In a further embodiment of the compounds I, R⁴ is a five-membered nitrogenous heteroaromatic ring selected from the group consisting of pyrrolyl, pyrazolyl, imidazolyl, oxazolyl, isoxazolyl, thiazolyl and isothiazolyl, where R⁴ is unsubstituted or substituted by one, two, three or four identical or different substituents R^{c}. According to one embodiment, R⁴ is here unsubstituted or substituted pyrrolyl, pyrazolyl, imidazolyl, oxazolyl, isoxazolyl or isothiazolyl.

According to one embodiment of the invention, R⁴ is optionally substituted pyrrolyl, preferably pyrrolyl which is attached via a ring carbon to the skeleton of the formula I, i.e. 2- or 3-pyrrolyl, particularly preferably 2-pyrrolyl. Preferably, the pyrrolyl radical has one, two, three or four substituents R^{c}. Preferably, one substituent is located at the nitrogen atom of the pyrrolyl ring.

According to a further embodiment of the invention, R⁴ is optionally substituted pyrazolyl, such as 1-, 3-, 4- or 5-pyrazolyl. Particularly preferably, R⁴ is 1-pyrazolyl or 4-pyrazolyl, more preferably 1-pyrazolyl. Preferably, the pyrazolyl radical has one, two or three substituents R^{c}.

According to one embodiment of the invention, R⁴ is optionally substituted imidazolyl, i.e. 1-, 2- or 4-imidazolyl, particularly preferably 1- or 2-imidazolyl. Preferably, the imidazolyl radical has one, two or three substituents R^{c}.

—Preferred substitution patterns of R⁴ are as stated in Table A below, where L¹, L² and L³ within a heteroaromatic radical R⁴ are each identical or different R^{c}, which are as defined above:

TABLE A

| | | |
|---|---|---|
| 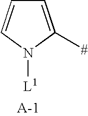 | 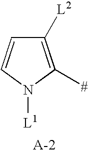 | 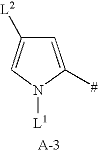 |
| 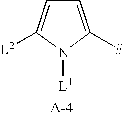 | 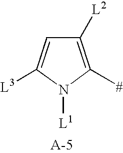 | 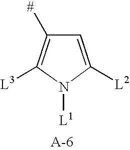 |
| 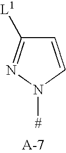 | 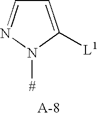 | 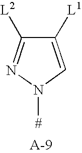 |
| 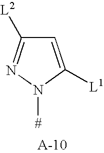 | 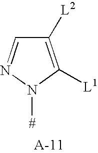 | 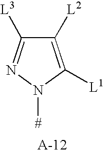 |
| 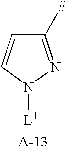 | 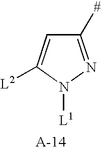 | 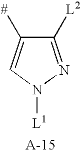 |
| 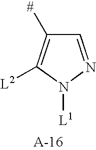 | 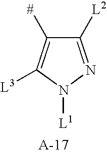 | 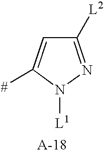 |
| 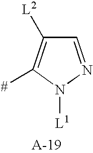 | 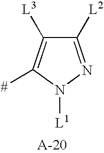 | 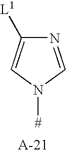 |
| 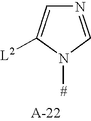 | 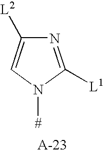 | 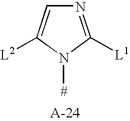 |
| 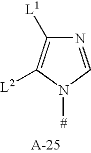 | 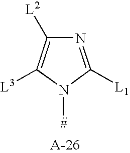 | 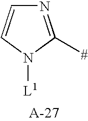 |
| 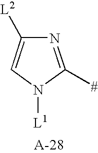 | 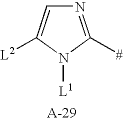 | 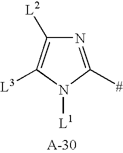 |
| 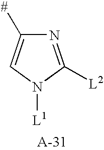 | 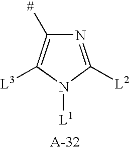 | 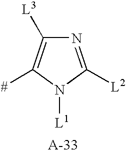 |
| 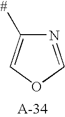 | 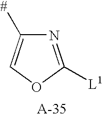 | 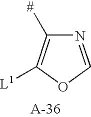 |
| 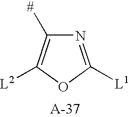 | 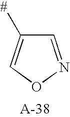 | 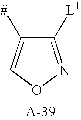 |
| 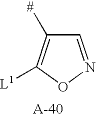 | 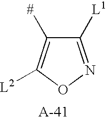 | 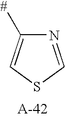 |
| 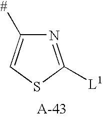 | 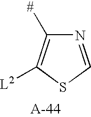 | 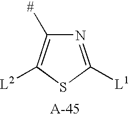 |
| 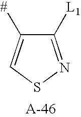 | 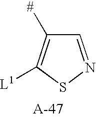 | 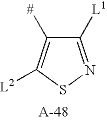 |

In the structures in Table A, # denotes in each case the point of attachment of the respective R⁴ to the skeleton of the compounds of the formula I or to the precursors thereof. L¹, L², L³ each independently have the meanings given here for R^{c}. If R⁴ is pyrrolyl, particular preference is given to a substitution pattern selected from the group consisting of A-1, A-2, A-3, A-4 and A-5, in particular selected from the group consisting of A-2 and A-4, according to Table A.

If R⁴ is pyrazolyl, a substitution pattern is selected from the group consisting of A-7, A-8, A-10, A-11, A-13, A-14, A-15, A-16 and A-19. Particularly preferred is A-10, in particular with L¹=L².

If R⁴ is imidazolyl, a substitution pattern is selected from the group consisting of A-21, A-22, A-23, A-24, A-25, A-26, A-27, A-28, A-29 and A-30, in particular selected from the group consisting of A-22, A-23, A-24, A-25 and A-27, A-28, and A-29.

If R⁴ is oxazolyl, particular preference is given to a substitution pattern selected from the group consisting of A-36 and A-37.

If R⁴ is isoxazolyl, particular preference is given to a substitution pattern selected from the group consisting of A-39, A-40 and A-41.

If R⁴ is thiazolyl, particular preference is given to a substitution pattern selected from the group consisting of A-44 and A-45.

If R⁴ is isothiazolyl, particular preference is given to a substitution pattern selected from the group consisting of A-46 and A-47.

Specific embodiments of R⁴ are: 1-methylpyrrol-2-yl, 1,4-dimethylpyrrol-2-yl, 1-methyl-5-chloropyrrol-2-yl, 1-methyl-3,5-dichloropyrrol-2-yl, 3-methyl-5-isopropylpyrazol-1-yl, 3,5-dimethylpyrazol-1-yl, 3-isopropyl-5-methylpyrazol-1-yl, 3-ethyl-5-methylpyrazol-1-yl, 3-methyl-5-methoxypyrazol-1-yl, 3,4,5-trimethylpyrazol-1-yl, 3,5-dimethyl-4-chloropyrazol-1-yl, 3-chloropyrazol-1-yl, 3,4-dichloro-5-trichloromethylpyrazol-1-yl, 1,3-methylpyrazol-1-yl, 3,5-dichloro-4-methylpyrazol-1-yl, 3-methyl-4-chloropyrazol-1-yl, 1,3-dimethylpyrazol-5-yl, 1-methyl-3-trifluoromethylpyrazol-5-yl, 1,5-dimethylpyrazol-3-yl, 1-methyl-5-methoxypyrazol-3-yl, 1,3,5-trimethylpyrazol-4-yl, 1-methyl-3-trifluoromethylpyrazol-4-yl, 1,3-dimethylpyrazol-4-yl, 1-methyl-5-trifluoromethylpyrazol-4-yl, 1,5-dimethylpyrazol-4-yl, 3,5-dichloropyrazol-1-yl, 3,5-dibromopyrazol-1-yl, 5-chloropyrazol-1-yl, 5-bromopyrazol-1-yl, 3,4,5-trichloropyrazol-1-yl, 3,4,5-tribromopyrazol-1-yl, 1-methyl-3,5-dichloropyrazol-4-yl, 1-methyl-3,5-dibromopyrazol-4-yl, 4,5-dichloroimidazol-1-yl, 4,5-dimethylimidazol-1-yl, oxazol-4-yl, 2,5-dimethyloxazol-4-yl, 2-methyloxazol-4-yl, isoxazol-4-yl, 3,5-dimethylisoxazol-4-yl, 3-chloroisoxazol-4-yl, 3-methylisoxazol-4-yl, 2-methylthiazol-4-yl, thiazol-4-yl, 2-methyl-5-chlorothiazol-4-yl, 2,5-dichlorothiazol-4-yl, 2-methyl-5-bromothiazol-4-yl, 3-methylisothiazol-4-yl, 3-methyl-5-chloroisothiazol-4-yl.

According to a further embodiment of the invention, R⁴ is a five-membered heteroaryl group which contains two nitrogen atoms as ring members and is selected from the group consisting of 1,2,3-thiadiazolyl, 1,2,4-thiadiazolyl, 1,3,4-thiadiazolyl, 1,2,5-thiadiazolyl, 1,2,4-oxadiazolyl, 1,3,4-oxadiazolyl and 1,2,5-oxadiazolyl, where R⁴ may be unsubstituted or substituted by one or two R^{c}, where R^{c} is defined or preferably defined as above.

Specific meanings of such R⁴ are: 1,3,4-thiadiazol-2-yl, 5-methyl-[1,3,4]-thiadiazol-2-yl, 5-chloro-[1,3,4]-thiadiazol-2-yl, 5-bromo-[1,3,4]-thiadiazol-2-yl, 1,2,3-thiadiazol-4-yl, 5-methyl-[1,2,3]-thiadiazol-4-yl, 5-chloro-[1,2,3]-thiadiazol-4-yl, 5-bromo-[1,2,3]-thiadiazol-4-yl, 1,2,3-thiadiazol-5-yl, 4-methyl-[1,2,3]-thiadiazol-5-yl, 4-chloro-[1,2,3]-thiadiazol-5-yl, 4-bromo-[1,2,3]-thiadiazol-5-yl, 3-methyl-[1,2,4]-thiadiazol-5-yl, 3-chloro-[1,2,4]-thiadiazol-5-yl, 3-bromo-[1,2,4]-thiadiazol-5-yl, 1,2,4-thiadiazol-3-yl, 5-methyl-[1,2,4]-thiadiazol-3-yl, 5-chloro-[1,2,4]-thiadiazol-3-yl, 5-bromo-[1,2,4]-thiadiazol-3-yl, 1,2,5-thiadiazol-3-yl, 4-methyl-[1,2,5]-thiadiazol-3-yl, 4-chloro-[1,2,5]-thiadiazol-3-yl, 4-bromo-[1,2,5]-thiadiazol-3-yl, 1,3,4-thiadiazol-2-yl, 5-methyl-[1,3,4]-thiadiazol-2-yl, 5-chloro-[1,3,4]-thiadiazol-2-yl, 5-bromo-[1,3,4]-thiadiazol-2-yl, 1,2,3-thiadiazol-4-yl, 5-methyl-[1,2,3]-thiadiazol-4-yl, 5-chloro-1,2,3′-thiadiazol-4-yl, 5-bromo-[1,2,3]-thiadiazol-4-yl, 1,2,3-thiadiazol-5-yl, 4-methyl-[1,2,3]-thiadiazol-5-yl, 4-chloro-[1,2,3]-thiadiazol-5-yl, 4-bromo-[1,2,3]-thiadiazol-5-yl, 1,2,4-thiadiazol-5-yl, 3-methyl-[1,2,4]-thiadiazol-5-yl, 3-chloro-[1,2,4]-thiadiazol-5-yl, 3-bromo-1,2,4-thiadiazol-5-yl, 1,2,4-thiadiazol-3-yl, 5-methyl-[1,2,4]-thiadiazol-3-yl, 5-chloro-[1,2,4]-thiadiazol-3-yl, 5-bromo-[1,2,4]-thiadiazol-3-yl, 1,2,5-thiadiazol-3-yl, 4-methyl-[1,2,5]-thiadiazol-3-yl, 4-chloro-[1,2,5]-thiadiazol-3-yl, 4-bromo-[1,2,5]-thiadiazol-3-yl, 1,3,4-thiadiazol-2-yl, 5-methyl-[1,3,4]-thiadiazol-2-yl, 5-chloro-[1,3,4]-thiadiazol-2-yl, 5-bromo-[1,3,4]-thiadiazol-2-yl, 1,2,3-thiadiazol-4-yl, 5-methyl-[1,2,3]-thiadiazol-4-yl, 5-chloro-[1,2,3]-thiadiazol-4-yl, 5-bromo-[1,2,3]-thiadiazol-4-yl, 1,2,3-thiadiazol-5-yl, 4-methyl-[1,2,3]-thiadiazol-5-yl, 4-chloro-[1,2,3]-thiadiazol-5-yl, 4-bromo-[1,2,3]-thiadiazol-5-yl, 1,2,4-thiadiazol-5-yl, 3-methyl-[1,2,4]-thiadiazol-5-yl, 3-chloro-[1,2,4]-thiadiazol-5-yl, 3-bromo-[1,2,4]-thiadiazol-5-yl, 1,2,4-thiadiazol-3-yl, 5-methyl-[1,2,4]-thiadiazol-3-yl, 5-chloro-[1,2,4]-thiadiazol-3-yl, 5-bromo-[1,2,4]-thiadiazol-3-yl, 1,2,5-thiadiazol-3-yl, 4-methyl-[1,2,5]-thiadiazol-3-yl, 4-chloro-[1,2,5]-thiadiazol-3-yl, 4-bromo-[1,2,5]-thiadiazol-3-yl, 1,3,4-thiadiazol-2-yl, 5-methyl-1,3,4′-thiadiazol-2-yl, 5-chloro-[1,3,4]-thiadiazol-2-yl, 5-bromo-[1,3,4]-thiadiazol-2-yl, 1,2,3-thiadiazol-4-yl, 5-methy-[1,2,3]-thiadiazol-4-yl, 5-chloro-[1,2,3]-thiadiazol-4-yl, 5-bromo-[1,2,3]-thiadiazol-4-yl, 1,2,3-thiadiazol-5-yl, 4-methyl-[1,2,3]-thiadiazol-5-yl, 4-chloro-[1,2,3]-thiadiazol-5-yl, 4-bromo-[1,2,3]-thiadiazol-5-yl, 1,2,4-thiadiazol-5-yl, 3-methyl-[1,2,4]-thiadiazol-5-yl, 3-chloro-[1,2,4]-thiadiazol-5-yl, 3-bromo-[1,2,4]-thiadiazol-5-yl, 1,2,4-thiadiazol-3-yl, 5-methyl-[1,2,4]-thiadiazol-3-yl, 5-chloro-[1,2,4]-thiadiazol-3-yl, 5-bromo-[1,2,4]-thiadiazol-3-yl, 1,2,5-thiadiazol-3-yl, 4-methyl-[1,2,5]-thiadiazol-3-yl, 4-chloro-[1,2,5]-thiadiazol-3-yl, 4-bromo-[1,2,5]-thiadiazol-3-yl, 1,3,4-thiadiazol-2-yl, 5-methyl-[1,3,4]-thiadiazol-2-yl, 5-chloro-[1,3,4]-thiadiazol-2-yl, 5-bromo-[1,3,4]-thiadiazol-2-yl, 1,2,3-thiadiazol-4-yl, 5-methyl-[1,2,3]-thiadiazol-4-yl, 5-chloro-[1,2,3]-thiadiazol-4-yl, 5-bromo-[1,2,3]-thiadiazol-4-yl, 1,2,3-thiadiazol-5-yl, 4-methyl-[1,2,3]-thiadiazol-5-yl, 4-chloro-[1,2,3]-thiadiazol-5-yl, 4-bromo-[1,2,3]-thiadiazol-5-yl, 1,2,4-thiadiazol-5-yl, 3-methyl-[1,2,4]-thiadiazol-5-yl, 3-chloro-[1,2,4]-thiadiazol-5-yl, 3-bromo-[1,2,4]-thiadiazol-5-yl, 1,2,4-thiadiazol-3-yl, 5-methyl-[1,2,4]-thiadiazol-3-yl, 5-chloro-[1,2,4]-thiadiazol-3-yl, 5-bromo-[1,2,4]-thiadiazol-3-yl, 1,2,5-thiadiazol-3-yl, 4-methyl-[1,2,5]-thiadiazol-3-yl, 4-chloro-[1,2,5]-thiadiazol-3-yl, 4-bromo-[1,2,5]-thiadiazol-3-yl, 1,3,4-thiadiazol-2-yl, 5-methyl-[1,3,4]-thiadiazol-2-yl, 5-chloro-[1,3,4]-thiadiazol-2-yl, 5-bromo-[1,3,4]-thiadiazol-2-yl, 1,2,3-thiadiazol-4-yl, 5-methyl-[1,2,3]-thiadiazol-4-yl, 5-chloro-[1,2,3]-thiadiazol-4-yl, 5-bromo-[1,2,3]-thiadiazol-4-yl, 1,2,3-thiadiazol-5-yl, 4-methyl-[1,2,3]-thiadiazol-5-yl, 4-chloro-[1,2,3]-thiadiazol-5-yl, 4-bromo-[1,2,3]-thiadiazol-5-yl, 1,2,4-thiadiazol-5-yl, 3-methyl-[1,2,4]-thiadiazol-5-yl, 3-chloro-[1,2,4]-thiadiazol-5-yl, 3-bromo-[1,2,4]-thiadiazol-5-yl, 1,2,4-thiadiazol-3-yl, 5-methyl-[1,2,4]-thiadiazol-3-yl, 5-chloro-[1,2,4]-thiadiazol-3-yl, 5-bromo-[1,2,4]-thiadiazol-3-yl, 1,2,5-thiadiazol-3-yl, 4-methyl-[1,2,5]-thiadiazol-3-yl, yl, 4-bromo-[1,2,5]-thiadiazol-3-yl.

Here, in a preferred embodiment of the invention, R⁴ is unsubstituted. In a further preferred embodiment, R⁴ has one, two, three or four, preferably one or two, identical or different substituents R^{c}.

One aspect of this embodiment of the invention relates to compounds of the formula I in which R⁴ is a 6-membered heteroaromatic radical which has 1, 2 or 3 nitrogen atoms as ring members and which is unsubstituted or carries 1, 2, 3 or 4, preferably 1, 2 or 3, substituents R^{c}. In this embodiment, R⁴ is preferably pyridinyl, pyrimidinyl, pyrazinyl, pyridazinyl or triazinyl (1,2,4-triazinyl or 1,3,5-triazinyl), in particular pyridinyl, pyrimidinyl or pyrazinyl. In a further preferred embodiment, R⁴ is selected from the group consisting of pyridinyl, pyridazinyl, pyrazinyl, 1,2,4-triazinyl and 1,3,5-triazinyl. In a further preferred embodiment, R⁴ is pyrimidyl. Here, the R⁴ are unsubstituted or carry 1, 2, 3 or 4 independently selected substituents R^{c}.

From among the compounds of this embodiment, preference is given to compounds of the general formula I in which R⁴ is pyridinyl which optionally has 1, 2, 3 or 4 substituents R^{c}. From among these, particular preference is given to compounds of the formula I in which R⁴ is 2-pyridinyl which has 1 or 2 substituents R^{c}. From among these, very particular preference is given to compounds in which one of the substituents R^{c} is located in the 5-position of the pyridinyl ring. From among these, very particular preference is furthermore given to compounds I in which one of the substituents R^{c} is located in the 3-position of the pyridinyl ring. Here, R^{c} has in particular the meanings given as being preferred.

From among the compounds of this embodiment, preference is furthermore given to compounds of the formula I in which R⁴ is 3-pyridinyl which optionally has 1 or 2 substituents R^{c}. From among these, preference is given to those compounds which have a substituent R^{c} in the 2-position and/or a substituent R^{c} in the 4-position of the pyridine ring.

From among the compounds of this embodiment, preference is furthermore given to compounds of the formula I in which R⁴ is 4-pyridinyl which optionally has 1 or 2 substituents R^{c}. From among these, preference is given to those compounds which have a substituent R^{c} in the 3-position and/or a substituent R^{c} in the 5-position of the pyridine ring.

From among the compounds of this embodiment, preference is furthermore given to compounds of the formula I in which R⁴ is pyrimidinyl and in particular 2- or 4-pyrimidinyl which optionally has 1, 2 or 3 substituents R^{c}. From among these, particular preference is given to compounds of the formula I in which R⁴ is 2-pyrimidinyl or 4-pyrimidinyl which has 1 or 2 substituents R^{c}. From among these, particular preference is given to those compounds in which one of the substituents R^{c} is located in the 5-position of the pyrimidinyl ring. Here, R^{c} has in particular the meanings mentioned as being preferred.

A further preferred embodiment of the invention relates to compounds of the formula I in which R⁴ is 2-pyrazinyl which optionally has 1, 2 or 3 substituents

A further preferred embodiment of the invention relates to compounds of the formula I in which R⁴ is 3-pyridazinyl which optionally has 1, 2 or 3 substituents R^{c}.

A further preferred embodiment of the invention relates to compounds of the formula I in which R⁴ is 1,3,5-triazinyl which optionally has 1 or 2 substituents R^{c}.

Examples of particularly preferred heterocyclic radicals R⁴ of this embodiment are the radicals Het-34 to Het-39 shown below:

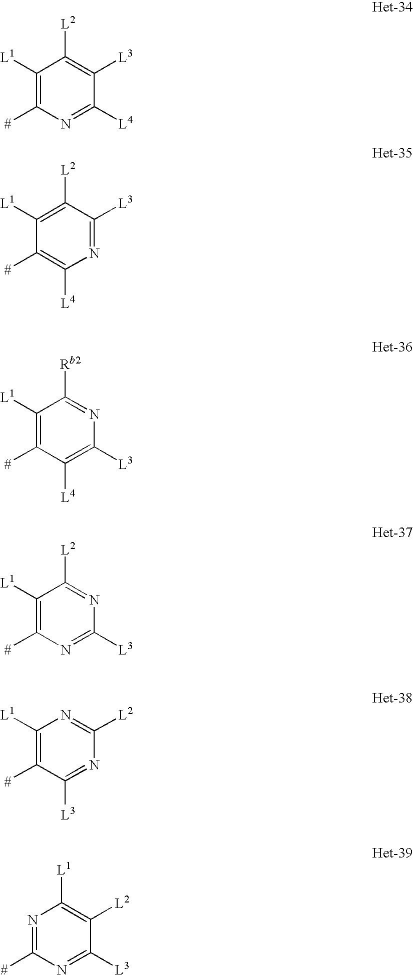

in which
# denotes the point of attachment; and
L¹, L², L³ and L⁴ independently of one another are hydrogen or have one of the meanings mentioned for R^{c}. Preferably, the radicals L¹, L², L³ and L⁴ independently of one another are selected from the group consisting of hydrogen, halogen, nitro, cyano, C₁-C₄-alkyl, C₁-C₄-haloalkyl, especially C₁-C₂-fluoroalkyl, C₁-C₄-alkoxy and C₁-C₄-alkoxycarbonyl. In particularly preferred embodiments, L¹, L², L³ and L⁴ independently of one another are selected from the group consisting of hydrogen, nitro, cyano, fluorine, chlorine, bromine, methyl, ethyl, isopropyl, trifluoromethyl, fluoromethyl, methoxy and methoxycarbonyl.

Examples of Het-34 are 2-pyridyl, 3-fluoropyridin-2-yl, 3-chloropyridin-2-yl, 3-bromo-2-pyridin-2-yl, 3-trifluoromethylpyridin-2-yl, 3-methylpyridin-2-yl, 3-ethylpyridin-2-yl 3,5-difluoropyridin-2-yl, 3,5-dichloropyridin-2-yl, 3,5-dibromopyridin-2-yl, 3-fluoro-5-trifluoromethylpyridin-2-yl, 3,5-dimethylpyridin-2-yl, 5-nitropyridin-2-yl, 5-cyanopyridin-2-yl, 5-methoxycarbonylpyridin-2-yl, 5-trifluoromethylpyridin-2-yl, 5-methylpyridin-2-yl, 4-methylpyridin-2-yl, and 6-methylpyridin-2-yl.

Examples of Het-35 are 3-pyridyl, 2-chloropyridin-3-yl, 2-bromopyridin-2-yl, 2-methylpyridin-3-yl, 2,4-dichloropyridin-3-yl, 2,4-dibromopyridin-3-yl, 2,4-dimethylpyridin-3-yl, 2,4,6-trichloropyridin-3-yl, 2,4,6-tribromopyridin-3-yl, 2,4,6-trimethylpyridin-3-yl and 2,4-dichloro-6-methylpyridin-3-yl.

Examples of Het-36 include 4-pyridyl, 3-chloropyridin-4-yl, 3-bromopyridin-4-yl, 3-methylpyridin-4-yl, 3,5-dichloropyridin-4-yl, 3,5-dibromopyridin-4-yl and 3,5-dimethylpyridin-4-yl.

Examples of Het-37 include 5-chloropyrimidin-4-yl, 5-fluoropyrimidin-4-yl, 5-fluoro-6-chloropyrimidin-4-yl, 2-methyl-6-trifluoromethylpyrimidin-4-yl, 2,5-dimethyl-6-trifluoromethylpyrimidin-4-yl, 5-methyl-6-trifluoromethylpyrimidin-4-yl, 6-trifluoromethylpyrimidin-4-yl, 2-methyl-5-fluoropyrimidin-4-yl, 2-methyl-5-chloropyrimidin-4-yl, 5-chloro-6-methylpyrimidin-4-yl, 5-chloro-6-ethylpyrimidin-4-yl, 5-chloro-6-isopropylpyrimidin-4-yl, 5-bromo-6-methylpyrimidin-4-yl, 5-fluoro-6-methylpyrimidin-4-yl, 5-fluoro-6-fluoromethylpyrimidin-4-yl, 2,6-dimethyl-5-chloropyrimidin-4-yl, 5,6-dimethylpyrimidin-4-yl, 2,5-dimethylpyrimidin-4-yl, 2,5,6-trimethylpyrimidin-4-yl and 5-methyl-6-methoxypyrimidin-4-yl.

Examples of Het-38 include 4-methylpyrimidin-5-yl, 4,6-dimethylpyrimidin-5-yl, 2,4,6-trimethylpyrimidin-5-yl and 4-trifluoromethyl-6-methylpyrimidin-5-yl.

Examples of Het-39 include 4,6-dimethylpyrimidin-2-yl, 4,5,6-trimethylpyrimidin-2-yl, 4,6-ditrifluoromethylpyrimidin-2-yl and 4,6-dimethyl-5-chloropyrimidin-2-yl.

According to one embodiment of the invention, R⁵ is C₁-C₄-alkyl, in particular methyl, ethyl, isopropyl or n-propyl. According to one special aspect, R⁵ is methyl.

According to a further embodiment, R⁵ is C₁-C₄-haloalkyl, in particular halomethyl.

According to a further embodiment, R⁵ is halogen, in particular chlorine or fluorine.

According to one embodiment of the invention, preference is given to compounds of the formula I in which E is N and A, B and D are as defined above.

According to a further embodiment, at least two of the variables A, B, D and E are N. According to one aspect, two of the variables are N, according to a further aspect, three of the variables are N.

In the compounds of the formula I, R⁶ is in each case independently preferably hydrogen, halogen, C₁-C₄-alkyl, C₁-C₄-alkoxy, C₁-C₄-haloalkyl or C₁-C₄-haloalkoxy, in particular hydrogen, halogen or C₁-C₄-alkyl. According to a preferred embodiment, all R⁶ are hydrogen.

According to one embodiment of the invention, preference is given to compounds Ia

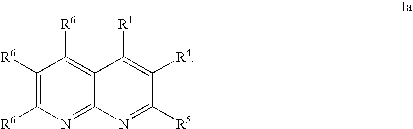

According to a further embodiment of the invention, preference is given to compounds Ib

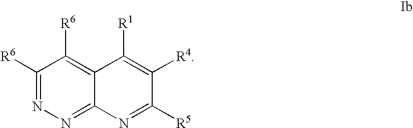

According to a further embodiment of the invention, preference is given to compounds Ic

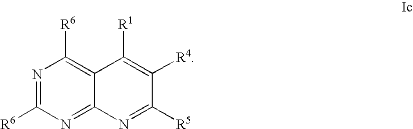

According to a further embodiment of the invention, preference is given to compounds Id

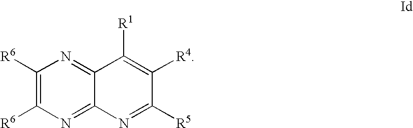

According to a further embodiment of the invention, preference is given to compounds Ie

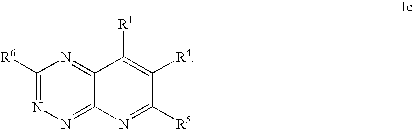

According to a further embodiment of the invention, preference is given to compounds If

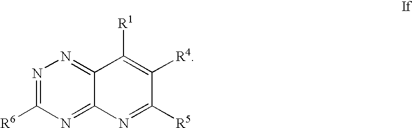

Here, the substituents R¹, R⁴, R⁵ and R⁶ in the formulae Ia, Ib, Ic, Id, Ie and If have in each case independently the meanings or preferred meanings described elsewhere herein for these radicals for the compounds of the formula I according to the invention. According to one embodiment, in each case all R⁶ are hydrogen; herein, these compounds are also referred to as compounds of the formula Ia.1, Ib.1, Ic.1, Id.1, Ie.1 and If.1, respectively.

Some of the compounds of the formula I used as component 1), their preparation and their activity against harmful fungi are, as mentioned above, known from the literature (cf., for example, WO 2004/056824, WO2004/056825, WO 2004/056826, WO 2004/056829, WO 2005/123698, WO 2005/123733), or they can be prepared in the manner described therein.

The active compounds II mentioned above as component 2), their preparation and their activity against harmful fungi are generally known, and most are commercially available (see above).

The compounds I can be used as synergists for a large number of different fungicidally active compounds. Simultaneous joint or separate application of a compound I/compounds I with at least one active compound II results in a superadditive increase of the fungicidal activity.

According to one embodiment of the invention, in the compositions according to the invention, the components 1) used are in particular compounds of the formula I in which the substituents have the meanings already described above for the compounds of the formula I according to the invention. Further embodiments of the compositions according to the invention are described below:

According to a further preferred embodiment of the inventive compositions, the components 1) used are compounds of the formula I in which R¹ is NR²R³, where R² and R³ are as defined above.

Here, according to one aspect of the invention, R² is C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₃-C₈-cycloalkyl, C₃-C₈-halocycloalkyl, C₃-C₈-cycloalkyl-C₁-C₈-alkyl, C₆-C₁₄-aryl, C₆-C₁₄-aryl-C₁-C₈-alkyl, five- or six-membered heteroaryl or five- or six-membered heteroaryl-C₁-C₈-alkyl. According to a further aspect, R³ is hydrogen, NR⁷R⁸ or one of the groups mentioned for R², where R⁷ and R⁸ independently of one another are hydrogen or have one of the meanings mentioned for R². The alkyl, alkenyl, alkynyl and cycloalkyl groups in R² and/or R³ may for their part carry one, two or three identical or different groups R^{d}, where R^{d} is in each case independently selected from the group consisting of halogen, cyano, C₁-C₈-alkoxy, C₁-C₈-alkyl, C₁-C₈-haloalkoxy, C₁-C₈-alkylcarbonyl, C₁-C₈-alkoxycarbonyl, C₁-C₈-alkylthio, tri-C₁-C₆-alkylsilyl, C₁-C₈-alkylamino and di-C₁-C₈-alkylamino.

According to one aspect of this embodiment, R² is C₁-C₈-alkyl, C₁-C₈-haloalkyl, C₂-C₈-alkenyl, C₂-C₈-haloalkenyl, C₃-C₈-cycloalkyl, C₃-C₈-halocycloalkyl or C₃-C₈-cycloalkyl-C₁-C₈-alkyl. According to a further aspect, R³ is hydrogen or one of the groups mentioned for R².

According to a further aspect, neither R² nor R³ is hydrogen, where R² and R³ are preferably independently of one another selected from the group consisting of C₁-C₆-alkyl, C₁-C₆-haloalkyl, C₂-C₆-alkenyl, C₂-C₆-haloalkenyl, C₃-C₆-cycloalkyl and C₃-C₆-halocycloalkyl, in particular selected from the group consisting of C₁-C₆-alkyl and C₁-C₆-haloalkyl.

According to yet another aspect, R² is as defined above or as defined as being preferred and R³ is hydrogen.

Here, R² and R³ may each independently of one another carry one, two or three identical or different groups R^{d}. R^{d} is as defined above and is preferably in each case independently halogen, cyano, C₁-C₄-haloalkyl, C₁-C₄-alkoxy or C₁-C₄-haloalkoxy.

According to special embodiments of the present invention, as component 1) use is made of compounds of the formula I in which R² is a group B:

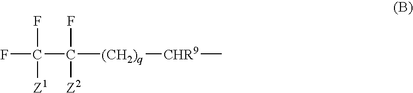

in which
Z¹ is hydrogen, fluorine or C₁-C₄-fluoroalkyl,
Z² is hydrogen or fluorine, or
Z¹ and Z² together form a double bond;
q is 0 or 1; and
R⁹ is hydrogen or methyl. According to a special aspect of this embodiment, R³ is hydrogen.

According to a further aspect of the invention, R³ in the compounds of the formula I is hydrogen and R² is C₃-C₆-cycloalkyl which may be substituted by C₁-C₄-alkyl.

According to a further aspect of the invention, R³ in the compounds of the formula I is selected from the group consisting of methyl, ethyl, CH(CH₃)CH₂CH₃, CH₂CH(CH₃)₂, CH(CH₃)CH(CH₃)₂, CH(CH₃)C(CH₃)₃, CH(CH₃)CF₃, CH(CH₃)CF₃, CH(CH₃)CCl₃, CH₂CF₂CF₃, CH₂C(CH₃)═CH₂, CH₂CH═CH₂, cyclopentyl, cyclohexyl, benzyl; and R² is hydrogen or methyl.

According to a further aspect of this embodiment, R² and R³ together with the nitrogen atom to which they are attached form a five- or six-membered saturated or partially unsaturated heterocycle which may contain one or two further heteroatoms from the group consisting of O, N and S as ring members, where the heterocycle may carry one, two or three identical or different groups R^{f}, as defined above. Preferably, R^{f} is in each case independently selected from the group consisting of halogen, cyano, C₁-C₄-alkyl, C₁-C₄-haloalkyl, C₁-C₄-alkoxy and C₁-C₄-haloalkoxy.

More preferably, R^{f} is in each case independently selected from the group consisting of C₁-C₈-alkyl, C₁-C₈-haloalkyl, C₁-C₈-alkoxy and C₁-C₈-haloalkoxy.

According to one aspect of this embodiment of the invention, R² and R³ together with the nitrogen atom to which they are attached form a piperidinyl, morpholinyl or thiomorpholinyl ring, especially a piperidinyl ring, which is optionally substituted by one, two or three identical or different groups R^{f}.

The heterocycle formed is in particular unsubstituted or substituted by one, two or three R^{f}, where R^{f} is in each case independently selected from the group consisting of halogen, C₁-C₄-alkyl and C₁-C₄-haloalkyl. From among these, particular preference is given to compounds I in which R² and R³ together with the nitrogen atom to which they are attached form a 4-methylpiperidine ring, a 4-trifluoromethylpiperidine ring, a morpholine ring or a 3,4-dimethylpiperidine ring and especially a 4-methylpiperidine ring or a 3,4-dimethylpiperidine ring.

In the compositions according to the invention, the components 1) used are in particular compounds in which R⁵ has the meanings given above. According to a further embodiment of the compositions according to the invention, R⁵ in the compounds of the formula I is halogen, cyano, C₁-C₄-alkoxy, C₁-C₄-haloalkoxy, C₁-C₄-alkyl or C₁-C₄-haloalkyl.

Here, according to one aspect, R⁵ is C₁-C₄-alkyl, such as methyl, ethyl or n-propyl. Here, according to a further aspect, R⁵ is C₁-C₄-haloalkyl, such as, for example, fluoromethyl, chloromethyl, bromomethyl, difluoromethyl, dichloromethyl, trifluoromethyl, trichloromethyl, chlorofluoromethyl, dichlorofluoromethyl or chlorodifluoromethyl.

A further aspect relates to fungicidal compositions in which R⁵ in the compounds of the formula I is halogen, in particular chlorine.

According to one aspect, R⁵ in the compounds of the formula I is C₁-C₈-alkoxy, preferably C₁-C₄-alkoxy, in particular methoxy or ethoxy. More preferably, R⁵ is C₁-C₈-haloalkoxy, preferably C₁-C₄-haloalkoxy.

According to one aspect, R⁵ in the compounds of the formula I is cyano.

According to one embodiment of the compositions according to the invention, the components 1) used are compounds of the formula Ia, as shown above. According to one aspect of this embodiment, the components 1) used are compounds of the formula Ia.1, as described above.

According to a further embodiment of the compositions according to the invention, the components 1) used are compounds of the formula Ib, as shown above. According to one aspect of this embodiment, the components 1) used are compounds of the formula Ib.1, as described above.

According to one embodiment of the compositions according to the invention, the components 1) used are compounds of the formula Ic, as shown above. According to one aspect of this embodiment, the components 1) used are compounds of the formula Ic.1, as described above.

According to one embodiment of the compositions according to the invention, the components 1) used are compounds of the formula Id, as shown above. According to one aspect of this embodiment, the components 1) used are compounds of the formula Id.1, as described above.

According to one embodiment of the compositions according to the invention, the components 1) used are compounds of the formula Ie, as shown above. According to one aspect of this embodiment, the components 1) used are compounds of the formula Ie.1, as described above.

According to one embodiment of the compositions according to the invention, the components 1) used are compounds of the formula If, as shown above. According to one aspect of this embodiment, the components 1) used are compounds of the formula If.1, as described above.

The substituents in the compounds Ia, Ib, Ic, Id, Ie, If or Ia.1, Ib.1, Ic.1, Id.1, Ie.1, If.1 used as component 1) in the compositions according to the invention have in each case the meanings given elsewhere herein for the substituent in question according to the compounds according to the invention or for the component 1) in the compositions according to the invention.

According to a further special embodiment of the fungicidal compositions according to the invention, component 1) corresponds to a compound of the formula Ia-1:

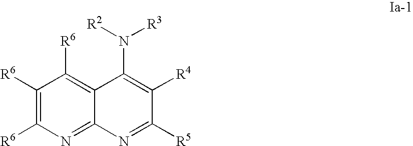

According to one aspect of this embodiment, the component 1) corresponds to a compound of the formula Ia-1 in which R⁶ is in each case hydrogen (compounds Ia.1-1).

According to a further special embodiment of the fungicidal compositions according to the invention, component 1) corresponds to a compound of the formula Ib-1:

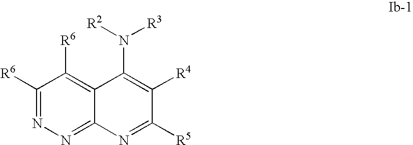

According to one aspect of this embodiment, the component 1) corresponds to a compound of the formula Ib-1 in which R⁶ is in each case hydrogen (compounds Ib.1-1).

According to a further special embodiment of the fungicidal compositions according to the invention, component 1) corresponds to a compound of the formula Ic-1:

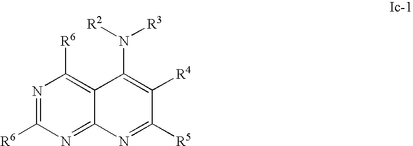

According to one aspect of this embodiment, the component 1) corresponds to a compound of the formula Ic-1 in which R⁶ is in each case hydrogen (compounds Ic.1-1).

According to a further special embodiment of the fungicidal compositions according to the invention, component 1) corresponds to a compound of the formula Id-1:

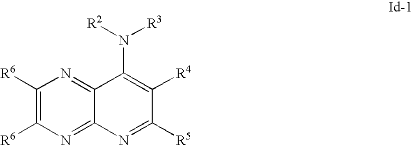

According to one aspect of this embodiment, the component 1) corresponds to a compound of the formula Id-1 in which R⁶ is in each case hydrogen (compounds Id.1-1).

According to a further special embodiment of the fungicidal compositions according to the invention, component 1) corresponds to a compound of the formula Ie-1:

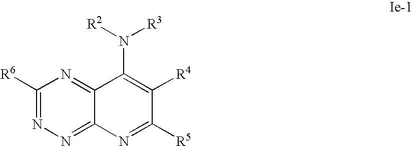

According to one aspect of this embodiment, the component 1) corresponds to a compound of the formula Ie-1 in which R⁶ is in each case hydrogen (compounds Ie.1-1).

According to a further special embodiment of the fungicidal compositions according to the invention, component 1) corresponds to a compound of the formula If-1:

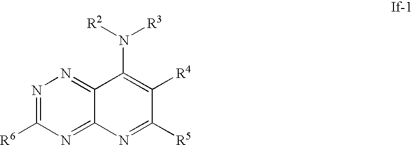

According to one aspect of this embodiment, the component 1) corresponds to a compound of the formula If-1 in which R⁶ is in each case hydrogen (compounds If.1-1).

The substituents in the compounds Ia-1, Ib-1, Ic-1, Id-1, Ie-1, If-1 or Ia.1-1, Ib.1-1, Ic.1-1, Id.1-1, Ie.1-1, If.1-1 used as component 1) in the compositions according to the invention have in each case the meanings given elsewhere herein for the corresponding substituent of the component 1).

In particular, the compounds I compiled in the Tables 1 to 110 below are preferably used in the compositions according to the invention. The groups mentioned for a substituent in the tables are furthermore per se, independently of the combination in which they are mentioned, a particularly preferred aspect of the substituent in question for a compound of the formula I. The compounds individually listed in these Tables 1 to 110 in combination with rows B-150 to B-157 and B-307 to B-314 of Table B are furthermore preferred compounds according to the invention and also form per se part of the subject matter of the present invention.

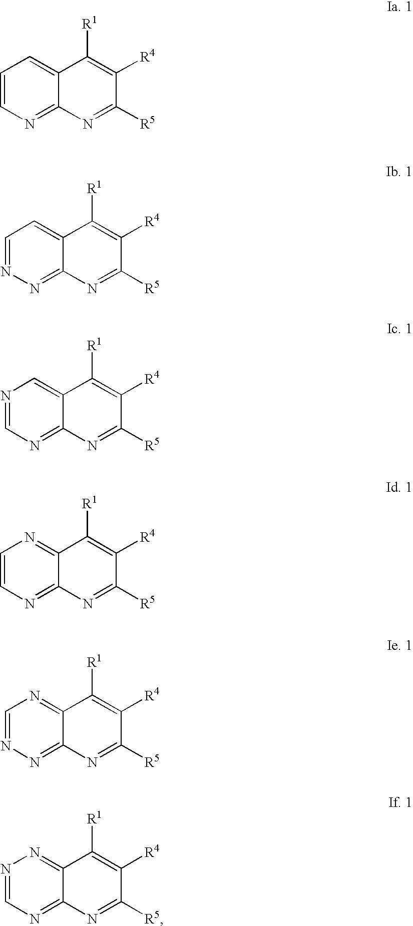

### Table 1

Compounds of the formulae Ia.1, Ib.1, Ic.1, Id.1, Ie.1 and If.1 in which R⁴ is 2,3,5,6-tetrafluorophenyl and the combination of R¹ and R⁵ for a compound corresponds in each case to one row of Table B

### Table 2

Compounds of the formulae Ia.1, Ib.1, Ic.1, Id.1, Ie.1 and If.1 in which R⁴ is 2-chloro-4,6-difluorophenyl and the combination of R¹ and R⁵ for a compound corresponds in each case to one row of Table B

### Table 3

Compounds of the formulae Ia.1, Ib.1, Ic.1, Id.1, Ie.1 and If.1 in which R⁴ is 2,3,6-trichlorophenyl and the combination of R¹ and R⁵ for a compound corresponds in each case to one row of Table B

### Table 4

Compounds of the formulae Ia.1, Ib.1, Ic.1, Id.1, Ie.1 and If.1 in which R⁴ is pentachlorophenyl and the combination of R¹ and R⁵ for a compound corresponds in each case to one row of Table B

### Table 5

Compounds of the formulae Ia.1, Ib.1, Ic.1, Id.1, Ie.1 and If.1 in which R⁴ is 2-fluoro-4,6-dichlorophenyl and the combination of R¹ and R⁵ for a compound corresponds in each case to one row of Table B

### Table 6

Compounds of the formulae Ia.1, Ib.1, Ic.1, Id.1, Ie.1 and If.1 in which R⁴ is 2-bromophenyl and the combination of R¹ and R⁵ for a compound corresponds in each case to one row of Table B

### Table 7

Compounds of the formulae Ia.1, Ib.1, Ic.1, Id.1, Ie.1 and If.1 in which R⁴ is 2-fluoro-6-bromophenyl and the combination of R¹ and R⁵ for a compound corresponds in each case to one row of Table B

### Table 8

Compounds of the formulae Ia.1, Ib.1, Ic.1, Id.1, Ie.1 and If.1 in which R⁴ is 2-bromo-4,6-difluorophenyl and the combination of R¹ and R⁵ for a compound corresponds in each case to one row of Table B

### Table 9

Compounds of the formulae Ia.1, Ib.1, Ic.1, Id.1, Ie.1 and If.1 in which R⁴ is 2-chloro-6-methylphenyl and the combination of R¹ and R⁵ for a compound corresponds in each case to one row of Table B

### Table 10

Compounds of the formulae Ia.1, Ib.1, Ic.1, Id.1, Ie.1 and If.1 in which R⁴ is 2-methoxyphenyl and the combination of R¹ and R⁵ for a compound corresponds in each case to one row of Table B

### Table 11

Compounds of the formulae Ia.1, Ib.1, Ic.1, Id.1, Ie.1 and If.1 in which R⁴ is 2,6-dimethoxyphenyl and the combination of R¹ and R⁵ for a compound corresponds in each case to one row of Table B

### Table 12

Compounds of the formulae Ia.1, Ib.1, Ic.1, Id.1, Ie.1 and If.1 in which R⁴ is 2-fluoro-6-methoxyphenyl and the combination of R¹ and R⁵ for a compound corresponds in each case to one row of Table B

### Table 13

Compounds of the formulae Ia.1, Ib.1, Ic.1, Id.1, Ie.1 and If.1 in which R⁴ is 2-fluoro-6-trifluoromethylphenyl and the combination of R¹ and R⁵ for a compound corresponds in each case to one row of Table B

### Table 14

Compounds of the formulae Ia.1, Ib.1, Ic.1, Id.1, Ie.1 and If.1 in which R⁴ is 2,6-di(trifluoromethyl)phenyl and the combination of R¹ and R⁵ for a compound corresponds in each case to one row of Table B

### Table 15

Compounds of the formulae Ia.1, Ib.1, Ic.1, Id.1, Ie.1 and If.1 in which R⁴ is 2-chloro-6-trifluoromethylphenyl and the combination of R¹ and R⁵ for a compound corresponds in each case to one row of Table B

### Table 16

Compounds of the formulae Ia.1, Ib.1, Ic.1, Id.1, Ie.1 and If.1 in which R⁴ is 2,4-difluoro-6-trifluoromethylphenyl and the combination of R¹ and R⁵ for a compound corresponds in each case to one row of Table B

### Table 17

Compounds of the formulae Ia.1, Ib.1, Ic.1, Id.1, Ie.1 and If.1 in which R⁴ is 2,4-difluoro-6-methoxyphenyl and the combination of R¹ and R⁵ for a compound corresponds in each case to one row of Table B

### Table 18

Compounds of the formulae Ia.1, Ib.1, Ic.1, Id.1, Ie.1 and If.1 in which R⁴ is 2,4-difluoro-6-methylphenyl and the combination of R¹ and R⁵ for a compound corresponds in each case to one row of Table B

### Table 19

Compounds of the formulae Ia.1, Ib.1, Ic.1, Id.1, Ie.1 and If.1 in which R⁴ is 2,4-difluoropyrid-3-yl and the combination of R¹ and R⁵ for a compound corresponds in each case to one row of Table B

### Table 20

Compounds of the formulae Ia.1, Ib.1, Ic.1, Id.1, Ie.1 and If.1 in which R⁴ is 3,5-difluoropyrid-4-yl and the combination of R¹ and R⁵ for a compound corresponds in each case to one row of Table B

### Table 21

Compounds of the formulae Ia.1, Ib.1, Ic.1, Id.1, Ie.1 and If.1 in which R⁴ is tetrafluoropyrid-4-yl and the combination of R¹ and R⁵ for a compound corresponds in each case to one row of Table B

### Table 22

Compounds of the formulae Ia.1, Ib.1, Ic.1, Id.1, Ie.1 and If.1 in which R⁴ is 3-fluoropyrid-2-yl and the combination of R¹ and R⁵ for a compound corresponds in each case to one row of Table B

### Table 23

Compounds of the formulae Ia.1, Ib.1, Ic.1, Id.1, Ie.1 and If.1 in which R⁴ is 4-fluoropyrid-3-yl and the combination of R¹ and R⁵ for a compound corresponds in each case to one row of Table B

### Table 24

Compounds of the formulae Ia.1, Ib.1, Ic.1, Id.1, Ie.1 and If.1 in which R⁴ is 3-fluoropyrid-4-yl and the combination of R¹ and R⁵ for a compound corresponds in each case to one row of Table B

### Table 25

Compounds of the formulae Ia.1, Ib.1, Ic.1, Id.1, Ie.1 and If.1 in which R⁴ is 2-fluoropyrid-3-yl and the combination of R¹ and R⁵ for a compound corresponds in each case to one row of Table B

### Table 26

Compounds of the formulae Ia.1, Ib.1, Ic.1, Id.1, Ie.1 and If.1 in which R⁴ is 2,4,6-trifluoropyrid-3-yl and the combination of R¹ and R⁵ for a compound corresponds in each case to one row of Table B

### Table 27

Compounds of the formulae Ia.1, Ib.1, Ic.1, Id.1, Ie.1 and If.1 in which R⁴ is 3,5-difluoropyrid-2-yl and the combination of R¹ and R⁵ for a compound corresponds in each case to one row of Table B

### Table 28

Compounds of the formulae Ia.1, Ib.1, Ic.1, Id.1, Ie.1 and If.1 in which R⁴ is 2,6-difluoropyrid-3-yl and die combination of R¹ and R⁵ for a compound corresponds in each case to one row of Table B

### Table 29

Compounds of the formulae Ia.1, Ib.1, Ic.1, Id.1, Ie.1 and If.1 in which R⁴ is 2,4-difluoro-6-methoxypyrid-3-yl and the combination of R¹ and R⁵ for a compound corresponds in each case to one row of Table B

### Table 30

Compounds of the formulae Ia.1, Ib.1, Ic.1, Id.1, Ie.1 and If.1 in which R⁴ is 2-fluoro-4-chloropyrid-3-yl and the combination of R¹ and R⁵ for a compound corresponds in each case to one row of Table B

### Table 31

Compounds of the formulae Ia.1, Ib.1, Ic.1, Id.1, Ie.1 and If.1 in which R⁴ is 3-fluoro-5-chloropyrid-4-yl and the combination of R¹ and R⁵ for a compound corresponds in each case to one row of Table B

### Table 32

Compounds of the formulae Ia.1, Ib.1, Ic.1, Id.1, Ie.1 and If.1 in which R⁴ is 2-chloro-4-fluoropyrid-3-yl and the combination of R¹ and R⁵ for a compound corresponds in each case to one row of Table B

### Table 33

Compounds of the formulae Ia.1, Ib.1, Ic.1, Id.1, Ie.1 and If.1 in which R⁴ is 2,4-dichloropyrid-3-yl and the combination of R¹ and R⁵ for a compound corresponds in each case to one row of Table B

### Table 34

Compounds of the formulae Ia.1, Ib.1, Ic.1, Id.1, Ie.1 and If.1 in which R⁴ is 3-chloropyrid-2-yl and the combination of R¹ and R⁵ for a compound corresponds in each case to one row of Table B

### Table 35

Compounds of the formulae Ia.1, Ib.1, Ic.1, Id.1, Ie.1 and If.1 in which R⁴ is 4-chloropyrid-3-yl and the combination of R¹ and R⁵ for a compound corresponds in each case to one row of Table B

### Table 36

Compounds of the formulae Ia.1, Ib.1, Ic.1, Id.1, Ie.1 and If.1 in which R⁴ is 3-chloropyrid-4-yl and the combination of R¹ and R⁵ for a compound corresponds in each case to one row of Table B

### Table 37

Compounds of the formulae Ia.1, Ib.1, Ic.1, Id.1, Ie.1 and If.1 in which R⁴ is 2-chloropyrid-3-yl and the combination of R¹ and R⁵ for a compound corresponds in each case to one row of Table B

### Table 38

Compounds of the formulae Ia.1, Ib.1, Ic.1, Id.1, Ie.1 and If.1 in which R⁴ is 3-trifluoromethylpyrid-2-yl and the combination of R¹ and R⁵ for a compound corresponds in each case to one row of Table B

### Table 39

Compounds of the formulae Ia.1, Ib.1, Ic.1, Id.1, Ie.1 and If.1 in which R⁴ is 4-trifluoromethylpyrid-3-yl and the combination of R¹ and R⁵ for a compound corresponds in each case to one row of Table B

### Table 40

Compounds of the formulae Ia.1, Ib.1, Ic.1, Id.1, Ie.1 and If.1 in which R⁴ is 3,5-dichloropyrid-2-yl and the combination of R¹ and R⁵ for a compound corresponds in each case to one row of Table B

### Table 41

Compounds of the formulae Ia.1, Ib.1, Ic.1, Id.1, Ie.1 and If.1 in which R⁴ is 4,6-dichloropyrid-3-yl and the combination of R¹ and R⁵ for a compound corresponds in each case to one row of Table B

### Table 42

Compounds of the formulae Ia.1, Ib.1, Ic.1, Id.1, Ie.1 and If.1 in which R⁴ is 3-trifluoromethylpyrid-4-yl and the combination of R¹ and R⁵ for a compound corresponds in each case to one row of Table B

### Table 43

Compounds of the formulae Ia.1, Ib.1, Ic.1, Id.1, Ie.1 and If.1 in which R⁴ is 2-trifluoromethylpyrid-3-yl and the combination of R¹ and R⁵ for a compound corresponds in each case to one row of Table B

### Table 44

Compounds of the formulae Ia.1, Ib.1, Ic.1, Id.1, Ie.1 and If.1 in which R⁴ is 2-fluoro-4-trifluoromethylpyrid-3-yl and the combination of R¹ and R⁵ for a compound corresponds in each case to one row of Table B

### Table 45

Compounds of the formulae Ia.1, Ib.1, Ic.1, Id.1, Ie.1 and If.1 in which R⁴ is 3-fluoro-5-trifluoromethylpyrid-4-yl and the combination of R¹ and R⁵ for a compound corresponds in each case to one row of Table B

### Table 46

Compounds of the formulae Ia.1, Ib.1, Ic.1, Id.1, Ie.1 and If.1 in which R⁴ is 4-fluoro-2-trifluoromethylpyrid-3-yl and the combination of R¹ and R⁵ for a compound corresponds in each case to one row of Table B

### Table 47

Compounds of the formulae Ia.1, Ib.1, Ic.1, Id.1, Ie.1 and If.1 in which R⁴ is 2,6-dichloropyrid-3-yl and the combination of R¹ and R⁵ for a compound corresponds in each case to one row of Table B

### Table 48

Compounds of the formulae Ia.1, Ib.1, Ic.1, Id.1, Ie.1 and If.1 in which R⁴ is 3,5-dichloropyrid-4-yl and the combination of R¹ and R⁵ for a compound corresponds in each case to one row of Table B

### Table 49

Compounds of the formulae Ia.1, Ib.1, Ic.1, Id.1, Ie.1 and If.1 in which R⁴ is 3-chloro-6-trifluoromethylpyrid-2-yl and the combination of R¹ and R⁵ for a compound corresponds in each case to one row of Table B

### Table 50

Compounds of the formulae Ia.1, Ib.1, Ic.1, Id.1, Ie.1 and If.1 in which R⁴ is 3-fluoro-6-trifluoromethylpyrid-2-yl and the combination of R¹ and R⁵ for a compound corresponds in each case to one row of Table B

### Table 51

Compounds of the formulae Ia.1, Ib.1, Ic.1, Id.1, Ie.1 and If.1 in which R⁴ is pyrid-2-yl and the combination of R¹ and R⁵ for a compound corresponds in each case to one row of Table B

### Table 52

Compounds of the formulae Ia.1, Ib.1, Ic.1, Id.1, Ie.1 and If.1 in which R⁴ is pyrid-3-yl and the combination of R¹ and R⁵ for a compound corresponds in each case to one row of Table B

### Table 53

Compounds of the formulae Ia.1, Ib.1, Ic.1, Id.1, Ie.1 and If.1 in which R⁴ is pyrid-4-yl and the combination of R¹ and R⁵ for a compound corresponds in each case to one row of Table B

### Table 54

Compounds of the formulae Ia.1, Ib.1, Ic.1, Id.1, Ie.1 and If.1 in which R⁴ is 3-fluorothien-2-yl and the combination of R¹ and R⁵ for a compound corresponds in each case to one row of Table B

### Table 55

Compounds of the formulae Ia.1, Ib.1, Ic.1, Id.1, Ie.1 and If.1 in which R⁴ is 3-chlorothien-2-yl and the combination of R¹ and R⁵ for a compound corresponds in each case to one row of Table B

### Table 56

Compounds of the formulae Ia.1, Ib.1, Ic.1, Id.1, Ie.1 and If.1 in which R⁴ is 2,4-difluorothien-3-yl and the combination of R¹ and R⁵ for a compound corresponds in each case to one row of Table B

### Table 57

Compounds of the formulae Ia.1, Ib.1, Ic.1, Id.1, Ie.1 and If.1 in which R⁴ is 2,4-dichlorothien-3-yl and the combination of R¹ and R⁵ for a compound corresponds in each case to one row of Table B

### Table 58

Compounds of the formulae Ia.1, Ib.1, Ic.1, Id.1, Ie.1 and If.1 in which R⁴ is 2,4,5-trichlorothien-3-yl and the combination of R¹ and R⁵ for a compound corresponds in each case to one row of Table B

### Table 59

Compounds of the formulae Ia.1, Ib.1, Ic.1, Id.1, Ie.1 and If.1 in which R⁴ is 2,5,6-trifluorophenyl and the combination of R¹ and R⁵ for a compound corresponds in each case to one row of Table B

### Table 60

Compounds of the formulae Ia.1, Ib.1, Ic.1, Id.1, Ie.1 and If.1 in which R⁴ is 2-fluoro-6-chlorophenyl and the combination of R¹ and R⁵ for a compound corresponds in each case to one row of Table B

### Table 61

Compounds of the formulae Ia.1, Ib.1, Ic.1, Id.1, Ie.1 and If.1 in which R⁴ is 2,6-difluorophenyl and the combination of R¹ and R⁵ for a compound corresponds in each case to one row of Table B

### Table 62

Compounds of the formulae Ia.1, Ib.1, Ic.1, Id.1, Ie.1 and If.1 in which R⁴ is 2,6-dichlorophenyl and the combination of R¹ and R⁵ for a compound corresponds in each case to one row of Table B

### Table 63

Compounds of the formulae Ia.1, Ib.1, Ic.1, Id.1, Ie.1 and Hi in which R⁴ is 2-fluoro-6-methylphenyl and the combination of R¹ and R⁵ for a compound corresponds in each case to one row of Table B

### Table 64

Compounds of the formulae Ia.1, Ib.1, Ic.1, Id.1, Ie.1 and If.1 in which R⁴ is 2,4,6-trifluorophenyl and the combination of R¹ and R⁵ for a compound corresponds in each case to one row of Table B

### Table 65

Compounds of the formulae Ia.1, Ib.1, Ic.1, Id.1, Ie.1 and If.1 in which R⁴ is 2,6-difluoro-4-methoxyphenyl and the combination of R¹ and R⁵ for a compound corresponds in each case to one row of Table B

### Table 66

Compounds of the formulae Ia.1, Ib.1, Ic.1, Id.1, Ie.1 and If.1 in which R⁴ is pentafluorophenyl and the combination of R¹ and R⁵ for a compound corresponds in each case to one row of Table B

### Table 67

Compounds of the formulae Ia.1, Ib.1, Ic.1, Id.1, Ie.1 and If.1 in which R⁴ is 2-methyl-4-fluorophenyl and the combination of R¹ and R⁵ for a compound corresponds in each case to one row of Table B

### Table 68

Compounds of the formulae Ia.1, Ib.1, Ic.1, Id.1, Ie.1 and If.1 in which R⁴ is 2-trifluoromethylphenyl and the combination of R¹ and R⁵ for a compound corresponds in each case to one row of Table B

### Table 69

Compounds of the formulae Ia.1, Ib.1, Ic.1, Id.1, Ie.1 and If.1 in which R⁴ is 2-methoxy-6-fluorophenyl and the combination of R¹ and R⁵ for a compound corresponds in each case to one row of Table B

### Table 70

Compounds of the formulae Ia.1, Ib.1, Ic.1, Id.1, Ie.1 and If.1 in which R⁴ is 2-chlorophenyl and the combination of R¹ and R⁵ for a compound corresponds in each case to one row of Table B

### Table 71

Compounds of the formulae Ia.1, Ib.1, Ic.1, Id.1, Ie.1 and If.1 in which R⁴ is 2-fluorophenyl and the combination of R¹ and R⁵ for a compound corresponds in each case to one row of Table B

### Table 72

Compounds of the formulae Ia.1, Ib.1, Ic.1, Id.1, Ie.1 and If.1 in which R⁴ is 2,4-difluorophenyl and the combination of R¹ and R⁵ for a compound corresponds in each case to one row of Table B

### Table 73

Compounds of the formulae Ia1, Ib.1, Ic.1, Id.1, Ie.1 and If.1 in which R⁴ is 2-fluoro-4-chlorophenyl and the combination of R¹ and R⁵ for a compound corresponds in each case to one row of Table B

### Table 74

Compounds of the formulae Ia.1, Ib.1, Ic.1, Id.1, Ie.1 and If.1 in which R⁴ is 2-chloro-4-fluorophenyl and the combination of R¹ and R⁵ for a compound corresponds in each case to one row of Table B

### Table 75

Compounds of the formulae Ia.1, Ib.1, Ic.1, Id.1, Ie.1 and If.1 in which R⁴ is 2,3-difluorophenyl and the combination of R¹ and R⁵ for a compound corresponds in each case to one row of Table B

### Table 76

Compounds of the formulae Ia.1, Ib.1, Ic.1, Id.1, Ie.1 and If.1 in which R⁴ is 2,5-difluorophenyl and the combination of R¹ and R⁵ for a compound corresponds in each case to one row of Table B

### Table 77

Compounds of the formulae Ia.1, Ib.1, Ic.1, Id.1, Ie.1 and If.1 in which R⁴ is 2,3,4-trifluorophenyl and the combination of R¹ and R⁵ for a compound corresponds in each case to one row of Table B

### Table 78

Compounds of the formulae Ia.1, Ib.1, Ic.1, Id.1, Ie.1 and If.1 in which R⁴ is 2-methylphenyl and the combination of R¹ and R⁵ for a compound corresponds in each case to one row of Table B

### Table 79

Compounds of the formulae Ia.1, Ib.1, Ic.1, Id.1, Ie.1 and If.1 in which R⁴ is 2,4-dimethylphenyl and the combination of R¹ and R⁵ for a compound corresponds in each case to one row of Table B

### Table 80

Compounds of the formulae Ia.1, Ib.1, Ic.1, Id.1, Ie.1 and If.1 in which R⁴ is 2-methyl-4-chlorophenyl and the combination of R¹ and R⁵ for a compound corresponds in each case to one row of Table B

### Table 81

Compounds of the formulae Ia.1, Ib.1, Ic.1, Id.1, Ie.1 and If.1 in which R⁴ is 2-fluoro-4-methylphenyl and the combination of R¹ and R⁵ for a compound corresponds in each case to one row of Table B

### Table 82

Compounds of the formulae Ia.1, Ib.1, Ic.1, Id.1, Ie.1 and If.1 in which R⁴ is 2,6-dimethylphenyl and the combination of R¹ and R⁵ for a compound corresponds in each case to one row of Table B

### Table 83

Compounds of the formulae Ia.1, Ib.1, Ic.1, Id.1, Ie.1 and If.1 in which R⁴ is 2,4,6-trimethylphenyl and the combination of R¹ and R⁵ for a compound corresponds in each case to one row of Table B

### Table 84

Compounds of the formulae Ia.1, Ib.1, Ic.1, Id.1, Ie.1 and If.1 in which R⁴ is 2,6-difluoro-4-methylphenyl and the combination of R¹ and R⁵ for a compound corresponds in each case to one row of Table B

### Table 85

Compounds of the formulae Ia.1, Ib.1, Ic.1, Id.1, Ie.1 and If.1 in which R⁴ is 2-trifluoromethyl-4-fluorophenyl and the combination of R¹ and R⁵ for a compound corresponds in each case to one row of Table B

### Table 86

Compounds of the formulae Ia.1, Ib.1, Ic.1, Id.1, Ie.1 and If.1 in which R⁴ is 2-trifluoromethyl-5-fluorophenyl and the combination of R¹ and R⁵ for a compound corresponds in each case to one row of Table B

### Table 87

Compounds of the formulae Ia.1, Ib.1, Ic.1, Id.1, Ie.1 and If.1 in which R⁴ is 2-trifluoromethyl-5-chlorophenyl and the combination of R¹ and R⁵ for a compound corresponds in each case to one row of Table B

### Table 88

Compounds of the formulae Ia.1, Ib.1, Ic.1, Id.1, Ie.1 and If.1 in which R⁴ is 2-chloro-4-fluorophenyl and the combination of R¹ and R⁵ for a compound corresponds in each case to one row of Table B

### Table 89

Compounds of the formulae Ia.1, Ib.1, Ic.1, Id.1, Ie.1 and If.1 in which R⁴ is 2-chloro-5-fluorophenyl and the combination of R¹ and R⁵ for a compound corresponds in each case to one row of Table B

### Table 90

Compounds of the formulae Ia.1, Ib.1, Ic.1, Id.1, Ie.1 and If.1 in which R⁴ is 2-methyl-5-fluorophenyl and the combination of R¹ and R⁵ for a compound corresponds in each case to one row of Table B

### Table 91

Compounds of the formulae Ia.1, Ib.1, Ic.1, Id.1, Ie.1 and If.1 in which R⁴ is 2-chloro-4-methoxyphenyl and the combination of R¹ and R⁵ for a compound corresponds in each case to one row of Table B

### Table 92

Compounds of the formulae Ia.1, Ib.1, Ic.1, Id.1, Ie.1 and If.1 in which R⁴ is 2-chloro-6-fluorophenyl and the combination of R¹ and R⁵ for a compound corresponds in each case to one row of Table B

### Table 93

Compounds of the formulae Ia.1, Ib.1, Ic.1, Id.1, Ie.1 and If.1 in which R⁴ is 2,6-dichloro-4-fluorophenyl and the combination of R¹ and R⁵ for a compound corresponds in each case to one row of Table B

### Table 94

Compounds of the formulae Ia.1, Ib.1, Ic.1, Id.1, Ie.1 and If.1 in which R⁴ is 2,4-dichlorophenyl and the combination of R¹ and R⁵ for a compound corresponds in each case to one row of Table B

### Table 95

Compounds of the formulae Ia.1, Ib.1, Ic.1, Id.1, Ie.1 and If.1 in which R⁴ is 2,3,6-trifluorophenyl and the combination of R¹ and R⁵ for a compound corresponds in each case to one row of Table B

### Table 96

Compounds of the formulae Ia.1, Ib.1, Ic.1, Id.1, Ie.1 and If.1 in which R⁴ is 2,4,5-trifluorophenyl and the combination of R¹ and R⁵ for a compound corresponds in each case to one row of Table B

### Table 97

Compounds of the formulae Ia.1, Ib.1, Ic.1, Id.1, Ie.1 and If.1 in which R⁴ is 2,3,4,6-tetrafluorophenyl and the combination of R¹ and R⁵ for a compound corresponds in each case to one row of Table B

### Table 98

Compounds of the formulae Ia.1, Ib.1, Ic.1, Id.1, Ie.1 and If.1 in which R⁴ is 2,4,6-trichlorophenyl and the combination of R¹ and R⁵ for a compound corresponds in each case to one row of Table B

### Table 99

Compounds of the formulae Ia.1, Ib.1, Ic.1, Id.1, Ie.1 and If.1 in which R⁴ is 2-chloro-4-methylphenyl and the combination of R¹ and R⁵ for a compound corresponds in each case to one row of Table B

### Table 100

Compounds of the formulae Ia.1, Ib.1, Ic.1, Id.1, Ie.1 and If.1 in which R⁴ is 2,4-dichloro-5-methylphenyl and the combination of R¹ and R⁵ for a compound corresponds in each case to one row of Table B

### Table 101

Compounds of the formulae Ia.1, Ib.1, Ic.1, Id.1, Ie.1 and If.1 in which R⁴ is 2,5-dichlorophenyl and the combination of R¹ and R⁵ for a compound corresponds in each case to one row of Table B

### Table 102

Compounds of the formulae Ia.1, Ib.1, Ic.1, Id.1, Ie.1 and If.1 in which R⁴ is 2,5-dimethylphenyl and the combination of R¹ and R⁵ for a compound corresponds in each case to one row of Table B

### Table 103

Compounds of the formulae Ia.1, Ib.1, Ic.1, Id.1, Ie.1 and If.1 in which R⁴ is 2-chloro-5-bromophenyl and the combination of R¹ and R⁵ for a compound corresponds in each case to one row of Table B

### Table 104

Compounds of the formulae Ia.1, Ib.1, Ic.1, Id.1, Ie.1 and If.1 in which R⁴ is 2-chloro-5-methoxyphenyl and the combination of R¹ and R⁵ for a compound corresponds in each case to one row of Table B

### Table 105

Compounds of the formulae Ia.1, Ib.1, Ic.1, Id.1, Ie.1 and If.1 in which R⁴ is 2-chloro-5-methylphenyl and the combination of R¹ and R⁵ for a compound corresponds in each case to one row of Table B

### Table 106

Compounds of the formulae Ia.1, Ib.1, Ic.1, Id.1, Ie.1 and If.1 in which R⁴ is 3,4-methylenedioxyphenyl and the combination of R¹ and R⁵ for a compound corresponds in each case to one row of Table B

### Table 107

Compounds of the formulae Ia.1, Ib.1, Ic.1, Id.1, Ie.1 and If.1 in which R⁴ is 2,4-difluoro-6-chlorophenyl and the combination of R¹ and R⁵ for a compound corresponds in each case to one row of Table B

### Table 108

Compounds of the formulae Ia.1, Ib.1, Ic.1, Id.1, Ie.1 and If.1 in which R⁴ is 2,6-difluoro-4-chlorophenyl and the combination of R¹ and R⁵ for a compound corresponds in each case to one row of Table B

### Table 109

Compounds of the formulae Ia.1, Ib.1, Ic.1, Id.1, Ie.1 and If.1 in which R⁴ is phenyl and the combination of R¹ and R⁵ for a compound corresponds in each case to one row of Table B

### Table 110

Compounds of the formulae Ia.1, Ib.1, Ic.1, Id.1, Ie.1 and If.1 in which R⁴ is 2-chloro-5-nitrophenyl and the combination of R¹ and R⁵ for a compound corresponds in each case to one row of Table B

TABLE B

| Row | R¹ | R⁵ |
|---|---|---|
| B-1 | NHCH₃ | Cl |
| B-2 | N(CH₃)₂ | Cl |
| B-3 | NH(CH₂CH₃) | Cl |
| B-4 | N(CH₂CH₃)(CH₃) | Cl |
| B-5 | N(CH₂CH₃)₂ | Cl |
| B-6 | NH(CH₂CF₃) | Cl |
| B-7 | N(CH₂CF₃)(CH₃) | Cl |
| B-8 | N(CH₂CF₃)(CH₂CH₃) | Cl |
| B-9 | NH(CH₂CCl₃) | Cl |
| B-10 | N(CH₂CCl₃)(CH₃) | Cl |
| B-11 | N(CH₂CCl₃)(CH₂CH₃) | Cl |
| B-12 | NH(CH₂CH₂CH₃) | Cl |
| B-13 | N(CH₂CH₂CH₃)(CH₃) | Cl |
| B-14 | N(CH₂CH₂CH₃)(CH₂CH₃) | Cl |
| B-15 | N(CH₂CH₂CH₃)(CH₂CH₂CH₃) | Cl |
| B-16 | NH(CH(CH₃)₂) | Cl |
| B-17 | N(CH(CH₃)₂)(CH₃) | Cl |
| B-18 | N(CH(CH₃)₂)(CH₂CH₃) | Cl |
| B-19 | NH(CH₂CH(CH₃)₂) | Cl |
| B-20 | N(CH₂CH(CH₃)₂)(CH₃) | Cl |
| B-21 | N(CH₂CH(CH₃)₂)(CH₂CH₃) | Cl |
| B-22 | N(CH₂CH(CH₃)₂)(CH₂CH₂CH₃) | Cl |
| B-23 | NH(CH₂CH₂CH₂CH₃) | Cl |
| B-24 | N(CH₂CH₂CH₂CH₃)(CH₃) | Cl |
| B-25 | N(CH₂CH₂CH₂CH₃)(CH₂CH₃) | Cl |
| B-26 | N(CH₂CH₂CH₂CH₃)(CH₂CH₂CH₃) | Cl |
| B-27 | N(CH₂CH₂CH₂CH₃)₂ | Cl |
| B-28 | NH(±CH(CH₃)—CH₂CH₃) | Cl |
| B-29 | N(±CH(CH₃)—CH₂CH₃)(CH₃) | Cl |
| B-30 | N(±CH(CH₃)—CH₂CH₃)(CH₂CH₃) | Cl |
| B-31 | NH((S)CH(CH₃)—CH₂CH₃) | Cl |
| B-32 | N((S)CH(CH₃)—CH₂CH₃)(CH₃) | Cl |
| B-33 | N((S)CH(CH₃)—CH₂CH₃)(CH₂CH₃) | Cl |
| B-34 | NH((R)CH(CH₃)—CH₂CH₃) | Cl |
| B-35 | N((R)CH(CH₃)—CH₂CH₃)(CH₃) | Cl |
| B-36 | N((R)CH(CH₃)—CH₂CH₃)(CH₂CH₃) | Cl |
| B-37 | NH(±CH(CH₃)—CH(CH₃)₂) | Cl |
| B-38 | N(±CH(CH₃)—CH(CH₃)₂)(CH₃) | Cl |
| B-39 | N(±CH(CH₃)—CH(CH₃)₂)(CH₂CH₃) | Cl |
| B-40 | NH((S)CH(CH₃)—CH(CH₃)₂) | Cl |
| B-41 | N((S)CH(CH₃)—CH(CH₃)₂)(CH₃) | Cl |
| B-42 | N((S)CH(CH₃)—CH(CH₃)₂)(CH₂CH₃) | Cl |
| B-43 | NH((R)CH(CH₃)—CH(CH₃)₂) | Cl |
| B-44 | N((R)CH(CH₃)—CH(CH₃)₂)(CH₃) | Cl |
| B-45 | N((R)CH(CH₃)—CH(CH₃)₂)(CH₂CH₃) | Cl |
| B-46 | NH(±CH(CH₃)—C(CH₃)₃) | Cl |
| B-47 | NH(±CH(CH₃)—C(CH₃)₃)(CH₃) | Cl |
| B-48 | N(±CH(CH₃)—C(CH₃)₃)(CH₂CH₃) | Cl |
| B-49 | NH((S)CH(CH₃)—C(CH₃)₃) | Cl |
| B-50 | N((S)CH(CH₃)—C(CH₃)₃)(CH₃) | Cl |
| B-51 | N((S)CH(CH₃)—C(CH₃)₃)(CH₂CH₃) | Cl |
| B-52 | NH((R)CH(CH₃)—C(CH₃)₃) | Cl |
| B-53 | N((R)CH(CH₃)—C(CH₃)₃)(CH₃) | Cl |
| B-54 | N((R)CH(CH₃)—C(CH₃)₃)(CH₂CH₃) | Cl |
| B-55 | NH(±CH(CH₃)—CF₃) | Cl |
| B-56 | N(±CH(CH₃)—CF₃)(CH₃) | Cl |
| B-57 | N(±CH(CH₃)—CF₃)(CH₂CH₃) | Cl |
| B-58 | NH((S)CH(CH₃)—CF₃) | Cl |
| B-59 | N((S)CH(CH₃)—CF₃)(CH₃) | Cl |
| B-60 | N((S)CH(CH₃)—CF₃)(CH₂CH₃) | Cl |
| B-61 | NH((R)CH(CH₃)—CF₃) | Cl |
| B-62 | N((R)CH(CH₃)—CF₃)(CH₃) | Cl |
| B-63 | N((R)CH(CH₃)—CF₃)(CH₂CH₃) | Cl |
| B-64 | NH(±CH(CH₃)—CCl₃) | Cl |
| B-65 | N(±CH(CH₃)—CCl₃)(CH₃) | Cl |
| B-66 | N(±CH(CH₃)—CCl₃)(CH₂CH₃) | Cl |
| B-67 | NH((S)CH(CH₃)—CCl₃) | Cl |
| B-68 | N((S)CH(CH₃)—CCl₃)(CH₃) | Cl |
| B-69 | N((S)CH(CH₃)—CCl₃)(CH₂CH₃) | Cl |
| B-70 | NH((R)CH(CH₃)—CCl₃) | Cl |
| B-71 | N((R)CH(CH₃)—CCl₃)(CH₃) | Cl |
| B-72 | N((R)CH(CH₃)—CCl₃)(CH₂CH₃) | Cl |
| B-73 | NH(CH₂CF₂CF₃) | Cl |
| B-74 | N(CH₂CF₂CF₃)(CH₃) | Cl |
| B-75 | N(CH₂CF₂CF₃)(CH₂CH₃) | Cl |
| B-76 | NH(CH₂(CF₂)₂CF₃) | Cl |
| B-77 | N(CH₂(CF₂)₂CF₃)(CH₃) | Cl |
| B-78 | N(CH₂(CF₂)₂CF₃)(CH₂CH₃) | Cl |
| B-79 | NH(CH₂C(CH₃)═CH₂) | Cl |
| B-80 | N(CH₂C(CH₃)═CH₂)(CH₃) | Cl |
| B-81 | N(CH₂C(CH₃)═CH₂)(CH₂CH₃) | Cl |
| B-82 | NH(CH₂CH═CH₂) | Cl |
| B-83 | N(CH₂CH═CH₂)(CH₃) | Cl |
| B-84 | N(CH₂CH═CH₂)(CH₂CH₃) | Cl |
| B-85 | NH((±)CH(CH₃)CH═CH₂) | Cl |
| B-86 | N((±)CH(CH₃)CH═CH₂)(CH₃) | Cl |
| B-87 | N((±)CH(CH₃)CH═CH₂)(CH₂CH₃) | Cl |
| B-88 | NH((S)CH(CH₃)CH═CH₂) | Cl |
| B-89 | N((S)CH(CH₃)CH═CH₂)(CH₃) | Cl |
| B-90 | N((S)CH(CH₃)CH═CH₂)(CH₂CH₃) | Cl |
| B-91 | NH((R)CH(CH₃)CH═CH₂) | Cl |
| B-92 | N((R)CH(CH₃)CH═CH₂)(CH₃) | Cl |
| B-93 | N((R)CH(CH₃)CH═CH₂)(CH₂CH₃) | Cl |
| B-94 | NH((±)CH(CH₃)C(CH₃)═CH₂) | Cl |
| B-95 | N((±)CH(CH₃)C(CH₃)═CH₂)(CH₃) | Cl |
| B-96 | N((±)CH(CH₃)C(CH₃)═CH₂)(CH₂CH₃) | Cl |
| B-97 | NH((S)CH(CH₃)C(CH₃)═CH₂) | Cl |
| B-98 | N((S)CH(CH₃)C(CH₃)═CH₂)(CH₃) | Cl |
| B-99 | N((S)CH(CH₃)C(CH₃)═CH₂)(CH₂CH₃) | Cl |
| B-100 | NH((R)CH(CH₃)C(CH₃)═CH₂) | Cl |
| B-101 | N((R)CH(CH₃)C(CH₃)═CH₂)(CH₃) | Cl |
| B-102 | N((R)CH(CH₃)C(CH₃)═CH₂)(CH₂CH₃) | Cl |
| B-103 | NH(CH₂—C≡CH) | Cl |
| B-104 | N(CH₂—C≡CH)(CH₃) | Cl |
| B-105 | N(CH₂—C≡CH)(CH₂CH₃) | Cl |
| B-106 | NH(cyclo-C₅H₉) | Cl |
| B-107 | N(cyclo-C₅H₉)(CH₃) | Cl |
| B-108 | N(cyclo-C₅H₉)(CH₂CH₃) | Cl |
| B-109 | NH(cyclo-C₆H₁₁) | Cl |
| B-110 | N(cyclo-C₆H₁₁)(CH₃) | Cl |
| B-111 | N(cyclo-C₆H₁₁)(CH₂CH₃) | Cl |
| B-112 | NH(CH₂—C₆H₅) | Cl |
| B-113 | N(CH₂—C₆H₅)(CH₃) | Cl |
| B-114 | N(CH₂—C₆H₅)(CH₂CH₃) | Cl |
| B-115 | N(NH₂)(CH₂—c—C₆H₁₁) | Cl |
| B-116 | N(NH₂)(CH₂CH₃) | Cl |
| B-117 | N(NH₂)(CH₂CH₂CH₃) | Cl |
| B-118 | NH(NH—CH₂—CH═CH₂) | Cl |
| B-119 | N(NH—CH₂—CH═CH₂)(CH₃) | Cl |
| B-120 | N(NH—CH₂—CH═CH₂)(CH₂CH₃) | Cl |
| B-121 | NH(NH—C(CH₃)₃) | Cl |
| B-122 | NH(N(CH₃)₂) | Cl |
| B-123 | NH(NH(CH₃)) | Cl |
| B-124 | NR²R³ where R² + R³: —(CH₂)₂CH═CHCH₂— | Cl |
| B-125 | NR²R³ where R² + R³: —(CH₂)₂C(CH₃)═CHCH₂— | Cl |
| B-126 | NR²R³ where R² + R³: —CH(CH₃)CH₂—CH═CHCH₂— | Cl |
| B-127 | NR²R³ where R² + R³: —(CH₂)₂CH(CH₃)(CH₂)₂— | Cl |
| B-128 | NR²R³ where R² + R³: —(CH₂)₃CHFCH₂— | Cl |
| B-129 | NR²R³ where R² + R³: —(CH₂)₂CHF(CH₂)₂— | Cl |
| B-130 | NR²R³ where R² + R³: —CH₂CHF(CH₂)₃— | Cl |
| B-131 | NR²R³ where R² + R³: —(CH₂)₂CH(CF₃)(CH₂)₂— | Cl |
| B-132 | NR²R³ where R² + R³: —(CH₂)₂O(CH₂)₂— | Cl |
| B-133 | NR²R³ where R² + R³: —(CH₂)₂S(CH₂)₂— | Cl |
| B-134 | NR²R³ where R² + R³: —(CH₂)₅— | Cl |
| B-135 | NR²R³ where R² + R³: —(CH₂)₄— | Cl |
| B-136 | NR²R³ where R² + R³: —CH₂CH═CHCH₂— | Cl |
| B-137 | NR²R³ where R² + R³: —CH(CH₃)(CH₂)₃— | Cl |
| B-138 | NR²R³ where R² + R³: —CH₂CH(CH₃)(CH₂)₂— | Cl |
| B-139 | NR²R³ where R² + R³: —CH(CH₃)—(CH₂)₂—CH(CH₃)— | Cl |
| B-140 | NR²R³ where R² + R³: —CH(CH₃)—(CH₂)₄— | Cl |
| B-141 | NR²R³ where R² + R³: —CH₂—CH(CH₃)—(CH₂)₃— | Cl |
| B-142 | NR²R³ where R² + R³: —(CH₂)—CH(CH₃)—CH₂—CH(CH₃)—CH₂— | Cl |
| B-143 | NR²R³ where R² + R³: —CH(CH₂CH₃)—(CH₂)₄— | Cl |
| B-144 | NR²R³ where R² + R³: —(CH₂)₆— | Cl |
| B-145 | NR²R³ where R² + R³: —CH(CH₃)—(CH₂)₅— | Cl |
| B-146 | NR²R³ where R² + R³: —(CH₂)₂—N(CH₃)—(CH₂)₂— | Cl |
| B-147 | NR²R³ where R² + R³: —N═CH—CH═CH— | Cl |
| B-148 | NR²R³ where R² + R³: —N═C(CH₃)—CH═C(CH₃)— | Cl |
| B-149 | NR²R³ where R² + R³: —N═C(CF₃)—CH═C(CF₃)— | Cl |
| B-150 | cyclo-C₆H₁₁ | Cl |
| B-151 | 4-CH₃-(cyclo-C₆H₁₁) | Cl |
| B-152 | CH₂(CH₂)₂CH₃ | Cl |
| B-153 | CH₂CH(CH₃)C₂H₅ | Cl |
| B-154 | CH₂C(CH₃)₂C₂H₅ | Cl |
| B-155 | CH₂CH₂CH═CH₂ | Cl |
| B-156 | CH₂CH(CH₃)CH═CH₂ | Cl |
| B-157 | CH₂C(CH₃)₂CH═CH₂ | Cl |
| B-158 | NHCH₃ | CH₃ |
| B-159 | N(CH₃)₂ | CH₃ |
| B-160 | NH(CH₂CH₃) | CH₃ |
| B-161 | N(CH₂CH₃)(CH₃) | CH₃ |
| B-162 | N(CH₂CH₃)₂ | CH₃ |
| B-163 | NH(CH₂CF₃) | CH₃ |
| B-164 | N(CH₂CF₃)(CH₃) | CH₃ |
| B-165 | N(CH₂CF₃)(CH₂CH₃) | CH₃ |
| B-166 | NH(CH₂CCl₃) | CH₃ |
| B-167 | N(CH₂CCl₃)(CH₃) | CH₃ |
| B-168 | N(CH₂CCl₃)(CH₂CH₃) | CH₃ |
| B-169 | NH(CH₂CH₂CH₃) | CH₃ |
| B-170 | N(CH₂CH₂CH₃)(CH₃) | CH₃ |
| B-171 | N(CH₂CH₂CH₃)(CH₂CH₃) | CH₃ |
| B-172 | N(CH₂CH₂CH₃)(CH₂CH₂CH₃) | CH₃ |
| B-173 | NH(CH(CH₃)₂) | CH₃ |
| B-174 | N(CH(CH₃)₂)(CH₃) | CH₃ |
| B-175 | N(CH(CH₃)₂)(CH₂CH₃) | CH₃ |
| B-176 | NH(CH₂CH(CH₃)₂) | CH₃ |
| B-177 | N(CH₂CH(CH₃)₂)(CH₃) | CH₃ |
| B-178 | N(CH₂CH(CH₃)₂)(CH₂CH₃) | CH₃ |
| B-179 | N(CH₂CH(CH₃)₂)(CH₂CH₂CH₃) | CH₃ |
| B-180 | NH(CH₂CH₂CH₂CH₃) | CH₃ |
| B-181 | N(CH₂CH₂CH₂CH₃)(CH₃) | CH₃ |
| B-182 | N(CH₂CH₂CH₂CH₃)(CH₂CH₃) | CH₃ |
| B-183 | N(CH₂CH₂CH₂CH₃)(CH₂CH₂CH₃) | CH₃ |
| B-184 | N(CH₂CH₂CH₂CH₃)₂ | CH₃ |
| B-185 | NH(±CH(CH₃)—CH₂CH₃) | CH₃ |
| B-186 | N(±CH(CH₃)—CH₂CH₃)(CH₃) | CH₃ |
| B-187 | N(±CH(CH₃)—CH₂CH₃)(CH₂CH₃) | CH₃ |
| B-188 | NH((S)CH(CH₃)—CH₂CH₃) | CH₃ |
| B-189 | N((S)CH(CH₃)—CH₂CH₃)(CH₃) | CH₃ |
| B-190 | N((S)CH(CH₃)—CH₂CH₃)(CH₂CH₃) | CH₃ |
| B-191 | NH((R)CH(CH₃)—CH₂CH₃) | CH₃ |
| B-192 | N((R)CH(CH₃)—CH₂CH₃)(CH₃) | CH₃ |
| B-193 | N((R)CH(CH₃)—CH₂CH₃)(CH₂CH₃) | CH₃ |
| B-194 | NH(±CH(CH₃)—CH(CH₃)₂) | CH₃ |
| B-195 | N(±CH(CH₃)—CH(CH₃)₂)(CH₃) | CH₃ |
| B-196 | N(±CH(CH₃)—CH(CH₃)₂)(CH₂CH₃) | CH₃ |
| B-197 | NH((S)CH(CH₃)—CH(CH₃)₂) | CH₃ |
| B-198 | N((S)CH(CH₃)—CH(CH₃)₂)(CH₃) | CH₃ |
| B-199 | N((S)CH(CH₃)—CH(CH₃)₂)(CH₂CH₃) | CH₃ |
| B-200 | NH((R)CH(CH₃)—CH(CH₃)₂) | CH₃ |
| B-201 | N((R)CH(CH₃)—CH(CH₃)₂)(CH₃) | CH₃ |
| B-202 | N((R)CH(CH₃)—CH(CH₃)₂)(CH₂CH₃) | CH₃ |
| B-203 | NH(±CH(CH₃)—C(CH₃)₃) | CH₃ |
| B-204 | NH(±CH(CH₃)—C(CH₃)₃)(CH₃) | CH₃ |
| B-205 | N(±CH(CH₃)—C(CH₃)₃)(CH₂CH₃) | CH₃ |
| B-206 | NH((S)CH(CH₃)—C(CH₃)₃) | CH₃ |
| B-207 | N((S)CH(CH₃)—C(CH₃)₃)(CH₃) | CH₃ |
| B-208 | N((S)CH(CH₃)—C(CH₃)₃)(CH₂CH₃) | CH₃ |
| B-209 | NH((R)CH(CH₃)—C(CH₃)₃) | CH₃ |
| B-210 | N((R)CH(CH₃)—C(CH₃)₃)(CH₃) | CH₃ |
| B-211 | N((R)CH(CH₃)—C(CH₃)₃)(CH₂CH₃) | CH₃ |
| B-212 | NH(±CH(CH₃)—CF₃) | CH₃ |
| B-213 | N(±CH(CH₃)—CF₃)(CH₃) | CH₃ |
| B-214 | N(±CH(CH₃)—CF₃)(CH₂CH₃) | CH₃ |
| B-215 | NH((S)CH(CH₃)—CF₃) | CH₃ |
| B-216 | N((S)CH(CH₃)—CF₃)(CH₃) | CH₃ |
| B-217 | N((S)CH(CH₃)—CF₃)(CH₂CH₃) | CH₃ |
| B-218 | NH((R)CH(CH₃)—CF₃) | CH₃ |
| B-219 | N((R)CH(CH₃)—CF₃)(CH₃) | CH₃ |
| B-220 | N((R)CH(CH₃)—CF₃)(CH₂CH₃) | CH₃ |
| B-221 | NH(±CH(CH₃)—CCl₃) | CH₃ |
| B-222 | N(±CH(CH₃)—CCl₃)(CH₃) | CH₃ |
| B-223 | N(±CH(CH₃)—CCl₃)(CH₂CH₃) | CH₃ |
| B-224 | NH((S)CH(CH₃)—CCl₃) | CH₃ |
| B-225 | N((S)CH(CH₃)—CCl₃)(CH₃) | CH₃ |
| B-226 | N((S)CH(CH₃)—CCl₃)(CH₂CH₃) | CH₃ |
| B-227 | NH((R)CH(CH₃)—CCl₃) | CH₃ |
| B-228 | N((R)CH(CH₃)—CCl₃)(CH₃) | CH₃ |
| B-229 | N((R)CH(CH₃)—CCl₃)(CH₂CH₃) | CH₃ |
| B-230 | NH(CH₂CF₂CF₃) | CH₃ |
| B-231 | N(CH₂CF₂CF₃)(CH₃) | CH₃ |
| B-232 | N(CH₂CF₂CF₃)(CH₂CH₃) | CH₃ |
| B-233 | NH(CH₂(CF₂)₂CF₃) | CH₃ |
| B-234 | N(CH₂(CF₂)₂CF₃)(CH₃) | CH₃ |
| B-235 | N(CH₂(CF₂)₂CF₃)(CH₂CH₃) | CH₃ |
| B-236 | NH(CH₂C(CH₃)═CH₂) | CH₃ |
| B-237 | N(CH₂C(CH₃)═CH₂)(CH₃) | CH₃ |
| B-238 | N(CH₂C(CH₃)═CH₂)(CH₂CH₃) | CH₃ |
| B-239 | NH(CH₂CH═CH₂) | CH₃ |
| B-240 | N(CH₂CH═CH₂)(CH₃) | CH₃ |
| B-241 | N(CH₂CH═CH₂)(CH₂CH₃) | CH₃ |
| B-242 | NH((±)CH(CH₃)CH═CH₂) | CH₃ |
| B-243 | N((±)CH(CH₃)CH═CH₂)(CH₃) | CH₃ |
| B-244 | N((±)CH(CH₃)CH═CH₂)(CH₂CH₃) | CH₃ |
| B-245 | NH((S)CH(CH₃)CH═CH₂) | CH₃ |
| B-246 | N((S)CH(CH₃)CH═CH₂)(CH₃) | CH₃ |
| B-247 | N((S)CH(CH₃)CH═CH₂)(CH₂CH₃) | CH₃ |
| B-248 | NH((R)CH(CH₃)CH═CH₂) | CH₃ |
| B-249 | N((R)CH(CH₃)CH═CH₂)(CH₃) | CH₃ |
| B-250 | N((R)CH(CH₃)CH═CH₂)(CH₂CH₃) | CH₃ |
| B-251 | NH((±)CH(CH₃)C(CH₃)═CH₂) | CH₃ |
| B-252 | N((±)CH(CH₃)C(CH₃)═CH₂)(CH₃) | CH₃ |
| B-253 | N((±)CH(CH₃)C(CH₃)═CH₂)(CH₂CH₃) | CH₃ |
| B-254 | NH((S)CH(CH₃)C(CH₃)═CH₂) | CH₃ |
| B-255 | N((S)CH(CH₃)C(CH₃)═CH₂)(CH₃) | CH₃ |
| B-256 | N((S)CH(CH₃)C(CH₃)═CH₂)(CH₂CH₃) | CH₃ |
| B-257 | NH((R)CH(CH₃)C(CH₃)═CH₂) | CH₃ |
| B-258 | N((R)CH(CH₃)C(CH₃)═CH₂)(CH₃) | CH₃ |
| B-259 | N((R)CH(CH₃)C(CH₃)═CH₂)(CH₂CH₃) | CH₃ |
| B-260 | NH(CH₂—C≡CH) | CH₃ |
| B-261 | N(CH₂—C≡CH)(CH₃) | CH₃ |
| B-262 | N(CH₂—C≡CH)(CH₂CH₃) | CH₃ |
| B-263 | NH(cyclo-C₅H₉) | CH₃ |
| B-264 | N(cyclo-C₅H₉)(CH₃) | CH₃ |
| B-265 | N(cyclo-C₅H₉)(CH₂CH₃) | CH₃ |
| B-266 | NH(cyclo-C₆H₁₁) | CH₃ |
| B-267 | N(cyclo-C₆H₁₁)(CH₃) | CH₃ |
| B-268 | N(cyclo-C₆H₁₁)(CH₂CH₃) | CH₃ |
| B-269 | NH(CH₂—C₆H₅) | CH₃ |
| B-270 | N(CH₂—C₆H₅)(CH₃) | CH₃ |
| B-271 | N(CH₂—C₆H₅)(CH₂CH₃) | CH₃ |
| B-272 | N(NH₂)(CH₂-c-C₆H₁₁) | CH₃ |
| B-273 | N(NH₂)(CH₂CH₃) | CH₃ |
| B-274 | N(NH₂)(CH₂CH₂CH₃) | CH₃ |
| B-275 | NH(NH—CH₂—CH═CH₂) | CH₃ |
| B-276 | N(NH—CH₂—CH═CH₂)(CH₃) | CH₃ |
| B-277 | N(NH—CH₂—CH═CH₂)(CH₂CH₃) | CH₃ |
| B-278 | NH(NH—C(CH₃)₃) | CH₃ |
| B-279 | NH(N(CH₃)₂) | CH₃ |
| B-280 | NH(NH(CH₃)) | CH₃ |
| B-281 | NR²R³ where R² + R³: —(CH₂)₂CH═CHCH₂— | CH₃ |
| B-282 | NR²R³ where R² + R³: —(CH₂)₂C(CH₃)═CHCH₂— | CH₃ |
| B-283 | NR²R³ where R² + R³: —CH(CH₃)CH₂—CH═CHCH₂— | CH₃ |
| B-284 | NR²R³ where R² + R³: —(CH₂)₂CH(CH₃)(CH₂)₂— | CH₃ |
| B-285 | NR²R³ where R² + R³: —(CH₂)₃CHFCH₂— | CH₃ |
| B-286 | NR²R³ where R² + R³: —(CH₂)₂CHF(CH₂)₂— | CH₃ |
| B-287 | NR²R³ where R² + R³: —CH₂CHF(CH₂)₃— | CH₃ |
| B-288 | NR²R³ where R² + R³: —(CH₂)₂CH(CF₃)(CH₂)₂— | CH₃ |
| B-289 | NR²R³ where R² + R³: —(CH₂)₂O(CH₂)₂— | CH₃ |
| B-290 | NR²R³ where R² + R³: —(CH₂)₂S(CH₂)₂— | CH₃ |
| B-291 | NR²R³ where R² + R³: —(CH₂)₅— | CH₃ |
| B-292 | NR²R³ where R² + R³: —(CH₂)₄— | CH₃ |
| B-293 | NR²R³ where R² + R³: —CH₂CH═CHCH₂— | CH₃ |
| B-294 | NR²R³ where R² + R³: —CH(CH₃)(CH₂)₃— | CH₃ |
| B-295 | NR²R³ where R² + R³: —CH₂CH(CH₃)(CH₂)₂— | CH₃ |
| B-296 | NR²R³ where R² + R³: —CH(CH₃)—(CH₂)₂—CH(CH₃)— | CH₃ |
| B-297 | NR²R³ where R² + R³: —CH(CH₃)—(CH₂)₄— | CH₃ |
| B-298 | NR²R³ where R² + R³: —CH₂—CH(CH₃)—(CH₂)₃— | CH₃ |
| B-299 | NR²R³ where R² + R³: —(CH₂)—CH(CH₃)—CH₂—CH(CH₃)—CH₂— | CH₃ |
| B-300 | NR²R³ where R² + R³: —CH(CH₂CH₃)—(CH₂)₄— | CH₃ |
| B-301 | NR²R³ where R² + R³: —(CH₂)₆— | CH₃ |
| B-302 | NR²R³ where R² + R³: —CH(CH₃)—(CH₂)₅— | CH₃ |
| B-303 | NR²R³ where R² + R³: —(CH₂)₂—N(CH₃)—(CH₂)₂— | CH₃ |
| B-304 | NR²R³ where R² + R³: —N═CH—CH═CH— | CH₃ |
| B-305 | NR²R³ where R² + R³: —N═C(CH₃)—CH═C(CH₃)— | CH₃ |
| B-306 | NR²R³ where R² + R³: —N═C(CF₃)—CH═C(CF₃)— | CH₃ |
| B-307 | cyclo-C₆H₁₁ | CH₃ |
| B-308 | 4-CH₃-(cyclo-C₆H₁₁) | CH₃ |
| B-309 | CH₂(CH₂)₂CH₃ | CH₃ |
| B-310 | CH₂CH(CH₃)C₂H₅ | CH₃ |
| B-311 | CH₂C(CH₃)₂C₂H₅ | CH₃ |
| B-312 | CH₂CH₂CH═CH₂ | CH₃ |
| B-313 | CH₂CH(CH₃)CH═CH₂ | CH₃ |
| B-314 | CH₂C(CH₃)₂CH═CH₂ | CH₃ |
| B-315 | NHCH₃ | F |
| B-316 | N(CH₃)₂ | F |
| B-317 | NH(CH₂CH₃) | F |
| B-318 | N(CH₂CH₃)(CH₃) | F |
| B-319 | N(CH₂CH₃)₂ | F |
| B-320 | NH(CH₂CF₃) | F |
| B-321 | N(CH₂CF₃)(CH₃) | F |
| B-322 | N(CH₂CF₃)(CH₂CH₃) | F |
| B-323 | NH(CH₂CCl₃) | F |
| B-324 | N(CH₂CCl₃)(CH₃) | F |
| B-325 | N(CH₂CCl₃)(CH₂CH₃) | F |
| B-326 | NH(CH₂CH₂CH₃) | F |
| B-327 | N(CH₂CH₂CH₃)(CH₃) | F |
| B-328 | N(CH₂CH₂CH₃)(CH₂CH₃) | F |
| B-329 | N(CH₂CH₂CH₃)(CH₂CH₂CH₃) | F |
| B-330 | NH(CH(CH₃)₂) | F |
| B-331 | N(CH(CH₃)₂)(CH₃) | F |
| B-332 | N(CH(CH₃)₂)(CH₂CH₃) | F |
| B-333 | NH(CH₂CH(CH₃)₂) | F |
| B-334 | N(CH₂CH(CH₃)₂)(CH₃) | F |
| B-335 | N(CH₂CH(CH₃)₂)(CH₂CH₃) | F |
| B-336 | N(CH₂CH(CH₃)₂)(CH₂CH₂CH₃) | F |
| B-337 | NH(CH₂CH₂CH₂CH₃) | F |
| B-338 | N(CH₂CH₂CH₂CH₃)(CH₃) | F |
| B-339 | N(CH₂CH₂CH₂CH₃)(CH₂CH₃) | F |
| B-340 | N(CH₂CH₂CH₂CH₃)(CH₂CH₂CH₃) | F |
| B-341 | N(CH₂CH₂CH₂CH₃)₂ | F |
| B-342 | NH(±CH(CH₃)—CH₂CH₃) | F |
| B-343 | N(±CH(CH₃)—CH₂CH₃)(CH₃) | F |
| B-344 | N(±CH(CH₃)—CH₂CH₃)(CH₂CH₃) | F |
| B-345 | NH((S)CH(CH₃)—CH₂CH₃) | F |
| B-346 | N((S)CH(CH₃)—CH₂CH₃)(CH₃) | F |
| B-347 | N((S)CH(CH₃)—CH₂CH₃)(CH₂CH₃) | F |
| B-348 | NH((R)CH(CH₃)—CH₂CH₃) | F |
| B-349 | N((R)CH(CH₃)—CH₂CH₃)(CH₃) | F |
| B-350 | N((R)CH(CH₃)—CH₂CH₃)(CH₂CH₃) | F |
| B-351 | NH(±CH(CH₃)—CH(CH₃)₂) | F |
| B-352 | N(±CH(CH₃)—CH(CH₃)₂)(CH₃) | F |
| B-353 | N(±CH(CH₃)—CH(CH₃)₂)(CH₂CH₃) | F |
| B-354 | NH((S)CH(CH₃)—CH(CH₃)₂) | F |
| B-355 | N((S)CH(CH₃)—CH(CH₃)₂)(CH₃) | F |
| B-356 | N((S)CH(CH₃)—CH(CH₃)₂)(CH₂CH₃) | F |
| B-357 | NH((R)CH(CH₃)—CH(CH₃)₂) | F |
| B-358 | N((R)CH(CH₃)—CH(CH₃)₂)(CH₃) | F |
| B-359 | N((R)CH(CH₃)—CH(CH₃)₂)(CH₂CH₃) | F |
| B-360 | NH(±CH(CH₃)—C(CH₃)₃) | F |
| B-361 | NH(±CH(CH₃)—C(CH₃)₃)(CH₃) | F |
| B-362 | N(±CH(CH₃)—C(CH₃)₃)(CH₂CH₃) | F |
| B-363 | NH((S)CH(CH₃)—C(CH₃)₃) | F |
| B-364 | N((S)CH(CH₃)—C(CH₃)₃)(CH₃) | F |
| B-365 | N((S)CH(CH₃)—C(CH₃)₃)(CH₂CH₃) | F |
| B-366 | NH((R)CH(CH₃)—C(CH₃)₃) | F |
| B-367 | N((R)CH(CH₃)—C(CH₃)₃)(CH₃) | F |
| B-368 | N((R)CH(CH₃)—C(CH₃)₃)(CH₂CH₃) | F |
| B-369 | NH(±CH(CH₃)—CF₃) | F |
| B-370 | N(±CH(CH₃)—CF₃)(CH₃) | F |
| B-371 | N(±CH(CH₃)—CF₃)(CH₂CH₃) | F |
| B-372 | NH((S)CH(CH₃)—CF₃) | F |
| B-373 | N((S)CH(CH₃)—CF₃)(CH₃) | F |
| B-374 | N((S)CH(CH₃)—CF₃)(CH₂CH₃) | F |
| B-375 | NH((R)CH(CH₃)—CF₃) | F |
| B-376 | N((R)CH(CH₃)—CF₃)(CH₃) | F |
| B-377 | N((R)CH(CH₃)—CF₃)(CH₂CH₃) | F |
| B-378 | NH(±CH(CH₃)—CCl₃) | F |
| B-379 | N(±CH(CH₃)—CCl₃)(CH₃) | F |
| B-380 | N(±CH(CH₃)—CCl₃)(CH₂CH₃) | F |
| B-381 | NH((S)CH(CH₃)—CCl₃) | F |
| B-382 | N((S)CH(CH₃)—CCl₃)(CH₃) | F |
| B-383 | N((S)CH(CH₃)—CCl₃)(CH₂CH₃) | F |
| B-384 | NH((R)CH(CH₃)—CCl₃) | F |
| B-385 | N((R)CH(CH₃)—CCl₃)(CH₃) | F |
| B-386 | N((R)CH(CH₃)—CCl₃)(CH₂CH₃) | F |
| B-387 | NH(CH₂CF₂CF₃) | F |
| B-388 | N(CH₂CF₂CF₃)(CH₃) | F |
| B-389 | N(CH₂CF₂CF₃)(CH₂CH₃) | F |
| B-390 | NH(CH₂(CF₂)₂CF₃) | F |
| B-391 | N(CH₂(CF₂)₂CF₃)(CH₃) | F |
| B-392 | N(CH₂(CF₂)₂CF₃)(CH₂CH₃) | F |
| B-393 | NH(CH₂C(CH₃)═CH₂) | F |
| B-394 | N(CH₂C(CH₃)═CH₂)(CH₃) | F |
| B-395 | N(CH₂C(CH₃)═CH₂)(CH₂CH₃) | F |
| B-396 | NH(CH₂CH═CH₂) | F |
| B-397 | N(CH₂CH═CH₂)(CH₃) | F |
| B-398 | N(CH₂CH═CH₂)(CH₂CH₃) | F |
| B-399 | NH((±)CH(CH₃)CH═CH₂) | F |
| B-400 | N((±)CH(CH₃)CH═CH₂)(CH₃) | F |
| B-401 | N((±)CH(CH₃)CH═CH₂)(CH₂CH₃) | F |
| B-402 | NH((S)CH(CH₃)CH═CH₂) | F |
| B-403 | N((S)CH(CH₃)CH═CH₂)(CH₃) | F |
| B-404 | N((S)CH(CH₃)CH═CH₂)(CH₂CH₃) | F |
| B-405 | NH((R)CH(CH₃)CH═CH₂) | F |
| B-406 | N((R)CH(CH₃)CH═CH₂)(CH₃) | F |
| B-407 | N((R)CH(CH₃)CH═CH₂)(CH₂CH₃) | F |
| B-408 | NH((±)CH(CH₃)C(CH₃)═CH₂) | F |
| B-409 | N((±)CH(CH₃)C(CH₃)═CH₂)(CH₃) | F |
| B-410 | N((±)CH(CH₃)C(CH₃)═CH₂)(CH₂CH₃) | F |
| B-411 | NH((S)CH(CH₃)C(CH₃)═CH₂) | F |
| B-412 | N((S)CH(CH₃)C(CH₃)═CH₂)(CH₃) | F |
| B-413 | N((S)CH(CH₃)C(CH₃)═CH₂)(CH₂CH₃) | F |
| B-414 | NH((R)CH(CH₃)C(CH₃)═CH₂) | F |
| B-415 | N((R)CH(CH₃)C(CH₃)═CH₂)(CH₃) | F |
| B-416 | N((R)CH(CH₃)C(CH₃)═CH₂)(CH₂CH₃) | F |
| B-417 | NH(CH₂—C≡CH) | F |
| B-418 | N(CH₂—C≡CH)(CH₃) | F |
| B-419 | N(CH₂—C≡CH)(CH₂CH₃) | F |
| B-420 | NH(cyclo-C₅H₉) | F |
| B-421 | N(cyclo-C₅H₉)(CH₃) | F |
| B-422 | N(cyclo-C₅H₉)(CH₂CH₃) | F |
| B-423 | NH(cyclo-C₆H₁₁) | F |
| B-424 | N(cyclo-C₆H₁₁)(CH₃) | F |
| B-425 | N(cyclo-C₆H₁₁)(CH₂CH₃) | F |
| B-426 | NH(CH₂—C₆H₅) | F |
| B-427 | N(CH₂—C₆H₅)(CH₃) | F |
| B-428 | N(CH₂—C₆H₅)(CH₂CH₃) | F |
| B-429 | N(NH₂)(CH₂-c-C₆H₁₁) | F |
| B-430 | N(NH₂)(CH₂CH₃) | F |
| B-431 | N(NH₂)(CH₂CH₂CH₃) | F |
| B-432 | NH(NH—CH₂—CH═CH₂) | F |
| B-433 | N(NH—CH₂—CH═CH₂)(CH₃) | F |
| B-434 | N(NH—CH₂—CH═CH₂)(CH₂CH₃) | F |
| B-435 | NH(NH—C(CH₃)₃) | F |
| B-436 | NH(N(CH₃)₂) | F |
| B-437 | NH(NH(CH₃)) | F |
| B-438 | NR²R³ where R² + R³: —(CH₂)₂CH═CHCH₂— | F |
| B-439 | NR²R³ where R² + R³: —(CH₂)₂C(CH₃)═CHCH₂— | F |
| B-440 | NR²R³ where R² + R³: —CH(CH₃)CH₂—CH═CHCH₂— | F |
| B-441 | NR²R³ where R² + R³: —(CH₂)₂CH(CH₃)(CH₂)₂— | F |
| B-442 | NR²R³ where R² + R³: —(CH₂)₃CHFCH₂— | F |
| B-443 | NR²R³ where R² + R³: —(CH₂)₂CHF(CH₂)₂— | F |
| B-444 | NR²R³ where R² + R³: —CH₂CHF(CH₂)₃— | F |
| B-445 | NR²R³ where R² + R³: —(CH₂)₂CH(CF₃)(CH₂)₂— | F |
| B-446 | NR²R³ where R² + R³: —(CH₂)₂O(CH₂)₂— | F |
| B-447 | NR²R³ where R² + R³: —(CH₂)₂S(CH₂)₂— | F |
| B-448 | NR²R³ where R² + R³: —(CH₂)₅— | F |
| B-449 | NR²R³ where R² + R³: —(CH₂)₄— | F |
| B-450 | NR²R³ where R² + R³: —CH₂CH═CHCH₂— | F |
| B-451 | NR²R³ where R² + R³: —CH(CH₃)(CH₂)₃— | F |
| B-452 | NR²R³ where R² + R³: —CH₂CH(CH₃)(CH₂)₂— | F |
| B-453 | NR²R³ where R² + R³: —CH(CH₃)—(CH₂)₂—CH(CH₃)— | F |
| B-454 | NR²R³ where R² + R³: —CH(CH₃)—(CH₂)₄— | F |
| B-455 | NR²R³ where R² + R³: —CH₂—CH(CH₃)—(CH₂)₃— | F |
| B-456 | NR²R³ where R² + R³: —(CH₂)—CH(CH₃)—CH₂—CH(CH₃)—CH₂— | F |
| B-457 | NR²R³ where R² + R³: —CH(CH₂CH₃)—(CH₂)₄— | F |
| B-458 | NR²R³ where R² + R³: —(CH₂)₆— | F |
| B-459 | NR²R³ where R² + R³: —CH(CH₃)—(CH₂)₅— | F |
| B-460 | NR²R³ where R² + R³: —(CH₂)₂—N(CH₃)—(CH₂)₂— | F |
| B-461 | NR²R³ where R² + R³: —N═CH—CH═CH— | F |
| B-462 | NR²R³ where R² + R³: —N═C(CH₃)—CH═C(CH₃)— | F |
| B-463 | NR²R³ where R² + R³: —N═C(CF₃)—CH═C(CF₃)— | F |
| B-464 | NHCH₃ | CN |
| B-465 | N(CH₃)₂ | CN |
| B-466 | NH(CH₂CH₃) | CN |
| B-467 | N(CH₂CH₃)(CH₃) | CN |
| B-468 | N(CH₂CH₃)₂ | CN |
| B-469 | NH(CH₂CF₃) | CN |
| B-470 | N(CH₂CF₃)(CH₃) | CN |
| B-471 | N(CH₂CF₃)(CH₂CH₃) | CN |
| B-472 | NH(CH₂CCl₃) | CN |
| B-473 | N(CH₂CCl₃)(CH₃) | CN |
| B-474 | N(CH₂CCl₃)(CH₂CH₃) | CN |
| B-475 | NH(CH₂CH₂CH₃) | CN |
| B-476 | N(CH₂CH₂CH₃)(CH₃) | CN |
| B-477 | N(CH₂CH₂CH₃)(CH₂CH₃) | CN |
| B-478 | N(CH₂CH₂CH₃)(CH₂CH₂CH₃) | CN |
| B-479 | NH(CH(CH₃)₂) | CN |
| B-480 | N(CH(CH₃)₂)(CH₃) | CN |
| B-481 | N(CH(CH₃)₂)(CH₂CH₃) | CN |
| B-482 | NH(CH₂CH(CH₃)₂) | CN |
| B-483 | N(CH₂CH(CH₃)₂)(CH₃) | CN |
| B-484 | N(CH₂CH(CH₃)₂)(CH₂CH₃) | CN |
| B-485 | N(CH₂CH(CH₃)₂)(CH₂CH₂CH₃) | CN |
| B-486 | NH(CH₂CH₂CH₂CH₃) | CN |
| B-487 | N(CH₂CH₂CH₂CH₃)(CH₃) | CN |
| B-488 | N(CH₂CH₂CH₂CH₃)(CH₂CH₃) | CN |
| B-489 | N(CH₂CH₂CH₂CH₃)(CH₂CH₂CH₃) | CN |
| B-490 | N(CH₂CH₂CH₂CH₃)₂ | CN |
| B-491 | NH(±CH(CH₃)—CH₂CH₃) | CN |
| B-492 | N(±CH(CH₃)—CH₂CH₃)(CH₃) | CN |
| B-493 | N(±CH(CH₃)—CH₂CH₃)(CH₂CH₃) | CN |
| B-494 | NH((S)CH(CH₃)—CH₂CH₃) | CN |
| B-495 | N((S)CH(CH₃)—CH₂CH₃)(CH₃) | CN |
| B-496 | N((S)CH(CH₃)—CH₂CH₃)(CH₂CH₃) | CN |
| B-497 | NH((R)CH(CH₃)—CH₂CH₃) | CN |
| B-498 | N((R)CH(CH₃)—CH₂CH₃)(CH₃) | CN |
| B-499 | N((R)CH(CH₃)—CH₂CH₃)(CH₂CH₃) | CN |
| B-500 | NH(±CH(CH₃)—CH(CH₃)₂) | CN |
| B-501 | N(±CH(CH₃)—CH(CH₃)₂)(CH₃) | CN |
| B-502 | N(±CH(CH₃)—CH(CH₃)₂)(CH₂CH₃) | CN |
| B-503 | NH((S)CH(CH₃)—CH(CH₃)₂) | CN |
| B-504 | N((S)CH(CH₃)—CH(CH₃)₂)(CH₃) | CN |
| B-505 | N((S)CH(CH₃)—CH(CH₃)₂)(CH₂CH₃) | CN |
| B-506 | NH((R)CH(CH₃)—CH(CH₃)₂) | CN |
| B-507 | N((R)CH(CH₃)—CH(CH₃)₂)(CH₃) | CN |
| B-508 | N((R)CH(CH₃)—CH(CH₃)₂)(CH₂CH₃) | CN |
| B-509 | NH(±CH(CH₃)—C(CH₃)₃) | CN |
| B-510 | NH(±CH(CH₃)—C(CH₃)₃)(CH₃) | CN |
| B-511 | N(±CH(CH₃)—C(CH₃)₃)(CH₂CH₃) | CN |
| B-512 | NH((S)CH(CH₃)—C(CH₃)₃) | CN |
| B-513 | N((S)CH(CH₃)—C(CH₃)₃)(CH₃) | CN |
| B-514 | N((S)CH(CH₃)—C(CH₃)₃)(CH₂CH₃) | CN |
| B-515 | NH((R)CH(CH₃)—C(CH₃)₃) | CN |
| B-516 | N((R)CH(CH₃)—C(CH₃)₃)(CH₃) | CN |
| B-517 | N((R)CH(CH₃)—C(CH₃)₃)(CH₂CH₃) | CN |
| B-518 | NH(±CH(CH₃)—CF₃) | CN |
| B-519 | N(±CH(CH₃)—CF₃)(CH₃) | CN |
| B-520 | N(±CH(CH₃)—CF₃)(CH₂CH₃) | CN |
| B-521 | NH((S)CH(CH₃)—CF₃) | CN |
| B-522 | N((S)CH(CH₃)—CF₃)(CH₃) | CN |
| B-523 | N((S)CH(CH₃)—CF₃)(CH₂CH₃) | CN |
| B-524 | NH((R)CH(CH₃)—CF₃) | CN |
| B-525 | N((R)CH(CH₃)—CF₃)(CH₃) | CN |
| B-526 | N((R)CH(CH₃)—CF₃)(CH₂CH₃) | CN |
| B-527 | NH(±CH(CH₃)—CCl₃) | CN |
| B-528 | N(±CH(CH₃)—CCl₃)(CH₃) | CN |
| B-529 | N(±CH(CH₃)—CCl₃)(CH₂CH₃) | CN |
| B-530 | NH((S)CH(CH₃)—CCl₃) | CN |
| B-531 | N((S)CH(CH₃)—CCl₃)(CH₃) | CN |
| B-532 | N((S)CH(CH₃)—CCl₃)(CH₂CH₃) | CN |
| B-533 | NH((R)CH(CH₃)—CCl₃) | CN |
| B-534 | N((R)CH(CH₃)—CCl₃)(CH₃) | CN |
| B-535 | N((R)CH(CH₃)—CCl₃)(CH₂CH₃) | CN |
| B-536 | NH(CH₂CF₂CF₃) | CN |
| B-537 | N(CH₂CF₂CF₃)(CH₃) | CN |
| B-538 | N(CH₂CF₂CF₃)(CH₂CH₃) | CN |
| B-539 | NH(CH₂(CF₂)₂CF₃) | CN |
| B-540 | N(CH₂(CF₂)₂CF₃)(CH₃) | CN |
| B-541 | N(CH₂(CF₂)₂CF₃)(CH₂CH₃) | CN |
| B-542 | NH(CH₂C(CH₃)═CH₂) | CN |
| B-543 | N(CH₂C(CH₃)═CH₂)(CH₃) | CN |
| B-544 | N(CH₂C(CH₃)═CH₂)(CH₂CH₃) | CN |
| B-545 | NH(CH₂CH═CH₂) | CN |
| B-546 | N(CH₂CH═CH₂)(CH₃) | CN |
| B-547 | N(CH₂CH═CH₂)(CH₂CH₃) | CN |
| B-548 | NH((±)CH(CH₃)CH═CH₂) | CN |
| B-549 | N((±)CH(CH₃)CH═CH₂)(CH₃) | CN |
| B-550 | N((±)CH(CH₃)CH═CH₂)(CH₂CH₃) | CN |
| B-551 | NH((S)CH(CH₃)CH═CH₂) | CN |
| B-552 | N((S)CH(CH₃)CH═CH₂)(CH₃) | CN |
| B-553 | N((S)CH(CH₃)CH═CH₂)(CH₂CH₃) | CN |
| B-554 | NH((R)CH(CH₃)CH═CH₂) | CN |
| B-555 | N((R)CH(CH₃)CH═CH₂)(CH₃) | CN |
| B-556 | N((R)CH(CH₃)CH═CH₂)(CH₂CH₃) | CN |
| B-557 | NH((±)CH(CH₃)C(CH₃)═CH₂) | CN |
| B-558 | N((±)CH(CH₃)C(CH₃)═CH₂)(CH₃) | CN |
| B-559 | N((±)CH(CH₃)C(CH₃)═CH₂)(CH₂CH₃) | CN |
| B-560 | NH((S)CH(CH₃)C(CH₃)═CH₂) | CN |
| B-561 | N((S)CH(CH₃)C(CH₃)═CH₂)(CH₃) | CN |
| B-562 | N((S)CH(CH₃)C(CH₃)═CH₂)(CH₂CH₃) | CN |
| B-563 | NH((R)CH(CH₃)C(CH₃)═CH₂) | CN |
| B-564 | N((R)CH(CH₃)C(CH₃)═CH₂)(CH₃) | CN |
| B-565 | N((R)CH(CH₃)C(CH₃)═CH₂)(CH₂CH₃) | CN |
| B-566 | NH(CH₂—C≡CH) | CN |
| B-567 | N(CH₂—C≡CH)(CH₃) | CN |
| B-568 | N(CH₂—C≡CH)(CH₂CH₃) | CN |
| B-569 | NH(cyclo-C₅H₉) | CN |
| B-570 | N(cyclo-C₅H₉)(CH₃) | CN |
| B-571 | N(cyclo-C₅H₉)(CH₂CH₃) | CN |
| B-572 | NH(cyclo-C₆H₁₁) | CN |
| B-573 | N(cyclo-C₆H₁₁)(CH₃) | CN |
| B-574 | N(cyclo-C₆H₁₁)(CH₂CH₃) | CN |
| B-575 | NH(CH₂—C₆H₅) | CN |
| B-576 | N(CH₂—C₆H₅)(CH₃) | CN |
| B-577 | N(CH₂—C₆H₅)(CH₂CH₃) | CN |
| B-578 | N(NH₂)(CH₂-c-C₆H₁₁) | CN |
| B-579 | N(NH₂)(CH₂CH₃) | CN |
| B-580 | N(NH₂)(CH₂CH₂CH₃) | CN |
| B-581 | NH(NH—CH₂—CH═CH₂) | CN |
| B-582 | N(NH—CH₂—CH═CH₂)(CH₃) | CN |
| B-583 | N(NH—CH₂—CH═CH₂)(CH₂CH₃) | CN |
| B-584 | NH(NH—C(CH₃)₃) | CN |
| B-585 | NH(N(CH₃)₂) | CN |
| B-586 | NH(NH(CH₃)) | CN |
| B-587 | NR²R³ where R² + R³: —(CH₂)₂CH═CHCH₂— | CN |
| B-588 | NR²R³ where R² + R³: —(CH₂)₂C(CH₃)═CHCH₂— | CN |
| B-589 | NR²R³ where R² + R³: —CH(CH₃)CH₂—CH═CHCH₂— | CN |
| B-590 | NR²R³ where R² + R³: —(CH₂)₂CH(CH₃)(CH₂)₂— | CN |
| B-591 | NR²R³ where R² + R³: —(CH₂)₃CHFCH₂— | CN |
| B-592 | NR²R³ where R² + R³: —(CH₂)₂CHF(CH₂)₂— | CN |
| B-593 | NR²R³ where R² + R³: —CH₂CHF(CH₂)₃— | CN |
| B-594 | NR²R³ where R² + R³: —(CH₂)₂CH(CF₃)(CH₂)₂— | CN |
| B-595 | NR²R³ where R² + R³: —(CH₂)₂O(CH₂)₂— | CN |
| B-596 | NR²R³ where R² + R³: —(CH₂)₂S(CH₂)₂— | CN |
| B-597 | NR²R³ where R² + R³: —(CH₂)₅— | CN |
| B-598 | NR²R³ where R² + R³: —(CH₂)₄— | CN |
| B-599 | NR²R³ where R² + R³: —CH₂CH═CHCH₂— | CN |
| B-600 | NR²R³ where R² + R³: —CH(CH₃)(CH₂)₃— | CN |
| B-601 | NR²R³ where R² + R³: —CH₂CH(CH₃)(CH₂)₂— | CN |
| B-602 | NR²R³ where R² + R³: —CH(CH₃)—(CH₂)₂—CH(CH₃)— | CN |
| B-603 | NR²R³ where R² + R³: —CH(CH₃)—(CH₂)₄— | CN |
| B-604 | NR²R³ where R² + R³: —CH₂—CH(CH₃)—(CH₂)₃— | CN |
| B-605 | NR²R³ where R² + R³: —(CH₂)—CH(CH₃)—CH₂—CH(CH₃)—CH₂— | CN |
| B-606 | NR²R³ where R² + R³: —CH(CH₂CH₃)—(CH₂)₄— | CN |
| B-607 | NR²R³ where R² + R³: —(CH₂)₆— | CN |
| B-608 | NR²R³ where R² + R³: —CH(CH₃)—(CH₂)₅— | CN |
| B-609 | NR²R³ where R² + R³: —(CH₂)₂—N(CH₃)—(CH₂)₂— | CN |
| B-610 | NR²R³ where R² + R³: —N═CH—CH═CH— | CN |
| B-611 | NR²R³ where R² + R³: —N═C(CH₃)—CH═C(CH₃)— | CN |
| B-612 | NR²R³ where R² + R³: —N═C(CF₃)—CH═C(CF₃)— | CN |
| B-613 | NHCH₃ | Br |
| B-614 | N(CH₃)₂ | Br |
| B-615 | NH(CH₂CH₃) | Br |
| B-616 | N(CH₂CH₃)(CH₃) | Br |
| B-617 | N(CH₂CH₃)₂ | Br |
| B-618 | NH(CH₂CF₃) | Br |
| B-619 | N(CH₂CF₃)(CH₃) | Br |
| B-620 | N(CH₂CF₃)(CH₂CH₃) | Br |
| B-621 | NH(CH₂CCl₃) | Br |
| B-622 | N(CH₂CCl₃)(CH₃) | Br |
| B-623 | N(CH₂CCl₃)(CH₂CH₃) | Br |
| B-624 | NH(CH₂CH₂CH₃) | Br |
| B-625 | N(CH₂CH₂CH₃)(CH₃) | Br |
| B-626 | N(CH₂CH₂CH₃)(CH₂CH₃) | Br |
| B-627 | N(CH₂CH₂CH₃)(CH₂CH₂CH₃) | Br |
| B-628 | NH(CH(CH₃)₂) | Br |
| B-629 | N(CH(CH₃)₂)(CH₃) | Br |
| B-630 | N(CH(CH₃)₂)(CH₂CH₃) | Br |
| B-631 | NH(CH₂CH(CH₃)₂) | Br |
| B-632 | N(CH₂CH(CH₃)₂)(CH₃) | Br |
| B-633 | N(CH₂CH(CH₃)₂)(CH₂CH₃) | Br |
| B-634 | N(CH₂CH(CH₃)₂)(CH₂CH₂CH₃) | Br |
| B-635 | NH(CH₂CH₂CH₂CH₃) | Br |
| B-636 | N(CH₂CH₂CH₂CH₃)(CH₃) | Br |
| B-637 | N(CH₂CH₂CH₂CH₃)(CH₂CH₃) | Br |
| B-638 | N(CH₂CH₂CH₂CH₃)(CH₂CH₂CH₃) | Br |
| B-639 | N(CH₂CH₂CH₂CH₃)₂ | Br |
| B-640 | NH(±CH(CH₃)—CH₂CH₃) | Br |
| B-641 | N(±CH(CH₃)—CH₂CH₃)(CH₃) | Br |
| B-642 | N(±CH(CH₃)—CH₂CH₃)(CH₂CH₃) | Br |
| B-643 | NH((S)CH(CH₃)—CH₂CH₃) | Br |
| B-644 | N((S)CH(CH₃)—CH₂CH₃)(CH₃) | Br |
| B-645 | N((S)CH(CH₃)—CH₂CH₃)(CH₂CH₃) | Br |
| B-646 | NH((R)CH(CH₃)—CH₂CH₃) | Br |
| B-647 | N((R)CH(CH₃)—CH₂CH₃)(CH₃) | Br |
| B-648 | N((R)CH(CH₃)—CH₂CH₃)(CH₂CH₃) | Br |
| B-649 | NH(±CH(CH₃)—CH(CH₃)₂) | Br |
| B-650 | N(±CH(CH₃)—CH(CH₃)₂)(CH₃) | Br |
| B-651 | N(±CH(CH₃)—CH(CH₃)₂)(CH₂CH₃) | Br |
| B-652 | NH((S)CH(CH₃)—CH(CH₃)₂) | Br |
| B-653 | N((S)CH(CH₃)—CH(CH₃)₂)(CH₃) | Br |
| B-654 | N((S)CH(CH₃)—CH(CH₃)₂)(CH₂CH₃) | Br |
| B-655 | NH((R)CH(CH₃)—CH(CH₃)₂) | Br |
| B-656 | N((R)CH(CH₃)—CH(CH₃)₂)(CH₃) | Br |
| B-657 | N((R)CH(CH₃)—CH(CH₃)₂)(CH₂CH₃) | Br |
| B-658 | NH(±CH(CH₃)—C(CH₃)₃) | Br |
| B-659 | NH(±CH(CH₃)—C(CH₃)₃)(CH₃) | Br |
| B-660 | N(±CH(CH₃)—C(CH₃)₃)(CH₂CH₃) | Br |
| B-661 | NH((S)CH(CH₃)—C(CH₃)₃) | Br |
| B-662 | N((S)CH(CH₃)—C(CH₃)₃)(CH₃) | Br |
| B-663 | N((S)CH(CH₃)—C(CH₃)₃)(CH₂CH₃) | Br |
| B-664 | NH((R)CH(CH₃)—C(CH₃)₃) | Br |
| B-665 | N((R)CH(CH₃)—C(CH₃)₃)(CH₃) | Br |
| B-666 | N((R)CH(CH₃)—C(CH₃)₃)(CH₂CH₃) | Br |
| B-667 | NH(±CH(CH₃)—CF₃) | Br |
| B-668 | N(±CH(CH₃)—CF₃)(CH₃) | Br |
| B-669 | N(±CH(CH₃)—CF₃)(CH₂CH₃) | Br |
| B-670 | NH((S)CH(CH₃)—CF₃) | Br |
| B-671 | N((S)CH(CH₃)—CF₃)(CH₃) | Br |
| B-672 | N((S)CH(CH₃)—CF₃)(CH₂CH₃) | Br |
| B-673 | NH((R)CH(CH₃)—CF₃) | Br |
| B-674 | N((R)CH(CH₃)—CF₃)(CH₃) | Br |
| B-675 | N((R)CH(CH₃)—CF₃)(CH₂CH₃) | Br |
| B-676 | NH(±CH(CH₃)—CCl₃) | Br |
| B-677 | N(±CH(CH₃)—CCl₃)(CH₃) | Br |
| B-678 | N(±CH(CH₃)—CCl₃)(CH₂CH₃) | Br |
| B-679 | NH((S)CH(CH₃)—CCl₃) | Br |
| B-680 | N((S)CH(CH₃)—CCl₃)(CH₃) | Br |
| B-681 | N((S)CH(CH₃)—CCl₃)(CH₂CH₃) | Br |
| B-682 | NH((R)CH(CH₃)—CCl₃) | Br |
| B-683 | N((R)CH(CH₃)—CCl₃)(CH₃) | Br |
| B-684 | N((R)CH(CH₃)—CCl₃)(CH₂CH₃) | Br |
| B-685 | NH(CH₂CF₂CF₃) | Br |
| B-686 | N(CH₂CF₂CF₃)(CH₃) | Br |
| B-687 | N(CH₂CF₂CF₃)(CH₂CH₃) | Br |
| B-688 | NH(CH₂(CF₂)₂CF₃) | Br |
| B-689 | N(CH₂(CF₂)₂CF₃)(CH₃) | Br |
| B-690 | N(CH₂(CF₂)₂CF₃)(CH₂CH₃) | Br |
| B-691 | NH(CH₂C(CH₃)═CH₂) | Br |
| B-692 | N(CH₂C(CH₃)═CH₂)(CH₃) | Br |
| B-693 | N(CH₂C(CH₃)═CH₂)(CH₂CH₃) | Br |
| B-694 | NH(CH₂CH═CH₂) | Br |
| B-695 | N(CH₂CH═CH₂)(CH₃) | Br |
| B-696 | N(CH₂CH═CH₂)(CH₂CH₃) | Br |
| B-697 | NH((±)CH(CH₃)CH═CH₂) | Br |
| B-698 | N((±)CH(CH₃)CH═CH₂)(CH₃) | Br |
| B-699 | N((±)CH(CH₃)CH═CH₂)(CH₂CH₃) | Br |
| B-700 | NH((S)CH(CH₃)CH═CH₂) | Br |
| B-701 | N((S)CH(CH₃)CH═CH₂)(CH₃) | Br |
| B-702 | N((S)CH(CH₃)CH═CH₂)(CH₂CH₃) | Br |
| B-703 | NH((R)CH(CH₃)CH═CH₂) | Br |
| B-704 | N((R)CH(CH₃)CH═CH₂)(CH₃) | Br |
| B-705 | N((R)CH(CH₃)CH═CH₂)(CH₂CH₃) | Br |
| B-706 | NH((±)CH(CH₃)C(CH₃)═CH₂) | Br |
| B-707 | N((±)CH(CH₃)C(CH₃)═CH₂)(CH₃) | Br |
| B-708 | N((±)CH(CH₃)C(CH₃)═CH₂)(CH₂CH₃) | Br |
| B-709 | NH((S)CH(CH₃)C(CH₃)═CH₂) | Br |
| B-710 | N((S)CH(CH₃)C(CH₃)═CH₂)(CH₃) | Br |
| B-711 | N((S)CH(CH₃)C(CH₃)═CH₂)(CH₂CH₃) | Br |
| B-712 | NH((R)CH(CH₃)C(CH₃)═CH₂) | Br |
| B-713 | N((R)CH(CH₃)C(CH₃)═CH₂)(CH₃) | Br |
| B-714 | N((R)CH(CH₃)C(CH₃)═CH₂)(CH₂CH₃) | Br |
| B-715 | NH(CH₂—C≡CH) | Br |
| B-716 | N(CH₂—C≡CH)(CH₃) | Br |
| B-717 | N(CH₂—C≡CH)(CH₂CH₃) | Br |
| B-718 | NH(cyclo-C₅H₉) | Br |
| B-719 | N(cyclo-C₅H₉)(CH₃) | Br |
| B-720 | N(cyclo-C₅H₉)(CH₂CH₃) | Br |
| B-721 | NH(cyclo-C₆H₁₁) | Br |
| B-722 | N(cyclo-C₆H₁₁)(CH₃) | Br |
| B-723 | N(cyclo-C₆H₁₁)(CH₂CH₃) | Br |
| B-724 | NH(CH₂—C₆H₅) | Br |
| B-725 | N(CH₂—C₆H₅)(CH₃) | Br |
| B-726 | N(CH₂—C₆H₅)(CH₂CH₃) | Br |
| B-727 | N(NH₂)(CH₂-c-C₆H₁₁) | Br |
| B-728 | N(NH₂)(CH₂CH₃) | Br |
| B-729 | N(NH₂)(CH₂CH₂CH₃) | Br |
| B-730 | NH(NH—CH₂—CH═CH₂) | Br |
| B-731 | N(NH—CH₂—CH═CH₂)(CH₃) | Br |
| B-732 | N(NH—CH₂—CH═CH₂)(CH₂CH₃) | Br |
| B-733 | NH(NH—C(CH₃)₃) | Br |
| B-734 | NH(N(CH₃)₂) | Br |
| B-735 | NH(NH(CH₃)) | Br |
| B-736 | NR²R³ where R² + R³: —(CH₂)₂CH═CHCH₂— | Br |
| B-737 | NR²R³ where R² + R³: —(CH₂)₂C(CH₃)═CHCH₂— | Br |
| B-738 | NR²R³ where R² + R³: —CH(CH₃)CH₂—CH═CHCH₂— | Br |
| B-739 | NR²R³ where R² + R³: —(CH₂)₂CH(CH₃)(CH₂)₂— | Br |
| B-740 | NR²R³ where R² + R³: —(CH₂)₃CHFCH₂— | Br |
| B-741 | NR²R³ where R² + R³: —(CH₂)₂CHF(CH₂)₂— | Br |
| B-742 | NR²R³ where R² + R³: —CH₂CHF(CH₂)₃— | Br |
| B-743 | NR²R³ where R² + R³: —(CH₂)₂CH(CF₃)(CH₂)₂— | Br |
| B-744 | NR²R³ where R² + R³: —(CH₂)₂O(CH₂)₂— | Br |
| B-745 | NR²R³ where R² + R³: —(CH₂)₂S(CH₂)₂— | Br |
| B-746 | NR²R³ where R² + R³: —(CH₂)₅— | Br |
| B-747 | NR²R³ where R² + R³: —(CH₂)₄— | Br |
| B-748 | NR²R³ where R² + R³: —CH₂CH═CHCH₂— | Br |
| B-749 | NR²R³ where R² + R³: —CH(CH₃)(CH₂)₃— | Br |
| B-750 | NR²R³ where R² + R³: —CH₂CH(CH₃)(CH₂)₂— | Br |
| B-751 | NR²R³ where R² + R³: —CH(CH₃)—(CH₂)₂—CH(CH₃)— | Br |
| B-752 | NR²R³ where R² + R³: —CH(CH₃)—(CH₂)₄— | Br |
| B-753 | NR²R³ where R² + R³: —CH₂—CH(CH₃)—(CH₂)₃— | Br |
| B-754 | NR²R³ where R² + R³: —(CH₂)—CH(CH₃)—CH₂—CH(CH₃)—CH₂— | Br |
| B-755 | NR²R³ where R² + R³: —CH(CH₂CH₃)—(CH₂)₄— | Br |
| B-756 | NR²R³ where R² + R³: —(CH₂)₆— | Br |
| B-757 | NR²R³ where R² + R³: —CH(CH₃)—(CH₂)₅— | Br |
| B-758 | NR²R³ where R² + R³: —(CH₂)₂—N(CH₃)—(CH₂)₂— | Br |
| B-759 | NR²R³ where R² + R³: —N═CH—CH═CH— | Br |
| B-760 | NR²R³ where R² + R³: —N═C(CH₃)—CH═C(CH₃)— | Br |
| B-761 | NR²R³ where R² + R³: —N═C(CF₃)—CH═C(CF₃)— | Br |
| B-762 | NHCH₃ | OCH₃ |
| B-763 | N(CH₃)₂ | OCH₃ |
| B-764 | NH(CH₂CH₃) | OCH₃ |
| B-765 | N(CH₂CH₃)(CH₃) | OCH₃ |
| B-766 | N(CH₂CH₃)₂ | OCH₃ |
| B-767 | NH(CH₂CF₃) | OCH₃ |
| B-768 | N(CH₂CF₃)(CH₃) | OCH₃ |
| B-769 | N(CH₂CF₃)(CH₂CH₃) | OCH₃ |
| B-770 | NH(CH₂CCl₃) | OCH₃ |
| B-771 | N(CH₂CCl₃)(CH₃) | OCH₃ |
| B-772 | N(CH₂CCl₃)(CH₂CH₃) | OCH₃ |
| B-773 | NH(CH₂CH₂CH₃) | OCH₃ |
| B-774 | N(CH₂CH₂CH₃)(CH₃) | OCH₃ |
| B-775 | N(CH₂CH₂CH₃)(CH₂CH₃) | OCH₃ |
| B-776 | N(CH₂CH₂CH₃)(CH₂CH₂CH₃) | OCH₃ |
| B-777 | NH(CH(CH₃)₂) | OCH₃ |
| B-778 | N(CH(CH₃)₂)(CH₃) | OCH₃ |
| B-779 | N(CH(CH₃)₂)(CH₂CH₃) | OCH₃ |
| B-780 | NH(CH₂CH(CH₃)₂) | OCH₃ |
| B-781 | N(CH₂CH(CH₃)₂)(CH₃) | OCH₃ |
| B-782 | N(CH₂CH(CH₃)₂)(CH₂CH₃) | OCH₃ |
| B-783 | N(CH₂CH(CH₃)₂)(CH₂CH₂CH₃) | OCH₃ |
| B-784 | NH(CH₂CH₂CH₂CH₃) | OCH₃ |
| B-785 | N(CH₂CH₂CH₂CH₃)(CH₃) | OCH₃ |
| B-786 | N(CH₂CH₂CH₂CH₃)(CH₂CH₃) | OCH₃ |
| B-787 | N(CH₂CH₂CH₂CH₃)(CH₂CH₂CH₃) | OCH₃ |
| B-788 | N(CH₂CH₂CH₂CH₃)₂ | OCH₃ |
| B-789 | NH(±CH(CH₃)—CH₂CH₃) | OCH₃ |
| B-790 | N(±CH(CH₃)—CH₂CH₃)(CH₃) | OCH₃ |
| B-791 | N(±CH(CH₃)—CH₂CH₃)(CH₂CH₃) | OCH₃ |
| B-792 | NH((S)CH(CH₃)—CH₂CH₃) | OCH₃ |
| B-793 | N((S)CH(CH₃)—CH₂CH₃)(CH₃) | OCH₃ |
| B-794 | N((S)CH(CH₃)—CH₂CH₃)(CH₂CH₃) | OCH₃ |
| B-795 | NH((R)CH(CH₃)—CH₂CH₃) | OCH₃ |
| B-796 | N((R)CH(CH₃)—CH₂CH₃)(CH₃) | OCH₃ |
| B-797 | N((R)CH(CH₃)—CH₂CH₃)(CH₂CH₃) | OCH₃ |
| B-798 | NH(±CH(CH₃)—CH(CH₃)₂) | OCH₃ |
| B-799 | N(±CH(CH₃)—CH(CH₃)₂)(CH₃) | OCH₃ |
| B-800 | N(±CH(CH₃)—CH(CH₃)₂)(CH₂CH₃) | OCH₃ |
| B-801 | NH((S)CH(CH₃)—CH(CH₃)₂) | OCH₃ |
| B-802 | N((S)CH(CH₃)—CH(CH₃)₂)(CH₃) | OCH₃ |
| B-803 | N((S)CH(CH₃)—CH(CH₃)₂)(CH₂CH₃) | OCH₃ |
| B-804 | NH((R)CH(CH₃)—CH(CH₃)₂) | OCH₃ |
| B-805 | N((R)CH(CH₃)—CH(CH₃)₂)(CH₃) | OCH₃ |
| B-806 | N((R)CH(CH₃)—CH(CH₃)₂)(CH₂CH₃) | OCH₃ |
| B-807 | NH(±CH(CH₃)—C(CH₃)₃) | OCH₃ |
| B-808 | NH(±CH(CH₃)—C(CH₃)₃)(CH₃) | OCH₃ |
| B-809 | N(±CH(CH₃)—C(CH₃)₃)(CH₂CH₃) | OCH₃ |
| B-810 | NH((S)CH(CH₃)—C(CH₃)₃) | OCH₃ |
| B-811 | N((S)CH(CH₃)—C(CH₃)₃)(CH₃) | OCH₃ |
| B-812 | N((S)CH(CH₃)—C(CH₃)₃)(CH₂CH₃) | OCH₃ |
| B-813 | NH((R)CH(CH₃)—C(CH₃)₃) | OCH₃ |
| B-814 | N((R)CH(CH₃)—C(CH₃)₃)(CH₃) | OCH₃ |
| B-815 | N((R)CH(CH₃)—C(CH₃)₃)(CH₂CH₃) | OCH₃ |
| B-816 | NH(±CH(CH₃)—CF₃) | OCH₃ |
| B-817 | N(±CH(CH₃)—CF₃)(CH₃) | OCH₃ |
| B-818 | N(±CH(CH₃)—CF₃)(CH₂CH₃) | OCH₃ |
| B-819 | NH((S)CH(CH₃)—CF₃) | OCH₃ |
| B-820 | N((S)CH(CH₃)—CF₃)(CH₃) | OCH₃ |
| B-821 | N((S)CH(CH₃)—CF₃)(CH₂CH₃) | OCH₃ |
| B-822 | NH((R)CH(CH₃)—CF₃) | OCH₃ |
| B-823 | N((R)CH(CH₃)—CF₃)(CH₃) | OCH₃ |
| B-824 | N((R)CH(CH₃)—CF₃)(CH₂CH₃) | OCH₃ |
| B-825 | NH(±CH(CH₃)—CCl₃) | OCH₃ |
| B-826 | N(±CH(CH₃)—CCl₃)(CH₃) | OCH₃ |
| B-827 | N(±CH(CH₃)—CCl₃)(CH₂CH₃) | OCH₃ |
| B-828 | NH((S)CH(CH₃)—CCl₃) | OCH₃ |
| B-829 | N((S)CH(CH₃)—CCl₃)(CH₃) | OCH₃ |
| B-830 | N((S)CH(CH₃)—CCl₃)(CH₂CH₃) | OCH₃ |
| B-831 | NH((R)CH(CH₃)—CCl₃) | OCH₃ |
| B-832 | N((R)CH(CH₃)—CCl₃)(CH₃) | OCH₃ |
| B-833 | N((R)CH(CH₃)—CCl₃)(CH₂CH₃) | OCH₃ |
| B-834 | NH(CH₂CF₂CF₃) | OCH₃ |
| B-835 | N(CH₂CF₂CF₃)(CH₃) | OCH₃ |
| B-836 | N(CH₂CF₂CF₃)(CH₂CH₃) | OCH₃ |
| B-837 | NH(CH₂(CF₂)₂CF₃) | OCH₃ |
| B-838 | N(CH₂(CF₂)₂CF₃)(CH₃) | OCH₃ |
| B-839 | N(CH₂(CF₂)₂CF₃)(CH₂CH₃) | OCH₃ |
| B-840 | NH(CH₂C(CH₃)═CH₂) | OCH₃ |
| B-841 | N(CH₂C(CH₃)═CH₂)(CH₃) | OCH₃ |
| B-842 | N(CH₂C(CH₃)═CH₂)(CH₂CH₃) | OCH₃ |
| B-843 | NH(CH₂CH═CH₂) | OCH₃ |
| B-844 | N(CH₂CH═CH₂)(CH₃) | OCH₃ |
| B-845 | N(CH₂CH═CH₂)(CH₂CH₃) | OCH₃ |
| B-846 | NH((±)CH(CH₃)CH═CH₂) | OCH₃ |
| B-847 | N((±)CH(CH₃)CH═CH₂)(CH₃) | OCH₃ |
| B-848 | N((±)CH(CH₃)CH═CH₂)(CH₂CH₃) | OCH₃ |
| B-849 | NH((S)CH(CH₃)CH═CH₂) | OCH₃ |
| B-850 | N((S)CH(CH₃)CH═CH₂)(CH₃) | OCH₃ |
| B-851 | N((S)CH(CH₃)CH═CH₂)(CH₂CH₃) | OCH₃ |
| B-852 | NH((R)CH(CH₃)CH═CH₂) | OCH₃ |
| B-853 | N((R)CH(CH₃)CH═CH₂)(CH₃) | OCH₃ |
| B-854 | N((R)CH(CH₃)CH═CH₂)(CH₂CH₃) | OCH₃ |
| B-855 | NH((±)CH(CH₃)C(CH₃)═CH₂) | OCH₃ |
| B-856 | N((±)CH(CH₃)C(CH₃)═CH₂)(CH₃) | OCH₃ |
| B-857 | N((±)CH(CH₃)C(CH₃)═CH₂)(CH₂CH₃) | OCH₃ |
| B-858 | NH((S)CH(CH₃)C(CH₃)═CH₂) | OCH₃ |
| B-859 | N((S)CH(CH₃)C(CH₃)═CH₂)(CH₃) | OCH₃ |
| B-860 | N((S)CH(CH₃)C(CH₃)═CH₂)(CH₂CH₃) | OCH₃ |
| B-861 | NH((R)CH(CH₃)C(CH₃)═CH₂) | OCH₃ |
| B-862 | N((R)CH(CH₃)C(CH₃)═CH₂)(CH₃) | OCH₃ |
| B-863 | N((R)CH(CH₃)C(CH₃)═CH₂)(CH₂CH₃) | OCH₃ |
| B-864 | NH(CH₂—C≡CH) | OCH₃ |
| B-865 | N(CH₂—C≡CH)(CH₃) | OCH₃ |
| B-866 | N(CH₂—C≡CH)(CH₂CH₃) | OCH₃ |
| B-867 | NH(cyclo-C₅H₉) | OCH₃ |
| B-868 | N(cyclo-C₅H₉)(CH₃) | OCH₃ |
| B-869 | N(cyclo-C₅H₉)(CH₂CH₃) | OCH₃ |
| B-870 | NH(cyclo-C₆H₁₁) | OCH₃ |
| B-871 | N(cyclo-C₆H₁₁)(CH₃) | OCH₃ |
| B-872 | N(cyclo-C₆H₁₁)(CH₂CH₃) | OCH₃ |
| B-873 | NH(CH₂—C₆H₅) | OCH₃ |
| B-874 | N(CH₂—C₆H₅)(CH₃) | OCH₃ |
| B-875 | N(CH₂—C₆H₅)(CH₂CH₃) | OCH₃ |
| B-876 | N(NH₂)(CH₂-c-C₆H₁₁) | OCH₃ |
| B-877 | N(NH₂)(CH₂CH₃) | OCH₃ |
| B-878 | N(NH₂)(CH₂CH₂CH₃) | OCH₃ |
| B-879 | NH(NH—CH₂—CH═CH₂) | OCH₃ |
| B-880 | N(NH—CH₂—CH═CH₂)(CH₃) | OCH₃ |
| B-881 | N(NH—CH₂—CH═CH₂)(CH₂CH₃) | OCH₃ |
| B-882 | NH(NH—C(CH₃)₃) | OCH₃ |
| B-883 | NH(N(CH₃)₂) | OCH₃ |
| B-884 | NH(NH(CH₃)) | OCH₃ |
| B-885 | NR²R³ where R² + R³: —(CH₂)₂CH═CHCH₂— | OCH₃ |
| B-886 | NR²R³ where R² + R³: —(CH₂)₂C(CH₃)═CHCH₂— | OCH₃ |
| B-887 | NR²R³ where R² + R³: —CH(CH₃)CH₂—CH═CHCH₂— | OCH₃ |
| B-888 | NR²R³ where R² + R³: —(CH₂)₂CH(CH₃)(CH₂)₂— | OCH₃ |
| B-889 | NR²R³ where R² + R³: —(CH₂)₃CHFCH₂— | OCH₃ |
| B-890 | NR²R³ where R² + R³: —(CH₂)₂CHF(CH₂)₂— | OCH₃ |
| B-891 | NR²R³ where R² + R³: —CH₂CHF(CH₂)₃— | OCH₃ |
| B-892 | NR²R³ where R² + R³: —(CH₂)₂CH(CF₃)(CH₂)₂— | OCH₃ |
| B-893 | NR²R³ where R² + R³: —(CH₂)₂O(CH₂)₂— | OCH₃ |
| B-894 | NR²R³ where R² + R³: —(CH₂)₂S(CH₂)₂— | OCH₃ |
| B-895 | NR²R³ where R² + R³: —(CH₂)₅— | OCH₃ |
| B-896 | NR²R³ where R² + R³: —(CH₂)₄— | OCH₃ |
| B-897 | NR²R³ where R² + R³: —CH₂CH═CHCH₂— | OCH₃ |
| B-898 | NR²R³ where R² + R³: —CH(CH₃)(CH₂)₃— | OCH₃ |
| B-899 | NR²R³ where R² + R³: —CH₂CH(CH₃)(CH₂)₂— | OCH₃ |
| B-900 | NR²R³ where R² + R³: —CH(CH₃)—(CH₂)₂—CH(CH₃)— | OCH₃ |
| B-901 | NR²R³ where R² + R³: —CH(CH₃)—(CH₂)₄— | OCH₃ |
| B-902 | NR²R³ where R² + R³: —CH₂—CH(CH₃)—(CH₂)₃— | OCH₃ |
| B-903 | NR²R³ where R² + R³: —(CH₂)—CH(CH₃)—CH₂—CH(CH₃)—CH₂— | OCH₃ |
| B-904 | NR²R³ where R² + R³: —CH(CH₂CH₃)—(CH₂)₄— | OCH₃ |
| B-905 | NR²R³ where R² + R³: —(CH₂)₆— | OCH₃ |
| B-906 | NR²R³ where R² + R³: —CH(CH₃)—(CH₂)₅— | OCH₃ |
| B-907 | NR²R³ where R² + R³: —(CH₂)₂—N(CH₃)—(CH₂)₂— | OCH₃ |
| B-908 | NR²R³ where R² + R³: —N═CH—CH═CH— | OCH₃ |
| B-909 | NR²R³ where R² + R³: —N═C(CH₃)—CH═C(CH₃)— | OCH₃ |
| B-910 | NR²R³ where R² + R³: —N═C(CF₃)—CH═C(CF₃)— | OCH₃ |

According to the present invention, preference is given to compositions of a compound of the formula I and at least one active compound selected from the group consisting of the A) azoles. In particular, the invention relates to compositions comprising one of the compounds of the formula I individually listed in Tables 1 to 110 and at least one active compound selected from the group consisting of the A) azoles.

According to the present invention, preference is also given to compositions of a compound of the formula Ia and at least one active compound selected from the group consisting of the A) azoles. According to the present invention, preference is also given to compositions of a compound of the formula Ia.1 and at least one active compound selected from the group consisting of the A) azoles. According to the present invention, preference is also given to compositions of a compound of the formula Ia-1 and at least one active compound selected from the group consisting of the A) azoles. According to the present invention, preference is also given to compositions of a compound of the formula Ia.1-1 and at least one active compound selected from the group consisting of the A) azoles.

According to the present invention, preference is also given to compositions of a compound of the formula Ib and at least one active compound selected from the group consisting of the A) azoles. According to the present invention, preference is also given to compositions of a compound of the formula Ib.1 and at least one active compound selected from the group consisting of the A) azoles. According to the present invention, preference is also given to compositions of a compound of the formula Ib-1 and at least one active compound selected from the group consisting of the A) azoles. According to the present invention, preference is also given to compositions of a compound of the formula Ib.1-1 and at least one active compound selected from the group consisting of the A) azoles.

According to the present invention, preference is also given to compositions of a compound of the formula Ic and at least one active compound selected from the group consisting of the A) azoles. According to the present invention, preference is also given to compositions of a compound of the formula Ic.1 and at least one active compound selected from the group consisting of the A) azoles. According to the present invention, preference is also given to compositions of a compound of the formula Ic-1 and at least one active compound selected from the group consisting of the A) azoles. According to the present invention, preference is also given to compositions of a compound of the formula Ic.1-1 and at least one active compound selected from the group consisting of the A) azoles.

According to the present invention, preference is also given to compositions of a compound of the formula Id and at least one active compound selected from the group consisting of the A) azoles. According to the present invention, preference is also given to compositions of a compound of the formula Id.1 and at least one active compound selected from the group consisting of the A) azoles. According to the present invention, preference is also given to compositions of a compound of the formula Id-1 and at least one active compound selected from the group consisting of the A) azoles. According to the present invention, preference is also given to compositions of a compound of the formula Id.1-1 and at least one active compound selected from the group consisting of the A) azoles.

According to the present invention, preference is also given to compositions of a compound of the formula Ie and at least one active compound selected from the group consisting of the A) azoles. According to the present invention, preference is also given to compositions of a compound of the formula Ie.1 and at least one active compound selected from the group consisting of the A) azoles. According to the present invention, preference is also given to compositions of a compound of the formula Ie-1 and at least one active compound selected from the group consisting of the A) azoles. According to the present invention, preference is also given to compositions of a compound of the formula Ie.1-1 and at least one active compound selected from the group consisting of the A) azoles.

According to the present invention, preference is also given to compositions of a compound of the formula If and at least one active compound selected from the group consisting of the A) azoles. According to the present invention, preference is also given to compositions of a compound of the formula If.1 and at least one active compound selected from the group consisting of the A) azoles. According to the present invention, preference is also given to compositions of a compound of the formula If-1 and at least one active compound selected from the group consisting of the A) azoles. According to the present invention, preference is also given to compositions of a compound of the formula If.1-1 and at least one active compound selected from the group consisting of the A) azoles.

Preference is also given to compositions of a compound of the formula I and at least one active compound selected from the group consisting of the B) strobilurins. In particular, the invention relates to compositions comprising one of the compounds of the formula I individually listed in Tables 1 to 110 and at least one active compound selected from the group consisting of the B) strobilurins.

According to the present invention, preference is also given to compositions of a compound of the formula Ia and at least one active compound selected from the group consisting of the B) strobilurins. According to the present invention, preference is also given to compositions of a compound of the formula Ia.1 and at least one active compound selected from the group consisting of the B) strobilurins. According to the present invention, preference is also given to compositions of a compound of the formula Ia-1 and at least one active compound selected from the group consisting of the B) strobilurins. According to the present invention, preference is also given to compositions of a compound of the formula Ia.1-1 and at least one active compound selected from the group consisting of the B) strobilurins.

According to the present invention, preference is also given to compositions of a compound of the formula Ib and at least one active compound selected from the group consisting of the B) strobilurins. According to the present invention, preference is also given to compositions of a compound of the formula Ib.1 and at least one active compound selected from the group consisting of the B) strobilurins. According to the present invention, preference is also given to compositions of a compound of the formula Ib-1 and at least one active compound selected from the group consisting of the B) strobilurins. According to the present invention, preference is also given to compositions of a compound of the formula Ib.1-1 and at least one active compound selected from the group consisting of the B) strobilurins.

According to the present invention, preference is also given to compositions of a compound of the formula Ic and at least one active compound selected from the group consisting of the B) strobilurins. According to the present invention, preference is also given to compositions of a compound of the formula Ic.1 and at least one active compound selected from the group consisting of the B) strobilurins. According to the present invention, preference is also given to compositions of a compound of the formula Ic-1 and at least one active compound selected from the group consisting of the B) strobilurins. According to the present invention, preference is also given to compositions of a compound of the formula Ic.1-1 and at least one active compound selected from the group consisting of the B) strobilurins.

According to the present invention, preference is also given to compositions of a compound of the formula Id and at least one active compound selected from the group consisting of the B) strobilurins. According to the present invention, preference is also given to compositions of a compound of the formula Id.1 and at least one active compound selected from the group consisting of the B) strobilurins. According to the present invention, preference is also given to compositions of a compound of the formula Id-1 and at least one active compound selected from the group consisting of the B) strobilurins. According to the present invention, preference is also given to compositions of a compound of the formula Id.1-1 and at least one active compound selected from the group consisting of the B) strobilurins.

According to the present invention, preference is also given to compositions of a compound of the formula Ie and at least one active compound selected from the group consisting of the B) strobilurins. According to the present invention, preference is also, given to compositions of a compound of the formula Ie.1 and at least one active compound selected from the group consisting of the B) strobilurins. According to the present invention, preference is also given to compositions of a compound of the formula Ie-1 and at least one active compound selected from the group consisting of the B) strobilurins. According to the present invention, preference is also given to compositions of a compound of the formula Ie.1-1 and at least one active compound selected from the group consisting of the B) strobilurins.

According to the present invention, preference is also given to compositions of a compound of the formula If and at least one active compound selected from the group consisting of the B) strobilurins. According to the present invention, preference is also given to compositions of a compound of the formula If.1 and at least one active compound selected from the group consisting of the B) strobilurins. According to the present invention, preference is also given to compositions of a compound of the formula If-1 and at least one active compound selected from the group consisting of the B) strobilurins. According to the present invention, preference is also given to compositions of a compound of the formula If.1-1 and at least one active compound selected from the group consisting of the B) strobilurins.

Preference is given to compositions of a compound of the formula I and at least one active compound selected from the group consisting of the C) carboxamides. In particular, the invention relates to compositions comprising one of the compounds of the formula I individually listed in Tables 1 to 110 and at least one active compound selected from the group consisting of the C) carboxamides.

According to the present invention, preference is also given to compositions of a compound of the formula Ia and at least one active compound selected from the group consisting of the C) carboxamides. According to the present invention, preference is also given to compositions of a compound of the formula Ia.1 and at least one active compound selected from the group consisting of the C) carboxamides. According to the present invention, preference is also given to compositions of a compound of the formula Ia-1 and at least one active compound selected from the group consisting of the C) carboxamides. According to the present invention, preference is also given to compositions of a compound of the formula Ia.1-1 and at least one active compound selected from the group consisting of the C) carboxamides.

According to the present invention, preference is also given to compositions of a compound of the formula Ib and at least one active compound selected from the group consisting of the C) carboxamides. According to the present invention, preference is also given to compositions of a compound of the formula Ib.1 and at least one active compound selected from the group consisting of the C) carboxamides. According to the present invention, preference is also given to compositions of a compound of the formula Ib-1 and at least one active compound selected from the group consisting of the C) carboxamides. According to the present invention, preference is also given to compositions of a compound of the formula Ib.1-1 and at least one active compound selected from the group consisting of the C) carboxamides.

According to the present invention, preference is also given to compositions of a compound of the formula Ic and at least one active compound selected from the group consisting of the C) carboxamides. According to the present invention, preference is also given to compositions of a compound of the formula Ic.1 and at least one active compound selected from the group consisting of the C) carboxamides. According to the present invention, preference is also given to compositions of a compound of the formula Ic-1 and at least one active compound selected from the group consisting of the C) carboxamides. According to the present invention, preference is also given to compositions of a compound of the formula Ic.1-1 and at least one active compound selected from the group consisting of the C) carboxamides.

According to the present invention, preference is also given to compositions of a compound of the formula Id and at least one active compound selected from the group consisting of the C) carboxamides. According to the present invention, preference is also given to compositions of a compound of the formula Id.1 and at least one active compound selected from the group consisting of the C) carboxamides. According to the present invention, preference is also given to compositions of a compound of the formula Id-1 and at least one active compound selected from the group consisting of the C) carboxamides. According to the present invention, preference is also given to compositions of a compound of the formula Id.1-1 and at least one active compound selected from the group consisting of the C) carboxamides.

According to the present invention, preference is also given to compositions of a compound of the formula Ie and at least one active compound selected from the group consisting of the C) carboxamides. According to the present invention, preference is also given to compositions of a compound of the formula Ie.1 and at least one active compound selected from the group consisting of the C) carboxamides. According to the present invention, preference is also given to compositions of a compound of the formula Ie-1 and at least one active compound selected from the group consisting of the C) carboxamides. According to the present invention, preference is also given to compositions of a compound of the formula Ie.1-1 and at least one active compound selected from the group consisting of the C) carboxamides.

According to the present invention, preference is also given to compositions of a compound of the formula If and at least one active compound selected from the group consisting of the C) carboxamides. According to the present invention, preference is also given to compositions of a compound of the formula If.1 and at least one active compound selected from the group consisting of the C) carboxamides. According to the present invention, preference is also given to compositions of a compound of the formula If-1 and at least one active compound selected from the group consisting of the C) carboxamides. According to the present invention, preference is also given to compositions of a compound of the formula If.1-1 and at least one active compound selected from the group consisting of the C) carboxamides.

Preference is furthermore also given to compositions of a compound of the formula I and at least one active compound selected from the group consisting of the D) heterocyclic compounds. In particular, the invention relates to compositions comprising one of the compounds of the formula I individually listed in Tables 1 to 110 and at least one active compound selected from the group consisting of the D) heterocyclic compounds.

According to the present invention, preference is also given to compositions of a compound of the formula Ia and at least one active compound selected from the group consisting of the D) heterocyclic compounds. According to the present invention, preference is also given to compositions of a compound of the formula Ia.1 and at least one active compound selected from the group consisting of the D) heterocyclic compounds. According to the present invention, preference is also given to compositions of a compound of the formula Ia-1 and at least one active compound selected from the group consisting of the D) heterocyclic compounds. According to the present invention, preference is also given to compositions of a compound of the formula Ia.1-1 and at least one active compound selected from the group consisting of the D) heterocyclic compounds.

According to the present invention, preference is also given to compositions of a compound of the formula Ib and at least one active compound selected from the group consisting of the D) heterocyclic compounds. According to the present invention, preference is also given to compositions of a compound of the formula Ib.1 and at least one active compound selected from the group consisting of the D) heterocyclic compounds. According to the present invention, preference is also given to compositions of a compound of the formula Ib-1 and at least one active compound selected from the group consisting of the D) heterocyclic compounds. According to the present invention, preference is also given to compositions of a compound of the formula Ib.1-1 and at least one active compound selected from the group consisting of the D) heterocyclic compounds.

According to the present invention, preference is also given to compositions of a compound of the formula is and at least one active compound selected from the group consisting of the D) heterocyclic compounds. According to the present invention, preference is also given to compositions of a compound of the formula Ic.1 and at least one active compound selected from the group consisting of the D) heterocyclic compounds. According to the present invention, preference is also given to compositions of a compound of the formula Ic-1 and at least one active compound selected from the group consisting of the D) heterocyclic compounds. According to the present invention, preference is also given to compositions of a compound of the formula Ic.1-1 and at least one active compound selected from the group consisting of the D) heterocyclic compounds.

According to the present invention, preference is also given to compositions of a compound of the formula Id and at least one active compound selected from the group consisting of the D) heterocyclic compounds. According to the present invention, preference is also given to compositions of a compound of the formula Id.1 and at least one active compound selected from the group consisting of the D) heterocyclic compounds. According to the present invention, preference is also given to compositions of a compound of the formula Id-1 and at least one active compound selected from the group consisting of the D) heterocyclic compounds. According to the present invention, preference is also given to compositions of a compound of the formula Id.1-1 and at least one active compound selected from the group consisting of the D) heterocyclic compounds.

According to the present invention, preference is also given to compositions of a compound of the formula Ie and at least one active compound selected from the group consisting of the D) heterocyclic compounds. According to the present invention, preference is also given to compositions of a compound of the formula Ie.1 and at least one active compound selected from the group consisting of the D) heterocyclic compounds. According to the present invention, preference is also given to compositions of a compound of the formula Ie-1 and at least one active compound selected from the group consisting of the D) heterocyclic compounds. According to the present invention, preference is also given to compositions of a compound of the formula Ie.1-1 and at least one active compound selected from the group consisting of the D) heterocyclic compounds.

According to the present invention, preference is also given to compositions of a compound of the formula If and at least one active compound selected from the group consisting of the D) heterocyclic compounds. According to the present invention, preference is also given to compositions of a compound of the formula If.1 and at least one active compound selected from the group consisting of the D) heterocyclic compounds. According to the present invention, preference is also given to compositions of a compound of the formula If-1 and at least one active compound selected from the group consisting of the D) heterocyclic compounds. According to the present invention, preference is also given to compositions of a compound of the formula If.1-1 and at least one active compound selected from the group consisting of the D) heterocyclic compounds.

Preference is furthermore also given to compositions of a compound of the formula I and at least one active compound selected from the group consisting of the E) carbamates. In particular, the invention relates to compositions comprising one of the compounds of the formula I individually listed in Tables 1 to 110 and at least one active compound selected from the group consisting of the E) carbamates.

According to the present invention, preference is also given to compositions of a compound of the formula Ia and at least one active compound selected from the group consisting of the E) carbamates. According to the present invention, preference is also given to compositions of a compound of the formula Ia.1 and at least one active compound selected from the group consisting of the E) carbamates. According to the present invention, preference is also given to compositions of a compound of the formula Ia-1 and at least one active compound selected from the group consisting of the E) carbamates. According to the present invention, preference is also given to compositions of a compound of the formula Ia.1-1 and at least one active compound selected from the group consisting of the E) carbamates.

According to the present invention, preference is also given to compositions of a compound of the formula Ib and at least one active compound selected from the group consisting of the E) carbamates. According to the present invention, preference is also given to compositions of a compound of the formula Ib.1 and at least one active compound selected from the group consisting of the E) carbamates. According to the present invention, preference is also given to compositions of a compound of the formula Ib-1 and at least one active compound selected from the group consisting of the E) carbamates. According to the present invention, preference is also given to compositions of a compound of the formula Ib.1-1 and at least one active compound selected from the group consisting of the E) carbamates.

According to the present invention, preference is also given to compositions of a compound of the formula Ic and at least one active compound selected from the group consisting of the E) carbamates. According to the present invention, preference is also given to compositions of a compound of the formula Ic.1 and at least one active compound selected from the group consisting of the E) carbamates. According to the present invention, preference is also given to compositions of a compound of the formula Ic-1 and at least one active compound selected from the group consisting of the E) carbamates. According to the present invention, preference is also given to compositions of a compound of the formula Ic.1-1 and at least one active compound selected from the group consisting of the E) carbamates.

According to the present invention, preference is also given to compositions of a compound of the formula Id and at least one active compound selected from the group consisting of the E) carbamates. According to the present invention, preference is also given to compositions of a compound of the formula Id.1 and at least one active compound selected from the group consisting of the E) carbamates. According to the present invention, preference is also given to compositions of a compound of the formula Id-1 and at least one active compound selected from the group consisting of the E) carbamates. According to the present invention, preference is also given to compositions of a compound of the formula Id.1-1 and at least one active compound selected from the group consisting of the E) carbamates.

According to the present invention, preference is also given to compositions of a compound of the formula Ie and at least one active compound selected from the group consisting of the E) carbamates. According to the present invention, preference is also given to compositions of a compound of the formula Ie.1 and at least one active compound selected from the group consisting of the E) carbamates. According to the present invention, preference is also given to compositions of a compound of the formula Ie-1 and at least one active compound selected from the group consisting of the E) carbamates. According to the present invention, preference is also given to compositions of a compound of the formula Ie.1-1 and at least one active compound selected from the group consisting of the E) carbamates.

According to the present invention, preference is also given to compositions of a compound of the formula If and at least one active compound selected from the group consisting of the E) carbamates. According to the present invention, preference is also given to compositions of a compound of the formula If.1 and at least one active compound selected from the group consisting of the E) carbamates. According to the present invention, preference is also given to compositions of a compound of the formula If-1 and at least one active compound selected from the group consisting of the E) carbamates. According to the present invention, preference is also given to compositions of a compound of the formula If.1-1 and at least one active compound selected from the group consisting of the E) carbamates.

Preference is furthermore also given to compositions of a compound of the formula I and at least one active compound selected from the group consisting of the F) other fungicides. In particular, the invention relates to compositions comprising one of the compounds of the formula I individually listed in Tables 1 to 110 and at least one active compound selected from the group consisting of the F) other fungicides.

According to the present invention, preference is also given to compositions of a compound of the formula Ia and at least one active compound selected from the group consisting of the F) other fungicides. According to the present invention, preference is also given to compositions of a compound of the formula Ia.1 and at least one active compound selected from the group consisting of the F) other fungicides. According to the present invention, preference is also given to compositions of a compound of the formula Ia-1 and at least one active compound selected from the group consisting of the F) other fungicides. According to the present invention, preference is also given to compositions of a compound of the formula Ia.1-1 and at least one active compound selected from the group consisting of the F) other fungicides.

According to the present invention, preference is also given to compositions of a compound of the formula Ib and at least one active compound selected from the group consisting of the F) other fungicides. According to the present invention, preference is also given to compositions of a compound of the formula Ib.1 and at least one active compound selected from the group consisting of the F) other fungicides. According to the present invention, preference is also given to compositions of a compound of the formula Ib-1 and at least one active compound selected from the group consisting of the F) other fungicides. According to the present invention, preference is also given to compositions of a compound of the formula Ib.1-1 and at least one active compound selected from the group consisting of the F) other fungicides.

According to the present invention, preference is also given to compositions of a compound of the formula Ic and at least one active compound selected from the group consisting of the F) other fungicides. According to the present invention, preference is also given to compositions of a compound of the formula Ic.1 and at least one active compound selected from the group consisting of the F) other fungicides. According to the present invention, preference is also given to compositions of a compound of the formula Ic-1 and at least one active compound selected from the group consisting of the F) other fungicides. According to the present invention, preference is also given to compositions of a compound of the formula Ic.1-1 and at least one active compound selected from the group consisting of the F) other fungicides.

According to the present invention, preference is also given to compositions of a compound of the formula Id and at least one active compound selected from the group consisting of the F) other fungicides. According to the present invention, preference is also given to compositions of a compound of the formula Id.1 and at least one active compound selected from the group consisting of the F) other fungicides. According to the present invention, preference is also given to compositions of a compound of the formula Id-1 and at least one active compound selected from the group consisting of the F) other fungicides. According to the present invention, preference is also given to compositions of a compound of the formula Id.1-1 and at least one active compound selected from the group consisting of the F) other fungicides.

According to the present invention, preference is also given to compositions of a compound of the formula Ie and at least one active compound selected from the group consisting of the F) other fungicides. According to the present invention, preference is also given to compositions of a compound of the formula Ie.1 and at least one active compound selected from the group consisting of the F) other fungicides. According to the present invention, preference is also given to compositions of a compound of the formula Ie-1 and at least one active compound selected from the group consisting of the F) other fungicides. According to the present invention, preference is also given to compositions of a compound of the formula Ie.1-1 and at least one active compound selected from the group consisting of the F) other fungicides.

According to the present invention, preference is also given to compositions of a compound of the formula If and at least one active compound selected from the group consisting of the F) other fungicides. According to the present invention, preference is also given to compositions of a compound of the formula If.1 and at least one active compound selected from the group consisting of the F) other fungicides. According to the present invention, preference is also given to compositions of a compound of the formula If-1 and at least one active compound selected from the group consisting of the F) other fungicides. According to the present invention, preference is also given to compositions of a compound of the formula If.1-1 and at least one active compound selected from the group consisting of the F) other fungicides.

Particular preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the A) azoles, selected from cyproconazole, difenoconazole, epoxiconazole, fluquinconazole, flusilazole, flutriafol, metconazole, myclobutanil, propiconazole, prothioconazole, triadimefon, triadimenol, tebuconazole, tetraconazole, triticonazole, prochloraz, cyazofamid, benomyl and carbendazim.

Preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the A) azoles, selected from cyazofamid, metconazole, fluquiconazole, triadimefon, triadimenol, penconazole, hexaconazole and thiabendazole, in particular selected from penconazole, hexaconazole and thiabendazole.

Preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the A) azoles, selected from epoxiconazole, fluquinconazole, flutriafol, metconazole, tebuconazole, triticonazole, prochloraz and carbendazim.

Preference is furthermore also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the A) azoles, selected from cyproconazole, difenoconazole, epoxiconazole, propiconazole, prochloraz, prothioconazole and tebuconazole, in particular selected from cyproconazole, difenoconazole and propiconazole.

Preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the B) strobilurins, selected from azoxystrobin, dimoxystrobin, fluoxastrobin, kresoxim-methyl, orysastrobin, picoxystrobin, pyraclostrobin and trifloxystrobin.

Particular preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the B) strobilurins, selected from kresoxim-methyl, orysastrobin and pyraclostrobin.

Very particular preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and pyraclostrobin.

Preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the B) strobilurins, selected from azoxystrobin, fluoxastrobin, orysastrobin, picoxystrobin, pyraclostrobin and trifloxystrobin, in particular selected from azoxystrobin and picoxystrobin.

Preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the C) carboxamides, selected from fenhexamid, metalaxyl, mefenoxam, ofurace, dimethomorph, flumorph, fluopicofid (picobenzamid), zoxamide, carpropamid and mandipropamid.

Particular preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the C) carboxamides, selected from fenhexamid, metalaxyl, mefenoxam, ofurace, dimethomorph, zoxamide and carpropamid.

Particular preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the C) carboxamides, selected from fenhexamid and oxadixyl.

Preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the C) carboxamides, selected from boscalid, dimethomorph, metalaxyl, mandipropamid, N-(3′,4′-dichloro-4-fluorobiphenyl-2-yl)-3-difluoromethyl-1-methylpyrazole-4-carboxamide, N-(3′,4′-dichloro-5-fluorobiphenyl-2-yl)-3-difluoromethyl-1-methylpyrazole-4-carboxamide and fluopicolid (picobenzamid), in particular selected from metalaxyl, mandipropamid, N-(3′,4′-dichloro-4-fluorobiphenyl-2-yl)-3-difluoromethyl-1-methylpyrazole-4-carboxamide, N-(3′,4′-dichloro-5-fluorobiphenyl-2-yl)-3-difluoromethyl-1-methylpyrazole-4-carboxamide and fluopicofid (picobenzamid).

Preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the D) heterocylic compounds, selected from fluazinam, cyprodinil, fenarimol, mepanipyrim, pyrimethanil, triforine, fludioxonil, dodemorph, fenpropimorph, tridemorph, fenpropidin, iprodione, vinclozolin, famoxadone, fenamidone, probenazole, 5-chloro-7-(4-methylpiperidin-1-yl)-6-(2,4,6-trifluorophenyl)-[1,2,4]triazolo[1,5-a]pyrimidine, proquinazid, acibenzolar-S-methyl, captafol, folpet, fenoxanil and quinoxyfen, in particular fluazinam, cyprodinil, fenarimol, mepanipyrim, pyrimethanil, triforine, fludioxonil, dodemorph, fenpropimorph, tridemorph, fenpropidin, iprodione, vinclozolin, famoxadone, fenamidone, probenazole, proquinazid, acibenzolar-S-methyl, captafol, folpet, fenoxanil and quinoxyfen.

Particular preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the D) heterocyclic compounds, selected from tridemorph, iprodione, pyrimethanil, fenpropimorph, fenpropidin, acibenzolar-S-methyl and pyrifenox, in particular selected from fenpropimorph, fenpropidin, acibenzolar-S-methyl and pyrifenox.

Particular preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the D) heterocyclic compounds, selected from pyrimethanil, dodemorph, fenpropimorph, tridemorph, iprodione, vinclozolin, 5-chloro-7-(4-methylpiperidin-1-yl)-6-(2,4,6-trifluoro-phenyl)-[1,2,4]triazolo[1,5-a]pyrimidine and quinoxyfen, in particular pyrimethanil, dodemorph, fenpropimorph, tridemorph, iprodione, vinclozolin and quinoxyfen.

Preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the D) heterocylic compounds, selected from fludioxonil, cyprodinil and 5-chloro-7-(4-methylpiperidin-1-yl)-6-(2,4,6-trifluorophenyl)-[1,2,4]triazolo[1,5-a]pyrimidine, in particular selected from fludioxonil and cyprodinil.

Preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the E) carbamates, selected from mancozeb, metiram, propineb, thiram, iprovalicarb, flubenthiavalicarb and propamocarb.

Particular preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the E) carbamates, selected from mancozeb and metiram.

Preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the E) carbamates, selected from iprovalicarb and metiram, preferably iprovalicarb.

Preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the F) other fungicides, selected from chlorothalonil, fosetyl, fosetyl-aluminum, metrafenone and spiroxamine.

Particular preference is also given to compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and at least one active compound selected from the group consisting of the F) other fungicides, selected from phosphorous acid and its salts, chlorothalonil and metrafenone.

Preference is also given to three-compound compositions of a compound of the formula I, in particular Ia, Ib, Ic, Id, Ie or If, especially Ia.1, Ib.1, Ic.1, Id.1, Ie.1 or If.1, in particular Ia-1, Ib-1, Ic-1, Id-1, Ie-1 or If-1, especially Ia.1-1, Ib.1-1, Ic.1-1, Id.1-1, Ie.1-1 or If.1-1, and two of the active compounds II mentioned above or mentioned as being preferred.

Preferred active compound combinations according to the present invention are listed in Tables M-1 to M-29 below:

### Table M-1

Fungicidal compositions in which component 1) is one of the compounds of the formula I given in Tables 1 to 110 and component 2) is propiconazole.

### Table M-2

Fungicidal compositions in which component 1) is one of the compounds of the formula I given in Tables 1 to 110 and component 2) is cyproconazole.

### Table M-3

Fungicidal compositions in which component 1) is one of the compounds of the formula I given in Tables 1 to 110 and component 2) is difenoconazole.

### Table M-4

Fungicidal compositions in which component 1) is one of the compounds of the formula I given in Tables 1 to 110 and component 2) is prochloraz.

### Table M-5

Fungicidal compositions in which component 1) is one of the compounds of the formula I given in Tables 1 to 110 and component 2) is prothioconazole.

### Table M-6

Fungicidal compositions in which component 1) is one of the compounds of the formula I given in Tables 1 to 110 and component 2) is tebuconazole.

### Table M-7

Fungicidal compositions in which component 1) is one of the compounds of the formula I given in Tables 1 to 110 and component 2) is epoxiconazole.

### Table M-8

Fungicidal compositions in which component 1) is one of the compounds of the formula I given in Tables 1 to 110 and component 2) is azoxystrobin.

### Table M-9

Fungicidal compositions in which component 1) is one of the compounds of the formula I given in Tables 1 to 110 and component 2) is picoxystrobin.

### Table M-10

Fungicidal compositions in which component 1) is one of the compounds of the formula I given in Tables 1 to 110 and component 2) is fluoxastrobin.

### Table M-11

Fungicidal compositions in which component 1) is one of the compounds of the formula I given in Tables 1 to 110 and component 2) is trifloxystrobin.

### Table M-12

Fungicidal compositions in which component 1) is one of the compounds of the formula I given in Tables 1 to 110 and component 2) is orysastrobin.

### Table M-13

Fungicidal compositions in which component 1) is one of the compounds of the formula I given in Tables 1 to 110 and component 2) is pyraclostrobin.

### Table M-14

Fungicidal compositions in which component 1) is one of the compounds of the formula I given in Tables 1 to 110 and component 2) is metalaxyl.

### Table M-15

Fungicidal compositions in which component 1) is one of the compounds of the formula I given in Tables 1 to 110 and component 2) is mandipropamid.

### Table M-16

Fungicidal compositions in which component 1) is one of the compounds of the formula I given in Tables 1 to 110 and component 2) is fluopicolid.

### Table M-17

Fungicidal compositions in which component 1) is one of the compounds of the formula I given in Tables 1 to 110 and component 2) is boscalid.

### Table M-18

Fungicidal compositions in which component 1) is one of the compounds of the formula I given in Tables 1 to 110 and component 2) is dimethomorph.

### Table M-19

Fungicidal compositions in which component 1) is one of the compounds of the formula I given in Tables 1 to 110 and component 2) is N-(3′,4′-dichloro-4-fluorobiphenyl-2-yl)-3-difluoromethyl-1-methylpyrazole-4-carboxamide.

### Table M-20

Fungicidal compositions in which component 1) is one of the compounds of the formula I given in Tables 1 to 110 and component 2) is N-(3′,4′-dichloro-5-fluorobiphenyl-2-yl)-3-difluoromethyl-1-methylpyrazole-4-carboxamide.

### Table M-21

Fungicidal compositions in which component 1) is one of the compounds of the formula I given in Tables 1 to 110 and component 2) is N-(2-bicyclopropyl-2-yl-phenyl)-3-difluoromethyl-1-methyl-1H-pyrazole-4-carboxamide.

### Table M-22

Fungicidal compositions in which component 1) is one of the compounds of the formula I given in Tables 1 to 110 and component 2) is cyprodinil.

### Table M-23

Fungicidal compositions in which component 1) is one of the compounds of the formula I given in Tables 1 to 110 and component 2) is fludioxonil.

### Table M-24

Fungicidal compositions in which component 1) is one of the compounds of the formula I given in Tables 1 to 110 and component 2) is 5-chloro-7-(4-methylpiperidin-1-yl)-6-(2,4,6-trifluorophenyl)[1,2,4]triazolo[1,5-a]pyrimidine.

### Table M-25

Fungicidal compositions in which component 1) is one of the compounds of the formula I given in Tables 1 to 110 and component 2) is iprovalicarb.

### Table M-26

Fungicidal compositions in which component 1) is one of the compounds of the formula I given in Tables 1 to 110 and component 2) is chlorothalonil.

### Table M-27

Fungicidal compositions in which component 1) is one of the compounds of the formula I given in Tables 1 to 110 and component 2) is fosetyl-Al.

### Table M-28

Fungicidal compositions in which component 1) is one of the compounds of the formula I given in Tables 1 to 110 and component 2) is spiroxamine.

### Table M-29

Fungicidal compositions in which component 1) is one of the compounds of the formula I given in Tables 1 to 110 and component 2) is metrafenone.

Special preference may be given to the fungicidal compositions described in Tables Z-1 to Z-29 below:

### Table Z-1

Fungicidal compositions in which component 1) is one of the compounds C-1 to C-348 listed in Table C and component 2) is propiconazole.

### Table Z-2

Fungicidal compositions in which component 1) is one of the compounds C-1 to C-348 listed in Table C and component 2) is cyproconazole.

### Table Z-3

Fungicidal compositions in which component 1) is one of the compounds C-1 to C-348 listed in Table C and component 2) is difenoconazole.

### Table Z-4

Fungicidal compositions in which component 1) is one of the compounds C-1 to C-348 listed in Table C and component 2) is prochloraz.

### Table Z-5

Fungicidal compositions in which component 1) is one of the compounds C-1 to C-348 listed in Table C and component 2) is prothioconazole.

### Table Z-6

Fungicidal compositions in which component 1) is one of the compounds C-1 to C-348 listed in Table C and component 2) is tebuconazole.

### Table Z-7

Fungicidal compositions in which component 1) is one of the compounds C-1 to C-348 listed in Table C and component 2) is epoxiconazole.

### Table Z-8

Fungicidal compositions in which component 1) is one of the compounds C-1 to C-348 listed in Table C and component 2) is azoxystrobin.

### Table Z-9

Fungicidal compositions in which component 1) is one of the compounds C-1 to C-348 listed in Table C and component 2) is picoxystrobin.

### Table Z-10

Fungicidal compositions in which component 1) is one of the compounds C-1 to C-348 listed in Table C and component 2) is fluoxastrobin.

### Table Z-11

Fungicidal compositions in which component 1) is one of the compounds C-1 to C-348 listed in Table C and component 2) is trifloxystrobin.

### Table Z-12

Fungicidal compositions in which component 1) is one of the compounds C-1 to C-348 listed in Table C and component 2) is orysastrobin.

### Table Z-13

Fungicidal compositions in which component 1) is one of the compounds C-1 to C-348 listed in Table C and component 2) is pyraclostrobin.

### Table Z-14

Fungicidal compositions in which component 1) is one of the compounds C-1 to C-348 listed in Table C and component 2) is metalaxyl.

### Table Z-15

Fungicidal compositions in which component 1) is one of the compounds C-1 to C-348 listed in Table C and component 2) is mandipropamid.

### Table Z-16

Fungicidal compositions in which component 1) is one of the compounds C-1 to C-348 listed in Table C and component 2) is fluopicolid.

### Table Z-17

Fungicidal compositions in which component 1) is one of the compounds C-1 to C-348 listed in Table C and component 2) is boscalid.

### Table Z-18

Fungicidal compositions in which component 1) is one of the compounds C-1 to C-348 listed in Table C and component 2) is dimethomorph.

### Table Z-19

Fungicidal compositions in which component 1) is one of the compounds C-1 to C-348 listed in Table C and component 2) is N-(3′,4′-dichloro-4-fluorobiphenyl-2-yl)-3-difluoromethyl-1-methylpyrazole-4-carboxamide.

### Table Z-20

Fungicidal compositions in which component 1) is one of the compounds C-1 to C-348 listed in Table C and component 2) is N-(3′,4′-dichloro-5-fluorobiphenyl-2-yl)-3-difluoromethyl-1-methylpyrazole-4-carboxamide.

### Table Z-21

Fungicidal compositions in which component 1) is one of the compounds C-1 to C-348 listed in Table C and component 2) is N-(2-bicyclopropyl-2-ylphenyl)-3-difluoromethyl-1-methyl-1H-pyrazole-4-carboxamide.

### Table Z-22

Fungicidal compositions in which component 1) is one of the compounds C-1 to C-348 listed in Table C and component 2) is cyprodinil.

### Table Z-23

Fungicidal compositions in which component 1) is one of the compounds C-1 to C-348 listed in Table C and component 2) is fludioxonil.

### Table Z-24

Fungicidal compositions in which component 1) is one of the compounds C-1 to C-348 listed in Table C and component 2) is 5-chloro-7-(4-methylpiperidin-1-yl)-6-(2,4,6-trifluorophenyl)[1,2,4]triazolo[1,5-a]pyrimidine.

### Table Z-25

Fungicidal compositions in which component 1) is one of the compounds C-1 to C-348 listed in Table C and component 2) is iprovalicarb.

### Table Z-26

Fungicidal compositions in which component 1) is one of the compounds C-1 to C-348 listed in Table C and component 2) is chlorothalonil.

### Table Z-27

Fungicidal compositions in which component 1) is one of the compounds C-1 to C-348 listed in Table C and component 2) isfosetyl-Al.

### Table Z-28

Fungicidal compositions in which component 1) is one of the compounds C-1 to C-348 listed in Table C and component 2) is spiroxamine.

### Table Z-29

Fungicidal compositions in which component 1) is one of the compounds C-1 to C-348 listed in Table C and component 2) is metrafenone.

TABLE C

| | | |
|---|---|---|
| 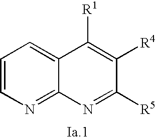 | 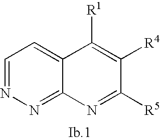 | 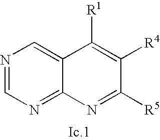 |
| 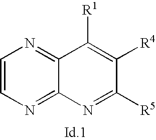 | 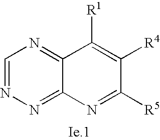 | 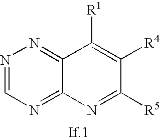 |

| No. | Compound |
|---|---|
| C-1 | formula Ia.1 where R¹ = C₆H₁₁; R⁴ = 2,4,6-F₃—(C₆H₂); R⁵ = Cl |
| C-2 | formula Ia.1 where R¹ = 4-CH₃—(C₆H₁₁); R⁴ = 2,4,6-F₃—(C₆H₂); R⁵ = Cl |
| C-3 | formula Ia.1 where R¹ = CH₂(CH₂)₂CH₃; R⁴ = 2,4,6-F₃—(C₆H₂); R⁵ = Cl |
| C-4 | formula Ia.1 where R¹ = CH₂CH(CH₃)C₂H₅; R⁴ = 2,4,6-F₃—(C₆H₂); R⁵ = Cl |
| C-5 | formula Ia.1 where R¹ = CH₂C(CH₃)₂C₂H₅; R⁴ = 2,4,6-F₃—(C₆H₂); R⁵ = Cl |
| C-6 | formula Ia.1 where R¹ = CH₂CH₂CH═CH₂; R⁴ = 2,4,6-F₃—(C₆H₂); R⁵ = Cl |
| C-7 | formula Ia.1 where R¹ = CH₂CH(CH₃)CH═CH₂; R⁴ = 2,4,6-F₃—(C₆H₂); R⁵ = Cl |
| C-8 | formula Ia.1 where R¹ = CH₂C(CH₃)₂CH═CH₂; R⁴ = 2,4,6-F₃—(C₆H₂); R⁵ = Cl |
| C-9 | formula Ia.1 where R¹ = C₆H₁₁; R⁴ = 2,4,6-F₃—(C₆H₂); R⁵ = CH₃ |
| C-10 | formula Ia.1 where R¹ = 4-CH₃—(C₆H₁₁); R⁴ = 2,4,6-F₃—(C₆H₂); R⁵ = CH₃ |
| C-11 | formula Ia.1 where R¹ = CH₂(CH₂)₂CH₃; R⁴ = 2,4,6-F₃—(C₆H₂); R⁵ = CH₃ |
| C-12 | formula Ia.1 where R¹ = CH₂CH(CH₃)C₂H₅; R⁴ = 2,4,6-F₃—(C₆H₂); R⁵ = CH₃ |
| C-13 | formula Ia.1 where R¹ = CH₂C(CH₃)₂C₂H₅; R⁴ = 2,4,6-F₃—(C₆H₂); R⁵ = CH₃ |
| C-14 | formula Ia.1 where R¹ = CH₂CH₂CH═CH₂; R⁴ = 2,4,6-F₃—(C₆H₂); R⁵ = CH₃ |
| C-15 | formula Ia.1 where R¹ = CH₂CH(CH₃)CH═CH₂; R⁴ = 2,4,6-F₃—(C₆H₂); R⁵ = CH₃ |
| C-16 | formula Ia.1 where R¹ = CH₂C(CH₃)₂CH═CH₂; R⁴ = 2,4,6-F₃—(C₆H₂); R⁵ = CH₃ |
| C-17 | formula Ia.1 where R¹ = NH(CH₂CF₃); R⁴ = 2,4,6-F₃—(C₆H₂); R⁵ = Cl |
| C-18 | formula Ia.1 where R¹ = NH(CH(CH₃)CF₃); R⁴ = 2,4,6-F₃—(C₆H₂); R⁵ = Cl |
| C-19 | formula Ia.1 where R¹ = NH(CH₂CF₃); R⁴ = 2,4,6-F₃—(C₆H₂); R⁵ = F |
| C-20 | formula Ia.1 where R¹ = NH(CH(CH₃)CF₃); R⁴ = 2,4,6-F₃—(C₆H₂); R⁵ = F |
| C-21 | formula Ia.1 where R¹ = NH(CH(CH₃)₂); R⁴ = 2,4,6-F₃—(C₆H₂); R⁵ = Cl |
| C-22 | formula Ia.1 where R¹ = NH(CH(CH₃)C₂H₅); R⁴ = 2,4,6-F₃—(C₆H₂); R⁵ = Cl |
| C-23 | formula Ia.1 where R¹ = NH(CH₂CH(CH₃)₂); R⁴ = 2,4,6-F₃—(C₆H₂); R⁵ = Cl |
| C-24 | formula Ia.1 where R¹ = NH(CH(CH₃)CH(CH₃)₂); R⁴ = 2,4,6-F₃—(C₆H₂); R⁵ = Cl |
| C-25 | formula Ia.1 where R¹ = NH(CH(CH₃)C(CH₃)₃); R⁴ = 2,4,6-F₃—(C₆H₂); R⁵ = Cl |
| C-26 | formula Ia.1 where R¹ = NH(CH(CH₃)₂); R⁴ = 2,4,6-F₃—(C₆H₂); R⁵ = F |
| C-27 | formula Ia.1 where R¹ = NH(CH(CH₃)C₂H₅); R⁴ = 2,4,6-F₃—(C₆H₂); R⁵ = F |
| C-28 | formula Ia.1 where R¹ = NH(CH₂CH(CH₃)₂); R⁴ = 2,4,6-F₃—(C₆H₂); R⁵ = F |
| C-29 | formula Ia.1 where R¹ = NH(CH(CH₃)CH(CH₃)₂); R⁴ = 2,4,6-F₃—(C₆H₂); R⁵ = F |
| C-30 | formula Ia.1 where R¹ = NH(CH(CH₃)C(CH₃)₃); R⁴ = 2,4,6-F₃—(C₆H₂); R⁵ = F |
| C-31 | formula Ia.1 where R¹ = N(CH₃)(CH(CH₃)₂); R⁴ = 2,4,6-F₃—(C₆H₂); R⁵ = Cl |
| C-32 | formula Ia.1 where R¹ = N(CH₃)(CH(CH₃)C₂H₅); R⁴ = 2,4,6-F₃—(C₆H₂); R⁵ = Cl |
| C-33 | formula Ia.1 where R¹ = N(CH₃)(CH₂CH(CH₃)₂); R⁴ = 2,4,6-F₃—(C₆H₂); R⁵ = Cl |
| C-34 | formula Ia.1 where R¹ = N(CH₃)(CH(CH₃)CH(CH₃)₂); R⁴ = 2,4,6-F₃—(C₆H₂); R⁵ = Cl |
| C-35 | formula Ia.1 where R¹ = N(CH₃)(CH(CH₃)C(CH₃)₃); R⁴ = 2,4,6-F₃—(C₆H₂); R⁵ = Cl |
| C-36 | formula Ia.1 where R¹ = N(CH₃)(CH(CH₃)₂); R⁴ = 2,4,6-F₃—(C₆H₂); R⁵ = F |
| C-37 | formula Ia.1 where R¹ = N(CH₃)(CH(CH₃)C₂H₅); R⁴ = 2,4,6-F₃—(C₆H₂); R⁵ = F |
| C-38 | formula Ia.1 where R¹ = N(CH₃)(CH₂CH(CH₃)₂); R⁴ = 2,4,6-F₃—(C₆H₂); R⁵ = F |
| C-39 | formula Ia.1 where R¹ = N(CH₃)(CH(CH₃)CH(CH₃)₂); R⁴ = 2,4,6-F₃—(C₆H₂); R⁵ = F |
| C-40 | formula Ia.1 where R¹ = N(CH₃)(CH(CH₃)C(CH₃)₃); R⁴ = 2,4,6-F₃—(C₆H₂); R⁵ = F |
| C-41 | formula Ia.1 where R¹ = N(C₂H₅)(CH(CH₃)₂); R⁴ = 2,4,6-F₃—(C₆H₂); R⁵ = Cl |
| C-42 | formula Ia.1 where R¹ = N(C₂H₅)(CH(CH₃)C₂H₅); R⁴ = 2,4,6-F₃—(C₆H₂); R⁵ = Cl |
| C-43 | formula Ia.1 where R¹ = N(C₂H₅)(CH₂CH(CH₃)₂); R⁴ = 2,4,6-F₃—(C₆H₂); R⁵ = Cl |
| C-44 | formula Ia.1 where R¹ = N(C₂H₅)(CH(CH₃)CH(CH₃)₂); R⁴ = 2,4,6-F₃—(C₆H₂); R⁵ = Cl |
| C-45 | formula Ia.1 where R¹ = N(C₂H₅)(CH(CH₃)C(CH₃)₃); R⁴ = 2,4,6-F₃—(C₆H₂); R⁵ = Cl |
| C-46 | formula Ia.1 where R¹ = N(C₂H₅)(CH(CH₃)C₂H₅); R⁴ = 2,4,6-F₃—(C₆H₂); R⁵ = F |
| C-47 | formula Ia.1 where R¹ = N(C₂H₅)(CH₂CH(CH₃)₂); R⁴ = 2,4,6-F₃—(C₆H₂); R⁵ = F |
| C-48 | formula Ia.1 where R¹ = N(C₂H₅)(CH(CH₃)CH(CH₃)₂); R⁴ = 2,4,6-F₃—(C₆H₂); R⁵ = F |
| C-49 | formula Ia.1 where R¹ = N(C₂H₅)(CH(CH₃)C(CH₃)₃); R⁴ = 2,4,6-F₃—(C₆H₂); R⁵ = F |
| C-50 | formula Ia.1 where R¹ = N(C₂H₅)(CH(CH₃)C₂H₅); R⁴ = 2,4,6-F₃—(C₆H₂); R⁵ = F |
| C-51 | formula Ia.1 where R¹ = piperidin-1-yl; R⁴ = 2,4,6-F₃—(C₆H₂); R⁵ = Cl |
| C-52 | formula Ia.1 where R¹ = 2-CH₃-piperidin-1-yl; R⁴ = 2,4,6-F₃—(C₆H₂); R⁵ = Cl |
| C-53 | formula Ia.1 where R¹ = 3-CH₃-piperidin-1-yl; R⁴ = 2,4,6-F₃—(C₆H₂); R⁵ = Cl |
| C-54 | formula Ia.1 where R¹ = 4-CH₃-piperidin-1-yl; R⁴ = 2,4,6-F₃—(C₆H₂); R⁵ = Cl |
| C-55 | formula Ia.1 where R¹ = piperidin-1-yl; R⁴ = 2,4,6-F₃—(C₆H₂); R⁵ = F |
| C-56 | formula Ia.1 where R¹ = 2-CH₃-piperidin-1-yl; R⁴ = 2,4,6-F₃—(C₆H₂); R⁵ = F |
| C-57 | formula Ia.1 where R¹ = 3-CH₃-piperidin-1-yl; R⁴ = 2,4,6-F₃—(C₆H₂); R⁵ = F |
| C-58 | formula Ia.1 where R¹ = 4-CH₃-piperidin-1-yl; R⁴ = 2,4,6-F₃—(C₆H₂); R⁵ = F |
| C-59 | formula Ib.1 where R¹ = C₆H₁₁; R⁴ = 2,4,6-F₃—(C₆H₂); R⁵ = Cl |
| C-60 | formula Ib.1 where R¹ = 4-CH₃—(C₆H₁₁); R⁴ = 2,4,6-F₃—(C₆H₂); R⁵ = Cl |
| C-61 | formula Ib.1 where R¹ = CH₂(CH₂)₂CH₃; R⁴ = 2,4,6-F₃—(C₆H₂); R⁵ = Cl |
| C-62 | formula Ib.1 where R¹ = CH₂CH(CH₃)C₂H₅; R⁴ = 2,4,6-F₃—(C₆H₂); R⁵ = Cl |
| C-63 | formula Ib.1 where R¹ = CH₂C(CH₃)₂C₂H₅; R⁴ = 2,4,6-F₃—(C₆H₂); R⁵ = Cl |
| C-64 | formula Ib.1 where R¹ = CH₂CH₂CH═CH₂; R⁴ = 2,4,6-F₃—(C₆H₂); R⁵ = Cl |
| C-65 | formula Ib.1 where R¹ = CH₂CH(CH₃)CH═CH₂; R⁴ = 2,4,6-F₃—(C₆H₂); R⁵ = Cl |
| C-66 | formula Ib.1 where R¹ = CH₂C(CH₃)₂CH═CH₂; R⁴ = 2,4,6-F₃—(C₆H₂); R⁵ = Cl |
| C-67 | formula Ib.1 where R¹ = C₆H₁₁; R⁴ = 2,4,6-F₃—(C₆H₂); R⁵ = CH₃ |
| C-68 | formula Ib.1 where R¹ = 4-CH₃—(C₆H₁₁); R⁴ = 2,4,6-F₃—(C₆H₂); R⁵ = CH₃ |
| C-69 | formula Ib.1 where R¹ = CH₂(CH₂)₂CH₃; R⁴ = 2,4,6-F₃—(C₆H₂); R⁵ = CH₃ |
| C-70 | formula Ib.1 where R¹ = CH₂CH(CH₃)C₂H₅; R⁴ = 2,4,6-F₃—(C₆H₂); R⁵ = CH₃ |
| C-71 | formula Ib.1 where R¹ = CH₂C(CH₃)₂C₂H₅; R⁴ = 2,4,6-F₃—(C₆H₂); R⁵ = CH₃ |
| C-72 | formula Ib.1 where R¹ = CH₂CH₂CH═CH₂; R⁴ = 2,4,6-F₃—(C₆H₂); R⁵ = CH₃ |
| C-73 | formula Ib.1 where R¹ = CH₂CH(CH₃)CH═CH₂; R⁴ = 2,4,6-F₃—(C₆H₂); R⁵ = CH₃ |
| C-74 | formula Ib.1 where R¹ = CH₂C(CH₃)₂CH═CH₂; R⁴ = 2,4,6-F₃—(C₆H₂); R⁵ = CH₃ |
| C-75 | formula Ib.1 where R¹ = NH(CH₂CF₃); R⁴ = 2,4,6-F₃—(C₆H₂); R⁵ = Cl |
| C-76 | formula Ib.1 where R¹ = NH(CH(CH₃)CF₃); R⁴ = 2,4,6-F₃—(C₆H₂); R⁵ = Cl |
| C-77 | formula Ib.1 where R¹ = NH(CH₂CF₃); R⁴ = 2,4,6-F₃—(C₆H₂); R⁵ = F |
| C-78 | formula Ib.1 where R¹ = NH(CH(CH₃)CF₃); R⁴ = 2,4,6-F₃—(C₆H₂); R⁵ = F |
| C-79 | formula Ib.1 where R¹ = NH(CH(CH₃)₂); R⁴ = 2,4,6-F₃—(C₆H₂); R⁵ = Cl |
| C-80 | formula Ib.1 where R¹ = NH(CH(CH₃)C₂H₅); R⁴ = 2,4,6-F₃—(C₆H₂); R⁵ = Cl |
| C-81 | formula Ib.1 where R¹ = NH(CH₂CH(CH₃)₂); R⁴ = 2,4,6-F₃—(C₆H₂); R⁵ = Cl |
| C-82 | formula Ib.1 where R¹ = NH(CH(CH₃)CH(CH₃)₂); R⁴ = 2,4,6-F₃—(C₆H₂); R⁵ = Cl |
| C-83 | formula Ib.1 where R¹ = NH(CH(CH₃)C(CH₃)₃); R⁴ = 2,4,6-F₃—(C₆H₂); R⁵ = Cl |
| C-84 | formula Ib.1 where R¹ = NH(CH(CH₃)₂); R⁴ = 2,4,6-F₃—(C₆H₂); R⁵ = F |
| C-85 | formula Ib.1 where R¹ = NH(CH(CH₃)C₂H₅); R⁴ = 2,4,6-F₃—(C₆H₂); R⁵ = F |
| C-86 | formula Ib.1 where R¹ = NH(CH₂CH(CH₃)₂); R⁴ = 2,4,6-F₃—(C₆H₂); R⁵ = F |
| C-87 | formula Ib.1 where R¹ = NH(CH(CH₃)CH(CH₃)₂); R⁴ = 2,4,6-F₃—(C₆H₂); R⁵ = F |
| C-88 | formula Ib.1 where R¹ = NH(CH(CH₃)C(CH₃)₃); R⁴ = 2,4,6-F₃—(C₆H₂); R⁵ = F |
| C-89 | formula Ib.1 where R¹ = N(CH₃)(CH(CH₃)₂); R⁴ = 2,4,6-F₃—(C₆H₂); R⁵ = Cl |
| C-90 | formula Ib.1 where R¹ = N(CH₃)(CH(CH₃)C₂H₅); R⁴ = 2,4,6-F₃—(C₆H₂); R⁵ = Cl |
| C-91 | formula Ib.1 where R¹ = N(CH₃)(CH₂CH(CH₃)₂); R⁴ = 2,4,6-F₃—(C₆H₂); R⁵ = Cl |
| C-92 | formula Ib.1 where R¹ = N(CH₃)(CH(CH₃)CH(CH₃)₂); R⁴ = 2,4,6-F₃—(C₆H₂); R⁵ = Cl |
| C-93 | formula Ib.1 where R¹ = N(CH₃)(CH(CH₃)C(CH₃)₃); R⁴ = 2,4,6-F₃—(C₆H₂); R⁵ = Cl |
| C-94 | formula Ib.1 where R¹ = N(CH₃)(CH(CH₃)₂); R⁴ = 2,4,6-F₃—(C₆H₂); R⁵ = F |
| C-95 | formula Ib.1 where R¹ = N(CH₃)(CH(CH₃)C₂H₅); R⁴ = 2,4,6-F₃—(C₆H₂); R⁵ = F |
| C-96 | formula Ib.1 where R¹ = N(CH₃)(CH₂CH(CH₃)₂); R⁴ = 2,4,6-F₃—(C₆H₂); R⁵ = F |
| C-97 | formula Ib.1 where R¹ = N(CH₃)(CH(CH₃)CH(CH₃)₂); R⁴ = 2,4,6-F₃—(C₆H₂); R⁵ = F |
| C-98 | formula Ib.1 where R¹ = N(CH₃)(CH(CH₃)C(CH₃)₃); R⁴ = 2,4,6-F₃—(C₆H₂); R⁵ = F |
| C-99 | formula Ib.1 where R¹ = N(C₂H₅)(CH(CH₃)₂); R⁴ = 2,4,6-F₃—(C₆H₂); R⁵ = Cl |
| C-100 | formula Ib.1 where R¹ = N(C₂H₅)(CH(CH₃)C₂H₅); R⁴ = 2,4,6-F₃—(C₆H₂); R⁵ = Cl |
| C-101 | formula Ib.1 where R¹ = N(C₂H₅)(CH₂CH(CH₃)₂); R⁴ = 2,4,6-F₃—(C₆H₂); R⁵ = Cl |
| C-102 | formula Ib.1 where R¹ = N(C₂H₅)(CH(CH₃)CH(CH₃)₂); R⁴ = 2,4,6-F₃—(C₆H₂); R⁵ = Cl |
| C-103 | formula Ib.1 where R¹ = N(C₂H₅)(CH(CH₃)C(CH₃)₃); R⁴ = 2,4,6-F₃—(C₆H₂); R⁵ = Cl |
| C-104 | formula Ib.1 where R¹ = N(C₂H₅)(CH(CH₃)C₂H₅); R⁴ = 2,4,6-F₃—(C₆H₂); R⁵ = F |
| C-105 | formula Ib.1 where R¹ = N(C₂H₅)(CH₂CH(CH₃)₂); R⁴ = 2,4,6-F₃—(C₆H₂); R⁵ = F |
| C-106 | formula Ib.1 where R¹ = N(C₂H₅)(CH(CH₃)CH(CH₃)₂); R⁴ = 2,4,6-F₃—(C₆H₂); R⁵ = F |
| C-107 | formula Ib.1 where R¹ = N(C₂H₅)(CH(CH₃)C(CH₃)₃); R⁴ = 2,4,6-F₃—(C₆H₂); R⁵ = F |
| C-108 | formula Ib.1 where R¹ = N(C₂H₅)(CH(CH₃)C₂H₅); R⁴ = 2,4,6-F₃—(C₆H₂); R⁵ = F |
| C-109 | formula Ib.1 where R¹ = piperidin-1-yl; R⁴ = 2,4,6-F₃—(C₆H₂); R⁵ = Cl |
| C-110 | formula Ib.1 where R¹ = 2-CH₃-piperidin-1-yl; R⁴ = 2,4,6-F₃—(C₆H₂); R⁵ = Cl |
| C-111 | formula Ib.1 where R¹ = 3-CH₃-piperidin-1-yl; R⁴ = 2,4,6-F₃—(C₆H₂); R⁵ = Cl |
| C-112 | formula Ib.1 where R¹ = 4-CH₃-piperidin-1-yl; R⁴ = 2,4,6-F₃—(C₆H₂); R⁵ = Cl |
| C-113 | formula Ib.1 where R¹ = piperidin-1-yl; R⁴ = 2,4,6-F₃—(C₆H₂); R⁵ = F |
| C-114 | formula Ib.1 where R¹ = 2-CH₃-piperidin-1-yl; R⁴ = 2,4,6-F₃—(C₆H₂); R⁵ = F |
| C-115 | formula Ib.1 where R¹ = 3-CH₃-piperidin-1-yl; R⁴ = 2,4,6-F₃—(C₆H₂); R⁵ = F |
| C-116 | formula Ib.1 where R¹ = 4-CH₃-piperidin-1-yl; R⁴ = 2,4,6-F₃—(C₆H₂); R⁵ = F |
| C-117 | formula Ic.1 where R¹ = C₆H₁₁; R⁴ = 2,4,6-F₃—(C₆H₂); R⁵ = Cl |
| C-118 | formula Ic.1 where R¹ = 4-CH₃—(C₆H₁₁); R⁴ = 2,4,6-F₃—(C₆H₂); R⁵ = Cl |
| C-119 | formula Ic.1 where R¹ = CH₂(CH₂)₂CH₃; R⁴ = 2,4,6-F₃—(C₆H₂); R⁵ = Cl |
| C-120 | formula Ic.1 where R¹ = CH₂CH(CH₃)C₂H₅; R⁴ = 2,4,6-F₃—(C₆H₂); R⁵ = Cl |
| C-121 | formula Ic.1 where R¹ = CH₂C(CH₃)₂C₂H₅; R⁴ = 2,4,6-F₃—(C₆H₂); R⁵ = Cl |
| C-122 | formula Ic.1 where R¹ = CH₂CH₂CH═CH₂; R⁴ = 2,4,6-F₃—(C₆H₂); R⁵ = Cl |
| C-123 | formula Ic.1 where R¹ = CH₂CH(CH₃)CH═CH₂; R⁴ = 2,4,6-F₃—(C₆H₂); R⁵ = Cl |
| C-124 | formula Ic.1 where R¹ = CH₂C(CH₃)₂CH═CH₂; R⁴ = 2,4,6-F₃—(C₆H₂); R⁵ = Cl |
| C-125 | formula Ic.1 where R¹ = C₆H₁₁; R⁴ = 2,4,6-F₃—(C₆H₂); R⁵ = CH₃ |
| C-126 | formula Ic.1 where R¹ = 4-CH₃—(C₆H₁₁); R⁴ = 2,4,6-F₃—(C₆H₂); R⁵ = CH₃ |
| C-127 | formula Ic.1 where R¹ = CH₂(CH₂)₂CH₃; R⁴ = 2,4,6-F₃—(C₆H₂); R⁵ = CH₃ |
| C-128 | formula Ic.1 where R¹ = CH₂CH(CH₃)C₂H₅; R⁴ = 2,4,6-F₃—(C₆H₂); R⁵ = CH₃ |
| C-129 | formula Ic.1 where R¹ = CH₂C(CH₃)₂C₂H₅; R⁴ = 2,4,6-F₃—(C₆H₂); R⁵ = CH₃ |
| C-130 | formula Ic.1 where R¹ = CH₂CH₂CH═CH₂; R⁴ = 2,4,6-F₃—(C₆H₂); R⁵ = CH₃ |
| C-131 | formula Ic.1 where R¹ = CH₂CH(CH₃)CH═CH₂; R⁴ = 2,4,6-F₃—(C₆H₂); R⁵ = CH₃ |
| C-132 | formula Ic.1 where R¹ = CH₂C(CH₃)₂CH═CH₂; R⁴ = 2,4,6-F₃—(C₆H₂); R⁵ = CH₃ |
| C-133 | formula Ic.1 where R¹ = NH(CH₂CF₃); R⁴ = 2,4,6-F₃—(C₆H₂); R⁵ = Cl |
| C-134 | formula Ic.1 where R¹ = NH(CH(CH₃)CF₃); R⁴ = 2,4,6-F₃—(C₆H₂); R⁵ = Cl |
| C-135 | formula Ic.1 where R¹ = NH(CH₂CF₃); R⁴ = 2,4,6-F₃—(C₆H₂); R⁵ = F |
| C-136 | formula Ic.1 where R¹ = NH(CH(CH₃)CF₃); R⁴ = 2,4,6-F₃—(C₆H₂); R⁵ = F |
| C-137 | formula Ic.1 where R¹ = NH(CH(CH₃)₂); R⁴ = 2,4,6-F₃—(C₆H₂); R⁵ = Cl |
| C-138 | formula Ic.1 where R¹ = NH(CH(CH₃)C₂H₅); R⁴ = 2,4,6-F₃—(C₆H₂); R⁵ = Cl |
| C-139 | formula Ic.1 where R¹ = NH(CH₂CH(CH₃)₂); R⁴ = 2,4,6-F₃—(C₆H₂); R⁵ = Cl |
| C-140 | formula Ic.1 where R¹ = NH(CH(CH₃)CH(CH₃)₂); R⁴ = 2,4,6-F₃—(C₆H₂); R⁵ = Cl |
| C-141 | formula Ic.1 where R¹ = NH(CH(CH₃)C(CH₃)₃); R⁴ = 2,4,6-F₃—(C₆H₂); R⁵ = Cl |
| C-142 | formula Ic.1 where R¹ = NH(CH(CH₃)₂); R⁴ = 2,4,6-F₃—(C₆H₂); R⁵ = F |
| C-143 | formula Ic.1 where R¹ = NH(CH(CH₃)C₂H₅); R⁴ = 2,4,6-F₃—(C₆H₂); R⁵ = F |
| C-144 | formula Ic.1 where R¹ = NH(CH₂CH(CH₃)₂); R⁴ = 2,4,6-F₃—(C₆H₂); R⁵ = F |
| C-145 | formula Ic.1 where R¹ = NH(CH(CH₃)CH(CH₃)₂); R⁴ = 2,4,6-F₃—(C₆H₂); R⁵ = F |
| C-146 | formula Ic.1 where R¹ = NH(CH(CH₃)C(CH₃)₃); R⁴ = 2,4,6-F₃—(C₆H₂); R⁵ = F |
| C-147 | formula Ic.1 where R¹ = N(CH₃)(CH(CH₃)₂); R⁴ = 2,4,6-F₃—(C₆H₂); R⁵ = Cl |
| C-148 | formula Ic.1 where R¹ = N(CH₃)(CH(CH₃)C₂H₅); R⁴ = 2,4,6-F₃—(C₆H₂); R⁵ = Cl |
| C-149 | formula Ic.1 where R¹ = N(CH₃)(CH₂CH(CH₃)₂); R⁴ = 2,4,6-F₃—(C₆H₂); R⁵ = Cl |
| C-150 | formula Ic.1 where R¹ = N(CH₃)(CH(CH₃)CH(CH₃)₂); R⁴ = 2,4,6-F₃—(C₆H₂); R⁵ = Cl |
| C-151 | formula Ic.1 where R¹ = N(CH₃)(CH(CH₃)C(CH₃)₃); R⁴ = 2,4,6-F₃—(C₆H₂); R⁵ = Cl |
| C-152 | formula Ic.1 where R¹ = N(CH₃)(CH(CH₃)₂); R⁴ = 2,4,6-F₃—(C₆H₂); R⁵ = F |
| C-153 | formula Ic.1 where R¹ = N(CH₃)(CH(CH₃)C₂H₅); R⁴ = 2,4,6-F₃—(C₆H₂); R⁵ = F |
| C-154 | formula Ic.1 where R¹ = N(CH₃)(CH₂CH(CH₃)₂); R⁴ = 2,4,6-F₃—(C₆H₂); R⁵ = F |
| C-155 | formula Ic.1 where R¹ = N(CH₃)(CH(CH₃)CH(CH₃)₂); R⁴ = 2,4,6-F₃—(C₆H₂); R⁵ = F |
| C-156 | formula Ic.1 where R¹ = N(CH₃)(CH(CH₃)C(CH₃)₃); R⁴ = 2,4,6-F₃—(C₆H₂); R⁵ = F |
| C-157 | formula Ic.1 where R¹ = N(C₂H₅)(CH(CH₃)₂); R⁴ = 2,4,6-F₃—(C₆H₂); R⁵ = Cl |
| C-158 | formula Ic.1 where R¹ = N(C₂H₅)(CH(CH₃)C₂H₅); R⁴ = 2,4,6-F₃—(C₆H₂); R⁵ = Cl |
| C-159 | formula Ic.1 where R¹ = N(C₂H₅)(CH₂CH(CH₃)₂); R⁴ = 2,4,6-F₃—(C₆H₂); R⁵ = Cl |
| C-160 | formula Ic.1 where R¹ = N(C₂H₅)(CH(CH₃)CH(CH₃)₂); R⁴ = 2,4,6-F₃—(C₆H₂); R⁵ = Cl |
| C-161 | formula Ic.1 where R¹ = N(C₂H₅)(CH(CH₃)C(CH₃)₃); R⁴ = 2,4,6-F₃—(C₆H₂); R⁵ = Cl |
| C-162 | formula Ic.1 where R¹ = N(C₂H₅)(CH(CH₃)C₂H₅); R⁴ = 2,4,6-F₃—(C₆H₂); R⁵ = F |
| C-163 | formula Ic.1 where R¹ = N(C₂H₅)(CH₂CH(CH₃)₂); R⁴ = 2,4,6-F₃—(C₆H₂); R⁵ = F |
| C-164 | formula Ic.1 where R¹ = N(C₂H₅)(CH(CH₃)CH(CH₃)₂); R⁴ = 2,4,6-F₃—(C₆H₂); R⁵ = F |
| C-165 | formula Ic.1 where R¹ = N(C₂H₅)(CH(CH₃)C(CH₃)₃); R⁴ = 2,4,6-F₃—(C₆H₂); R⁵ = F |
| C-166 | formula Ic.1 where R¹ = N(C₂H₅)(CH(CH₃)C₂H₅); R⁴ = 2,4,6-F₃—(C₆H₂); R⁵ = F |
| C-167 | formula Ic.1 where R¹ = piperidin-1-yl; R⁴ = 2,4,6-F₃—(C₆H₂); R⁵ = Cl |
| C-168 | formula Ic.1 where R¹ = 2-CH₃-piperidin-1-yl; R⁴ = 2,4,6-F₃—(C₆H₂); R⁵ = Cl |
| C-169 | formula Ic.1 where R¹ = 3-CH₃-piperidin-1-yl; R⁴ = 2,4,6-F₃—(C₆H₂); R⁵ = Cl |
| C-170 | formula Ic.1 where R¹ = 4-CH₃-piperidin-1-yl; R⁴ = 2,4,6-F₃—(C₆H₂); R⁵ = Cl |
| C-171 | formula Ic.1 where R¹ = piperidin-1-yl; R⁴ = 2,4,6-F₃—(C₆H₂); R⁵ = F |
| C-172 | formula Ic.1 where R¹ = 2-CH₃-piperidin-1-yl; R⁴ = 2,4,6-F₃—(C₆H₂); R⁵ = F |
| C-173 | formula Ic.1 where R¹ = 3-CH₃-piperidin-1-yl; R⁴ = 2,4,6-F₃—(C₆H₂); R⁵ = F |
| C-174 | formula Ic.1 where R¹ = 4-CH₃-piperidin-1-yl; R⁴ = 2,4,6-F₃—(C₆H₂); R⁵ = F |
| C-175 | formula Id.1 where R¹ = C₆H₁₁; R⁴ = 2,4,6-F₃—(C₆H₂); R⁵ = Cl |
| C-176 | formula Id.1 where R¹ = 4-CH₃—(C₆H₁₁); R⁴ = 2,4,6-F₃—(C₆H₂); R⁵ = Cl |
| C-177 | formula Id.1 where R¹ = CH₂(CH₂)₂CH₃; R⁴ = 2,4,6-F₃—(C₆H₂); R⁵ = Cl |
| C-178 | formula Id.1 where R¹ = CH₂CH(CH₃)C₂H₅; R⁴ = 2,4,6-F₃—(C₆H₂); R⁵ = Cl |
| C-179 | formula Id.1 where R¹ = CH₂C(CH₃)₂C₂H₅; R⁴ = 2,4,6-F₃—(C₆H₂); R⁵ = Cl |
| C-180 | formula Id.1 where R¹ = CH₂CH₂CH═CH₂; R⁴ = 2,4,6-F₃—(C₆H₂); R⁵ = Cl |
| C-181 | formula Id.1 where R¹ = CH₂CH(CH₃)CH═CH₂; R⁴ = 2,4,6-F₃—(C₆H₂); R⁵ = Cl |
| C-182 | formula Id.1 where R¹ = CH₂C(CH₃)₂CH═CH₂; R⁴ = 2,4,6-F₃—(C₆H₂); R⁵ = Cl |
| C-183 | formula Id.1 where R¹ = C₆H₁₁; R⁴ = 2,4,6-F₃—(C₆H₂); R⁵ = CH₃ |
| C-184 | formula Id.1 where R¹ = 4-CH₃—(C₆H₁₁); R⁴ = 2,4,6-F₃—(C₆H₂); R⁵ = CH₃ |
| C-185 | formula Id.1 where R¹ = CH₂(CH₂)₂CH₃; R⁴ = 2,4,6-F₃—(C₆H₂); R⁵ = CH₃ |
| C-186 | formula Id.1 where R¹ = CH₂CH(CH₃)C₂H₅; R⁴ = 2,4,6-F₃—(C₆H₂); R⁵ = CH₃ |
| C-187 | formula Id.1 where R¹ = CH₂C(CH₃)₂C₂H₅; R⁴ = 2,4,6-F₃—(C₆H₂); R⁵ = CH₃ |
| C-188 | formula Id.1 where R¹ = CH₂CH₂CH═CH₂; R⁴ = 2,4,6-F₃—(C₆H₂); R⁵ = CH₃ |
| C-189 | formula Id.1 where R¹ = CH₂CH(CH₃)CH═CH₂; R⁴ = 2,4,6-F₃—(C₆H₂); R⁵ = CH₃ |
| C-190 | formula Id.1 where R¹ = CH₂C(CH₃)₂CH═CH₂; R⁴ = 2,4,6-F₃—(C₆H₂); R⁵ = CH₃ |
| C-191 | formula Id.1 where R¹ = NH(CH₂CF₃); R⁴ = 2,4,6-F₃—(C₆H₂); R⁵ = Cl |
| C-192 | formula Id.1 where R¹ = NH(CH(CH₃)CF₃); R⁴ = 2,4,6-F₃—(C₆H₂); R⁵ = Cl |
| C-193 | formula Id.1 where R¹ = NH(CH₂CF₃); R⁴ = 2,4,6-F₃—(C₆H₂); R⁵ = F |
| C-194 | formula Id.1 where R¹ = NH(CH(CH₃)CF₃); R⁴ = 2,4,6-F₃—(C₆H₂); R⁵ = F |
| C-195 | formula Id.1 where R¹ = NH(CH(CH₃)₂); R⁴ = 2,4,6-F₃—(C₆H₂); R⁵ = Cl |
| C-196 | formula Id.1 where R¹ = NH(CH(CH₃)C₂H₅); R⁴ = 2,4,6-F₃—(C₆H₂); R⁵ = Cl |
| C-197 | formula Id.1 where R¹ = NH(CH₂CH(CH₃)₂); R⁴ = 2,4,6-F₃—(C₆H₂); R⁵ = Cl |
| C-198 | formula Id.1 where R¹ = NH(CH(CH₃)CH(CH₃)₂); R⁴ = 2,4,6-F₃—(C₆H₂); R⁵ = Cl |
| C-199 | formula Id.1 where R¹ = NH(CH(CH₃)C(CH₃)₃); R⁴ = 2,4,6-F₃—(C₆H₂); R⁵ = Cl |
| C-200 | formula Id.1 where R¹ = NH(CH(CH₃)₂); R⁴ = 2,4,6-F₃—(C₆H₂); R⁵ = F |
| C-201 | formula Id.1 where R¹ = NH(CH(CH₃)C₂H₅); R⁴ = 2,4,6-F₃—(C₆H₂); R⁵ = F |
| C-202 | formula Id.1 where R¹ = NH(CH₂CH(CH₃)₂); R⁴ = 2,4,6-F₃—(C₆H₂); R⁵ = F |
| C-203 | formula Id.1 where R¹ = NH(CH(CH₃)CH(CH₃)₂); R⁴ = 2,4,6-F₃—(C₆H₂); R⁵ = F |
| C-204 | formula Id.1 where R¹ = NH(CH(CH₃)C(CH₃)₃); R⁴ = 2,4,6-F₃—(C₆H₂); R⁵ = F |
| C-205 | formula Id.1 where R¹ = N(CH₃)(CH(CH₃)₂); R⁴ = 2,4,6-F₃—(C₆H₂); R⁵ = Cl |
| C-206 | formula Id.1 where R¹ = N(CH₃)(CH(CH₃)C₂H₅); R⁴ = 2,4,6-F₃—(C₆H₂); R⁵ = Cl |
| C-207 | formula Id.1 where R¹ = N(CH₃)(CH₂CH(CH₃)₂); R⁴ = 2,4,6-F₃—(C₆H₂); R⁵ = Cl |
| C-208 | formula Id.1 where R¹ = N(CH₃)(CH(CH₃)CH(CH₃)₂); R⁴ = 2,4,6-F₃—(C₆H₂); R⁵ = Cl |
| C-209 | formula Id.1 where R¹ = N(CH₃)(CH(CH₃)C(CH₃)₃); R⁴ = 2,4,6-F₃—(C₆H₂); R⁵ = Cl |
| C-210 | formula Id.1 where R¹ = N(CH₃)(CH(CH₃)₂); R⁴ = 2,4,6-F₃—(C₆H₂); R⁵ = F |
| C-211 | formula Id.1 where R¹ = N(CH₃)(CH(CH₃)C₂H₅); R⁴ = 2,4,6-F₃—(C₆H₂); R⁵ = F |
| C-212 | formula Id.1 where R¹ = N(CH₃)(CH₂CH(CH₃)₂); R⁴ = 2,4,6-F₃—(C₆H₂); R⁵ = F |
| C-213 | formula Id.1 where R¹ = N(CH₃)(CH(CH₃)CH(CH₃)₂); R⁴ = 2,4,6-F₃—(C₆H₂); R⁵ = F |
| C-214 | formula Id.1 where R¹ = N(CH₃)(CH(CH₃)C(CH₃)₃); R⁴ = 2,4,6-F₃—(C₆H₂); R⁵ = F |
| C-215 | formula Id.1 where R¹ = N(C₂H₅)(CH(CH₃)₂); R⁴ = 2,4,6-F₃—(C₆H₂); R⁵ = Cl |
| C-216 | formula Id.1 where R¹ = N(C₂H₅)(CH(CH₃)C₂H₅); R⁴ = 2,4,6-F₃—(C₆H₂); R⁵ = Cl |
| C-217 | formula Id.1 where R¹ = N(C₂H₅)(CH₂CH(CH₃)₂); R⁴ = 2,4,6-F₃—(C₆H₂); R⁵ = Cl |
| C-218 | formula Id.1 where R¹ = N(C₂H₅)(CH(CH₃)CH(CH₃)₂); R⁴ = 2,4,6-F₃—(C₆H₂); R⁵ = Cl |
| C-219 | formula Id.1 where R¹ = N(C₂H₅)(CH(CH₃)C(CH₃)₃); R⁴ = 2,4,6-F₃—(C₆H₂); R⁵ = Cl |
| C-220 | formula Id.1 where R¹ = N(C₂H₅)(CH(CH₃)C₂H₅); R⁴ = 2,4,6-F₃—(C₆H₂); R⁵ = F |
| C-221 | formula Id.1 where R¹ = N(C₂H₅)(CH₂CH(CH₃)₂); R⁴ = 2,4,6-F₃—(C₆H₂); R⁵ = F |
| C-222 | formula Id.1 where R¹ = N(C₂H₅)(CH(CH₃)CH(CH₃)₂); R⁴ = 2,4,6-F₃—(C₆H₂); R⁵ = F |
| C-223 | formula Id.1 where R¹ = N(C₂H₅)(CH(CH₃)C(CH₃)₃); R⁴ = 2,4,6-F₃—(C₆H₂); R⁵ = F |
| C-224 | formula Id.1 where R¹ = N(C₂H₅)(CH(CH₃)C₂H₅); R⁴ = 2,4,6-F₃—(C₆H₂); R⁵ = F |
| C-225 | formula Id.1 where R¹ = piperidin-1-yl; R⁴ = 2,4,6-F₃—(C₆H₂); R⁵ = Cl |
| C-226 | formula Id.1 where R¹ = 2-CH₃-piperidin-1-yl; R⁴ = 2,4,6-F₃—(C₆H₂); R⁵ = Cl |
| C-227 | formula Id.1 where R¹ = 3-CH₃-piperidin-1-yl; R⁴ = 2,4,6-F₃—(C₆H₂); R⁵ = Cl |
| C-228 | formula Id.1 where R¹ = 4-CH₃-piperidin-1-yl; R⁴ = 2,4,6-F₃—(C₆H₂); R⁵ = Cl |
| C-229 | formula Id.1 where R¹ = piperidin-1-yl; R⁴ = 2,4,6-F₃—(C₆H₂); R⁵ = F |
| C-230 | formula Id.1 where R¹ = 2-CH₃-piperidin-1-yl; R⁴ = 2,4,6-F₃—(C₆H₂); R⁵ = F |
| C-231 | formula Id.1 where R¹ = 3-CH₃-piperidin-1-yl; R⁴ = 2,4,6-F₃—(C₆H₂); R⁵ = F |
| C-232 | formula Id.1 where R¹ = 4-CH₃-piperidin-1-yl; R⁴ = 2,4,6-F₃—(C₆H₂); R⁵ = F |
| C-233 | formula Ie.1 where R¹ = C₆H₁₁; R⁴ = 2,4,6-F₃—(C₆H₂); R⁵ = Cl |
| C-234 | formula Ie.1 where R¹ = 4-CH₃—(C₆H₁₁); R⁴ = 2,4,6-F₃—(C₆H₂); R⁵ = Cl |
| C-235 | formula Ie.1 where R¹ = CH₂(CH₂)₂CH₃; R⁴ = 2,4,6-F₃—(C₆H₂); R⁵ = Cl |
| C-236 | formula Ie.1 where R¹ = CH₂CH(CH₃)C₂H₅; R⁴ = 2,4,6-F₃—(C₆H₂); R⁵ = Cl |
| C-237 | formula Ie.1 where R¹ = CH₂C(CH₃)₂C₂H₅; R⁴ = 2,4,6-F₃—(C₆H₂); R⁵ = Cl |
| C-238 | formula Ie.1 where R¹ = CH₂CH₂CH═CH₂; R⁴ = 2,4,6-F₃—(C₆H₂); R⁵ = Cl |
| C-239 | formula Ie.1 where R¹ = CH₂CH(CH₃)CH═CH₂; R⁴ = 2,4,6-F₃—(C₆H₂); R⁵ = Cl |
| C-240 | formula Ie.1 where R¹ = CH₂C(CH₃)₂CH═CH₂; R⁴ = 2,4,6-F₃—(C₆H₂); R⁵ = Cl |
| C-241 | formula Ie.1 where R¹ = C₆H₁₁; R⁴ = 2,4,6-F₃—(C₆H₂); R⁵ = CH₃ |
| C-242 | formula Ie.1 where R¹ = 4-CH₃—(C₆H₁₁); R⁴ = 2,4,6-F₃—(C₆H₂); R⁵ = CH₃ |
| C-243 | formula Ie.1 where R¹ = CH₂(CH₂)₂CH₃; R⁴ = 2,4,6-F₃—(C₆H₂); R⁵ = CH₃ |
| C-244 | formula Ie.1 where R¹ = CH₂CH(CH₃)C₂H₅; R⁴ = 2,4,6-F₃—(C₆H₂); R⁵ = CH₃ |
| C-245 | formula Ie.1 where R¹ = CH₂C(CH₃)₂C₂H₅; R⁴ = 2,4,6-F₃—(C₆H₂); R⁵ = CH₃ |
| C-246 | formula Ie.1 where R¹ = CH₂CH₂CH═CH₂; R⁴ = 2,4,6-F₃—(C₆H₂); R⁵ = CH₃ |
| C-247 | formula Ie.1 where R¹ = CH₂CH(CH₃)CH═CH₂; R⁴ = 2,4,6-F₃—(C₆H₂); R⁵ = CH₃ |
| C-248 | formula Ie.1 where R¹ = CH₂C(CH₃)₂CH═CH₂; R⁴ = 2,4,6-F₃—(C₆H₂); R⁵ = CH₃ |
| C-249 | formula Ie.1 where R¹ = NH(CH₂CF₃); R⁴ = 2,4,6-F₃—(C₆H₂); R⁵ = Cl |
| C-250 | formula Ie.1 where R¹ = NH(CH(CH₃)CF₃); R⁴ = 2,4,6-F₃—(C₆H₂); R⁵ = Cl |
| C-251 | formula Ie.1 where R¹ = NH(CH₂CF₃); R⁴ = 2,4,6-F₃—(C₆H₂); R⁵ = F |
| C-252 | formula Ie.1 where R¹ = NH(CH(CH₃)CF₃); R⁴ = 2,4,6-F₃—(C₆H₂); R⁵ = F |
| C-253 | formula Ie.1 where R¹ = NH(CH(CH₃)₂); R⁴ = 2,4,6-F₃—(C₆H₂); R⁵ = Cl |
| C-254 | formula Ie.1 where R¹ = NH(CH(CH₃)C₂H₅); R⁴ = 2,4,6-F₃—(C₆H₂); R⁵ = Cl |
| C-255 | formula Ie.1 where R¹ = NH(CH₂CH(CH₃)₂); R⁴ = 2,4,6-F₃—(C₆H₂); R⁵ = Cl |
| C-256 | formula Ie.1 where R¹ = NH(CH(CH₃)CH(CH₃)₂); R⁴ = 2,4,6-F₃—(C₆H₂); R⁵ = Cl |
| C-257 | formula Ie.1 where R¹ = NH(CH(CH₃)C(CH₃)₃); R⁴ = 2,4,6-F₃—(C₆H₂); R⁵ = Cl |
| C-258 | formula Ie.1 where R¹ = NH(CH(CH₃)₂); R⁴ = 2,4,6-F₃—(C₆H₂); R⁵ = F |
| C-259 | formula Ie.1 where R¹ = NH(CH(CH₃)C₂H₅); R⁴ = 2,4,6-F₃—(C₆H₂); R⁵ = F |
| C-260 | formula Ie.1 where R¹ = NH(CH₂CH(CH₃)₂); R⁴ = 2,4,6-F₃—(C₆H₂); R⁵ = F |
| C-261 | formula Ie.1 where R¹ = NH(CH(CH₃)CH(CH₃)₂); R⁴ = 2,4,6-F₃—(C₆H₂); R⁵ = F |
| C-262 | formula Ie.1 where R¹ = NH(CH(CH₃)C(CH₃)₃); R⁴ = 2,4,6-F₃—(C₆H₂); R⁵ = F |
| C-263 | formula Ie.1 where R¹ = N(CH₃)(CH(CH₃)₂); R⁴ = 2,4,6-F₃—(C₆H₂); R⁵ = Cl |
| C-264 | formula Ie.1 where R¹ = N(CH₃)(CH(CH₃)C₂H₅); R⁴ = 2,4,6-F₃—(C₆H₂); R⁵ = Cl |
| C-265 | formula Ie.1 where R¹ = N(CH₃)(CH₂CH(CH₃)₂); R⁴ = 2,4,6-F₃—(C₆H₂); R⁵ = Cl |
| C-266 | formula Ie.1 where R¹ = N(CH₃)(CH(CH₃)CH(CH₃)₂); R⁴ = 2,4,6-F₃—(C₆H₂); R⁵ = Cl |
| C-267 | formula Ie.1 where R¹ = N(CH₃)(CH(CH₃)C(CH₃)₃); R⁴ = 2,4,6-F₃—(C₆H₂); R⁵ = Cl |
| C-268 | formula Ie.1 where R¹ = N(CH₃)(CH(CH₃)₂); R⁴ = 2,4,6-F₃—(C₆H₂); R⁵ = F |
| C-269 | formula Ie.1 where R¹ = N(CH₃)(CH(CH₃)C₂H₅); R⁴ = 2,4,6-F₃—(C₆H₂); R⁵ = F |
| C-270 | formula Ie.1 where R¹ = N(CH₃)(CH₂CH(CH₃)₂); R⁴ = 2,4,6-F₃—(C₆H₂); R⁵ = F |
| C-271 | formula Ie.1 where R¹ = N(CH₃)(CH(CH₃)CH(CH₃)₂); R⁴ = 2,4,6-F₃—(C₆H₂); R⁵ = F |
| C-272 | formula Ie.1 where R¹ = N(CH₃)(CH(CH₃)C(CH₃)₃); R⁴ = 2,4,6-F₃—(C₆H₂); R⁵ = F |
| C-273 | formula Ie.1 where R¹ = N(C₂H₅)(CH(CH₃)₂); R⁴ = 2,4,6-F₃—(C₆H₂); R⁵ = Cl |
| C-274 | formula Ie.1 where R¹ = N(C₂H₅)(CH(CH₃)C₂H₅); R⁴ = 2,4,6-F₃—(C₆H₂); R⁵ = Cl |
| C-275 | formula Ie.1 where R¹ = N(C₂H₅)(CH₂CH(CH₃)₂); R⁴ = 2,4,6-F₃—(C₆H₂); R⁵ = Cl |
| C-276 | formula Ie.1 where R¹ = N(C₂H₅)(CH(CH₃)CH(CH₃)₂); R⁴ = 2,4,6-F₃—(C₆H₂); R⁵ = Cl |
| C-277 | formula Ie.1 where R¹ = N(C₂H₅)(CH(CH₃)C(CH₃)₃); R⁴ = 2,4,6-F₃—(C₆H₂); R⁵ = Cl |
| C-278 | formula Ie.1 where R¹ = N(C₂H₅)(CH(CH₃)C₂H₅); R⁴ = 2,4,6-F₃—(C₆H₂); R⁵ = F |
| C-279 | formula Ie.1 where R¹ = N(C₂H₅)(CH₂CH(CH₃)₂); R⁴ = 2,4,6-F₃—(C₆H₂); R⁵ = F |
| C-280 | formula Ie.1 where R¹ = N(C₂H₅)(CH(CH₃)CH(CH₃)₂); R⁴ = 2,4,6-F₃—(C₆H₂); R⁵ = F |
| C-281 | formula Ie.1 where R¹ = N(C₂H₅)(CH(CH₃)C(CH₃)₃); R⁴ = 2,4,6-F₃—(C₆H₂); R⁵ = F |
| C-282 | formula Ie.1 where R¹ = N(C₂H₅)(CH(CH₃)C₂H₅); R⁴ = 2,4,6-F₃—(C₆H₂); R⁵ = F |
| C-283 | formula Ie.1 where R¹ = piperidin-1-yl; R⁴ = 2,4,6-F₃—(C₆H₂); R⁵ = Cl |
| C-284 | formula Ie.1 where R¹ = 2-CH₃-piperidin-1-yl; R⁴ = 2,4,6-F₃—(C₆H₂); R⁵ = Cl |
| C-285 | formula Ie.1 where R¹ = 3-CH₃-piperidin-1-yl; R⁴ = 2,4,6-F₃—(C₆H₂); R⁵ = Cl |
| C-286 | formula Ie.1 where R¹ = 4-CH₃-piperidin-1-yl; R⁴ = 2,4,6-F₃—(C₆H₂); R⁵ = Cl |
| C-287 | formula Ie.1 where R¹ = piperidin-1-yl; R⁴ = 2,4,6-F₃—(C₆H₂); R⁵ = F |
| C-288 | formula Ie.1 where R¹ = 2-CH₃-piperidin-1-yl; R⁴ = 2,4,6-F₃—(C₆H₂); R⁵ = F |
| C-289 | formula Ie.1 where R¹ = 3-CH₃-piperidin-1-yl; R⁴ = 2,4,6-F₃—(C₆H₂); R⁵ = F |
| C-290 | formula Ie.1 where R¹ = 4-CH₃-piperidin-1-yl; R⁴ = 2,4,6-F₃—(C₆H₂); R⁵ = F |
| C-291 | formula If.1 where R¹ = C₆H₁₁; R⁴ = 2,4,6-F₃—(C₆H₂); R⁵ = Cl |
| C-292 | formula If.1 where R¹ = 4-CH₃—(C₆H₁₁); R⁴ = 2,4,6-F₃—(C₆H₂); R⁵ = Cl |
| C-293 | formula If.1 where R¹ = CH₂(CH₂)₂CH₃; R⁴ = 2,4,6-F₃—(C₆H₂); R⁵ = Cl |
| C-294 | formula If.1 where R¹ = CH₂CH(CH₃)C₂H₅; R⁴ = 2,4,6-F₃—(C₆H₂); R⁵ = Cl |
| C-295 | formula If.1 where R¹ = CH₂C(CH₃)₂C₂H₅; R⁴ = 2,4,6-F₃—(C₆H₂); R⁵ = Cl |
| C-296 | formula If.1 where R¹ = CH₂CH₂CH═CH₂; R⁴ = 2,4,6-F₃—(C₆H₂); R⁵ = Cl |
| C-297 | formula If.1 where R¹ = CH₂CH(CH₃)CH═CH₂; R⁴ = 2,4,6-F₃—(C₆H₂); R⁵ = Cl |
| C-298 | formula If.1 where R¹ = CH₂C(CH₃)₂CH═CH₂; R⁴ = 2,4,6-F₃—(C₆H₂); R⁵ = Cl |
| C-299 | formula If.1 where R¹ = C₆H₁₁; R⁴ = 2,4,6-F₃—(C₆H₂); R⁵ = CH₃ |
| C-300 | formula If.1 where R¹ = 4-CH₃—(C₆H₁₁); R⁴ = 2,4,6-F₃—(C₆H₂); R⁵ = CH₃ |
| C-301 | formula If.1 where R¹ = CH₂(CH₂)₂CH₃; R⁴ = 2,4,6-F₃—(C₆H₂); R⁵ = CH₃ |
| C-302 | formula If.1 where R¹ = CH₂CH(CH₃)C₂H₅; R⁴ = 2,4,6-F₃—(C₆H₂); R⁵ = CH₃ |
| C-303 | formula If.1 where R¹ = CH₂C(CH₃)₂C₂H₅; R⁴ = 2,4,6-F₃—(C₆H₂); R⁵ = CH₃ |
| C-304 | formula If.1 where R¹ = CH₂CH₂CH═CH₂; R⁴ = 2,4,6-F₃—(C₆H₂); R⁵ = CH₃ |
| C-305 | formula If.1 where R¹ = CH₂CH(CH₃)CH═CH₂; R⁴ = 2,4,6-F₃—(C₆H₂); R⁵ = CH₃ |
| C-306 | formula If.1 where R¹ = CH₂C(CH₃)₂CH═CH₂; R⁴ = 2,4,6-F₃—(C₆H₂); R⁵ = CH₃ |
| C-307 | formula If.1 where R¹ = NH(CH₂CF₃); R⁴ = 2,4,6-F₃—(C₆H₂); R⁵ = Cl |
| C-308 | formula If.1 where R¹ = NH(CH(CH₃)CF₃); R⁴ = 2,4,6-F₃—(C₆H₂); R⁵ = Cl |
| C-309 | formula If.1 where R¹ = NH(CH₂CF₃); R⁴ = 2,4,6-F₃—(C₆H₂); R⁵ = F |
| C-310 | formula If.1 where R¹ = NH(CH(CH₃)CF₃); R⁴ = 2,4,6-F₃—(C₆H₂); R⁵ = F |
| C-311 | formula If.1 where R¹ = NH(CH(CH₃)₂); R⁴ = 2,4,6-F₃—(C₆H₂); R⁵ = Cl |
| C-312 | formula If.1 where R¹ = NH(CH(CH₃)C₂H₅); R⁴ = 2,4,6-F₃—(C₆H₂); R⁵ = Cl |
| C-313 | formula If.1 where R¹ = NH(CH₂CH(CH₃)₂); R⁴ = 2,4,6-F₃—(C₆H₂); R⁵ = Cl |
| C-314 | formula If.1 where R¹ = NH(CH(CH₃)CH(CH₃)₂); R⁴ = 2,4,6-F₃—(C₆H₂); R⁵ = Cl |
| C-315 | formula If.1 where R¹ = NH(CH(CH₃)C(CH₃)₃); R⁴ = 2,4,6-F₃—(C₆H₂); R⁵ = Cl |
| C-316 | formula If.1 where R¹ = NH(CH(CH₃)₂); R⁴ = 2,4,6-F₃—(C₆H₂); R⁵ = F |
| C-317 | formula If.1 where R¹ = NH(CH(CH₃)C₂H₅); R⁴ = 2,4,6-F₃—(C₆H₂); R⁵ = F |
| C-318 | formula If.1 where R¹ = NH(CH₂CH(CH₃)₂); R⁴ = 2,4,6-F₃—(C₆H₂); R⁵ = F |
| C-319 | formula If.1 where R¹ = NH(CH(CH₃)CH(CH₃)₂); R⁴ = 2,4,6-F₃—(C₆H₂); R⁵ = F |
| C-320 | formula If.1 where R¹ = NH(CH(CH₃)C(CH₃)₃); R⁴ = 2,4,6-F₃—(C₆H₂); R⁵ = F |
| C-321 | formula If.1 where R¹ = N(CH₃)(CH(CH₃)₂); R⁴ = 2,4,6-F₃—(C₆H₂); R⁵ = Cl |
| C-322 | formula If.1 where R¹ = N(CH₃)(CH(CH₃)C₂H₅); R⁴ = 2,4,6-F₃—(C₆H₂); R⁵ = Cl |
| C-323 | formula If.1 where R¹ = N(CH₃)(CH₂CH(CH₃)₂); R⁴ = 2,4,6-F₃—(C₆H₂); R⁵ = Cl |
| C-324 | formula If.1 where R¹ = N(CH₃)(CH(CH₃)CH(CH₃)₂); R⁴ = 2,4,6-F₃—(C₆H₂); R⁵ = Cl |
| C-325 | formula If.1 where R¹ = N(CH₃)(CH(CH₃)C(CH₃)₃); R⁴ = 2,4,6-F₃—(C₆H₂); R⁵ = Cl |
| C-326 | formula If.1 where R¹ = N(CH₃)(CH(CH₃)₂); R⁴ = 2,4,6-F₃—(C₆H₂); R⁵ = F |
| C-327 | formula If.1 where R¹ = N(CH₃)(CH(CH₃)C₂H₅); R⁴ = 2,4,6-F₃—(C₆H₂); R⁵ = F |
| C-328 | formula If.1 where R¹ = N(CH₃)(CH₂CH(CH₃)₂); R⁴ = 2,4,6-F₃—(C₆H₂); R⁵ = F |
| C-329 | formula If.1 where R¹ = N(CH₃)(CH(CH₃)CH(CH₃)₂); R⁴ = 2,4,6-F₃—(C₆H₂); R⁵ = F |
| C-330 | formula If.1 where R¹ = N(CH₃)(CH(CH₃)C(CH₃)₃); R⁴ = 2,4,6-F₃—(C₆H₂); R⁵ = F |
| C-331 | formula If.1 where R¹ = N(C₂H₅)(CH(CH₃)₂); R⁴ = 2,4,6-F₃—(C₆H₂); R⁵ = Cl |
| C-332 | formula If.1 where R¹ = N(C₂H₅)(CH(CH₃)C₂H₅); R⁴ = 2,4,6-F₃—(C₆H₂); R⁵ = Cl |
| C-333 | formula If.1 where R¹ = N(C₂H₅)(CH₂CH(CH₃)₂); R⁴ = 2,4,6-F₃—(C₆H₂); R⁵ = Cl |
| C-334 | formula If.1 where R¹ = N(C₂H₅)(CH(CH₃)CH(CH₃)₂); R⁴ = 2,4,6-F₃—(C₆H₂); R⁵ = Cl |
| C-335 | formula If.1 where R¹ = N(C₂H₅)(CH(CH₃)C(CH₃)₃); R⁴ = 2,4,6-F₃—(C₆H₂); R⁵ = Cl |
| C-336 | formula If.1 where R¹ = N(C₂H₅)(CH(CH₃)C₂H₅); R⁴ = 2,4,6-F₃—(C₆H₂); R⁵ = F |
| C-337 | formula If.1 where R¹ = N(C₂H₅)(CH₂CH(CH₃)₂); R⁴ = 2,4,6-F₃—(C₆H₂); R⁵ = F |
| C-338 | formula If.1 where R¹ = N(C₂H₅)(CH(CH₃)CH(CH₃)₂); R⁴ = 2,4,6-F₃—(C₆H₂); R⁵ = F |
| C-339 | formula If.1 where R¹ = N(C₂H₅)(CH(CH₃)C(CH₃)₃); R⁴ = 2,4,6-F₃—(C₆H₂); R⁵ = F |
| C-340 | formula If.1 where R¹ = N(C₂H₅)(CH(CH₃)C₂H₅); R⁴ = 2,4,6-F₃—(C₆H₂); R⁵ = F |
| C-341 | formula If.1 where R¹ = piperidin-1-yl; R⁴ = 2,4,6-F₃—(C₆H₂); R⁵ = Cl |
| C-342 | formula If.1 where R¹ = 2-CH₃-piperidin-1-yl; R⁴ = 2,4,6-F₃—(C₆H₂); R⁵ = Cl |
| C-343 | formula If.1 where R¹ = 3-CH₃-piperidin-1-yl; R⁴ = 2,4,6-F₃—(C₆H₂); R⁵ = Cl |
| C-344 | formula If.1 where R¹ = 4-CH₃-piperidin-1-yl; R⁴ = 2,4,6-F₃—(C₆H₂); R⁵ = Cl |
| C-345 | formula If.1 where R¹ = piperidin-1-yl; R⁴ = 2,4,6-F₃—(C₆H₂); R⁵ = F |
| C-346 | formula If.1 where R¹ = 2-CH₃-piperidin-1-yl; R⁴ = 2,4,6-F₃—(C₆H₂); R⁵ = F |
| C-347 | formula If.1 where R¹ = 3-CH₃-piperidin-1-yl; R⁴ = 2,4,6-F₃—(C₆H₂); R⁵ = F |
| C-348 | formula If.1 where R¹ = 4-CH₃-piperidin-1-yl; R⁴ = 2,4,6-F₃—(C₆H₂); R⁵ = F |

The compounds according to the invention and the compositions of compound(s) I, in particular a compound Ia, Ib, Ic, Id, Ie or If, and at least one of the active compounds II, or at least one compound I and at least one of the active compounds II applied simultaneously, that is jointly or separately, have excellent activity against a broad spectrum of phytopathogenic fungi from the classes of the Ascomycetes, Deuteromycetes, Basidiomycetes, Peronosporomycetes (syn. Oomycetes) and Fungi imperfecti. Some of them are systemically effective and can be employed in crop protection as foliar fungicides, as fungicides for seed dressing and as soil fungicides.

They are particularly important in the control of a multitude of fungi on various crop plants, such as wheat, rye, barley, triticale, oats, rice, corn, grass, bananas, cotton, soya, coffee, sugar cane, vines, fruit and ornamental plants, and vegetable plants, such as cucumbers, beans, tomatoes, potatoes and cucurbits, and on the seeds of these plants. They can also be used in crops which, owing to breeding including genetic engineering methods, are tolerant to attack by insects or fungi or to herbicide applications. In addition, they are suitable for controlling Botryosphaeria species, Cylindrocarpon species, Eutypa lata, Neonectria liriodendri and Stereum hirsutum which, inter glia, attack the wood or the roots of grapevines.

Specifically, they are suitable for controlling the following plant diseases:

Alternaria species on vegetables, oilseed rape, sugar beet, fruit, rice, soybeans, and also on potatoes (for example A. solani or A. alternate) and tomatoes (for example A. solani or A. alternate) and Alternaria ssp. (black head) on wheat,
Aphanomyces species on sugar beet and vegetables,
Ascochyta species on cereals and vegetables, for example Ascochyta tritici (leaf spot) on wheat,
Bipolaris and Drechslera species on corn (for example D. maydis), cereals, rice and lawn,
Blumeria graminis (powdery mildew) on cereals (for example wheat or barley),
Botrytis cinerea (gray mold) on strawberries, vegetables, flowers, grapevines and wheat (ear mold),
Bremia lactucae on lettuce,
Cercospora species on corn, rice, sugar beet and, for example, Cercospora sojina (leaf blotch) or Cercospora kikuchii (leaf blotch) on soybeans,
Cladosporium herbarum (black mold) on wheat,
Cochliobolus species on corn, cereals (for example Cochliobolus sativus) and rice (for example Cochliobolus miyabeanus),
Colletotricum species on cotton and, for example, Colletotrichum truncatum (antracnose) on soybeans,
Corynespora cassilcola (leaf blotch) on soybeans,
Dematophora necatrix (root/stem rot) on soybeans,
Diaporthe phaseolorum (stem disease) on soybeans,
Drechslera species, Pyrenophora species on corn, cereals, rice and lawn, on barley (for example D. teres) and on wheat (for example D. tritici-repentis),
Esca on grapevines, caused by Phaeoacremonium chlamydosporium, Ph. Aleophilum, and Formitipora punctata (syn. Pheffinus punctatus),
Elsinoe ampelina on grapevines,
Epicoccum spp. (black mold) on wheat,
Exserohilum species on corn,
Erysiphe cichoracearum and Sphaerotheca fuliginea on cucumbers,
Fusarium and Verticillium species on various plants: for example F. graminearum or F. culmorum (root rot) on cereals (for example wheat or barley) or, for example,
F. oxysporum on tomatoes and Fusarium solani (stem disease) on soybeans,
Gaeumanomyces graminis (root black) on cereals (for example wheat or barley),
Gibberella species on cereals and rice (for example Gibberella fujikurol),
Glomerella cingulata on grapevines and other plants,
Grainstaining complex on rice,
Guignardia budwelli on grapevines,
Helminthosporium species on corn and rice,
Isariopsis clavispora on grapevines,
Macrophomina phaseolina (root/stem rot) on soybeans,
Michrodochium nivale (pink snow mold) on cereals (for example wheat or barley),
Microsphaera diffuse (powdery mildew) on soybeans,
Mycosphaerella species on cereals, bananas and peanuts, such as, for example,
M. graminicola on wheat or M. fijiensis on bananas,
Peronospora species on cabbage (for example P. brassicae), bulbous plants (for example P. destructor) and, for example, Peronospora manshurica (downy mildew) on soybeans,
Phakopsara pachyrhizi (soybean rust) and Phakopsara meibomiae (soybean rust) on soybeans,
Phialophora gregata (stem disease) on soybeans,
Phomopsis species on sunflowers, grapevines (for example P. viticola) and soybeans (for example Phomopsis phaseoli),
Phytophthora species on various plants, for example P. capsici on bell peppers,
Phytophthora megasperma (leaf/stem rot) on soybeans, Phytophthora infestans on potatoes and tomatoes,
Plasmopara viticola on grapevines,
Podosphaera leucotricha on apples,
Pseudocercosporella herpotrichoides (strawbreaker) on cereals (wheat or barley),
Pseudoperonospora on various plants, for example P. cubensis on cucumbers or P. humili on hops,
Pseudopezicula tracheiphilai on grapevines,
Puccinia species on various plants, for example P. triticina, P. striformins, P. hordei or P. graminis on cereals (for example wheat or barley) or on asparagus (for example P. asparagi),
Pyricularia oryzae, Corticium sasakii, Sarocladium oryzae, S. attenuatum,
Pyrenophora tritici-repentis (leaf spot) on wheat or Pyrenophora teres (net blotch) on barley,
Entyloma oryzae on rice,
Pyricularia grisea on lawn and cereals,
Pythium spp. on lawn, rice, corn, wheat, cotton, oilseed rape, sunflowers, sugar beet, vegetables and other plants (for example P. ultimum or P. aphanidermatum),
Ramularia collo-cygni (physiological leaf spots) on barley,
Rhizoctonia species on cotton, rice, potatoes, lawn, corn, oilseed rape, potatoes, sugar beet, vegetables and on various other plants, for example Rhizoctonia solani (root/stem rot) on soybeans or Rhizoctonia cerealis (yellow patch) on wheat or barley,
Rhynchosporium secalis on barley (leaf blotch), rye and triticale,
Sclerotinia species on oilseed rape, sunflowers and, for example, Sclerotinia sclerotiorum (stem disease) or Sclerotinia rolfsii (stem disease) on soybeans,
Septoria glycines (leaf blotch) on soybeans,
Septoria tritici (leaf spot) and Stagonospora nodorum on wheat,
Erysiphe (syn. Uncinula) necator on grapevines,
Setospaeria species on corn and lawn,
Sphacelotheca reilinia on corn,
Stagonospora nodorum (glume blotch) on wheat,
Thievaliopsis species on soybeans and cotton,
Tilletia species on cereals,
Typhula incarnate (snow mold) on wheat or barley,
Ustilago species on cereals, corn (for example U. maydis) and sugar cane,
Venturia species (scab) on apples (for example V. inaequalis) and pears.

The compounds and compositions of the invention are furthermore suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compound(s) I, in particular compounds Ia, Ib, Ic, Id, Ie or If, especially Ia-1, Ib-1, Ic-1, Id-1, Ie-1 or If-1, and at least one of the active compounds II can be applied simultaneously, that is jointly or separately, or in succession, the sequence, in the case of separate application, generally not having any effect on the result of the control measures.

When preparing the compositions, it is preferred to employ the pure active compounds I and II, to which further compounds active against harmful fungi or other pests, such as insects, arachnids or nematodes, or else herbicidal or growth-regulating active compounds or fertilizers can be added.

Such compositions of three active compounds comprise, for example, a compound of the formula I, in particular a compound Ia, Ib, Ic, Id, Ie or If, especially Ia-1, Ib-1, Ic-1, Id-1, Ie-1 or If-1, an azole from group A), in particular epoxiconazole, metconazole, triticonazole or fluquinconazole, and an insecticide, suitable insecticides being in particular fipronil and neonicotinoids, such as acetamiprid, clothianidin, dinotefuran, imidacloprid, nitenpyram, thiacloprid and thiamethoxam.

Usually, compositions of at least one compound I, in particular a compound Ia, Ib, Ic, Id, Ie or If, especially Ia-1, Ib-1, Ic-1, Id-1, Ie-1 or If-1, and at least one active compound II are employed. However, mixtures of at least one compound I with two or, if desired, more active components may also offer particular advantages.

Suitable further active components in the above sense are particularly the active compounds II mentioned at the outset and in particular the preferred active compounds II mentioned above.

Compound(s) I, in particular a compound Ia, Ib, Ic, Id, Ie or If, especially Ia-1, Ib-1, Ic-1, Id-1, Ie-1 or If-1, and active compound(s) II are usually employed in a weight ratio of from 100:1 to 1:100, preferably from 20:1 to 1:20, in particular from 10:1 to 1:10.

The further active components are, if desired, mixed in a ratio of from 20:1 to 1:20 to the compound I, in particular a compound Ia, Ib, Ic, Id, Ie or If, especially Ia-1, Ib-1, Ic-1, Id-1, Ie-1 or If-1.

Depending on the type of the compounds I, in particular a compound Ia, Ib, Ic, Id, Ie or If, especially Ia-1, Ib-1, Ic-1, Id-1, Ie-1 or If-1, and II and the desired effect, the application rates of the compositions according to the invention, especially on agricultural crop areas, are from 5 g/ha to 2000 g/ha, preferably from 20 to 1500 g/ha, in particular from 50 to 1000 g/ha.

Correspondingly, the application rates for the compound(s) I, in particular a compound Ia, Ib, Ic, Id, Ie or If, especially Ia-1, Ib-1, Ic-1, Id-1, Ie-1 or If-1, are generally from 1 to 1000 g/ha, preferably from 10 to 900 g/ha, in particular from 20 to 750 g/ha.

Correspondingly, the application rates for the active compound II are generally from 1 to 2000 g/ha, preferably from 10 to 1500 g/ha, in particular from 40 to 1000 g/ha.

In the treatment of seed, application rates of composition are generally from 1 to 1000 g per 100 kg of seed, preferably from 1 to 750 g per 100 kg, in particular from 5 to 500 g per 100 kg of seed.

The method for controlling harmful fungi is carried out by the separate or joint application of compound(s) I, in particular a compound Ia, Ib, Ic, Id, Ie or If, especially Ia-1, Ib-1, Ic-1, Id-1, Ie-1 or If-1, and at least one of the active compounds II, or a composition of compound(s) I and at least one of the active compounds II, by spraying or dusting the seeds, the plants or the soils before or after sowing of the plants or before or after emergence of the plants.

The fungicidal compositions according to the invention, or the compound(s) I, in particular a compound Ia, Ib, Ic, Id, Ie or If, especially Ia-1, Ib-1, Ic-1, Id-1, Ie-1 or If-1, and at least one of the active compounds II, can be converted into the customary formulations, for example solutions, emulsions, suspensions, dusts, powders, pastes and granules. The use form depends on the particular intended purpose; in each case, it should ensure as fine and even a distribution of the composition according to the invention as possible.

The formulations are prepared in a known manner, for example by extending the active compounds with solvents and carriers and/or solvents or carriers, if desired using other auxiliaries such as emulsifiers or dispersants. In this case individual substances may also perform various functions. Solvents, carriers or auxiliaries suitable for this purpose are essentially:
water, aromatic solvents (for example Solvesso® products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.
carriers such as ground natural minerals (for example kaolins, clays, talc, chalk) and ground synthetic minerals (for example highly disperse silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

The compositions according to the invention can be formulated in solid form or in liquid form. Depending on the embodiment, the compositions according to the invention may also comprise auxiliaries and/or carriers customary in crop protection compositions or in compositions for the protection of materials. The auxiliaries include in particular conventional surface-active substances and other additives and carriers customary in crop protection and in the protection of materials, which compounds may be solid or liquid. The surface-active substances include in particular surfactants, especially those having wetting agent properties. The other auxiliaries (additives) include in particular thickeners, antifoams, preservatives, antifreeze agents, stabilizers, anticaking agents or powder-flow aids and buffers.

Conventional surface-active substances which are usable in principle are anionic, nonionic and amphoteric surfactants including polymer surfactants, and the molecular weight of the surfactants will typically not exceed a value of 2000 Dalton and in particular 1000 Dalton (number-average).

The anionic surfactants include, for example, carboxylates, in particular alkali metal, alkaline earth metal, and ammonium salts of fatty acids, for example potassium stearate, which are usually also referred to as soaps; acyl glutamates; sarcosinates, for example sodium lauroyl sarcosinate; taurates; methylcelluloses; alkyl phosphates, in particular alkyl esters of mono- and diphosphoric acid; sulfates, in particular alkyl sulfates and alkyl ether sulfates; sulfonates, further alkyl sulfonates and alkylaryl sulfonates, in particular alkali metal, alkaline earth metal and ammonium salts of arylsulfonic acids and of alkyl-substituted arylsulfonic acids, alkylbenzenesulfonic acids, such as, for example, lignol and phenolsulfonic acid, naphthalene- and dibutylnaphthalenesulfonic acids, or dodecylbenzenesulfonates, alkylnaphthalenesulfonates, alkyl methyl ester sulfonates, condensates of sulfonated naphthalene and derivatives thereof with formaldehyde, condensates of naphthalene sulfonic acids, phenol- and/or phenolsulfonic acids with formaldehyde or with formaldehyde and urea, mono- or dialkyl sulfosuccinates; and also protein hydrolysates and lignosulfite waste liquors. The abovementioned sulfonic acids are advantageously used in the form of their neutral or, if appropriate, basic salts.

The nonionic surfactants include, for example:
fatty alcohol alkoxylates and oxoalcohol alkoxylates, in particular ethoxylates and propoxylates having degrees of alkoxylation of usually from 2 to 100 and in particular from 3 to 50, for example alkoxylates of C₈-C₃₀-alkanols or alk(adi)enols, for example of isotridecyl alcohol, lauryl alcohol, oleyl alcohol or stearyl alcohol, and their C₁-C₄-alkyl ethers and C₁-C₄-alkyl esters, for example their acetates;
alkoxylated animal and/or vegetable fats and/or oils, for example corn oil ethoxylates, castor oil ethoxylates, tallow fat ethoxylates,
glycerol esters, such as, for example, glycerol monostearate,
alkylphenol alkoxylates, such as, for example, ethoxylated isooctylphenol, octylphenol or nonylphenol, tributylphenol polyoxyethylene ether,
fatty amine alkoxylates, fatty acid amide alkoxylates and fatty acid diethanolamide alkoxylates, in particular their ethoxylates,
sugar surfactants, sorbitol esters, such as, for example, sorbitan fatty acid esters (sorbitan monooleate, sorbitan tristearate), polyoxyethylene sorbitan fatty acid esters, alkyl polyglycosides, N-alkylgluconamides,
alkyl methyl sulfoxides,
alkyldimethylphosphine oxides, such as, for example, tetradecyldimethylphosphine oxide.

The amphoteric surfactants include, for example, sulfobetaines, carboxybetaines and alkyldimethylamine oxides, for example tetradecyldimethylamine oxide.

Other surfactants which are to be mentioned here by way of example are perfluoro surfactants, silicone surfactants, phospholipids, such as, for example, lecithin or chemically modified lecithins, amino acid surfactants, for example N-lauroylglutamate.

Substances which are suitable for the preparation of directly sprayable solutions, emulsions, pastes or oil dispersions are mineral oil fractions of medium to high boiling point, such as kerosine or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone and water.

Powders, materials for spreading and dustable products can be prepared by mixing or concomitantly grinding the active components A and B and, if present, other active substances with at least one solid carrier.

Granules, for example coated granules, impregnated granules and homogeneous granules, can be prepared by binding the active compounds to at least one solid carrier. Examples of solid carriers are mineral earths such as silica gels, silicates, talc, kaolin; attaclay, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the compound(s) I, in particular a compound Ia, Ib, Ic, Id, Ie or If, and at least one of the active compounds II or the composition of compound(s) I with at least one of the active compounds II. The active compounds are employed in a purity of from 90% to 100%, preferably 95% to 100% (according to NMR or HPLC spectrum).

For the treatment of seeds, the formulations in question yield, after two- to tenfold dilution, concentrations of active substance from 0.01 to 60% by weight, preferably 0.1 to 40% by weight, in the ready-to-use preparations.

The following are examples of formulations: 1. Products for dilution with water

### A) Water-Soluble Concentrates (SL)

10 parts by weight of a compound or composition according to the invention are dissolved in 90 parts by weight of water or in a water-soluble solvent. As an alternative, wetting agents, or other auxiliaries are added. The active compounds dissolve upon dilution with water. In this way, a formulation having a total content of 10% by weight of active compound is obtained.

### B) Dispersible Concentrates (DC)

20 parts by weight of a compound or composition according to the invention are dissolved in 70 parts by weight of cyclohexanone with addition of 10 parts by weight of a dispersant, for example polyvinylpyrrolidone. Dilution with water gives a dispersion. The active compound content is 20% by weight.

### C) Emulsifiable Concentrates (EC)

15 parts by weight of a compound or composition according to the invention are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D) Emulsions (EW, EO)

25 parts by weight of a compound or composition according to the invention are dissolved in 35 parts by weight of xylene with addition of calcium dodecylbenzenesulfonate and castor oil ethoxylate (in each case 5 parts by weight). This composition is introduced into 30 parts by weight of water by means of an emulsifying machine (e.g. Ultraturrax) and made into a homogeneous emulsion. Dilution with water gives an emulsion. The formulation has an active compound content of 25% by weight.

### E) Suspensions (SC, OD)

In an agitated ball mill, 20 parts by weight of a compound or composition according to the invention are comminuted with addition of 10 parts by weight of dispersants and wetting agents and 70 parts by weight of water or an organic solvent to give a fine active compound suspension. Dilution with water gives a stable suspension of the active compounds. The active compound content in the formulation is 20% by weight.

### F) Water-Dispersible Granules and Water-Soluble Granules (WG, SG)

50 parts by weight of a compound or composition according to the invention are ground finely with addition of 50 parts by weight of dispersants and wetting agents and prepared as water-dispersible or water-soluble granules by means of technical appliances (for example extrusion, spray tower, fluidized bed). Dilution with water gives a stable dispersion or solution of the active compounds. The formulation has an active compound content of 50% by weight.

### G) Water-Dispersible Powders and Water-Soluble Powders (WP, SP)

75 parts by weight of a compound or composition according to the invention are ground in a rotor-stator mill with addition of 25 parts by weight of dispersants, wetting agents and silica gel. Dilution with water gives a stable dispersion or solution of the active compounds. The active compound content of the formulation is 75% by weight.

### H) Gel Formulations (GF)

In a ball mill, 20 parts by weight of the active compounds, 10 parts by weight of dispersant, 1 part by weight of gelling agent and 70 parts by weight of water or an organic solvent are ground to give a fine suspension. On dilution with water, a stable suspension having an active compound content of 20% by weight is obtained.

### 2. Products to be Applied Undiluted

### I) Dustable Powders (DP)

5 parts by weight of a compound or composition according to the invention are ground finely and mixed intimately with 95 parts by weight of finely divided kaolin. This gives a dustable product having an active compound content of 5% by weight.

### J) Granules (GR, FG, GG, MG)

0.5 part by weight of a compound or composition according to the invention is ground finely and associated with 99.5 parts by weight of carriers. Current methods are extrusion, spray-drying or the fluidized bed. This gives granules to be applied undiluted having an active compound content of 0.5% by weight.

### K) ULV Solutions (UL)

10 parts by weight of a compound or composition according to the invention are dissolved in 90 parts by weight of an organic solvent, for example xylene. This gives a product to be applied undiluted having an active compound content of 10% by weight.

For seed treatment, use is usually made of water-soluble concentrates (LS), suspensions (FS), dustable powders (DS), water-dispersible and water-soluble powders (WS, SS), emulsions (ES), emulsifiable concentrates (EC) and gel formulations (GF). These formulations can be applied to the seed in undiluted form or, preferably, diluted. Application can be carried out prior to sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetting agent, tackifier, dispersant or emulsifier. However, it is also possible to prepare concentrates composed of active substance, wetting agent, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), it being possible to apply formulations comprising over 95% by weight of active compound, or even to apply the active compound without additives.

Oils of various types, wetting agents, adjuvants., herbicides, other pest control agents or bactericides may be added to the active compounds, even, if desired, not until immediately prior to use (tank mix). These agents are typically mixed with the compositions according to the invention in a weight ratio of from 1:100 to 100:1, preferably from 1:10 to 10:1.

Suitable adjuvants in this context are, in particular: organically modified polysiloxanes, e.g. Break Thru S 240®; alcohol alkoxylates, e.g. Atplus 245®, Atplus MBA 1303®, Plurafac LF 300® and Lutensol ON 30®; EO/PO block polymers, e.g. Pluronic RPE 2035® and Genapol B®; alcohol ethoxylates, e.g. Lutensol XP 80®; and sodium dioctylsulfosuccinate, e.g. Leophen RA®.

The compounds I and II or the compositions according to the invention or the corresponding formulations are applied by treating the harmful fungi, their habitat or the plants, seeds, soils, areas, materials or spaces to be kept free from them with a fungicidally effective amount of the composition or, in the case of separate application, of the compounds I and II. Application can be before or after the infection by harmful fungi.

The present invention furthermore relates to the pharmaceutical use of the compounds of the formula I according to the invention, in particular the compounds I described in the above description as being preferred, and/or the pharmaceutically acceptable salts thereof, in particular their use for the treatment of tumors in mammals such as for example, man.

### USE EXAMPLES

The fungicidal action of the individual compounds and of the compositions according to the invention can be demonstrated by the tests below.

The following active substances were used:

TABLE D
I

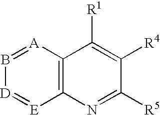

| No. | A | B | D | E | R¹ | R⁴ | R⁵ |
|---|---|---|---|---|---|---|---|
| I.1 | N | CH | CH | N | 4-methylpiperidin-1-yl | 2,4,6-trifluorophenyl | Cl |
| I.2 | N | CH | N | N | 4-methylpiperidin-1-yl | 2,4,6-trifluorophenyl | Cl |
| I.3 | CH | CH | CH | N | 4-methylpiperidin-1-yl | 2,6-difluorophenyl | Cl |
| I.4 | CH | CH | C(CH₃) | N | NH(CH(CH₃)₂) | 2,4,6-trifluorophenyl | Cl |
| I.5 | CH | N | CH | N | CH₂(CF₃) | 2,4,6-trifluorophenyl | Cl |

TABLE E

| No. | |
|---|---|
| II.1 | pyraclostrobin |
| II.2 | cyprodinil |
| II.3 | boscalid |
| II.4 | cyproconazol |
| II.5 | epoxiconazol |

The visually determined percentages of infected leaf areas were converted into efficacies in % of the untreated control:

The efficacy (R⁴) was calculated as follows using Abbot's formula:

R⁴=(1−α/β)·100

α corresponds to the fungal infection of the treated plants in % and
β corresponds to the fungal infection of the untreated (control) plants in

An efficacy of 0 means that the infection level of the treated plants corresponds to that of the untreated control plants; an efficacy of 100 means that the treated plants are not infected.

The expected efficacies of active compound combinations were determined using Colby's formula (Colby, S. R. “Calculating synergistic and antagonistic responses of herbicide combinations”, Weeds, 15, pp. 20-22, 1967) and compared with the observed efficacies.

E=x+y−x·y/100 Colby's formula
E expected efficacy, expressed in % of the untreated control, when using the composition of the active compounds A and B at the concentrations a and b;
x efficacy, expressed in % of the untreated control, when using the active compound A at the concentration a;
y efficacy, expressed in % of the untreated control, when using the active compound B at the concentration b.

### Microtest

The active compounds were formulated separately as a stock solution having a concentration of 10 000 ppm in DMSO. The active compounds epoxiconazole and boscalid were used as a commercial formulation.

### Use Example No. 1

### Activity Against the Gray Mold Pathogen Botrytis cinerea in the Microtiter Test

The stock solution was pipetted into a microtiter plate (MTP) and diluted to the stated active compound concentration using a malt-based aqueous nutrient medium for fungi. An aqueous spore suspension of Botrytis cinerea was then added. The plates were placed in a water vapor-saturated chamber at temperatures of 18° C. Using an absorption photometer, the MTPs were measured at 405 nm on day 7 after the inoculation. The measured parameters were compared to the growth of the active compound-free control variant and the fungus- and active compound-free blank value to determine the relative growth in % of the pathogens in the individual active compounds. Initially, the means of the visually determined values for the percentage of infected leaf area were calculated, and the mean values were then converted into efficacies in % of the untreated control. An efficacy of 0 means the same degree of infection as in the untreated control, and an efficacy of 100 means 0% infection. The expected efficacies for active compound combinations were determined using Colby's formula (Colby, S. R. “Calculating synergistic and antagonistic responses of herbicide combinations”, Weeds, 15, pp. 20-22, 1967) and compared to the observed efficacies.

| Active | | | | Activity | | Degree |
|---|---|---|---|---|---|---|
| compound/ | | | | calculated | | of |
| active | | | Observed | according | | syn- |
| compound | Conc. | | activity | to Colby | Syn- | ergism |
| combination | (ppm) | Ratio | (%) | (%) | ergism | (%) |
| I.1 | 1 | | 0 | | | |
| I.2 | 0.004 | | 2 | | | |
| I.3 | 1 | | 60 | | | |
| | 0.063 | | 1 | | | |
| II.1 | 1 | | 19 | | | |
| II.2 | 0.063 | | 72 | | | |
| II.3 | 1 | | 58 | | | |
| I.1 | 1 | 1:1 | 35 | 19 | Yes | 16 |
| II.1 | 1 | | | | | |
| I.2 | 0.004 | 1:16 | 100 | 73 | Yes | 27 |
| II.2 | 0.063 | | | | | |
| I.3 | 1 | 1:1 | 83 | 67 | Yes | 16 |
| II.2 | 1 | | | | | |
| I.3 | 0.063 | 1:16 | 76 | 59 | Yes | 17 |
| II.3 | 1 | | | | | |

### Use Example No. 2

### Activity Against the Rice Blast Pathogen Pyricularia oryzae in the Microtiter Test

The stock solution was pipetted into a microtiter plate (MTP) and diluted to the stated active compound concentration using a malt-based aqueous nutrient medium for fungi. An aqueous spore suspension of Pyricularia oryzae was then added. The plates were placed in a water vapor-saturated chamber at temperatures of 18° C. Using an absorption photometer, the MTPs were measured at 405 nm on day 7 after the inoculation. The measured parameters were compared to the growth of the active compound-free control variant (=100%) and the fungus- and active compound-free blank value to determine the relative growth in % of the pathogens in the individual active compounds. Initially, the means of the visually determined values for the percentage of infected leaf area were calculated, and the mean values were then converted into efficacies in % of the untreated control. An efficacy of 0 means the same degree of infection as in the untreated control, and an efficacy of 100 means 0% infection. The expected efficacies for active compound combinations were determined using Colby's formula (Colby, S. R. “Calculating synergistic and antagonistic responses of herbicide combinations”, Weeds, 15, pp. 20-22, 1967) and compared to the observed efficacies.

| Active | | | | Activity | | Degree |
|---|---|---|---|---|---|---|
| compound/- | | | | calculated | | of |
| active | | | Observed | according | | syn- |
| compound | Conc. | | activity | to Colby | Syn- | ergism |
| combination | (ppm) | Ratio | (%) | (%) | ergism | (%) |
| I.4 | 16 | | 21 | | | |
| | 1 | | 12 | | | |
| I.5 | 4 | | 43 | | | |
| II.4 | 0.25 | | 0 | | | |
| II.5 | 0.25 | | 4 | | | |
| II.2 | 0.25 | | 63 | | | |
| I.4 | 1 | 4:1 | 50 | 12 | Yes | 38 |
| II.4 | 0.25 | | | | | |
| I.4 | 16 | 63:1 | 60 | 24 | Yes | 36 |
| II.5 | 0.25 | | | | | |
| I.4 | 1 | 4:1 | 85 | 67 | Yes | 18 |
| II.2 | 0.25 | | | | | |
| I.5 | 4 | 4:1 | 60 | 43 | Yes | 17 |
| II.3 | 1 | | | | | |

1-40. (canceled)

41. A compound of formula I
wherein: 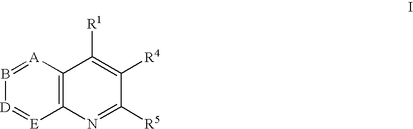
R¹ is C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₂-cycloalkyl, C₃-C₁₂-halocycloalkyl, C₃-C₁₀-cycloalkenyl, C₃-C₁₀-halocycloalkenyl, phenyl, halophenyl, naphthyl, halonaphthyl or a five-, six-, seven-, eight-, nine- or ten-membered saturated, partially unsaturated or aromatic heterocycle which is attached via carbon and which contains one, two, three or four heteroatoms from the group consisting of oxygen, nitrogen and sulfur,
wherein R¹ may carry one, two, three or four identical or different groups R^{a}, wherein R^{a} is as defined below:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₂-C₆-alkenyloxy, C₃-C₆-alkynyloxy, C₁-C₈-alkoximino, C₂-C₁₀-alkenyloximino, C₂-C₁₀-alkynyloximino, C₆-C₁₄-aryl-C₁-C₈-alkyloximino, C₂-C₁₀-alkynyl, C₂-C₁₀-alkenyloxycarbonyl, C₂-C₁₀-alkynyloxycarbonyl, tri-C₁-C₆-alkylsilyl, phenyl, naphthyl, a five-, six-, seven-, eight-, nine- or ten-membered saturated, partially unsaturated or aromatic heterocycle which contains one, two, three or four heteroatoms from the group consisting of oxygen, nitrogen and sulfur;
wherein the aliphatic, alicyclic and aromatic groups in R^{a} for their part may be partially or fully halogenated or may carry one, two or three groups R^{b}, wherein R^{b} is as defined below:
R^{b} is halogen, cyano, nitro, hydroxyl, mercapto, amino, carboxyl, aminocarbonyl, aminothiocarbonyl, C₁-C₆-alkyl, C₁-C₆-haloalkyl, C₂-C₈-alkenyl, C₂-C₈-haloalkenyl, C₂-C₈-alkenyloxy, C₂-C₈-alkynyloxy, C₁-C₆-alkoxy, C₁-C₆-haloalkoxy, C₁-C₆-alkylthio, C₁-C₆-alkylamino, di-C₁-C₆-alkylamino, formyl, C₁-C₆-alkylcarbonyl, C₁-C₆-alkylsulfonyl, C₁-C₆-alkylsulfoxyl, C₁-C₆-alkoxycarbonyl, C₁-C₆-alkylcarbonyloxy, C₁-C₆-alkylaminocarbonyl, di-C₁-C₆-alkylaminocarbonyl, C₁-C₆-alkylaminothiocarbonyl, di-C₁-C₆-alkylaminocarbonyl, C₃-C₁₀-cycloalkyl, C₃-C₁₀-cycloalkoxy, saturated or partially unsaturated 5- or 6-membered heterocyclyl, saturated or unsaturated 5- or 6-membered heterocyclyloxy, 5- or 6-membered heteroaryl, 5- or 6-membered heteroaryloxy, 5- or 6-membered heteroarylthio, 5- or 6-membered heteroarylthio, phenyl, naphthyl, phenoxy, naphthoxy, phenylthio, naphthylthio, phenyl-C₁-C₆-alkoxyl, phenyl-C₁-C₆-alkyl, wherein the cyclic systems may be partially or fully halogenated and/or substituted by alkyl and/or haloalkyl groups;
R⁴ is halogen, C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₁-C₈-alkoxy, C₁-C₈-haloalkoxy, C₃-C₈-cycloalkyl, C₃-C₈-halocycloalkyl, C₃-C₈-cycloalkyl-C₁-C₈-alkyl, C₁-C₈-alkylthio, C₆-C₁₄-aryl, C₆-C₁₄-aryloxy, C₆-C₁₄-arylthio, C₆-C₁₄-aryl-C₁-C₈-alkyl, C₆-C₁₄-aryl-C₁-C₈-alkoxy, C₆-C₁₄-aryl-C₁-C₈-alkylthio, 5- or 6-membered heteroaryl, heteroaryloxy, heteroarylthio, heteroaryl-C₁-C₈-alkyl, heteroaryl-C₁-C₈-alkoxy or heteroaryl-C₁-C₈-alkylthio, morpholino, piperidino or pyrrolidino;
wherein R⁴ may carry one, two, three, four or five identical or different groups R^{c}, wherein R^{c} is as defined below:
R^{c} is halogen, cyano, isocyano, hydroxyl, cyanato (OCN), thiocyanato, isothiocyanato, mercapto, C₁-C₈-alkyl, C₁-C₈-haloalkyl, C₁-C₈-hydroxyalkyl, C₁-C₈-alkoxy-C₁-C₈-alkyl, C₃-C₆-cycloalkyl-C₁-C₈-alkyl, C₂-C₁₀-alkenyl, C₂-C₁₀-haloalkenyl, C₂-C₁₀-alkynyl, C₂-C₁₀-haloalkynyl, C₁-C₆-alkoxy, C₁-C₆-haloalkoxy, C₂-C₁₀-alkenyloxy, C₂-C₁₀-haloalkenyloxy, C₂-C₁₀-alkynyloxy, C₂-C₁₀-haloalkynyloxy, C₃-C₆-cycloalkyl, C₃-C₆-halocycloalkyl, C₃-C₆-cycloalkenyl, C₃-C₆-halocycloalkenyl, C₃-C₆-cycloalkoxy, C₃-C₆-halocycloalkoxy, C₁-C₈-alkoxycarbonyl, C₁-C₈-haloalkoxycarbonyl, C₂-C₁₀-alkenyloxycarbonyl, C₂-C₁₀-haloalkenyloxycarbonyl, C₂-C₁₀-alkynyloxycarbonyl, C₂-C₁₀-haloalkynyloxycarbonyl, aminocarbonyl, C₁-C₈-alkylaminocarbonyl, di-C₁-C₈-alkylaminocarbonyl, hydroxyimino-C₁-C₈-alkyl, C₁-C₈-alkoximino-C₁-C₈-alkyl, C₂-C₁₀-alkenyloximinocarbonyl, C₂-C₁₀-alkenyloximino-C₁-C₈-alkyl, C₂-C₁₀-alkynyloximino-C₁-C₈-alkyl, C₂-C₁₀-alkenylcarbonyl, C₂-C₁₀-alkenylthiocarbonyl, C₂-C₁₀-alkynylcarbonyl, C₂-C₁₀-alkynylthiocarbonyl, tri-C₁-C₈-alkylsilyl, C₁-C₈-alkylthio, C₁-C₈-haloalkylthio, C₁-C₈-alkylsulfinyl, C₁-C₈-haloalkylsulfinyl, carboxyl-C₁-C₈-alkyl, phenyl, naphthyl, a five-, six-, seven-, eight-, nine- or ten-membered saturated, partially unsaturated or aromatic heterocycle which contains one, two, three or four heteroatoms from the group consisting of oxygen, nitrogen and sulfur, NR^{A}R^{B}, NHCOR^{A}, NHCONR^{A}R^{B}, CONR^{A}R^{B}, —OSO₂R^{A}, —COR^{A}, —CSR^{A}, —CR^{A}═NR^{B} or N═CR^{A}R^{B}, wherein R^{A} and R^{B} are as defined below:
R^{A}, R^{B} independently of one another are hydrogen, C₁-C₈-alkyl, C₁-C₈-haloalkyl, C₁-C₈-alkoxy, C₁-C₈-haloalkoxy, C₁-C₈-alkylthio, C₃-C₈-cycloalkyl, C₃-C₈-halocycloalkyl, C₃-C₈-cycloalkyl-C₁-C₈-alkyl, phenyl or benzyl, where the phenyl and benzyl groups may be substituted by one, two, three, four or five groups independently selected from the group consisting of halogen, C₁-C₈-alkyl and C₁-C₈-alkoxy; phenoxy, benzyloxy, benzoyloxy, a five-, six-, seven-, eight-, nine- or ten-membered saturated, partially unsaturated or aromatic heterocycle which contains one, two, three or four heteroatoms from the group consisting of oxygen, nitrogen and sulfur;
wherein R^{c} may carry one, two or three identical or different groups R^{cL}, wherein R^{cL} is as defined below:
R^{cL} is halogen, cyano, nitro, hydroxyl, amino, carboxyl, C₁-C₆-alkyl, C₁-C₆-haloalkyl, C₂-C₆-alkenyl, C₂-C₆-haloalkenyl, C₁-C₈-alkoxy, C₁-C₈-haloalkoxy, C₂-C₈-alkenyloxy, C₂-C₈-alkynyloxy, C₃-C₈-cycloalkyl, C₃-C₈-halocycloalkyl, C₃-C₈-cycloalkenyl, C₃-C₈-cycloalkyloxy, C₃-C₈-cycloalkenyloxy, C₁-C₈-alkoximino, C₂-C₈-alkenyloxyimino, C₂-C₈-alkynyloxyimino, C₁-C₈-alkoximino-C₁-C₈-alkyl, C₂-C₈-alkenyloximino-C₁-C₈-alkyl, C₂-C₈-alkynyloximino-C₁-C₈-alkyl, C₁-C₆-alkylcarbonyl, C₁-C₆-haloalkoxy, C₁-C₆-alkoxycarbonyl, C₁-C₆-alkylthio, C₁-C₆-alkylamino, di-C₁-C₆-alkylamino, C₆-C₁₄-aryl-C₁-C₈-alkyloximino, C₂-C₁₀-alkynyl, C₂-C₁₀-alkenyloxycarbonyl, C₂-C₁₀-alkynyloxycarbonyl, tri-C₁-C₆-alkylsilyl, phenyl, naphthyl, a five-, six-, seven-, eight-, nine- or ten-membered saturated, partially unsaturated or aromatic heterocycle which contains one, two, three or four heteroatoms from the group consisting of oxygen, nitrogen and sulfur;
R⁵ is halogen, cyano, C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₁-C₈-alkoxy, C₁-C₈-haloalkoxy;
wherein R⁵ may carry one, two or three identical or different groups independently selected from the group consisting of cyano, nitro, C₁-C₂-alkoxy and C₁-C₄-alkoxycarbonyl;
A, B, D, E are each N or C—R⁶, wherein at least one and at most three variables are N; provided that when A, B and D are N, E is other than C—R⁶, and when B, D and E are N, E is other than C—R⁶; wherein R⁶ is as defined below:
R⁶ is hydrogen, halogen, C₁-C₈-alkyl, C₁-C₈-alkoxy, C₁-C₈-haloalkyl, C₁-C₈-haloalkoxy;
or an agriculturally acceptable salt thereof.

42. The compound of claim 41, wherein R⁴ is phenyl or 5- or 6-membered heteroaryl, where the phenyl or heteroaryl group is unsubstituted or substituted by one, two, three, four or five identical or different groups R^{c}, wherein R^{c} is selected from the group consisting of halogen, C₁-C₄-alkyl, C₁-C₄-haloalkyl, C₁-C₄-alkoxy, C₁-C₄-haloalkoxy and C₁-C₄-alkoxycarbonyl.

43. The compound of claim 42, wherein R⁴ is unsubstituted or substituted phenyl.

44. The compound of claim 42, wherein R⁴ is unsubstituted or substituted 5- or 6-membered heteroaryl.

45. The compound of claim 41, wherein R⁵ is halogen.

46. The compound of claim 41, wherein R⁵ is C₁-C₄-alkyl.

47. The compound of claim 41, wherein R¹ is C₃-C₈-alkyl, C₃-C₈-haloalkyl, C₃-C₈-alkenyl, C₃-C₈-haloalkenyl, C₃-C₈-alkynyl, C₃-C₈-haloalkynyl, C₅-C₆-cycloalkyl, C₅-C₆-halocycloalkyl, C₅-C₆-cycloalkenyl or C₅-C₆-halocycloalkenyl, wherein R¹ may carry one, two, three or four identical or different groups R^{a} selected from the group consisting of halogen, cyano, C₁-C₆-alkyl, C₂-C₆-alkenyl, C₂-C₆-alkynyl, C₁-C₆-alkoxy, C₁-C₆-alkoxycarbonyl, C₁-C₆-alkoximino, C₂-C₆-alkenyloximino and C₂-C₆-alkynyloximino.

48. A fungicidal composition for controlling phytopathogenic harmful fungi, comprising:
1) at least one compound of formula I 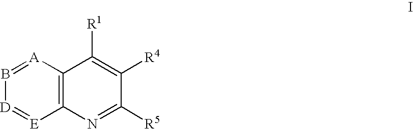
wherein
R¹ is halogen, cyano, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₂-cycloalkyl, C₃-C₁₂-halocycloalkyl, C₃-C₁₀-cycloalkenyl, C₃-C₁₀-halocycloalkenyl, phenyl, halophenyl, naphthyl, halonaphthyl, a five-, six-, seven-, eight-, nine- or ten-membered saturated, partially unsaturated or aromatic heterocycle which is attached via carbon and which contains one, two, three or four heteroatoms from the group consisting of oxygen, nitrogen and sulfur, C₁-C₈-alkoxy, C₁-C₈-haloalkoxy, C₁-C₈-alkylthio; wherein this R¹ may carry one, two, three or four identical or different groups R^{a}, wherein
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₂-C₆-alkenyloxy, C₃-C₆-alkynyloxy, C₁-C₈-alkoximino, C₂-C₁₀-alkenyloximino, C₂-C₁₀-alkynyloximino, C₆-C₁₄-aryl-C₁-C₈-alkyloximino, C₂-C₁₀-alkynyl, C₂-C₁₀-alkenyloxycarbonyl, C₂-C₁₀-alkynyloxycarbonyl, tri-C₁-C₆-alkylsilyl, phenyl, naphthyl, a five-, six-, seven-, eight-, nine- or ten-membered saturated, partially unsaturated or aromatic heterocycle which contains one, two, three or four heteroatoms from the group consisting of oxygen, nitrogen and sulfur;
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₂-C₆-alkenyloxy, C₃-C₆-alkynyloxy, C₁-C₈-alkoximino, C₂-C₁₀-alkenyloximino, C₂-C₁₀-alkynyloximino, C₆-C₁₄-aryl-C₁-C₈-alkyloximino, C₂-C₁₀-alkynyl, C₂-C₁₀-alkenyloxycarbonyl, C₂-C₁₀-alkynyloxycarbonyl, tri-C₁-C₆-alkylsilyl, phenyl, naphthyl, a five-, six-, seven-, eight-, nine- or ten-membered saturated, partially unsaturated or aromatic heterocycle which contains one, two, three or four heteroatoms from the group consisting of oxygen, nitrogen and sulfur;
wherein the aliphatic, alicyclic and aromatic groups in R^{a} for their part may be partially or fully halogenated or may carry one, two or three groups R^{b}, wherein R^{b} is as defined below:
R^{b} is halogen, cyano, nitro, hydroxyl, mercapto, amino, carboxyl, aminocarbonyl, aminothiocarbonyl, C₁-C₆-alkyl, C₁-C₆-haloalkyl, C₂-C₈-alkenyl, C₂-C₈-haloalkenyl, C₂-C₈-alkenyloxy, C₂-C₈-alkynyloxy, C₁-C₆-alkoxy, C₁-C₆-haloalkoxy, C₁-C₆-alkylthio, C₁-C₆-alkylamino, di-C₁-C₆-alkylamino, formyl, C₁-C₆-alkylcarbonyl, C₁-C₆-alkylsulfonyl, C₁-C₆-alkylsulfoxyl, C₁-C₆-alkoxycarbonyl, C₁-C₆-alkylcarbonyloxy, C₁-C₆-alkylaminocarbonyl, di-C₁-C₆-alkylaminocarbonyl, C₁-C₆-alkylaminothiocarbonyl, di-C₁-C₆-alkylaminocarbonyl, C₃-C₁₀-cycloalkyl, C₃-C₁₀-cycloalkoxy, saturated or partially unsaturated 5- or 6-membered heterocyclyl, saturated or unsaturated 5- or 6-membered heterocyclyloxy, 5- or 6-membered heteroaryl, 5- or 6-membered heteroaryloxy, 5- or 6-membered heteroarylthio, 5- or 6-membered heteroarylthio, phenyl, naphthyl, phenoxy, naphthoxy, phenylthio, naphthylthio, phenyl-C₁-C₆-alkoxy, phenyl-C₁-C₆-alkyl, wherein the cyclic systems may be partially or fully halogenated and/or substituted by alkyl and/or haloalkyl groups;
or R¹ is NR²R³, where R² and R³ are as defined below:
R² is C₁-C₈-alkyl, C₁-C₈-haloalkyl, C₂-C₈-alkenyl, C₂-C₈-haloalkenyl, C₂-C₈-alkynyl, C₂-Cₛ-haloalkynyl, C₄-C₁₀-alkadienyl, C₄-C₁₀-haloalkadienyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₈-halocycloalkoxy, C₅-C₁₀-bicycloalkyl, C₃-C₈-cycloalkyl-C₁-C₈-alkyl, C₆-C₁₄-aryl, C₆-C₁₄-aryl-C₁-C₈-alkyl, amino, C₁-C₈-alkylamino, di-C₁-C₈-alkylamino, five- or six-membered heteroaryl which contains one, two, three or four heteroatoms from the group consisting of O, N and S, five- or six-membered heteroaryl-C₁-C₈-alkyl, where the heteroaryl contains one, two, three or four heteroatoms from the group consisting of O, N and S;
R³ is hydrogen, NR⁷R⁸ or one of the groups mentioned for R²; where R⁷ and R⁸ are as defined below:
R⁷, R⁸ independently of one another are hydrogen, C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₃-C₈-cycloalkyl, C₃-C₈-halocycloalkyl, C₃-C₈-cycloalkyl-C₁-C₈-alkyl, C₆-C₁₄-aryl, C₆-C₁₄-aryl-C₁-C₈-alkyl, five- or six-membered heteroaryl or five- or six-membered heteroaryl-C₁-C₈-alkyl;
wherein the alkyl, alkenyl, alkynyl and cycloalkyl groups in R² and/or R³ may carry one, two or three identical or different groups R^{d}, wherein R^{d} is as defined below:
R^{d} is halogen, cyano, hydroxyl, C₁-C₈-alkyl, C₁-C₈-haloalkyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkoxy, C₃-C₈-halocycloalkoxy, C₃-C₈-cycloalkenyloxy, C₁-C₈-alkylcarbonyl, C₁-C₈-alkoxycarbonyl, C₁-C₈-alkylcarbonyloxy, C₁-C₈-alkoxycarbonyloxy, aminocarbonyloxy, C₁-C₈-alkylaminocarbonyloxy, di-C₁-C₈-alkylaminocarbonyloxy, C₁-C₈-alkylthiocarbonyloxy, C₁-C₈-alkoxythiocarbonyloxy, aminothiocarbonyloxy, C₁-C₈-alkylaminothiocarbonyloxy, di-C₁-C₈-alkylaminothiocarbonyloxy, C₁-C₈-alkylaminothiocarbonyl, di-C₁-C₈-alkylaminothiocarbonyl, C₁-C₈-alkylthio, tri-C₁-C₆-alkylsilyl, C₁-C₈-alkylamino, or di-C₁-C₈-alkylamino;
wherein the alkyl, alkenyl, alkynyl and cycloalkyl groups in R^{d} for their part may carry one, two or three identical or different groups R^{dL}, wherein R^{dL} has the meanings given for R^{cL};
R^{cL} is halogen, cyano, nitro, hydroxyl, amino, carboxyl, C₁-C₆-alkyl, C₁-C₆-haloalkyl, C₂-C₆-alkenyl, C₂-C₆-haloalkenyl, C₁-C₈-alkoxy, C₁-C₈-haloalkoxy, C₂-C₈-alkenyloxy, C₂-C₈-alkynyloxy, C₃-C₈-cycloalkyl, C₃-C₈-halocycloalkyl, C₃-C₈-cycloalkenyl, C₃-C₈-cycloalkyloxy, C₃-C₈-cycloalkenyloxy, C₁-C₈-alkoximino, C₂-C₈-alkenyloxyimino, C₂-C₈-alkynyloxyimino, C₁-C₈-alkenyloximino-C₁-C₈-alkyl, C₂-C₈-alkynyloximino-C₁-C₈-alkyl, C₁-C₆-alkylcarbonyl, C₁-C₆-haloalkoxy, C₁-C₆-alkoxycarbonyl, C₁-C₆-alkylthio, C₁-C₆-alkylamino, di-C₁-C₆-alkylamino, C₆-C₁₄-aryl-C₁-C₈-alkyloximino, C₂-C₁₀-alkynyl, C₂-C₁₀-alkenyloxycarbonyl, C₂-C₁₀-alkynyloxycarbonyl, tri-C₁-C₆-alkylsilyl, phenyl, naphthyl, a five-, six-, seven-, eight-, nine- or ten-membered saturated, partially unsaturated or aromatic heterocycle which contains one, two, three or four heteroatoms from the group consisting of oxygen, nitrogen and sulfur;
and wherein the aryl and heteroaryl groups in R² and/or R³ for their part may carry one, two or three identical or different groups R^{e}, wherein R^{e} is as defined below:
R^{e} is halogen, hydroxyl, cyano, isocyano, thiocyanato, isothiocyanato, nitro, mercapto, C₁-C₈-haloalkyl, C₁-C₈-hydroxyalkyl, C₁-C₈-alkoxy-C₁-C₈-alkyl, C₂-C₈-alkenyl, C₂-C₈-haloalkenyl, C₂-C₈-alkynyl, C₃-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₃-C₈-haloalkynyloxy, C₃-C₈-cycloalkyl, C₃-C₈-halocycloalkyl, C₃-C₈-cycloalkyl-C₁-C₈-alkyl, C₁-C₈-alkylthio, C₁-C₈-haloalkylthio, phenoxy, benzyloxy, benzoyloxy, NR^{A}R^{B}, NHCOR^{A}, NHCONR^{A}R^{B}, CONR^{A}R^{B}, SO₂R^{A}, —OSO₂R^{A}, —COR^{A}, —CR^{A}═NR^{B} or N═CR^{A}R^{B}, wherein R^{A} and R^{B} are as defined below: R^{A}, R^{B} independently of one another are hydrogen, C₁-C₈-alkyl, C₁-C₈-haloalkyl, C₁-C₈-alkoxy, C₁-C₈-haloalkoxy, C₁-C₈-alkylthio, C₃-C₈-cycloalkyl, C₃-C₈-halocycloalkyl, C₃-C₈-cycloalkyl-C₁-C₈-alkyl, phenyl or benzyl, where the phenyl and benzyl groups may be substituted by one, two, three, four or five groups independently selected from the group consisting of halogen, C₁-C₈-alkyl and C₁-C₈-alkoxy; phenoxy, benzyloxy, benzoyloxy, a five-, six-, seven-, eight-, nine- or ten-membered saturated, partially unsaturated or aromatic heterocycle which contains one, two, three or four heteroatoms from the group consisting of oxygen, nitrogen and sulfur;
R² and R³ together with the nitrogen atom to which they are attached may also form a five- or six-membered saturated, partially unsaturated or aromatic heterocycle which may contain one, two or three further heteroatoms from the group consisting of O, N and S as ring members, wherein the heterocycle may carry one, two or three identical or different groups R^{f}, wherein R^{f} has the meanings given for R^{d};
R⁴ is halogen, C₁-C₈-alkyl, C₁-C₈-alkenyl, C₂-C₈-haloalkenyl, C₂-C₈-alkynyl, C₃-C₈-haloalkynyl, C₁-C₈-alkoxy, C₁-C₈-haloalkoxy, C₃-C₈-cycloalkyl, C₃-C₈-halocycloalkyl, C₃-C₈-cycloalkyl-C₁-C₈-alkyl, C₁-C₈-alkylthio, C₆-C₁₄-aryl, C₆-C₁₄-aryloxy, C₆-C₁₄-arylthio, C₆-C₁₄-aryl-C₁-C₈-alkyl, C₆-C₁₄-aryl-C₁-C₈-alkoxy, C₆-C₁₄-aryl-C₁-C₈-alkylthio, 5- or 6-membered heteroaryl, heteroaryloxy, heteroarylthio, heteroaryl-C₁-C₈-alkyl, heteroaryl-C₁-C₈-alkoxy or heteroaryl-C₁-C₈-alkylthio, morpholino, piperidino, pyrrolidino;
wherein R⁴ may carry one, two, three, four or five identical or different groups R^{c}, where R^{c} is as defined below:
R^{c} is halogen, cyano, isocyano, hydroxyl, cyanato (OCN), thiocyanato, isothiocyanato, mercapto, C₁-C₈-haloalkyl, C₁-C₈-hydroxyalkyl, C₁-C₈-alkoxy-C₁-C₈-alkyl, C₃-C₆-cycloalkyl-C₁-C₈-alkyl, C₂-C₁₀-alkenyl, C₂-C₁₀-haloalkenyl, C₂-C₁₀-alkynyl, C₂-C₁₀-haloalkynyl, C₁-C₆-alkoxy, C₁-C₆-haloalkoxy, C₂-C₁₀-alkenyloxy, C₂-C₁₀-haloalkenyloxy, C₂-C₁₀-alkynyloxy, C₂-C₁₀-haloalkynyloxy, C₃-C₆-cycloalkyl, C₃-C₆-halocycloalkyl, C₃-C₆-cycloalkenyl, C₃-C₆-halocycloalkenyl, C₃-C₆-cycloalkoxy, C₃-C₆-halocycloalkoxy, C₁-C₈-alkoxycarbonyl, C₁-C₈-haloalkoxycarbonyl, C₂-C₁₀-alkenyloxycarbonyl, C₂-C₁₀-haloalkenyloxycarbonyl, C₂-C₁₀-alkynyloxycarbonyl, C₂-C₁₀-haloalkynyloxycarbonyl, aminocarbonyl, C₁-C₈-alkylaminocarbonyl, di-C₁-C₈-alkylaminocarbonyl, hydroxyimino-C₁-C₈-alkyl, C₁-C₈-alkoximino-C₁-C₈-alkyl, C₂-C₁₀-alkenyloximinocarbonyl, C₂-C₁₀-alkenyloximino-C₁-C₈-alkyl, C₂-C₁₀-alkynyloximino-C₁-C₈-alkyl, C₂-C₁₀-alkenylcarbonyl, C₂-C₁₀-alkenylthiocarbonyl, C₂-C₁₀-alkynylcarbonyl, C₂-C₁₀-alkynylthiocarbonyl, tri-C₁-C₈-alkylsilyl, C₁-C₈-alkylthio, C₁-C₈-haloalkylthio, C₁-C₈-alkylsulfinyl, C₁-C₈-haloalkylsulfinyl, carboxyl-C₁-C₈-alkyl, phenyl, naphthyl, a five-, six-, seven-, eight-, nine- or ten-membered saturated, partially unsaturated or aromatic heterocycle which contains one, two, three or four heteroatoms from the group consisting of oxygen, nitrogen and sulfur, NR^{A}R^{B}, NHCOR^{A}, NHCONR^{A}R^{B}, CONR^{A}R^{B}, —OSO₂R^{A}, —COR^{A}, —CSR^{A}, —CR^{A}═NR^{B} or N═CR^{A}R^{B}, wherein R^{A} and R^{B} are as defined above R⁵ is halogen, cyano, C₁-C₈alkyl, C₁-C₈-haloalkyl, C₂-C₈-alkenyl, C₂-C₈-haloalkenyl, C₂-C₈-alkynyl, C₃-C₈-haloalkynyl, C₁-C₈-alkoxy, C₁-C₈-haloalkoxy or C₁-C₈-alkylthio;
wherein R⁵ may carry one, two or three identical or different groups independently of one another selected from the group consisting of halogen, cyano, nitro, C₁-C₂-alkoxy and C₁-C₄-alkoxycarbonyl;
A, B, D, E are each N or C—R⁶, wherein at least one and at most three variables are N; provided that when A, B and D are N, E is other than C—R⁶, and when B, D and E are N, E is other than C—R⁶; wherein R⁶ is as defined below:
R⁶ is hydrogen, halogen, C₁-C₈-alkyl, C₁-C₈-alkoxy, C₁-C₈-haloalkyl, C₁-C₈-haloalkoxy;
or an agriculturally acceptable salt thereof; and
2) at least one active compound II selected from the groups A) to F):
A) azoles selected from the group consisting of bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thiabendazole, fuberidazole, ethaboxam, etridiazole and hymexazole;
B) strobilurins selected from the group consisting of azoxystrobin, dimoxystrobin, enestroburin, fluoxastrobin, kresoxim-methyl, methominostrobin, orysastrobin, picoxystrobin, pyraclostrobin, trifloxystrobin, enestroburin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate and methyl 2-(ortho-(2,5-dimethylphenyloxymethylene)phenyl)-3-methoxyacrylate;
C) carboxamides selected from the group consisting of carboxin, benalaxyl, benodanil, boscalid, fenfuram, fenhexamid, flutolanil, furametpyr, mepronil, metalaxyl, mefenoxam, ofurace, oxadixyl, oxycarboxin, penthiopyrad, thifluzamide, tiadinil, 3,4-dichloro-N-(2-cyanophenyl)isothiazole-5-carboxamide, dimethomorph, flumorph, flumetover, fluopicolide (picobenzamid), zoxamide, carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide, methyl 3-(4-chlorophenyl)-3-(2-isopropoxycarbonylamino-3-methylbutyrylamino)propionate, compounds of the formula III 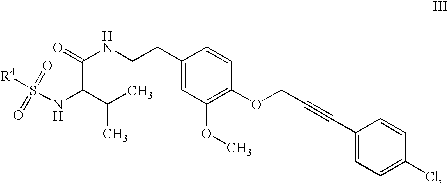
in which R⁴ is methyl or ethyl, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(3′,4′-dichloro-5-fluorobiphenyl-2-yl)-3-difluoromethyl-1-methylpyrazole-4-carboxamide, N-(2-cyanophenyl)-3,4-dichloroisothiazole-5-carboxamide and N-(2-bicyclopropyl-2-ylphenyl)-3-difluoromethyl-1-methyl-1H-pyrazole-4-carboxamide;
D) heterocyclic compounds selected from the group consisting of fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, probenazole, 5-chloro-7-(4-methylpiperidin-1-yl)-6-(2,4,6-trifluorophenyl)-[1,2,4]triazolo[1,5-a]pyrimidine, anilazine, diclomezine, pyroquilon, proquinazid, tricyclazole, the compound of the formula IV (2-butoxy-6-iodo-3-propylchromen-4-one) 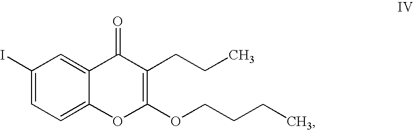
acibenzolar-S-methyl, captafol, captan, dazomet, folpet, fenoxanil, quinoxyfen and N,N-dimethyl-3-(3-bromo-6-fluoro-2-methylindole-1-sulfonyl)-[1,2,4]triazole-1-sulfonamide of the formula V 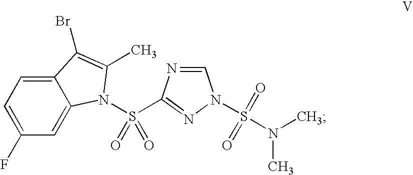
E) carbamates selected from the group consisting of mancozeb, maneb, metam, metiram, ferbam, propineb, thiram, zineb, ziram, diethofencarb, iprovalicarb, flubenthiavalicarb, propamocarb, 4-fluorophenyl N-(1-(1-(4-cyanophenyl)ethanesulfonyl)but-2-yl)carbamate, methyl 3-(4-chlorophenyl)-3-(2-isopropoxycarbonylamino-3-methylbutyrylamino)propanoate of the formula VI 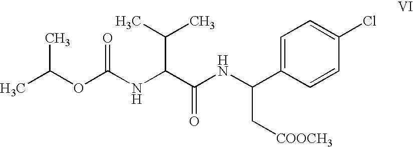
and carbamate oxime ethers of the formula VII 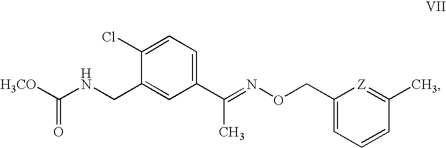
in which Z is N or CH;
F) other fungicides selected from the group consisting of guanidine, dodine, iminoctadine, guazatine, antibiotics: kasugamycin, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds: chlorothalonil, dichlofluanid, flusulfamide, hexachlorobenzene, phthalide, pencycuron, quintozene, thiophanate-methyl, tolylfluanid, inorganic active compounds: Bordeaux mixture, copper acetate, copper hydroxide, copper-oxychloride, basic copper sulfate, sulfur, others: cyflufenamid, cymoxanil, dimethirimol, ethirimol, furalaxyl, metrafenone and spiroxamine;
in a synergistically effective amount.

49. The fungicidal composition of claim 48, wherein component 1) is a compound of the formula I: 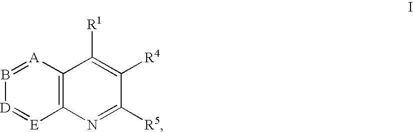
wherein:
R¹ is C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₃-C₁₂-cycloalkyl, C₃-C₁₂-halocycloalkyl, C₃-C₁₀-cycloalkenyl, C₃-C₁₀-halocycloalkenyl, phenyl, halophenyl, naphthyl, halonaphthyl or a five-, six-, seven-, eight-, nine- or ten-membered saturated, partially unsaturated or aromatic heterocycle which is attached via carbon and which contains one, two, three or four heteroatoms from the group consisting of oxygen, nitrogen and sulfur,
wherein R¹ may carry one, two, three or four identical or different groups R^{a}, wherein R^{a} is as defined below:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₂-C₆-alkenyloxy, C₃-C₆-alkynyloxy, C₁-C₈-alkoximino, C₂-C₁₀-alkenyloximino, C₂-C₁₀-alkynyloximino, C₆-C₁₄-aryl-C₁-C₈-alkyloximino, C₂-C₁₀-alkynyl, C₂-C₁₀-alkenyloxycarbonyl, C₂-C₁₀-alkynyloxycarbonyl, tri-C₁-C₆-alkylsilyl, phenyl, naphthyl, a five-, six-, seven-, eight-, nine- or ten-membered saturated, partially unsaturated or aromatic heterocycle which contains one, two, three or four heteroatoms from the group consisting of oxygen, nitrogen and sulfur;
wherein the aliphatic, alicyclic and aromatic groups in R^{a} for their part may be partially or fully halogenated or may carry one, two or three groups R^{b}, wherein R^{b} is as defined below:
R^{b} is halogen, cyano, nitro, hydroxyl, mercapto, amino, carboxyl, aminocarbonyl, aminothiocarbonyl, C₁-C₆-alkyl, C₁-C₆-haloalkyl, C₂-C₈-alkenyl, C₂-C₈-haloalkenyl, C₂-C₈-alkenyloxy, C₂-C₈-alkynyloxy, C₁-C₆-alkoxy, C₁-C₆-haloalkoxy, C₁-C₆-alkylthio, C₁-C₆-alkylamino, di-C₁-C₆-alkylamino, formyl, C₁-C₆-alkylcarbonyl, C₁-C₆-alkylsulfonyl, C₁-C₆-alkylsulfoxyl, C₁-C₆-alkoxycarbonyl, C₁-C₆-alkylcarbonyloxy, C₁-C₆-alkylaminocarbonyl, di-C₁-C₆-alkylaminocarbonyl, C₁-C₆-alkylaminothiocarbonyl, di-C₁-C₆-alkylaminocarbonyl, C₃-C₁₀-cycloalkyl, C₃-C₁₀-cycloalkoxy, saturated or partially unsaturated 5- or 6-membered heterocyclyl, saturated or unsaturated 5- or 6-membered heterocyclyloxy, 5- or 6-membered heteroaryl, 5- or 6-membered heteroaryloxy, 5- or 6-membered heteroarylthio, 5- or 6-membered heteroarylthio, phenyl, naphthyl, phenoxy, naphthoxy, phenylthio, naphthylthio, phenyl-C₁-C₆-alkoxy, phenyl-C₁-C₆-alkyl, wherein the cyclic systems may be partially or fully halogenated and/or substituted by alkyl and/or haloalkyl groups;
R⁴ is halogen, C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₁-C₈-alkoxy, C₁-C₈-haloalkoxy, C₃-C₈-cycloalkyl, C₃-C₈-halocycloalkyl, C₃-C₈-cycloalkyl-C₁-C₈-alkyl, C₁-C₈-alkylthio, C₆-C₁₄-aryl, C₆-C₁₄-aryloxy, C₆-C₁₄-arylthio, C₆-C₁₄-aryl-C₁-C₈-alkyl, C₆-C₁₄-aryl-C₁-C₈-alkoxy, C₆-C₁₄-aryl-C₁-C₈-alkylthio, 5- or 6-membered heteroaryl, heteroaryloxy, heteroarylthio, heteroaryl-C₁-C₈-alkyl, heteroaryl-C₁-C₈-alkoxy or heteroaryl-C₁-C₈-alkylthio, morpholino, piperidino or pyrrolidino;
wherein R⁴ may carry one, two, three, four or five identical or different groups R^{c}, wherein R^{c} is as defined below:
R^{c} is halogen, cyano, isocyano, hydroxyl, cyanato (OCN), thiocyanato, isothiocyanato, mercapto, C₁-C₈-alkyl, C₁-C₈-haloalkyl, C₁-C₈-hydroxyalkyl, C₁-C₈-alkoxy-C₁-C₈-alkyl, C₃-C₆-cycloalkyl-C₁-C₈-alkyl, C₂-C₁₀-alkenyl, C₂-C₁₀-haloalkenyl, C₂-C₁₀-alkynyl, C₂-C₁₀-haloalkynyl, C₁-C₆-alkoxy, C₁-C₆-haloalkoxy, C₂-C₁₀-alkenyloxy, C₂-C₁₀-haloalkenyloxy, C₂-C₁₀-alkynyloxy, C₂-C₁₀-haloalkynyloxy, C₃-C₆-cycloalkyl, C₃-C₆-halocycloalkyl, C₃-C₆-cycloalkenyl, C₃-C₆-halocycloalkenyl, C₃-C₆-cycloalkoxy, C₃-C₆-halocycloalkoxy, C₁-C₈-alkoxycarbonyl, C₁-C₈-haloalkoxycarbonyl, C₂-C₁₀-alkenyloxycarbonyl, C₂-C₁₀-haloalkenyloxycarbonyl, C₂-C₁₀-alkynyloxycarbonyl, C₂-C₁₀-haloalkynyloxycarbonyl, aminocarbonyl, C₁-C₈-alkylaminocarbonyl, di-C₁-C₈-alkylaminocarbonyl, hydroxyimino-C₁-C₈-alkyl, C₁-C₈-alkoximino-C₁-C₈-alkyl, C₂-C₁₀-alkenyloximinocarbonyl, C₂-C₁₀-alkenyloximino-C₁-C₈-alkyl, C₂-C₁₀-alkynyloximino-C₁-C₈-alkyl, C₂-C₁₀-alkenylcarbonyl, C₂-C₁₀-alkenylthiocarbonyl, C₂-C₁₀-alkynylcarbonyl, C₂-C₁₀-alkynylthiocarbonyl, tri-C₁-C₈-alkylsilyl, C₁-C₈-alkylthio, C₁-C₈-haloalkylthio, C₁-C₈-alkylsulfinyl, C₁-C₈-haloalkylsulfinyl, carboxyl-C₁-C₈-alkyl, phenyl, naphthyl, a five-, six-, seven-, eight-, nine- or ten-membered saturated, partially unsaturated or aromatic heterocycle which contains one, two, three or four heteroatoms from the group consisting of oxygen, nitrogen and sulfur, NR^{A}R^{B}, NHCOR^{A}, NHCONR^{A}R^{B}, CONR^{A}R^{B}, —OSO₂R^{A}, —COR^{A}, —CSR^{A}, —CR^{A}═NR^{B} or N═CR^{A}R^{B}, wherein R^{A} and R^{B} are as defined below:
R^{A}, R^{B} independently of one another are hydrogen, C₁-C₈-alkyl, C₁-C₈-haloalkyl, C₁-C₈-alkoxy, C₁-C₈-haloalkoxy, C₁-C₈-alkylthio, C₃-C₈-cycloalkyl, C₃-C₈-halocycloalkyl, C₃-C₈-cycloalkyl-C₁-C₈-alkyl, phenyl or benzyl, where the phenyl and benzyl groups may be substituted by one, two, three, four or five groups independently selected from the group consisting of halogen, C₁-C₈-alkyl and C₁-C₈-alkoxy; phenoxy, benzyloxy, benzoyloxy, a five-, six-, seven-, eight-, nine- or ten-membered saturated, partially unsaturated or aromatic heterocycle which contains one, two, three or four heteroatoms from the group consisting of oxygen, nitrogen and sulfur;
wherein R^{c} may carry one, two or three identical or different groups R^{cL}, wherein R^{cL} is as defined below:
R^{cL} is halogen, cyano, nitro, hydroxyl, amino, carboxyl, C₁-C₆-alkyl, C₁-C₆-haloalkyl, C₂-C₆-alkenyl, C₂-C₆-haloalkenyl, C₁-C₈-alkoxy, C₁-C₈-haloalkoxy, C₂-C₈-alkenyloxy, C₂-C₈-alkynyloxy, C₃-C₈-cycloalkyl, C₃-C₈-halocycloalkyl, C₃-C₈-cycloalkenyl, C₃-C₈-cycloalkyloxy, C₃-C₈-cycloalkenyloxy, C₁-C₈-alkoximino, C₂-C₈-alkenyloxyimino, C₂-C₈-alkynyloxyimino, C₁-C₈-alkoximino -C₁-C₈-alkyl, C₂-C₈-alkenyloximino-C₁-C₈-alkyl, C₂-C₈-alkynyloximino-C₁-C₈-alkyl, C₁-C₆-alkylcarbonyl, C₁-C₆-haloalkoxy, C₁-C₆-alkoxycarbonyl, C₁-C₆-alkylthio, C₁-C₆-alkylamino, di-C₁-C₆-alkylamino, C₆-C₁₄-aryl-C₁-C₈-alkyl -oximino, C₂-C₁₀-alkynyl, C₂-C₁₀-alkenyloxycarbonyl, C₂-C₁₀-alkynyloxycarbonyl, tri-C₁-C₆-alkylsilyl, phenyl, naphthyl, a five-, six-, seven-, eight-, nine- or ten-membered saturated, partially unsaturated or aromatic heterocycle which contains one, two, three or four heteroatoms from the group consisting of oxygen, nitrogen and sulfur;
R⁵ is halogen, cyano, C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₁-C₈-alkoxy, C₁-C₈-haloalkoxy;
wherein R⁵ may carry one, two or three identical or different groups independently selected from the group consisting of cyano, nitro, C₁-C₂-alkoxy and C₁-C₄-alkoxycarbonyl;
A, B, D, E are each N or C—R⁶, wherein at least one and at most three variables are N; provided that when A, B and D are N, E is other than C—R⁶ and B, D and E are N, E is other than C—R⁶; wherein R⁶ is as defined below:
R⁶ is hydrogen, halogen, C₁-C₈-alkyl, C₁-C₈-alkoxy, C₁-C₈-haloalkyl, C₁-C₈-haloalkoxy;
or an agriculturally acceptable salt thereof.

50. The fungicidal composition of claim 48, wherein in component 1) R¹ is NR²R³.

51. The fungicidal composition of claim 48, wherein in component 1) R⁵ is selected from the group consisting of halogen, cyano, C₁-C₄-alkyl and C₁-C₄-alkoxy.

52. The fungicidal composition of claim 48, wherein component 1) corresponds to a compound of the formula Ia.1:

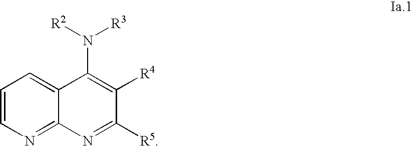

53. The fungicidal composition of claim 48, wherein component 1) corresponds to a compound of the formula Ib.1:

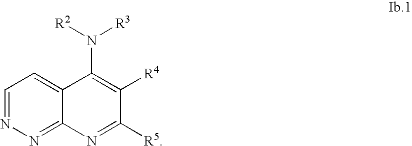

54. The fungicidal composition of claim 48, wherein component 1) corresponds to a compound of the formula Ic.1:

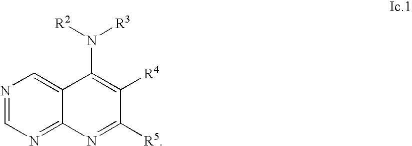

55. The fungicidal composition of claim 48, wherein component 1) corresponds to a compound of the formula Id.1:

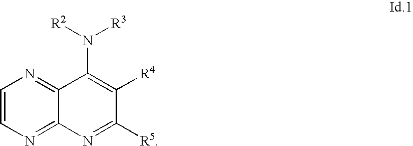

56. The fungicidal composition of claim 48, wherein component 1) corresponds to a compound of the formula Ie.1:

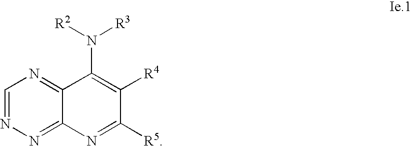

57. The fungicidal composition of claim 48, wherein component 1) corresponds to a compound of the formula If:

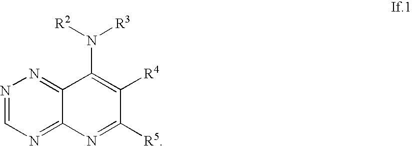

58. The fungicidal composition of claim 48, wherein in component 1) R² and R³ are as defined below:
R² is C₁-C₈-alkyl, C₁-C₈-haloalkyl, C₂-C₈-alkenyl, C₂-C₈-haloalkenyl, C₃-C₈-cycloalkyl, C₃-C₈-halocycloalkyl or C₃-C₈-cycloalkyl-C₁-C₈-alkyl,
R³ is hydrogen or one of the groups mentioned for R²;
wherein said alkyl, alkenyl and cycloalkyl groups for their part may carry one, two or three identical or different groups R^{d}.

59. The fungicidal composition of claim 48, wherein in component 1) R³ is not hydrogen.

60. The fungicidal composition of claim 48, wherein in component 1) R² and R³ together with the nitrogen atom to which they are attached form a five- or six-membered saturated or partially unsaturated heterocycle which may contain one or two further heteroatoms selected from the group consisting of O, N and S as ring members, wherein the heterocycle may carry one, two or three identical or different groups R^{f}.

61. The fungicidal composition of claim 48, wherein in component 1) R⁴ is phenyl which may contain one, two, three, four or five groups R^{c} independently selected from the group consisting of halogen, hydroxyl, cyano, isocyano, thiocyanato, isothiocyanato, nitro, mercapto, C₁-C₄-haloalkyl, C₁-C₄-alkoxy, C₁-C₄-haloalkoxy, C₃-C₆-cycloalkyl, C₃-C₆-halocycloalkyl and C₃-C₆-cycloalkyl-C₁-C₄-alkyl.

62. The fungicidal composition of claim 48, wherein in component 1) R⁴ is 5- or 6-membered heteroaryl which may contain one, two, three or four groups R^{c} independently selected from the group consisting of halogen, hydroxyl, cyano, isocyano, thiocyanato, isothiocyanato, nitro, mercapto, C₁-C₄-alkyl, C₁-C₄-haloalkyl, C₁-C₄-alkoxy, C₁-C₄-haloalkoxy, C₃-C₆-cycloalkyl, C₃-C₆-halocycloalkyl and C₃-C₆-cycloalkyl-C₁-C₄-alkyl.

63. The fungicidal composition of claim 48, wherein component 2) is selected from the group consisting of azoxystrobin, dimoxystrobin, fluoxastrobin, orysastrobin, picoxystrobin, pyraclostrobin and trifloxystrobin.

64. The fungicidal composition of claim 48, wherein component 2) is selected from the group consisting of cyproconazole, difenoconazole, epoxiconazole, prochloraz, propiconazole, prothioconazole and tebuconazole.

65. The fungicidal composition of claim 48, wherein component 2) is selected from the group consisting of chlorothalonil, fosetyl-aluminum, metrafenone and spiroxamine.

66. The fungicidal composition of claim 48, wherein component 2) is selected from the group consisting of boscalid, dimethomorph, fluopicolide, mandipropamid, metalaxyl, N-(2-bicyclopropyl-2-ylphenyl)-3-difluoromethyl-1-methyl-1H-pyrazole-4-carboxamide, N-(3′,4′-dichloro-4-fluorobiphenyl-2-yl)-3-difluoromethyl-1-methylpyrazole-4-carboxamide and N-(3′,4′-dichloro-5-fluoro-biphenyl-2-yl)-3-difluoromethyl-1-methylpyrazole-4-carboxamide.

67. The fungicidal composition of claim 48, wherein component 2) is selected from the group consisting of cyprodinil, fludioxonil, iprovalicarb and 5-chloro-7-(4-methylpiperidin-1-yl)-6-(2,4,6-trifluorophenyl)-[1,2,4]triazolo[1,5-a]pyrimidine.

68. The fungicidal composition of claim 48 comprising components 1) and 2) in a weight ratio of from 100:1 to 1:100.

69. The fungicidal composition of claim 48, further comprising at least one liquid or solid carrier.

70. A composition comprising at least one liquid or solid carrier and at least one compound of claim 41.

71. A method for controlling phytopathogenic harmful fungi, wherein said fungi, their habitat or the plants, the soil, seeds, areas, materials or spaces to be protected from fungal infection are treated with an effective amount of at least one compound I and at least one compound II of claim 48.

72. A method for controlling phytopathogenic harmful fungi, wherein said fungi, their habitat or the plants, the soil, seeds, areas, materials or spaces to be protected from fungal infection are treated with an effective amount of at least one compound I of claim 41.

73. The method of claim 71 wherein components 1) and 2) are applied jointly simultaneously, separately simultaneously or in succession.

74. The method of claim 71, wherein components 1) and 2) are applied in an amount of from 5 g/ha to 2000 g/ha.

75. The method of claim 71, wherein components 1) and 2) are applied in an amount of from 1 g to 1000 g per 100 kg of seed.

76. Seed comprising at least one composition of claim 48, wherein in an amount of from 1 g to 1000 g per 100 kg of seed.

77. Seed comprising at least one compound I of claim 41, wherein in an amount of from 1 g to 1000 g per 100 kg of seed.

78. A pharmaceutical composition comprising at least one compound of formula I of claim 41 and/or a pharmaceutically acceptable salt thereof.

79. A method for the treatment of cancer comprising administering to a mammal a compound of claim 41.
